SEQUENCE LISTING (57 sequences)

SEQ ID NO: 1 - DNA, 2949 bp, Artificial Sequence
  Description of Artificial Sequence Synthetic - M.tb antigen-PhaC fusion polynucleotide
  CDS (1)..(870): Ag85A antigen
  CDS (871)..(1176): ESAT6 antigen
  CDS (1177)..(1182): Linker
  CDS (1183)..(2946): PhaC synthase
atgttttcccggccgggcttgccggtggagtacctgcaggtgccgtcgccgtcgatgggccgtgacatcaaggtccaattccaaagtggtggtgccaactcgcccgccctgtacctgctcgacggcctgcgcgcgcaggacgacttcagcggctgggacatcaacaccccggcgttcgagtggtacgaccagtcgggcctgtcggtggtcatgccggtgggtggccagtcaagcttctactccgactggtaccagcccgcctgcggcaaggccggttgccagacttacaagtgggagaccttcctgaccagcgagctgccggggtggctgcaggccaacaggcacgtcaagcccaccggaagcgccgtcgtcggtctttcgatggctgcttcttcggcgctgacgctggcgatctatcacccccagcagttcgtctacgcgggagcgatgtcgggcctgttggacccctcccaggcgatgggtcccaccctgatcggcctggcgatgggtgacgctggcggctacaaggcctccgacatgtggggcccgaaggaggacccggcgtggcagcgcaacgacccgctgttgaacgtcgggaagctgatcgccaacaacacccgcgtctgggtgtactgcggcaacggcaagccgtcggatctgggtggcaacaacctgccggccaagttcctcgagggcttcgtgcggaccagcaacatcaagttccaagacgcctacaacgccggtggcggccacaacggcgtgttcgacttcccggacagcggtacgcacagctgggagtactggggggcgcagctcaacgctatgaagcccgacctgcaacgggcactgggtgccacgcccaacaccgggcccgcgccccagggcgccggatccacagagcagcagtggaatttcgcgggtatcgaggccgcggcaagcgcaatccagggtaatgtcacctccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcaactagtgcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcatga

SEQ ID NO: 2 - protein, 982 aa, Artificial Sequence
  Description of Artificial Sequence Synthetic construct polypeptide
MFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGAGSTEQQWNFAGIEAAASAIQGNVTSIHSLLDEGKQSLTKLAAAWGGSGSEAYQGVQQKWDATATELNNALQNLARTISEAGQAMASTEGNVTGMFATSATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 3 - DNA, 2958 bp, Artificial Sequence
  Description of Artificial Sequence Synthetic polynucleotide
  promoter (1)..(9): 3′OH terminus of nisA promoter
  CDS (10)..(1185): Ag85A-ESAT6 antigen fusion
  CDS (1186)..(2955): PhaC synthase
tgtacacatatgtttagtagaccaggtttacctgttgaatatttacaagttccttctccatctatgggtagagatattaaagttcaatttcaatcaggaggtgcaaattctccagctttatatttattagatggtttacgtgctcaagatgatttttctggttgggatattaatactccagcatttgaatggtatgatcaatcaggtttatctgttgttatgccagttggtggacaatcatctttttattcagattggtatcaacctgcttgtggaaaagcaggttgtcaaacatataaatgggaaacatttttaacatcagaattaccaggatggttacaagcaaatcgtcatgttaaaccaactggttcagctgttgttggtttatctatggctgcttcatcagctttaacattagctatttatcatccacaacaatttgtttatgcaggtgctatgtcaggtttattagatccatcacaagctatgggacctactttaattggtttagcaatgggtgatgctggaggatataaagctagtgatatgtggggaccaaaagaagatccagcatggcaacgtaatgatccattattaaatgttggaaaattaattgcaaataatactcgtgtttgggtttattgtggaaatggtaaaccatctgatttaggaggtaataatttaccagcaaaatttttagaaggatttgttcgtacatcaaatattaaatttcaagatgcttataatgctggtggaggacataatggtgtttttgattttccagattctggtacacattcatgggaatattggggtgcacaattaaatgctatgaaaccagatttacaacgtgctttaggtgctactcctaatacaggtccagctcctcaaggtgcaggatcaacagaacaacaatggaattttgctggaattgaagctgcagcttctgctattcaaggtaatgttacaagtattcattcattattagatgaaggaaaacaatcattaacaaaattagctgcagcttggggtggtagtggttcagaagcttatcaaggtgttcaacaaaaatgggatgcaactgctactgaattaaataatgctttacaaaatttagctcgtactatttcagaagctggtcaagctatggcttcaactgaaggtaatgttacaggtatgtttgcaactagtgcaacaggaaaaggtgccgcagcttcaacgcaagaaggaaaatcacaaccatttaaagttaccccaggcccatttgatccagcaacatggttggaatggtcaagacaatggcaaggaactgaaggaaatggacatgctgctgctagcggtattcctggtttagatgcacttgctggagtcaaaattgctccagctcaattaggtgatattcaacaacgatatatgaaagatttttcagctttgtggcaagcaatggccgaaggaaaagctgaagctacaggaccacttcatgatcgacgttttgcaggagatgcctggcgtacaaatttgccttacagatttgcagctgctttttatttattaaatgctcgtgctttaacagaattggcagatgctgtggaagctgatgctaaaactcgtcaacgtattagatttgcaattagtcaatgggttgatgctatgagtcctgcaaatttcttggcaaccaatcctgaagcacaacgattacttatcgaatcaggcggtgaatcacttcgtgctggtgttagaaatatgatggaagatttaactcgaggtaaaattagtcaaaccgatgaatcagcatttgaagtgggtcgaaatgtagctgttacggaaggtgctgttgttttcgaaaatgaatattttcaattgttacaatataaacctttaacagataaagttcatgcccgtcctttgcttatggttcctccttgtattaataaatattacattttggatcttcaaccagaaagctcacttgttcgacatgttgtcgaacaaggtcataccgtctttttggttagttggcgaaatcctgacgctagtatggcaggtagtacgtgggatgattatattgaacacgctgccattcgagcaattgaagttgcacgagatatttctggtcaagacaaaattaatgtacttggcttttgtgttggtggtacaattgtttctacggcattagctgtccttgctgctcgaggagaacatcctgccgcttctgtcacattgttgacaacattattagattttgctgatactggcattcttgatgtgtttgtcgatgaaggtcacgtacaattaagagaagcaaccttaggtggaggagctggcgctccatgtgctttgttaagaggtttggaacttgctaatacatttagctttcttcgaccaaatgatttggtctggaactacgtggttgacaattatttaaaaggtaatacgccagttcctttcgatttattgttctggaacggtgatgcaactaatttaccaggaccttggtactgttggtatttaagacacacatatttacaaaatgaacttaaagtcccaggaaaattaacagtctgtggtgttcctgtagatttagcatcaatcgacgtacctacttatatttatggtagtcgtgaagatcatattgtgccttggacagcagcatatgcttcaacagcacttttggccaataaattacgtttcgttcttggagctagtggacacattgctggagttattaatcctccagctaaaaataaacgttctcattggacaaatgatgctttgccagaaagtcctcaacaatggttagccggagcaatcgaacatcatggttcatggtggccagattggactgcatggttggctggtcaagccggtgcaaaacgtgcagcaccagccaattatggcaatgctcgatatagagctattgaacctgcaccaggccgttatgtcaaagcaaaagcatga

SEQ ID NO: 4 - protein, 982 aa, Artificial Sequence; identical to SEQ ID NO: 2
  Description of Artificial Sequence Synthetic construct polypeptide

SEQ ID NO: 5 - DNA, 48 bp, Artificial Sequence
  Description of Artificial Sequence Synthetic primer
gctactagtaataaggagatatacatatgttttcccggccgggcttgc

SEQ ID NO: 6 - DNA, 30 bp, Artificial Sequence
  Description of Artificial Sequence Synthetic primer
tgcactagttgcgaacatcccagtgacgtt

SEQ ID NO: 7 - DNA, 2361 bp, Artificial Sequence
  Description of Artificial Sequence Synthetic polynucleotide construct from pET-14b-HepC-PhaC
atgtctactaatcctaaacctcagcgtaaaaccaagcgtagcaccaaccgtcgtccgcaggacgttaagtttccgggtggtggccagattgtgggtggcgtctacctgctgccgcgtcgtggtccgcgtttgggtgttcgcgcgacccgcaaaacgagcgaacgctcccaaccgcgtggccgtcgtcaaccgatcccaaaagcgcgccaaccggaaggtcgcgcatgggcccagccaggctacccatggccgctgtatggcaacgagggtatgggctgggctggttggctgttgagcccgcgtggtagccgtccgagctggggtccgaccgacccgcgtcgccgctctcgtaatctgggtaaggtgattgataccttgacgtgcggtttcgcggatctgatgggttacatcccgctggtgggcgcaccgctgggcggtgcagcccgtgcactggcgcacggcgtccgcgttctggaggacggtgtcaactatgcgacgggcaatctgccgggttgtagcttttcgatcttcctgctggccctgctgagctgcctgaccattccggcgagcgcacgtacgggtggcggtggcggtgcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcatga

SEQ ID NO: 8 - protein, 786 aa, Artificial Sequence
  Description of Artificial Sequence Synthetic Hep C-PhaC fusion polypeptide encoded by pET-14b-HepC-PhaC
MSTNPKPQRKTKRSTNRRPQDVKFPGGGQIVGGVYLLPRRGPRLGVRATRKTSERSQPRGRRQPIPKARQPEGRAWAQPGYPWPLYGNEGMGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASARTGGGGGATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 9 - DNA, 2361 bp, Artificial Sequence
  Description of Artificial Sequence Synthetic construct pNZ-HepC-PhaCAB
atggcaagtacaaatccaaaacctcaaagaaaaacaaaacgttcaactaatcgtagaccacaagatgttaaatttcctggtggaggtcaaattgttggaggtgtttatttacttccacgtagaggacctagattaggtgttagagctacacgtaaaacatcagaaagatcacaaccacgtggtcgtagacaaccaattcctaaagcaagacaaccagaaggtcgtgcttgggcacaacctggatatccttggcctttatatggtaatgaaggaatgggttgggctggatggttactttctccaagaggatctcgtcctagttggggtccaactgatcctcgtagacgttcacgtaatcttggtaaagttattgatacacttacttgtggatttgctgatcttatgggttatattccattagttggagcacctcttggaggtgctgcaagagctcttgcacatggagttcgtgttttagaagatggtgttaattatgctacaggaaatcttccaggttgttcattttctatttttcttttagctttactttcatgtttaactattcctgcttctgcaactagtggaggtggaggtggagcaacaggaaaaggtgccgcagcttcaacgcaagaaggaaaatcacaaccatttaaagttaccccaggcccatttgatccagcaacatggttggaatggtcaagacaatggcaaggaactgaaggaaatggacatgctgctgctagcggtattcctggtttagatgcacttgctggagtcaaaattgctccagctcaattaggtgatattcaacaacgatatatgaaagatttttcagctttgtggcaagcaatggccgaaggaaaagctgaagctacaggaccacttcatgatcgacgttttgcaggagatgcctggcgtacaaatttgccttacagatttgcagctgctttttatttattaaatgctcgtgctttaacagaattggcagatgctgtggaagctgatgctaaaactcgtcaacgtattagatttgcaattagtcaatgggttgatgctatgagtcctgcaaatttcttggcaaccaatcctgaagcacaacgattacttatcgaatcaggcggtgaatcacttcgtgctggtgttagaaatatgatggaagatttaactcgaggtaaaattagtcaaaccgatgaatcagcatttgaagtgggtcgaaatgtagctgttacggaaggtgctgttgttttcgaaaatgaatattttcaattgttacaatataaacctttaacagataaagttcatgcccgtcctttgcttatggttcctccttgtattaataaatattacattttggatcttcaaccagaaagctcacttgttcgacatgttgtcgaacaaggtcataccgtctttttggttagttggcgaaatcctgacgctagtatggcaggtagtacgtgggatgattatattgaacacgctgccattcgagcaattgaagttgcacgagatatttctggtcaagacaaaattaatgtacttggcttttgtgttggtggtacaattgtttctacggcattagctgtccttgctgctcgaggagaacatcctgccgcttctgtcacattgttgacaacattattagattttgctgatactggcattcttgatgtgtttgtcgatgaaggtcacgtacaattaagagaagcaaccttaggtggaggagctggcgctccatgtgctttgttaagaggtttggaacttgctaatacatttagctttcttcgaccaaatgatttggtctggaactacgtggttgacaattatttaaaaggtaatacgccagttcctttcgatttattgttctggaacggtgatgcaactaatttaccaggaccttggtactgttggtatttaagacacacatatttacaaaatgaacttaaagtcccaggaaaattaacagtctgtggtgttcctgtagatttagcatcaatcgacgtacctacttatatttatggtagtcgtgaagatcatattgtgccttggacagcagcatatgcttcaacagcacttttggccaataaattacgtttcgttcttggagctagtggacacattgctggagttattaatcctccagctaaaaataaacgttctcattggacaaatgatgctttgccagaaagtcctcaacaatggttagccggagcaatcgaacatcatggttcatggtggccagattggactgcatggttggctggtcaagccggtgcaaaacgtgcagcaccagccaattatggcaatgctcgatatagagctattgaacctgcaccaggccgttatgtcaaagcaaaagca

SEQ ID NO: 10 - protein, 787 aa, Artificial Sequence
  Description of Artificial Sequence Synthetic HepC-PhaC fusion polypeptide encoded by pNZ-HepC-PhaCAB
MASTNPKPQRKTKRSTNRRPQDVKFPGGGQIVGGVYLLPRRGPRLGVRATRKTSERSQPRGRRQPIPKARQPEGRAWAQPGYPWPLYGNEGMGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASATSGGGGGATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 11 - DNA, 2775 bp, Artificial Sequence
  Description of Artificial Sequence Synthetic construct - pET-14b-PhaC-HA1 of H3-PhaC
atgcagaaactgccgggtaacgataatagtaccgcaacgctgtgcctgggtcatcacgcagtgccgaacggtaccattgttaaaaccatcacgaacgatcagattgaagtgaccaatgcgacggaactggttcagagctctagtaccggtgaaatctgcgatagcccgcatcagattctggatggcgaaaattgtacgctgatcgatgccctgctgggcgatccgcagtgcgatggttttcagaacaaaaaatgggatctgttcgtggaacgctctaaagcatacagtaactgttacccgtatgatgtgccggattacgccagcctgcgttctctggttgcaagctctggcaccctggaatttaacaatgaaagcttcaactggaccggcgttacgcagaatggtacgagtagcgcgtgcattcgtcgcagtaacaatagctttttctctcgtctgaactggctgacccacctgaaatttaaatatccggccctgaatgtgacgatgccgaacaacgaaaaattcgataaactgtacatctggggcgttcatcacccgggtaccgataacgatcagatttttccgtatgcccaggcaagcggtcgcatcaccgtgagcacgaaacgttctcagcagaccgttattccgaacatcggctctcgtccgcgcgtgcgtaatattccgagtcgcattagcatctactggaccatcgttaaaccgggcgatattctgctgatcaacagcacgggcaatctgattgcaccgcgcggttattttaaaatccgttctggcaaatctagtattatgcgtagtgatgcgccgatcggtaaatgcaatagcgaatgtatcaccccgaacggctctattccgaatgataaaccgttccagaacgtgaatcgcattacgtatggtgcctgcccgcgttacgttaaacagaacaccctgaaactggcaacgggcatgcgcaatgtgccggaaaaacagacccgtacgggtggcggtggcggtgcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcatga

SEQ ID NO: 12 - protein, 924 aa, Artificial Sequence
  Description of Artificial Sequence Synthetic HA1 of H3-PhaC fusion polypeptide encoded by pET-14b-HA1 of H3-PhaC
MQKLPGNDNSTATLCLGHHAVPNGTIVKTITNDQIEVTNATELVQSSSTGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTHLKFKYPALNVTMPNNEKFDKLYIWGVHHPGTDNDQIFPYAQASGRITVSTKRSQQTVIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRTGGGGGATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 13 - DNA, 43 bp, Artificial Sequence
  Description of Artificial Sequence Synthetic primer
agatactagtatgcagaaactgccgggtaacgataatagtacc

SEQ ID NO: 14 - DNA, 40 bp, Artificial Sequence
  Description of Artificial Sequence Synthetic primer
gatgcgtacgggtctgtttttccggcacattgcgcatgcc

SEQ ID NO: 15 - DNA, 40 bp, Artificial Sequence
  Description of Artificial Sequence Synthetic primer
agatctcgagcagaaactgccgggtaacgataatagtacc

SEQ ID NO: 16 - DNA, 43 bp, Artificial Sequence
  Description of Artificial Sequence Synthetic primer
gatgggatcctcaggtctgtttttccggcacattgcgcatgcc

SEQ ID NO: 17 - DNA, 3195 bp, Artificial Sequence
  Description of Artificial Sequence Synthetic construct - pET-14b-NA-PhaC
atgaatccaaatcaaaagataataacgattggctctgtttctctcaccatttccacaatatgcttcttcatgcaaattgccatcttgataactactgtaacattgcatttcaagcaatatgaattcaactcccccccaaacaaccaagtgatgctgtgtgaaccaacaataatagaaagaaacataacagagatagtgtatctgaccaacaccaccatagagaaggaaatatgccccaaactagcagaatacagaaattggtcaaagccgcaatgtgacattacaggatttgcacctttttctaaggacaattcgattaggctttccgctggtggggacatctgggtgacaagagaaccttatgtgtcatgcgatcctgacaagtgttatcaatttgcccttggacagggaacaacactaaacaacgtgcattcaaatgacacagtacgtgataggaccccttatcggaccctattgatgaatgagttaggtgttccttttcatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtattgtttcatggtccaaagaaatcctcaggacccaggagtcagaatgcgtttgtatcaatggaacttgtacagtagtaatgactgatgggagtgcttcaggaaaagctgatactaaaatactattcattgaggaggggaaaatcgttcatactagcacattgtcaggaagtgctcagcatgtcgaggagtgctcctgctatcctcgatatcctggtgtcagatgtgtctgcagagacaactggaaaggctccaataggcccatcgtagatataaacataaaggatcatagcactgtttccagttatgtgtgttcaggacttgttggagacacacccagaaaaaacgacagctccagcagtagccattgtttagatcctaacaatgaagaaggtggtcatggagtgaaaggctgggcctttgatgatggaaatgacgtgtggatgggaagaacgatcagcgagaagtcgcgcttagggtatgaaaccttcaaagtcattgaaggctggtccaaccctaagtccaaattgcagataaataggcaagtcatagttgacagaggtaataggtccggttattctggtattttctctgttgaaggcaaaagctgcatcaatcggtgcttttatgtggagttgataaggggaagaaaagaggaaactgaagtcttgtggacctcaaacagtattgttgtgttttgtggcacctcaggtacatatggaacaggctcatggcctgatggggcggacatcaatctcatgcctatacgtacgggtggcggtggcggtgcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcatga

SEQ ID NO: 18 - protein, 1064 aa, Artificial Sequence
  Description of Artificial Sequence Synthetic NA-PhaC fusion polypeptide encoded by pET-14b-NA-PhaC
MNPNQKIITIGSVSLTISTICFFMQIAILITTVTLHFKQYEFNSPPNNQVMLCEPTIIERNITEIVYLTNTTIEKEICPKLAEYRNWSKPQCDITGFAPFSKDNSIRLSAGGDIWVTREPYVSCDPDKCYQFALGQGTTLNNVHSNDTVRDRTPYRTLLMNELGVPFHLGTKQVCIAWSSSSCHDGKAWLHVCITGDDKNATASFIYNGRLVDSIVSWSKEILRTQESECVCINGTCTVVMTDGSASGKADTKILFIEEGKIVHTSTLSGSAQHVEECSCYPRYPGVRCVCRDNWKGSNRPIVDINIKDHSTVSSYVCSGLVGDTPRKNDSSSSSHCLDPNNEEGGHGVKGWAFDDGNDVWMGRTISEKSRLGYETFKVIEGWSNPKSKLQINRQVIVDRGNRSGYSGIFSVEGKSCINRCFYVELIRGRKEETEVLWTSNSIVVFCGTSGTYGTGSWPDGADINLMPIRTGGGGGATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 19 - DNA, 3228 bp, Artificial Sequence
  Description of Artificial Sequence Synthetic construct - pET-14b-PhaC-linker-NA
atggcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcacatatggtgctggcggtggcgattgataaacgcggaggcggtggaggcctcgagaatccaaatcaaaagataataacgattggctctgtttctctcaccatttccacaatatgcttcttcatgcaaattgccatcttgataactactgtaacattgcatttcaagcaatatgaattcaactcccccccaaacaaccaagtgatgctgtgtgaaccaacaataatagaaagaaacataacagagatagtgtatctgaccaacaccaccatagagaaggaaatatgccccaaactagcagaatacagaaattggtcaaagccgcaatgtgacattacaggatttgcacctttttctaaggacaattcgattaggctttccgctggtggggacatctgggtgacaagagaaccttatgtgtcatgcgatcctgacaagtgttatcaatttgcccttggacagggaacaacactaaacaacgtgcattcaaatgacacagtacgtgataggaccccttatcggaccctattgatgaatgagttaggtgttccttttcatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtattgtttcatggtccaaagaaatcctcaggacccaggagtcagaatgcgtttgtatcaatggaacttgtacagtagtaatgactgatgggagtgcttcaggaaaagctgatactaaaatactattcattgaggaggggaaaatcgttcatactagcacattgtcaggaagtgctcagcatgtcgaggagtgctcctgctatcctcgatatcctggtgtcagatgtgtctgcagagacaactggaaaggctccaataggcccatcgtagatataaacataaaggatcatagcactgtttccagttatgtgtgttcaggacttgttggagacacacccagaaaaaacgacagctccagcagtagccattgtttggatcctaacaatgaagaaggtggtcatggagtgaaaggctgggcctttgatgatggaaatgacgtgtggatgggaagaacgatcagcgagaagtcgcgcttagggtatgaaaccttcaaagtcattgaaggctggtccaaccctaagtccaaattgcagataaataggcaagtcatagttgacagaggtaataggtccggttattctggtattttctctgttgaaggcaaaagctgcatcaatcggtgcttttatgtggagttgataaggggaagaaaagaggaaactgaagtcttgtggacctcaaacagtattgttgtgttttgtggcacctcaggtacatatggaacaggctcatggcctgatggggcggacatcaatctcatgcctatataa

SEQ ID NO: 20 - protein, 1075 aa, Artificial Sequence
  Description of Artificial Sequence Synthetic PhaC-linker-NA fusion polypeptide encoded by pET-14b-PhaC-linker-NA
MATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKAHMVLAVAIDKRGGGGGLENPNQKIITIGSVSLTISTICFFMQIAILITTVTLHFKQYEFNSPPNNQVMLCEPTIIERNITEIVYLTNTTIEKEICPKLAEYRNWSKPQCDITGFAPFSKDNSIRLSAGGDIWVTREPYVSCDPDKCYQFALGQGTTLNNVHSNDTVRDRTPYRTLLMNELGVPFHLGTKQVCIAWSSSSCHDGKAWLHVCITGDDKNATASFIYNGRLVDSIVSWSKEILRTQESECVCINGTCTVVMTDGSASGKADTKILFIEEGKIVHTSTLSGSAQHVEECSCYPRYPGVRCVCRDNWKGSNRPIVDINIKDHSTVSSYVCSGLVGDTPRKNDSSSSSHCLDPNNEEGGHGVKGWAFDDGNDVWMGRTISEKSRLGYETFKVIEGWSNPKSKLQINRQVIVDRGNRSGYSGIFSVEGKSCINRCFYVELIRGRKEETEVLWTSNSIVVFCGTSGTYGTGSWPDGADINLMPI

SEQ ID NO: 21 - DNA, 2544 bp, Artificial Sequence
  Description of Artificial Sequence Synthetic construct - pET-14b-M1-PhaC
atgagccttctaaccgaggtcgaaacgtatgttctctctatcgttccatcaggccccctcaaagccgagatcgcgcagagacttgaagatgtctttgctgggaaaaacacagatcttgaggctctcatggaatggctaaagacaagaccaattctgtcacctctgactaaggggattttggggtttgtgttcacgctcaccgtgcccagtgagcgaggactgcagcgtagacgctttgtccaaaatgccctcaatgggaatggagatccaaataacatggacaaagcagttaaactgtataggaaacttaagagggagataacgttccatggggccaaagaaatagctctcagttattctgctggtgcacttgccagttgcatgggcctcatatacaatagaatgggggctgtaaccactgaagtggcatttggcctggtatgtgcaacatgtgagcaaattgctgactcccagcacaggtctcataggcaaatggtggcaacaaccaatccattaataaaacatgagaacagaatggttttggccagcactacagctaaggctatggagcaaatggctggatcaagtgagcaggcagcggaggccatggagattgctagtcaggccaggcagatggtgcaggcaatgagagccattgggactcatcctagttccagtactggtctaagagatgatcttcttgaaaatttgcagacctatcagaaacgaatgggggtgcagatgcaacgattcaagcgtacgggtggcggtggcggtgcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcatga

SEQ ID NO: 22 - protein, 847 aa, Artificial Sequence
  Description of Artificial Sequence Synthetic M1-PhaC fusion polypeptide encoded by pET-14b-M1-PhaC
MSLLTEVETYVLSIVPSGPLKAEIAQRLEDVFAGKNTDLEALMEWLKTRPILSPLTKGILGFVFTLTVPSERGLQRRRFVQNALNGNGDPNNMDKAVKLYRKLKREITFHGAKEIALSYSAGALASCMGLIYNRMGAVTTEVAFGLVCATCEQIADSQHRSHRQMVATTNPLIKHENRMVLASTTAKAMEQMAGSSEQAAEAMEIASQARQMVQAMRAIGTHPSSSTGLRDDLLENLQTYQKRMGVQMQRFKRTGGGGGATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 23 - DNA, 2577 bp, Artificial Sequence
  Description of Artificial Sequence Synthetic construct - pET-14b-PhaC-linker-M1
atggcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcacatatggtgctggcggtggcgattgataaacgcggaggcggtggaggcctcgagagccttctaaccgaggtcgaaacgtatgttctctctatcgttccatcaggccccctcaaagccgagatcgcgcagagacttgaagatgtctttgctgggaaaaacacagatcttgaggctctcatggaatggctaaagacaagaccaattctgtcacctctgactaaggggattttggggtttgtgttcacgctcaccgtgcccagtgagcgaggactgcagcgtagacgctttgtccaaaatgccctcaatgggaatggagatccaaataacatggacaaagcagttaaactgtataggaaacttaagagggagataacgttccatggggccaaagaaatagctctcagttattctgctggtgcacttgccagttgcatgggcctcatatacaatagaatgggggctgtaaccactgaagtggcatttggcctggtatgtgcaacatgtgagcaaattgctgactcccagcacaggtctcataggcaaatggtggcaacaaccaatccattaataaaacatgagaacagaatggttttggccagcactacagctaaggctatggagcaaatggctggatcaagtgagcaggcagcggaggccatggagattgctagtcaggccaggcagatggtgcaggcaatgagagccattgggactcatcctagttccagtactggtctaagagatgatcttcttgaaaatttgcagacctatcagaaacgaatgggggtgcagatgcaacgattcaagtga

SEQ ID NO: 24 - protein, 858 aa, Artificial Sequence
  Description of Artificial Sequence Synthetic PhaC-linker-M1 fusion polypeptide encoded by pET-14b-PhaC-linker-M1
MATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKAHMVLAVAIDKRGGGGGLESLLTEVETYVLSIVPSGPLKAEIAQRLEDVFAGKNTDLEALMEWLKTRPILSPLTKGILGFVFTLTVPSERGLQRRRFVQNALNGNGDPNNMDKAVKLYRKLKREITFHGAKEIALSYSAGALASCMGLIYNRMGAVTTEVAFGLVCATCEQIADSQHRSHRQMVATTNPLIKHENRMVLASTTAKAMEQMAGSSEQAAEAMEIASQARQMVQAMRAIGTHPSSSTGLRDDLLENLQTYQKRMGVQMQRFK

SEQ ID NO: 25 - DNA, 4005 bp, Artificial Sequence
  Description of Artificial Sequence Synthetic construct - pET14b-NA-PhaC-linker-M1
atgaatccaaatcaaaagataataacgattggctctgtttctctcaccatttccacaatatgcttcttcatgcaaattgccatcttgataactactgtaacattgcatttcaagcaatatgaattcaactcccccccaaacaaccaagtgatgctgtgtgaaccaacaataatagaaagaaacataacagagatagtgtatctgaccaacaccaccatagagaaggaaatatgccccaaactagcagaatacagaaattggtcaaagccgcaatgtgacattacaggatttgcacctttttctaaggacaattcgattaggctttccgctggtggggacatctgggtgacaagagaaccttatgtgtcatgcgatcctgacaagtgttatcaatttgcccttggacagggaacaacactaaacaacgtgcattcaaatgacacagtacgtgataggaccccttatcggaccctattgatgaatgagttaggtgttccttttcatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtattgtttcatggtccaaagaaatcctcaggacccaggagtcagaatgcgtttgtatcaatggaacttgtacagtagtaatgactgatgggagtgcttcaggaaaagctgatactaaaatactattcattgaggaggggaaaatcgttcatactagcacattgtcaggaagtgctcagcatgtcgaggagtgctcctgctatcctcgatatcctggtgtcagatgtgtctgcagagacaactggaaaggctccaataggcccatcgtagatataaacataaaggatcatagcactgtttccagttatgtgtgttcaggacttgttggagacacacccagaaaaaacgacagctccagcagtagccattgtttagatcctaacaatgaagaaggtggtcatggagtgaaaggctgggcctttgatgatggaaatgacgtgtggatgggaagaacgatcagcgagaagtcgcgcttagggtatgaaaccttcaaagtcattgaaggctggtccaaccctaagtccaaattgcagataaataggcaagtcatagttgacagaggtaataggtccggttattctggtattttctctgttgaaggcaaaagctgcatcaatcggtgcttttatgtggagttgataaggggaagaaaagaggaaactgaagtcttgtggacctcaaacagtattgttgtgttttgtggcacctcaggtacatatggaacaggctcatggcctgatggggcggacatcaatctcatgcctatacgtacgggtggcggtggcggtatggcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcacatatggtgctggcggtggcgattgataaacgcggaggcggtggaggcctcgagagccttctaaccgaggtcgaaacgtatgttctctctatcgttccatcaggccccctcaaagccgagatcgcgcagagacttgaagatgtctttgctgggaaaaacacagatcttgaggctctcatggaatggctaaagacaagaccaattctgtcacctctgactaaggggattttggggtttgtgttcacgctcaccgtgcccagtgagcgaggactgcagcgtagacgctttgtccaaaatgccctcaatgggaatggagatccaaataacatggacaaagcagttaaactgtataggaaacttaagagggagataacgttccatggggccaaagaaatagctctcagttattctgctggtgcacttgccagttgcatgggcctcatatacaatagaatgggggctgtaaccactgaagtggcatttggcctggtatgtgcaacatgtgagcaaattgctgactcccagcacaggtctcataggcaaatggtggcaacaaccaatccattaataaaacatgagaacagaatggttttggccagcactacagctaaggctatggagcaaatggctggatcaagtgagcaggcagcggaggccatggagattgctagtcaggccaggcagatggtgcaggcaatgagagccattgggactcatcctagttccagtactggtctaagagatgatcttcttgaaaatttgcagacctatcagaaacgaatgggggtgcagatgcaacgattcaagtga

SEQ ID NO: 26 - protein, 1334 aa, Artificial Sequence
  Description of Artificial Sequence Synthetic NA-PhaC-linker-M1 fusion polypeptide encoded by pET-14b-NA-PhaC-linker-M1
MNPNQKIITIGSVSLTISTICFFMQIAILITTVTLHFKQYEFNSPPNNQVMLCEPTIIERNITEIVYLTNTTIEKEICPKLAEYRNWSKPQCDITGFAPFSKDNSIRLSAGGDIWVTREPYVSCDPDKCYQFALGQGTTLNNVHSNDTVRDRTPYRTLLMNELGVPFHLGTKQVCIAWSSSSCHDGKAWLHVCITGDDKNATASFIYNGRLVDSIVSWSKEILRTQESECVCINGTCTVVMTDGSASGKADTKILFIEEGKIVHTSTLSGSAQHVEECSCYPRYPGVRCVCRDNWKGSNRPIVDINIKDHSTVSSYVCSGLVGDTPRKNDSSSSSHCLDPNNEEGGHGVKGWAFDDGNDVWMGRTISEKSRLGYETFKVIEGWSNPKSKLQINRQVIVDRGNRSGYSGIFSVEGKSCINRCFYVELIRGRKEETEVLWTSNSIVVFCGTSGTYGTGSWPDGADINLMPIRTGGGGGMATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKAHMVLAVAIDKRGGGGGLESLLTEVETYVLSIVPSGPLKAEIAQRLEDVFAGKNTDLEALMEWLKTRPILSPLTKGILGFVFTLTVPSERGLQRRRFVQNALNGNGDPNNMDKAVKLYRKLKREITFHGAKEIALSYSAGALASCMGLIYNRMGAVTTEVAFGLVCATCEQIADSQHRSHRQMVATTNPLIKHENRMVLASTTAKAMEQMAGSSEQAAEAMEIASQARQMVQAMRAIGTHPSSSTGLRDDLLENLQTYQKRMGVQMQRFK

SEQ ID NO: 27 - DNA, 3486 bp, Artificial Sequence
  Description of Artificial Sequence Synthetic construct - pET-14b-NA-PhaC-linker-M1/hemagglutinin-PhaC
atgaaaaccattatcgcgctgtcttatattctgtgtctggtgtttacgcagaaactgccgggtaacgataatagtaccgcaacgctgtgcctgggtcatcacgcagtgccgaacggtaccattgttaaaaccatcacgaacgatcagattgaagtgaccaatgcgacggaactggttcagagctctagtaccggtgaaatctgcgatagcccgcatcagattctggatggcgaaaattgtacgctgatcgatgccctgctgggcgatccgcagtgcgatggttttcagaacaaaaaatgggatctgttcgtggaacgctctaaagcatacagtaactgttacccgtatgatgtgccggattacgccagcctgcgttctctggttgcaagctctggcaccctggaatttaacaatgaaagcttcaactggaccggcgttacgcagaatggtacgagtagcgcgtgcattcgtcgcagtaacaatagctttttctctcgtctgaactggctgacccacctgaaatttaaatatccggccctgaatgtgacgatgccgaacaacgaaaaattcgataaactgtacatctggggcgttcatcacccgggtaccgataacgatcagatttttccgtatgcccaggcaagcggtcgcatcaccgtgagcacgaaacgttctcagcagaccgttattccgaacatcggctctcgtccgcgcgtgcgtaatattccgagtcgcattagcatctactggaccatcgttaaaccgggcgatattctgctgatcaacagcacgggcaatctgattgcaccgcgcggttattttaaaatccgttctggcaaatctagtattatgcgtagtgatgcgccgatcggtaaatgcaatagcgaatgtatcaccccgaacggctctattccgaatgataaaccgttccagaacgtgaatcgcattacgtatggtgcctgcccgcgttacgttaaacagaacaccctgaaactggcaacgggcatgcgcaatgtgccggaaaaacagacccgtggcatttttggtgcgatcgccggtttcattgaaaacggctgggagggtatggttgatggctggtatggttttcgccatcagaatagtgaaggcattggtcaggcggccgatctgaaaagcacccaggcagcgattgatcagatcaacggtaaactgaatcgcctgattggcaaaacgaacgaaaaattccaccagatcgaaaaagaatttagcgaagtggaaggccgtattcaggatctggaaaaatacgttgaagataccaaaatcgatctgtggagttacaacgcagaactgctggttgcgctggaaaatcagcataccattgatctgacggatagcgaaatgaacaaactgttcgaaaaaaccaaaaaacagctgcgcgaaaacgcggaagatatgggcaatggttgtttcaaaatctaccataaatgcgataacgcctgtattggctctatccgtaatggtacctatgatcacgatgtgtaccgcgatgaagcactgaacaatcgttttcagattaaaggcgttgaactgaaaagcggttataaagattggattctgtggatcagttttgccatcagctgcttcctgctgtgtgttgcactgctgggtttcattatgtgggcgtgccagaaaggcaacatccgttgcaatatttgtatccgtacgggtggcggtggcggtgcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcatga

SEQ ID NO: 28 - protein, 1161 aa, Artificial Sequence
  Description of Artificial Sequence Synthetic Hemagglutinin-PhaC fusion polypeptide encoded by pET-14b-NA-PhaC-linker-M1/hemagglutinin-PhaC
MKTIIALSYILCLVFTQKLPGNDNSTATLCLGHHAVPNGTIVKTITNDQIEVTNATELVQSSSTGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTHLKFKYPALNVTMPNNEKFDKLYIWGVHHPGTDNDQIFPYAQASGRITVSTKRSQQTVIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICIRTGGGGGATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 29 - DNA, 8587 bp, Artificial Sequence
  Description of Artificial Sequence Synthetic construct - pET14b-AcpA-PhaC-IgIC
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaatataggagataccagtatgaaactgaacaaaattaccctgggcattctgagcctgagcattgcgaccaccacctttgcgaccgatgtgaacaacagcaaaccgaacgattatggcaccctggtgaaaattaaacagaaactgtttaacaacgcgaacaccctgaaaaccaccaccccgattaaacatgtggtgattatttttcaggaaaacaacagctttgatcgctattttggcatgtatccgaacgcgaaaaacccggaaggcgaaccgaaatttgtggcgaaagaaaacaccccgaacgtgaacggcctgaccaaacagctgctggaaaacaacccgaacaccaaaaacccgtatcgcctggatcgcaactttcagccgtgcagccagaaccatgaatatcatcaggaaattagcagctttaacggcggcctgatgaacaaatttgtggaacatggcggccatgataacgatacctataaacagaactgcgatggccaggtgatgggctattatgatggcaacaccgtgaccgcgctgtggaactatgcgcagaactttgcgctgaacgataacacctttggcaccacctttggcccgagcaccccgggcgcgctgaacctggtggcgggcgcgaacggcccggcgatgagcccgagcggcaacctggaaaacattgaaaacagctatattattgatgatccgaacccgtattatgatgattgcagctatggcaccagcaaaagcggcgataccaacaccgcggtggcgaaaattaccgatggctataacattggccattatctgacccagaaaggcattacctggggctggtttcagggcggctttaaaccgaccagctatagcggcaaaaccgcgatttgcgatgcgatgagcaccaacaaatttggcattaaaagccgcgattatattccgcatcatgaaccgtttaactattggaaagaaaccagcaacccgcatcatctggcgccgagcgatgataaatatattggcagcaacgatcaggcgaaccatcagtatgatattagcgaattttggaaagcgctggatcagaacaccatgccggcggtgagctatctgaaagcgccgggctatcaggatggccatggcggctatagcaacccgctggatgaacaggaatggctggtgaacaccattaaccgcattaaacagagcaaagattgggatagcaccgcgattattattatttatgatgatagcgatggcgattatgatcatgtgtatagcccgaaaagccagtttagcgatattaaaggccgccagggctatggcccgcgcctgccgatgctggtgattagcccgtataccaaagcgaactatattgatcatagcctgctgaaccaggcgagcgtgctgaaatttattgaatataactggggcattggcagcgtgagcaaatatagcaacgataaatatagcaacaacattctgaacatgtttgattttaacaaaaaacagaaaaccccgaaactgattctggatccgaaaaccggcctggtggtggataaactgaacactagtgcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcacatatggtgctggcggtggcgattgataaacgcggaggcggtggaggcctcgagatgattatgagcgaaatgattacccgccagcaggtgaccagcggcgaaaccattcatgtgcgcaccgatccgaccgcgtgcattggcagccatccgaactgccgcctgtttattgatagcctgaccattgcgggcgaaaaactggataaaaacattgtggcgattgaaggcggcgaagatgtgaccaaagcggatagcgcgaccgcggcggcgagcgtgattcgcctgagcattaccccgggcagcattaacccgaccattagcattaccctgggcgtgctgattaaaagcaacgtgcgcaccaaaattgaagaaaaagtgagcagcattctgcaggcgagcgcgaccgatatgaaaattaaactgggcaacagcaacaaaaaacaggaatataaaaccgatgaagcgtggggcattatgattgatctgagcaacctggaactgtatccgattagcgcgaaagcgtttagcattagcattgaaccgaccgaactgatgggcgtgagcaaagatggcatgagctatcatattattagcattgatggcctgaccaccagccagggcagcctgccggtgtgctgcgcggcgagcaccgataaaggcgtggcgaaaattggctatattgcggcggcgtgaggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 30 - protein, 1331 aa, Artificial Sequence
  Description of Artificial Sequence Synthetic AcpA-PhaC-IgIC fusion polypeptide encoded by pET-14b-AcpA-PhaC-IgIC
MKLNKITLGILSLSIATTTFATDVNNSKPNDYGTLVKIKQKLFNNANTLKTTTPIKHVVIIFQENNSFDRYFGMYPNAKNPEGEPKFVAKENTPNVNGLTKQLLENNPNTKNPYRLDRNFQPCSQNHEYHQEISSFNGGLMNKFVEHGGHDNDTYKQNCDGQVMGYYDGNTVTALWNYAQNFALNDNTFGTTFGPSTPGALNLVAGANGPAMSPSGNLENIENSYIIDDPNPYYDDCSYGTSKSGDTNTAVAKITDGYNIGHYLTQKGITWGWFQGGFKPTSYSGKTAICDAMSTNKFGIKSRDYIPHHEPFNYWKETSNPHHLAPSDDKYIGSNDQANHQYDISEFWKALDQNTMPAVSYLKAPGYQDGHGGYSNPLDEQEWLVNTINRIKQSKDWDSTAIIIIYDDSDGDYDHVYSPKSQFSDIKGRQGYGPRLPMLVISPYTKANYIDHSLLNQASVLKFIEYNWGIGSVSKYSNDKYSNNILNMFDFNKKQKTPKLILDPKTGLVVDKLNATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKAHMVLAVAIDKRGGGGGLEMIMSEMITRQQVTSGETIHVRTDPTACIGSHPNCRLFIDSLTIAGEKLDKNIVAIEGGEDVTKADSATAAASVIRLSITPGSINPTISITLGVLIKSNVRTKIEEKVSSILQASATDMKIKLGNSNKKQEYKTDEAWGIMIDLSNLELYPISAKAFSISIEPTELMGVSKDGMSYHIISIDGLTTSQGSLPVCCAASTDKGVAKIGYIAAA

SEQ ID NO: 31 - DNA, 6844 bp, Artificial Sequence
  Description of Artificial Sequence Synthetic construct - pET-14b-PhaC-omp16
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataaggagatactagtgcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcacatatggtgctggcggtggcgattgataaacgcggaggcggtggaggcctcgagatgggctgcgcgagcaaaaaaaacctgccgaacaacgcgggcgatctgggcctgggcgcgggcgcggcgaccccgggcagcagccaggattttaccgtgaacgtgggcgatcgcattttttttgatctggatagcagcctgattcgcgcggatgcgcagcagaccctgagcaaacaggcgcagtggctgcagcgctatccgcagtatagcattaccattgaaggccatgcggatgaacgcggcacccgcgaatataacctggcgctgggccagcgccgcgcggcggcgacccgcgattttctggcgagccgcggcgtgccgaccaaccgcatgcgcaccattagctatggcaacgaacgcccggtggcggtgtgcgatgcggatacctgctggagccagaaccgccgcgcggtgaccgtgctgaacggcgcgggccgctgaggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 32 - protein, 752 aa, Artificial Sequence
  Description of Artificial Sequence Synthetic PhaC-omp16 fusion polypeptide encoded by pET-14b-PhaC-omp16
MATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKAHMVLAVAIDKRGGGGGLEGCASKKNLPNNAGDLGLGAGAATPGSSQDFTVNVGDRIFFDLDSSLIRADAQQTLSKQAQWLQRYPQYSITIEGHADERGTREYNLALGQRRAAATRDFLASRGVPTNRMRTISYGNERPVAVCDADTCWSQNRRAVTVLNGAGR

SEQ ID NO: 33 - DNA, 6375 bp, Artificial Sequence
  Description of Artificial Sequence Synthetic construct - pET-14b-6Cys-PhaC
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataaggagatactagtatgtgttgttgttgttgttgtgcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcatgtaaggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 34 - protein, 595 aa, Artificial Sequence
  Description of Artificial Sequence Synthetic Cys6-PhaC fusion polypeptide encoded by pET-14b-6Cys-PhaC
MCCCCCCATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 35 - DNA, 8464 bp, Artificial Sequence
  Description of Artificial Sequence Synthetic construct - pET-14b-PorA-PhaC-PorB
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatatccggatatagttcctcctttcagcaaaaaacccctcaagacccgtttagaggccccaaggggttatgctagttattgctcagcggtggcagcagccaactcagcttcctttcgggctttgttagcagccggatccttagaatttgtgacgcagaccgacaccgccggcagtcgatacgaatttgttttcgcctttgccttcttgcaaccaaccggcagaaaccaaggcagaagtgcgtttggagaagtcgtattccgcaccgacaaccacttggtcgtaatcgttgcttaagtctgcatcatcaaacgagcctttgaagccgtgggcgtaagaaacgcggggcgttacgttgccgaagcggtatgccaaggtagcggcaacttcggtttgagagttgtgcgaattgtcgtttggcaaagccaatttcgcatcttgttgttgtacggctacggaggcgtacagggcatcattgtcgtaaccgctgaccaaacggtgaatctggtatttctcaatattcacgttctcttgcacttgatgatgtcttttataggcaccgccatattgcacgaagaagccgccgtttttgtagttgaagccggcgtggtaagattcgctgttatgtctgcctgcattgtcgttaagcgcgtattgtacgctgccgctgaggccggcaaattcgggagaatcgtagcgtacggaaatgaggcgtgcttcgggttcggcaattttgtttacacccaaatagtcgcttttgctatcccaaggattgatgtcgccggtatctttcaggacgctgttcaaacggccgacgcgcaatttaccgaagccgcctttcaagccgatgaaggattggcggttgccccaaccggagtcagtaccggcgatagatgctttttgctcaacctgccaaatggctttcaggccgttaccgaggtcttcttggcctttgaagccgattttcgaacccaaatcaacgatgccggtaccggtttcaacgctagccgcctgagctccattgtgagctacggagcgggaagtttctacgccggctttgatggtgccgtacagggtaacgtcctcgaggcctccaccgcctccgcgtttatcaatcgccaccgccagcaccatatgtgccttggctttgacgtatcgcccaggcgcgggttcgattgcgcgatagcgcgcattgccatagttggcgggcgcggcgcgtttcgcgccggcctgcccggccagccatgcggtccagtccggccaccagctgccgtgatgctcgatggcgccggccagccattgctgcggcgactccggcagcgcatcgttagtccagtggctgcgcttgttcttggccggcgggttgatcacaccggcgatatggcccgacgcacccagcacgaagcgcagcttgttcgccagcagcgcggtcgaggcataggccgcggtccacggcacgatatggtcttcgcgcgagccgtagatataggtcggcacgtcgatgctggccaggtccaccggcacgccgcacacggtcagcttgcccggtaccttgagctcgttctgcaggtaggtgtggcgcaggtaccagcagtaccacggccccggcaggttggtggcgtcgccgttccagaacagcaggtcgaacggcaccggcgtgttgcccttcaggtagttgtcgaccacgtagttccacaccaggtcgttcgggcgcaagaacgagaaggtattggccagctcaaggccgcgcagcagcgcgcacggcgcgccggcgccgccgcccagcgtggcctcgcgcaactgcacatggccctcgtcgacaaagacgtcgaggatgcccgtgtcggcaaagtccagcagcgtggtcagcagcgtgacgctggcggccgggtgctcgccgcgcgcggccagcaccgccagcgcggtcgagacaatggtgccgcccacgcagaagccgagcacgttgatcttgtcctggccgctgatgtcgcgcgcgacttcgatggcgcggatggccgcgtgctcgatgtagtcgtcccaggtgctgccggccatgctggcgtccggattgcgccacgacaccagaaacaccgtatgtccctgctccaccacatggcgcaccagcgagctctccggctgcaggtccaggatgtagtacttgttgatgcacggcggcaccatcagcagcgggcgcgcgtgcaccttgtcggtcagcggcttgtactgcaacagctggaagtactcgttctcgaagaccacggcgccttcggtcaccgcgacattgcggccgacctcaaacgcgctctcgtcggtctgcgagatcttgccgcgtgtcaggtcttccatcatgttgcgcacgccggcacgcagcgattcgccgcccgactcgatcagcaggcgctgcgcctcgggattggtggcaaggaagttggcgggcgacatcgcatcgacccattgcgagatcgcgaagcggatgcgctggcgggtcttggcatcggcctcgacggcatcggccagctcggtcaaggcgcgcgcattgagcaggtagaacgcggcagcgaagcgatatgggaggttggtgcgccatgcgtcgccggcgaagcgccggtcgtgcagcggaccggtggcctcggccttgccctcggccatggcctgccacagcgctgagaagtccttcatgtagcgctgctggatatcacccagctgcgccggcgcgatcttgacgcctgccagcgcatccaggcccggaatgccggacgcggccgcgtggccgttgccttcagtgccctgccactggcgggaccattccagccatgtggctggatcgaatggccccggcgtgaccttgaatggttgggacttgccttcctgcgtggaagctgccgcgcctttgccggtcgcaaatttatggcgcaggccaatgctcgccgcgttaatctgggtatagttgccaatgccggtgttgcgtttcagccacgcgccgctcacaatcgcgctggtgcgtttgctaaaatcataatccacgcccgcaataatctgatcatagctggtgttttcgccttttttgccgcgttcaataaaatcaaagccatgcgcatagctaatgcgcggcaccgcgttgccaaagcgatagctcgcggtcgccgcaatttcggtggtgctgtttttggttttatcgccgttttcgctcagatccagctgcgccgccagcgccaggttcaggccgccttcttcatagccgccggtcaggcgatgcacctgatggtttttcagcggatcggtgcctttcgcttcatcatcatcgccaatgcggcccagcagaaacaggttaaacgcatcgcggcccacgttcgcatggcgcgcatatttaaacgcatagttgcccgcaaagccgccgtttttatagttcaggcccgcataatacacatcgctgcccggtttgcccaccaccgccggcacgcgaataatggtgttgttcgcgccgttcgccagggtcgccggggtatacgcgcttttgctgttctgcgccggcacaaactgcacgctgccgctaaagccgctaaattccgggctatcatagcgcacgctcaccggcatatcatcatggcgtttaaaaatgcccagctggctcgccacatcgttgttgctatcccacggatcaatcgcctggctcgcatcatcaaactggttcgccacgcggcccgcgcgcagggtgccaaattcgcccgccaggccaataaagctttcgcggttgccccactgggtcgcgccgccgcccgccacgctcacatcctgttccagctgccacaccgctttcaggccatcgcccagatcttcgctgcctttaaagccaataaagctgccaaaatcgctaattttggtgcgaatgcggcttttcgctttggtcactttcacctggccgctcgcgccgccgttcgccgcctgcgcttcggtcagctgcagctgatagttgcggccttccacgcccgctttaatttcgccatacaggctcacatccatactggtatctcctatattctagagggaaaccgttgtggtctccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 36 - protein, 1292 aa, Artificial Sequence
  Description of Artificial Sequence Synthetic PorA-PhaC-PorB fusion polypeptide encoded by pET-14b-PorA-PhaC-PorB
MDVSLYGEIKAGVEGRNYQLQLTEAQAANGGASGQVKVTKAKSRIRTKISDFGSFIGFKGSEDLGDGLKAVWQLEQDVSVAGGGATQWGNRESFIGLAGEFGTLRAGRVANQFDDASQAIDPWDSNNDVASQLGIFKRHDDMPVSVRYDSPEFSGFSGSVQFVPAQNSKSAYTPATLANGANNTIIRVPAVVGKPGSDVYYAGLNYKNGGFAGNYAFKYARHANVGRDAFNLFLLGRIGDDDEAKGTDPLKNHQVHRLTGGYEEGGLNLALAAQLDLSENGDKTKNSTTEIAATASYRFGNAVPRISYAHGFDFIERGKKGENTSYDQIIAGVDYDFSKRTSAIVSGAWLKRNTGIGNYTQINAASIGLRHKFATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKAHMVLAVAIDKRGGGGGLEDVTLYGTIKAGVETSRSVAHNGAQAASVETGTGIVDLGSKIGFKGQEDLGNGLKAIWQVEQKASIAGTDSGWGNRQSFIGLKGGFGKLRVGRLNSVLKDTGDINPWDSKSDYLGVNKIAEPEARLISVRYDSPEFAGLSGSVQYALNDNAGRHNSESYHAGFNYKNGGFFVQYGGAYKRHHQVQENVNIEKYQIHRLVSGYDNDALYASVAVQQQDAKLALPNDNSHNSQTEVAATLAYRFGNVTPRVSYAHGFKGSFDDADLSNDYDQVVVGAEYDFSKRTSALVSAGWLQEGKGENKFVSTAGGVGLRHKF

SEQ ID NO: 37 - DNA, 10693 bp, Artificial Sequence
  Description of Artificial Sequence Synthetic construct - pET-14b-FetA-PhaC-ZnuD
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatatccggatatagttcctcctttcagcaaaaaacccctcaagacccgtttagaggccccaaggggttatgctagttattgctcagcggtggcagcagccaactcagcttcctttcgggctttgttagcagccggatcctcaaaatttcacgttcacgccgccggtaaagctgcggcccatctgcggggtatcgctcagaaagctgctatgcgcatacacgctctggttcagcaggttatccgctttcacataccagttccattcgccatagcgggtgttgcggcgatagttcgcgcccaggttcagcatatgatggcccggggtgcgggtttcatagcgcgccagtttgttctgcgcaaacacgcgataataatccaggttcgcatcaatgcgatcggtcaggctcgctttcagatgaaagcccaggcgcgccgccggcacgcgcggcgcgttctgatcatcctgcgcaataaacgggcggttgccatacgcatcttcgcggcccggcaggctcggcaggtttttcaggcggccgcgcacataatcgccgctcacgccaatgcgatagcgcggggtcggtttaaaataaatttcgccttccgcgccataaaaatccgcgccgctctggttatagcgcaccagtttcatttcgctatcatcttcaatgcttttcgggccgcggccatcgttcagggtctgcgcataaatatagttgccaaagcggttgcgatacagcgccaggttatactgccagcgatcgccttcatagcccagcgccagttcaatgttgttgctgcgttctttgttcagatgtttgttgcccacttcaaaggtgttggtcgccacatgtttgccatgcgcatacagttcctgggtgctcggcaggcgttcctgatggctcgcggtcaggctcagtttatgctgcggggtaaaataccagttgccgctcagcgcaaagctgcgcgcggtctggcgatgcgcgcccagatccggcagcggatggttataatagttttcgcgatcaatcagcgctttatcatactgaatgctcgctttctgtttttccacgcgcacgccgccttccagggtaaagttatcccagttcgcctgttccacgccaaaaaagctataatgctgcactttgttatccagcagcatcggctgtttcaccgcttcgctaatcgcgctcagcgcgctgcttttctgctgcagatactgcacgccccagctgcctttcaggcggccaatcggctgatggcgcagttcaatgcgcgcgttctgggtctggttgttaaaaaagttttccaccgcatcgcccgctttttcatcatggcgataatcgttgcggttcagatgcacgcgcagcgcttcaaagcccggaaacggctgtttccattccgcgcgcagttcatagcgtttgttgcgcagatcaatccacgggcggccgctatgggtatgcgcatgcgcgttatcatcatcatgaaagccgcagctcaggcccgggttatcataatcaatatcttcttcggtcagcagatgcggatacagctgcagatagcgtttgttaatcaggcttttctgccaaataatatccgcatggcaatcatcatattcatggctatgcgccggcaggccatactgatcgcggcgatcgctatacgccacgccaataaagcctttttcgcccacccagctcaggccaatgctgccggtctggctatccgcatggctatccggcaggcgtttcaggttgcgatagcgcggcaccgcataatcgccgcttttgcgatacaggccttcggtatgcagcacaaagtttttgcccaggccaatgttaatgccgccgctggtcagtttttccaggttgccgctgctcaggcgcaggcccagttcgccgctcacgccgttttccggcattttttccggaattttgccatccgccacatccaccaggcccgccacgttgccgctgctatacagcagggtcaccgggccgcgcagaatttccacctgctggctcagcgcggtatccaccataatcgcatgatccgggctaaaatccgccatatcgccggtttcgccatgatggttcagcactttaatgcggcggccggtctggccgcgaatcaccggcgcgctcgcgccgccgccatactggctcgcatgaatgcccggcacgccatccagcgcatcgcccaggttcaccgctttctggcgcagggtatcgccgctaataattttatcgctcgcggtgctggtatgcagcaggccgctggtcgcgcgcgggcggcttttgcccaccacgctcacggtttccagatccacgctctgttcggtttcatgctcgaggcctccaccgcctccgcgtttatcaatcgccaccgccagcaccatatgtgccttggctttgacgtatcgcccaggcgcgggttcgattgcgcgatagcgcgcattgccatagttggcgggcgcggcgcgtttcgcgccggcctgcccggccagccatgcggtccagtccggccaccagctgccgtgatgctcgatggcgccggccagccattgctgcggcgactccggcagcgcatcgttagtccagtggctgcgcttgttcttggccggcgggttgatcacaccggcgatatggcccgacgcacccagcacgaagcgcagcttgttcgccagcagcgcggtcgaggcataggccgcggtccacggcacgatatggtcttcgcgcgagccgtagatataggtcggcacgtcgatgctggccaggtccaccggcacgccgcacacggtcagcttgcccggtaccttgagctcgttctgcaggtaggtgtggcgcaggtaccagcagtaccacggccccggcaggttggtggcgtcgccgttccagaacagcaggtcgaacggcaccggcgtgttgcccttcaggtagttgtcgaccacgtagttccacaccaggtcgttcgggcgcaagaacgagaaggtattggccagctcaaggccgcgcagcagcgcgcacggcgcgccggcgccgccgcccagcgtggcctcgcgcaactgcacatggccctcgtcgacaaagacgtcgaggatgcccgtgtcggcaaagtccagcagcgtggtcagcagcgtgacgctggcggccgggtgctcgccgcgcgcggccagcaccgccagcgcggtcgagacaatggtgccgcccacgcagaagccgagcacgttgatcttgtcctggccgctgatgtcgcgcgcgacttcgatggcgcggatggccgcgtgctcgatgtagtcgtcccaggtgctgccggccatgctggcgtccggattgcgccacgacaccagaaacaccgtatgtccctgctccaccacatggcgcaccagcgagctctccggctgcaggtccaggatgtagtacttgttgatgcacggcggcaccatcagcagcgggcgcgcgtgcaccttgtcggtcagcggcttgtactgcaacagctggaagtactcgttctcgaagaccacggcgccttcggtcaccgcgacattgcggccgacctcaaacgcgctctcgtcggtctgcgagatcttgccgcgtgtcaggtcttccatcatgttgcgcacgccggcacgcagcgattcgccgcccgactcgatcagcaggcgctgcgcctcgggattggtggcaaggaagttggcgggcgacatcgcatcgacccattgcgagatcgcgaagcggatgcgctggcgggtcttggcatcggcctcgacggcatcggccagctcggtcaaggcgcgcgcattgagcaggtagaacgcggcagcgaagcgatatgggaggttggtgcgccatgcgtcgccggcgaagcgccggtcgtgcagcggaccggtggcctcggccttgccctcggccatggcctgccacagcgctgagaagtccttcatgtagcgctgctggatatcacccagctgcgccggcgcgatcttgacgcctgccagcgcatccaggcccggaatgccggacgcggccgcgtggccgttgccttcagtgccctgccactggcgggaccattccagccatgtggctggatcgaatggccccggcgtgaccttgaatggttgggacttgccttcctgcgtggaagctgccgcgcctttgccggtcgcactagtaaatttatagttcacgcccaggcgcacatcgcggcccacgcccggcagggtgttggtccagcgctggctatgcggataataaaaggtgttaaacacgttgttcacgctcaggttcacgttcagggtatctttgcccagcggtttccagttcgcaaacacatcgttcacgccaaagcctttgcgcaccacgttttccaggttgccctggcgatctttctggcccgccaccagaatgctgcccaccgctttctgcacatagcggccgcgccagccaatttccaggttcgggtttttaaagcgatacgccaggctcgcggtccaggtgcggcccacctgcgcgccaaattccgggttcgcgctcagcagtttatctttatgggtatcataaaagcgcggtttgctatggctcacgcccactttcgcggtcaggccgccggtgcgatagctcgcgcccagttcatagccatggtttttaatatagcccgcgttcaccgcttcgcgcaccgccacgctatcatggcggttctgcgggttcgccagcgcatctttaatggtctgccaaaaatagctgccgttcgccgcaaaggtgccatcgttatagttaaagccaatttcggtgttgcgcgcgcgttccgctttggtgccatccgcaatgctaataatgccgcgtttgccatgggtctgcagcgcatcatacaggcgcgggctgcggctcgcatagttatggctcgcgctaaagctccaatattcatgcggctgccaaatcacgccaaagctcgggttcaggctgctgctgctcacggttttgccatcatgggttttcactttaaagcgatcatagcgcaggccgccggtcagggtaaagccatcaatttcatgaatcgcttcaatatacgcgccggtatcggttttggtcgggttggtcaggcgatacgctttcacaatgttttcatcatcgcggtttttctgtttttccgcctgggtcgcgccgcttttatctttaattttaaattcgccgttcagaaacgcctgcggtttaatttcctgatggcgatagttaatgccatatttcagcagggtctgttccgccaggcggctatcaaagttaaagttcgcgccatgggtggtaatgcgggtatggttcgggccttccacgttgcccgcatagccgctgcctttatcatccgcgctatagcgttttttttccagcacatacgcgttcgcatccagtttttccacaaagcccaggtttttgccggtatacgccaggttggtgttgctctgggtggtttcgcgatacgccggcgcctggcgttccatgctaatgcgttctttatcgccgcccacggtaaattcttcgcgcacggtgcgaatgccgcgatgctgatctttcatatggctcagcacaatgcgatgatcgccatcgccaaagctggtgccaattttcgccagatagctgcgtttatccagcgcgctatacggcacggttttgccgccgttaaagttgttgcgaaagcctttgcccgcttcataatctttttcgttgttgcggttatagctaaacaggccatcaaagttgccttctttgccaaacacgctcgcgccatagctcacgccttcgttgctcgcaaagccgctgttcaggcgcacgccccagtttttatccaggcctttcagcagatcctgcgcatccacggttttcgcaataatcgcgccgttggtcgcgccaatgcccgcgctcgcgctgcccgcgcctttctgcacgctcaccactttcaccagcgccggatccacaataaagcggccctgatgatacagaatctggctatcgctatacgcgttatccactttaatatccacgctgttctggcccatgccgcgcagggtcagaaactggctggtgccgttgccgccgccaaaatcaatgctcggttcttctttcagcagttcgcgcatatcggtcgcggtgctttcatctttctgctgcagggtcacaatgttggtgcgaattttgctgccctggcgatcgcctttcacggtaatggtatccagcaccactttcgcgttgttttccgccatactggtatctcctatattctagagggaaaccgttgtggtctccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 38 - protein, 2029 aa, Artificial Sequence
  Description of Artificial Sequence Synthetic fetA-PhaC-ZnuD fusion polypeptide encoded by pET-14b-FetA-PhaC-ZnuD
MAENNAKVVLDTITVKGDRQGSKIRTNIVTLQQKDESTATDMRELLKEEPSIDFGGGNGTSQFLTLRGMGQNSVDIKVDNAYSDSQILYHQGRFIVDPALVKVVSVQKGAGSASAGIGATNGAIIAKTVDAQDLLKGLDKNWGVRLNSGFASNEGVSYGASVFGKEGNFDGLFSYNRNNEKDYEAGKGFRNNFNGGKTVPYSALDKRSYLAKIGTSFGDGDHRIVLSHMKDQHRGIRTVREEFTVGGDKERISMERQAPAYRETTQSNTNLAYTGKNLGFVEKLDANAYVLEKKRYSADDKGSGYAGNVEGPNHTRITTHGANFNFDSRLAEQTLLKYGINYRHQEIKPQAFLNGEFKIKDKSGATQAEKQKNRDDENIVKAYRLTNPTKTDTGAYIEAIHEIDGFTLTGGLRYDRFKVKTHDGKTVSSSSLNPSFGVIWQPHEYWSFSASHNYASRSPRLYDALQTHGKRGIISIADGTKAERARNTEIGFNYNDGTFAANGSYFWQTIKDALANPQNRHDSVAVREAVNAGYIKNHGYELGASYRTGGLTAKVGVSHSKPRFYDTHKDKLLSANPEFGAQVGRTWTASLAYRFKNPNLEIGWRGRYVQKAVGSILVAGQKDRQGNLENVVRKGFGVNDVFANWKPLGKDTLNVNLSVNNVFNTFYYPHSQRWTNTLPGVGRDVRLGVNYKFATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKAVLAVAIDKRGGGGGHETEQSVDLETVSVVGKSRPRATSGLLHTSTASDKIISGDTLRQKAVNLGDALDGVPGIHASQYGGGASAPVIRGQTGRRIKVLNHHGETGDMADFSPDHAIMVDTALSQQVEILRGPVTLLYSSGNVAGLVDVADGKIPEKMPENGVSGELGLRLSSGNLEKLTSGGINIGLGKNFVLHTEGLYRKSGDYAVPRYRNLKRLPDSHADSQTGSIGLSWVGEKGFIGVAYSDRRDQYGLPAHSHEYDDCHADIIWQKSLINKRYLQLYPHLLTEEDIDYDNPGLSCGFHDDDNAHAHTHSGRPWIDLRNKRYELRAEWKQPFPGFEALRVHLNRNDYRHDEKAGDAVENFFNNQTQNARIELRHQPIGRLKGSWGVQYLQQKSSALSAISEAVKQPMLLDNKVQHYSFFGVEQANWDNFTLEGGVRVEKQKASIQYDKALIDRENYYNHPLPDLGAHRQTARSFALSGNWYFTPQHKLSLTASHQERLPSTQELYAHGKHVATNTFEVGNKHLNKERSNNIELALGYEGDRWQYNLALYRNRFGNYIYAQTLNDGRGPKSIEDDSEMKLVRYNQSGADFYGAEGEIYFKPTPRYRIGVSGDYVRGRLKNLPSLPGREDAYGNRPFIAQDDQNAPRVPAARLGFHLKASLTDRIDANLDYYRVFAQNKLARYETRTPGHHMLNLGANYRRNTRYGEWNWYVKADNLLNQSVYAHSSFLSDTPQMGRSFTGGVNVKF

SEQ ID NO: 39 - DNA, 8665 bp, Artificial Sequence
  Description of Artificial Sequence Synthetic construct - pET-14b-PhaC-PA83
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataaggagatactagtatggcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcacatatggtgctggcggtggcgattgataaacgcggaggcggtggaggcctcgagatggatgcgatgaaacgcggcctgtgctgcgtgctgctgctgtgcggcgcggtgtttgtgagcccgagcgaagtgaaacaggaaaaccgcctgctgaacgaaagcgaaagcagcagccagggcctgctgggctattattttagcgatctgaactttcaggcgccgatggtggtgaccagcagcaccaccggcgatctgagcattccgagcagcgaactggaaaacattccgagcgaaaaccagtattttcagagcgcgatttggagcggctttattaaagtgaaaaaaagcgatgaatatacctttgcgaccagcgcggataaccatgtgaccatgtgggtggatgatcaggaagtgattaacaaagcgagcaacagcaacaaaattcgcctggaaaaaggccgcctgtatcagattaaaattcagtatcagcgcgaaaacccgaccgaaaaaggcctggattttaaactgtattggaccgatagccagaacaaaaaagaagtgattagcagcgataacctgcagctgccggaactgaaacagaaaagcagcaacaccagcgcgggcccgaccgtgccggatcgcgataacgatggcattccggatagcctggaagtggaaggctataccgtggatgtgaaaaacaaacgcacctttctgagcccgtggattagcaacattcatgaaaaaaaaggcctgaccaaatataaaagcagcccggaaaaatggagcaccgcgagcgatccgtatagcgattttgaaaaagtgaccggccgcattgataaaaacgtgagcccggaagcgcgccatccgctggtggcggcgtatccgattgtgcatgtggatatggaaaacattattctgagcaaaaacgaagatcagagcacccagaacaccgatagcgaaacccgcaccattagcaaaaacaccagcaccagccgcacccataccagcgaagtgcatggcaacgcggaagtgcatgcgagcttttttgatattggcggcagcgtgagcgcgggctttagcaacagcaacagcagcaccgtggcgattgatcatagcctgagcctggcgggcgaacgcacctgggcggaaaccatgggcctgaacaccgcggataccgcgcgcctgaacgcgaacattcgctatgtgaacaccggcaccgcgccgatttataacgtgctgccgaccaccagcctggtgctgggcaaaaaccagaccctggcgaccattaaagcgaaagaaaaccagctgagccagattctggcgccgaacaactattatccgagcaaaaacctggcgccgattgcgctgaacgcgcaggatgattttagcagcaccccgattaccatgaactataaccagtttctggaactggaaaaaaccaaacagctgcgcctggataccgatcaggtgtatggcaacattgcgacctataactttgaaaacggccgcgtgcgcgtggataccggcagcaactggagcgaagtgctgccgcagattcaggaaaccaccgcgcgcattatttttaacggcaaagatctgaacctggtggaacgccgcattgcggcggtgaacccgagcgatccgctggaaaccaccaaaccggatatgaccctgaaagaagcgctgaaaattgcgtttggctttaacgaaccgaacggcaacctgcagtatcagggcaaagatattaccgaatttgattttaactttgatcagcagaccagccagaacattaaaaaccagctggcggaactgaacgcgaccaacatttataccgtgctggataaaattaaactgaacgcgaaaatgaacattctgattcgcgataaacgctttcattatgatcgcaacaacattgcggtgggcgcggatgaaagcgtggtgaaagaagcgcatcgcgaagtgattaacagcagcaccgaaggcctgctgctgaacattgataaagatattcgcaaaattctgagcggctatattgtggaaattgaagataccgaaggcctgaaagaagtgattaacgatcgctatgatatgctgaacattagcagcctgcgccaggatggcaaaacctttattgattttaaaaaatataacgataaactgccgctgtatattagcaacccgaactataaagtgaacgtgtatgcggtgaccaaagaaaacaccattattaacccgagcgaaaacggcgataccagcaccaacggcattaaaaaaattctgatttttagcaaaaaaggctatgaaattggctgaggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 40 - protein, 1359 aa, Artificial Sequence
  Description of Artificial Sequence Synthetic PhaC-PA83 fusion polypeptide encoded by pET-14b-PhaC-PA83
MATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKAHMVLAVAIDKRGGGGGLEMDAMKRGLCCVLLLCGAVFVSPSEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSETRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 41 - DNA, 8122 bp, Artificial Sequence
  Description of Artificial Sequence Synthetic construct - pET-14b-E1-PhaC-M1
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaatataggagataccagtatgcgctgcgtgggcattggcaaccgcgattttgtggaaggcctgagcggcgcgacctgggtggatgtggtgctggaacatggcagctgcgtgaccaccatggcgaaagataaaccgaccctggatattgaactgctgaaaaccgaagtgaccaacccggcggtgctgcgcaaactgtgcattgaagcgaaaattagcaacaccaccaccgatagccgctgcccgacccagggcgaagcgaccctggtggaagaacaggataccaactttgtgtgccgccgcacctttgtggatcgcggctggggcaacggctgcggcctgtttggcaaaggcagcctgattacctgcgcgaaatttaaatgcgtgaccaaactggaaggcaaaattgtgcagtatgaaaacctgaaatatagcgtgattgtgaccgtgcataccggcgatcagcatcaggtgggcaacgaaaccaccgaacatggcaccaccgcgaccattaccccgcaggcgccgaccagcgaaattcagctgaccgattatggcgcgctgaccctggattgcagcccgcgcaccggcctggattttaacgaaatggtgctgctgaccatgaaaaaaaaaagctggctggtgcataaacagtggtttctggatctgccgctgccgtggaccagcggcgcgagcaccagccaggaaacctggaaccgccaggatctgctggtgacctttaaaaccgcgcatgcgaaaaaacaggaagtggtggtgctgggcagccaggaaggcgcgatgcataccgcgctgaccggcgcgaccgaaattcagaccagcggcaccaccaccatttttgcgggccatctgaaatgccgcctgaaaatggataaactgattctgaaaggcatgagctatgtgatgtgcaccggcagctttaaactggaaaaagaagtggcggaaacccagcatggcaccgtgctggtgcaggtgaaatatgaaggcaccgatgcgccgtgcaaaattccgtttagcagccaggatgaaaaaggcgtgacccagaacggccgcctgattaccgcgaacccgattgtgaccgataaagaaaaaccggtgaacattgaagcggaaccgccgtttggcgaaagctatattgtggtgggcgcgggcgaaaaagcgctgaaactgagctggtttaaaaaaggcagcagcattggcaaaatgtttgaagcgaccgcgcgcggcgcgcgccgcatggcgattctgggcgataccgcgtgggattttggcagcattggcggcgtgtttaccagcgtgggcaaactgattcatcagatttttggcaccgcgtatggcgtgctgtttagcggcgtgagctggaccatgaaaattggcattggcattctgctgacctggctgggcctgaacagccgcagcaccagcctgagcatgacctgcattgcggtgggcatggtgaccctgtatctgggcgtgatggtgcaggcgactagtgcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcacatatggtgctggcggtggcgattgataaacgcggaggcggtggaggcctcgagagcgtggcgctggcgccgcatgtgggcctgggcctggaaacccgcaccgaaacctggatgagcagcgaaggcgcgtggaaacagattcagaaagtggaaacctgggcgctgcgccatccgggctttaccgtgattgcgctgtttctggcgcatgcgattggcaccagcattacccagaaaggcattatttttattctgctgatgctggtgaccccgagcatggcgtaaggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 42 - protein, 1178 aa, Artificial Sequence
  Description of Artificial Sequence Synthetic E1-PhaC-M1 fusion polypeptide encoded by pET-14b-E1-PhaC-M1
MRCVGIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWGNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMKKKSWLVHKQWFLDLPLPWTSGASTSQETWNRQDLLVTFKTAHAKKQEVVVLGSQEGAMHTALTGATEIQTSGTTTIFAGHLKCRLKMDKLILKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQATSATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKAHMVLAVAIDKRGGGGGLESVALAPHVGLGLETRTETWMSSEGAWKQIQKVETWALRHPGFTVIALFLAHAIGTSITQKGIIFILLMLVTPSMA

SEQ ID NO: 43 - DNA, 8122 bp, Artificial Sequence
  Description of Artificial Sequence Synthetic construct - pET-14b-E2-PhaC-M2
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaatataggagataccagtatgcgctgcattggcatgagcaaccgcgattttgtggaaggcgtgagcggcggcagctgggtggatattgtgctggaacatggcagctgcgtgaccaccatggcgaaaaacaaaccgaccctggattttgaactgattaaaaccgaagcgaaacagccggcgaccctgcgcaaatattgcattgaagcgaaactgaccaacaccaccaccgaaagccgctgcccgacccagggcgaaccgagcctgaacgaagaacaggataaacgctttgtgtgcaaacatagcatggtggatcgcggctggggcaacggctgcggcctgtttggcaaaggcggcattgtgacctgcgcgatgtttcgctgcaaaaaaaacatggaaggcaaagtggtgcagccggaaaacctggaatataccattgtgattaccccgcatagcggcgaagaacatgcggtgggcaacgataccggcaaacatggcaaagaaattaaaattaccccgcagagcagcattaccgaagcggaactgaccggctatggcaccgtgaccatggaatgcagcccgcgcaccggcctggattttaacgaaatggtgctgctgcagatggaaaacaaagcgtggctggtgcatcgccagtggtttctggatctgccgctgccgtggctgccgggcgcggatacccagggcagcaactggattcagaaagaaaccctggtgacctttaaaaacccgcatgcgaaaaaacaggatgtggtggtgctgggcagccaggaaggcgcgatgcataccgcgctgaccggcgcgaccgaaattcagatgagcagcggcaacctgctgtttaccggccatctgaaatgccgcctgcgcatggataaactgcagctgaaaggcatgagctatagcatgtgcaccggcaaatttaaagtggtgaaagaaattgcggaaacccagcatggcaccattgtgattcgcgtgcagtatgaaggcgatggcagcccgtgcaaaattccgtttgaaattatggatctggaaaaacgccatgtgctgggccgcctgattaccgtgaacccgattgtgaccgaaaaagatagcccggtgaacattgaagcggaaccgccgtttggcgatagctatattattattggcgtggaaccgggccagctgaaactgaactggtttaaaaaaggcagcagcattggccagatgtttgaaaccaccatgcgcggcgcgaaacgcatggcgattctgggcgataccgcgtgggattttggcagcctgggcggcgtgtttaccagcattggcaaagcgctgcatcaggtgtttggcgcgatttatggcgcggcgtttagcggcgtgagctggaccatgaaaattctgattggcgtgattattacctggattggcatgaacagccgcagcaccagcctgagcgtgaccctggtgctggtgggcattgtgaccctgtatctgggcgtgatggtgcaggcgactagtgcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcacatatggtgctggcggtggcgattgataaacgcggaggcggtggaggcctcgagagcgtggcgctggtgccgcatgtgggcatgggcctggaaacccgcaccgaaacctggatgagcagcgaaggcgcgtggaaacatgtgcagcgcattgaaacctggattctgcgccatccgggctttaccatgatggcggcgattctggcgtataccattggcaccacccattttcagcgcgcgctgatttttattctgctgaccgcggtgaccccgagcatgacctaaggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 44 - protein, 1178 aa, Artificial Sequence
  Description of Artificial Sequence Synthetic E2-PhaC-M2 fusion polypeptide encoded by pET-14B-E2-PhaC-M2
MRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVTLVLVGIVTLYLGVMVQATSATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKAHMVLAVAIDKRGGGGGLESVALVPHVGMGLETRTETWMSSEGAWKHVQRIETWILRHPGFTMMAAILAYTIGTTHFQRALIFILLTAVTPSMT

SEQ ID NO: 45 - DNA, 8116 bp, Artificial Sequence
  Description of Artificial Sequence Synthetic construct - pET-14b-E3-PhaC-M3
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaatataggagataccagtatgcgctgcgtgggcgtgggcaaccgcgattttgtggaaggcctgagcggcgcgacctgggtggatgtggtgctggaacatggcggctgcgtgaccaccatggcgaaaaacaaaccgaccctggatattgaactgcagaaaaccgaagcgacccagctggcgaccctgcgcaaactgtgcattgaaggcaaaattaccaacattaccaccgatagccgctgcccgacccagggcgaagcggtgctgccggaagaacaggatcagaactatgtgtgcaaacatacctatgtggatcgcggctggggcaacggctgcggcctgtttggcaaaggcagcctggtgacctgcgcgaaatttcagtgcctggaaccgattgaaggcaaagtggtgcagtatgaaaacctgaaatataccgtgattattaccgtgcataccggcgatcagcatcaggtgggcaacgaaacccagggcgtgaccgcggaaattaccccgcaggcgagcaccaccgaagcgattctgccggaatatggcaccctgggcctggaatgcagcccgcgcaccggcctggattttaacgaaatgattctgctgaccatgaaaaacaaagcgtggatggtgcatcgccagtggttttttgatctgccgctgccgtgggcgagcggcgcgaccaccgaaaccccgacctggaaccgcaaagaactgctggtgacctttaaaaacgcgcatgcgaaaaaacaggaagtggtggtgctgggcagccaggaaggcgcgatgcataccgcgctgaccggcgcgaccgaaattcagaacagcggcggcaccagcatttttgcgggccatctgaaatgccgcctgaaaatggataaactggaactgaaaggcatgagctatgcgatgtgcaccaacacctttgtgctgaaaaaagaagtgagcgaaacccagcatggcaccattctgattaaagtggaatataaaggcgaagatgcgccgtgcaaaattccgtttagcaccgaagatggccagggcaaagcgcataacggccgcctgattaccgcgaacccggtggtgaccaaaaaagaagaaccggtgaacattgaagcggaaccgccgtttggcgaaagcaacattgtgattggcattggcgataacgcgctgaaaattaactggtataaaaaaggcagcagcattggcaaaatgtttgaagcgaccgaacgcggcgcgcgccgcatggcgattctgggcgataccgcgtgggattttggcagcgtgggcggcgtgctgaacagcctgggcaaaatggtgcatcagatttttggcagcgcgtataccgcgctgtttagcggcgtgagctgggtgatgaaaattggcattggcgtgctgctgacctggattggcctgaacagcaaaaacaccagcatgagctttagctgcattgcgattggcattattaccctgtatctgggcgcggtggtgcaggcgactagtgcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcacatatggtgctggcggtggcgattgataaacgcggaggcggtggaggcctcgagagcgtggcgctggcgccgcatgtgggcatgggcctggatacccgcacccagacctggatgagcgcggaaggcgcgtggcgccaggtggaaaaagtggaaacctgggcgctgcgccatccgggctttaccattctggcgctgtttctggcgcattatattggcaccagcctgacccagaaagtggtgatttttattctgctgatgctggtgaccccgagcatgacctaaggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 46 - protein, 1176 aa, Artificial Sequence
  Description of Artificial Sequence Synthetic E3-PhaC-M3 fusion polypeptide encoded by pET-14B-E3-PhaC-M3
MRCVGVGNRDFVEGLSGATWVDVVLEHGGCVTTMAKNKPTLDIELQKTEATQLATLRKLCIEGKITNITTDSRCPTQGEAVLPEEQDQNYVCKHTYVDRGWGNGCGLFGKGSLVTCAKFQCLEPIEGKVVQYENLKYTVIITVHTGDQHQVGNETQGVTAEITPQASTTEAILPEYGTLGLECSPRTGLDFNEMILLTMKNKAWMVHRQWFFDLPLPWASGATTETPTWNRKELLVTFKNAHAKKQEVVVLGSQEGAMHTALTGATEIQNSGGTSIFAGHLKCRLKMDKLELKGMSYAMCTNTFVLKKEVSETQHGTILIKVEYKGEDAPCKIPFSTEDGQGKAHNGRLITANPVVTKKEEPVNIEAEPPFGESNIVIGIGDNALKINWYKKGSSIGKMFEATERGARRMAILGDTAWDFGSVGGVLNSLGKMVHQIFGSAYTALFSGVSWVMKIGIGVLLTWIGLNSKNTSMSFSCIAIGIITLYLGAVVQATSATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKAHMVLAVAIDKRGGGGGLESVALAPHVGMGLDTRTQTWMSAEGAWRQVEKVETWALRHPGFTILALFLAHYIGTSLTQKVVIFILLMLVTPSMT

SEQ ID NO: 47 - DNA, 8119 bp, Artificial Sequence
  Description of Artificial Sequence Synthetic construct - pET-14b-E4-PhaC-M4
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatatccggatatagttcctcctttcagcaaaaaacccctcaagacccgtttagaggccccaaggggttatgctagttattgctcagcggtggcagcagccaactcagcttcctttcgggctttgttagcagccggatccttaatagctcggcgccaccagcatcatcagcacaaaaaacacggtgcgctgaatgccggtctggccaatcatatacgccataaagcccgccagcagcgcaaagcccgggttgcgcagaatccagctttccacgcgctgcgcatgtttccacgcgccttcgctgctcatccaggtttccgcgcgggtttccaggcccatgccgctatgcggggtcagcgccacgctctcgaggcctccaccgcctccgcgtttatcaatcgccaccgccagcaccatatgtgccttggctttgacgtatcgcccaggcgcgggttcgattgcgcgatagcgcgcattgccatagttggcgggcgcggcgcgtttcgcgccggcctgcccggccagccatgcggtccagtccggccaccagctgccgtgatgctcgatggcgccggccagccattgctgcggcgactccggcagcgcatcgttagtccagtggctgcgcttgttcttggccggcgggttgatcacaccggcgatatggcccgacgcacccagcacgaagcgcagcttgttcgccagcagcgcggtcgaggcataggccgcggtccacggcacgatatggtcttcgcgcgagccgtagatataggtcggcacgtcgatgctggccaggtccaccggcacgccgcacacggtcagcttgcccggtaccttgagctcgttctgcaggtaggtgtggcgcaggtaccagcagtaccacggccccggcaggttggtggcgtcgccgttccagaacagcaggtcgaacggcaccggcgtgttgcccttcaggtagttgtcgaccacgtagttccacaccaggtcgttcgggcgcaagaacgagaaggtattggccagctcaaggccgcgcagcagcgcgcacggcgcgccggcgccgccgcccagcgtggcctcgcgcaactgcacatggccctcgtcgacaaagacgtcgaggatgcccgtgtcggcaaagtccagcagcgtggtcagcagcgtgacgctggcggccgggtgctcgccgcgcgcggccagcaccgccagcgcggtcgagacaatggtgccgcccacgcagaagccgagcacgttgatcttgtcctggccgctgatgtcgcgcgcgacttcgatggcgcggatggccgcgtgctcgatgtagtcgtcccaggtgctgccggccatgctggcgtccggattgcgccacgacaccagaaacaccgtatgtccctgctccaccacatggcgcaccagcgagctctccggctgcaggtccaggatgtagtacttgttgatgcacggcggcaccatcagcagcgggcgcgcgtgcaccttgtcggtcagcggcttgtactgcaacagctggaagtactcgttctcgaagaccacggcgccttcggtcaccgcgacattgcggccgacctcaaacgcgctctcgtcggtctgcgagatcttgccgcgtgtcaggtcttccatcatgttgcgcacgccggcacgcagcgattcgccgcccgactcgatcagcaggcgctgcgcctcgggattggtggcaaggaagttggcgggcgacatcgcatcgacccattgcgagatcgcgaagcggatgcgctggcgggtcttggcatcggcctcgacggcatcggccagctcggtcaaggcgcgcgcattgagcaggtagaacgcggcagcgaagcgatatgggaggttggtgcgccatgcgtcgccggcgaagcgccggtcgtgcagcggaccggtggcctcggccttgccctcggccatggcctgccacagcgctgagaagtccttcatgtagcgctgctggatatcacccagctgcgccggcgcgatcttgacgcctgccagcgcatccaggcccggaatgccggacgcggccgcgtggccgttgccttcagtgccctgccactggcgggaccattccagccatgtggctggatcgaatggccccggcgtgaccttgaatggttgggacttgccttcctgcgtggaagctgccgcgcctttgccggtcgcactagtcgcctgcacggtaaagcccagaaacagggtaatgccgcccaccgcaatgcaggtcatcgccatgctggtgttgcggctgttggtgccaatccacagcaccagaaagccaatcagaatgcgaatcatccagctcacgccgccaaacatggtggtatacacgctgccaaacacctgatgcaccgctttgcccaggctggtaaacaggccgcccacgctgccaaaatcccacgcggtttcgcccagaatcgccatgcgtttcgcgccgcgataggtgctttcaaacattttgccaatgctgctgcctttgcgaaaccaatgcagggtcagcgcgctgttgcccacgccaatcacaatatagctatcgccaaacggcggttccagttcaatgttggtcacgctgttggtgttttccgccagcggggtgctgctaataatgcggcccaccactttttctttgttcacatcgcgaatttcaatcggcactttgcacggcgcgcccgcgccttcatatttcactttcaccacggtggtgccatgctgggtttccgccatttctttatcaatgctaaatttgccgctgcacatggtatagctcatgcctttaatgcgcagtttttccatgcgcactttgcatttcagatggcccgcaaacatatggttgccatcgccgctatccacttcggtcgcgcccgccagcgcgctatgcatcgcgccttcctggctgcccagcacggtcacatcctggcgtttcgcatgcggcactttaaaggtcaccatgcgttctttatagttccaatgcacttcgctggtatccgcgcccgcggtccacggcagcggcagatccagaaaccactgtttatgcaccagccaggttttttttttcattttcatcagaatcatttcgttaaaatcaatgccgctgcgcggttcgcaatccagggtcagttcgccataatccggcagtttcacttccacgctcgggctgcgcggggtaatcatcgcggtcacgccatggttgctggtatcgttgcccaccgcatgggtatcgccgttatgcacggtcaccaccacggtatattccaggttttcaatctgcaccaggttgccggtaattttgccgctgcagctaaatttcgcgcaggtcaccacgccgcctttgccaaacaggccgcagccgttgccccagccgcgatccaccacatcgcggcggcaaatatactgctgatcctgttcttctttcagatacggttcgccctgggtcgggcagcgggtcgcggtggtaatgttgctaatgctcgcttcaatgcaataggtgcgcagcagcgccacttctttcgcggtggttttggtcagttcaaaatccagggtcggtttgccctgcgccatggtggtcacgcagccgccatgttccagcaccagatccacccacgcgccgccgctcacgccttccacaaaatcgcggttgcccacgcccacgcagcgcatactggtatctcctatattctagagggaaaccgttgtggtctccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 48 - protein, 1177 aa, Artificial Sequence
  Description of Artificial Sequence Synthetic E4-PhaC-M4 fusion polypeptide encoded by pET-14b-E4-PhaC-M4
MRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTAMITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRIISSTPLAENTNSVTNIELEPPFGDSYIVIGVGNSALTLHWFRKGSSIGKMFESTYRGAKRMAILGETAWDFGSVGGLFTSLGKAVHQVFGSVYTTMFGGVSWMIRILIGFLVLWIGTNSRNTSMAMTCIAVGGITLFLGFTVQATSATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKAHMVLAVAIDKRGGGGGLESVALTPHSGMGLETRAETWMSSEGAWKHAQRVESWILRNPGFALLAGFMAYMIGQTGIQRTVFFVLMMLVAPSY

SEQ ID NO: 49 - DNA, 10468 bp, Artificial Sequence
  Description of Artificial Sequence Synthetic construct - pET-14b-SEBOVGP-PhaC-ZEBOVGP
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatatccggatatagttcctcctttcagcaaaaaacccctcaagacccgtttagaggccccaaggggttatgctagttattgctcagcggtggcagcagccaactcagcttcctttcgggctttgttagcagccggatccttagcacagcagtttgcacacgcacagcagcgcaataatcgcaataataatgccggtaatgccaatgcccgccggaatccactggcgccagccggtccaccagttatcatcgttatcctggttcggcagcgggttatcaataaaatcatgaataatctggttaattttatcggtaatgtttttggtccaatcatgcggttcaatgcagcaatccgggcccagaatgcggcaggtgccgccccagcggcgcagcagaaaatcaatcgctttgcggttcagaatggtataggtgcgcagttcggtggtcgcgcgcagaaacagctgcagcgcctgggtggtttcgttcgccagctggcgcaggccgcacaccagcgcgttctggttatgcatcaggccttcggtataaatgccttccgcgcccgggccaaaatacggaatccacgcaatgcccgccgcgttatgctgttcctgcgcggtccaataatgcaggttcgggttgcatttgccggtcgctttggtgttggtctggcggcggctgcgtttgcgcaggcccaggctgcccagaatgccggtcacggtgctggtaatcaggccgttgctggtgctttcctgcggcagcacggttttcaccgcggtggtaatgttttccggggtggtcagggtcggcgcttcggtggtcgggcccgggctgctgcccggcggggtggtcggttcttcggtcgccatcacgctcgggccgctggtatgggtggtcggggtctgcgcttccgggctcggcgcggtggtcgggctgctgctcggaatctggctgctgctcgggcgaatgccaatggtgctaatctgcatatggttgccgttggtgccaataatggtcgccgcggtttcggtaatggtttcctgggtgttcacgcccacgcggcggccttcggtgctgttctggctcggcagggtggtttcgccttccggaatatgcagcggcaccatgcccgggctgtttttcggcaccagatcgctatatttgcgggtcgcgcgatcgctaatgcggcctttggtaatgcggctgctcgccgcatcatcatcttcggtttcgttcaggctcagcgcttcaaagctcagttcttcgccgcgcagctgttcgctcaggttttttttgttttcccaaaacgcccattcgccaatatccgcgttaatgttcgcatccagggtccaaatcaggcggccggtggtgttgctcagctgctgatgcagatgaatggtatcgttcagctgaaacagaaactgcggggtatgcgggcgatccaggcgcacaaaggtgttgttatcaattttaaacagggtggtgctatgctgcgcgccaaagttttcaatttcatattccagatagctggtcgcataatagctgctggtgttttcggtatagttcaccgcttcgcgaatcggcgggctctgcagaaaggtttctttcggtttcgccagaatcagaaacgcaatcacgccttccgcaaagttcacgccgcgataaatcacggtgctcgccaggcgatcatacagaaaaaacgcgccatctttatgaaacgcataatcgcccgggcacgggccggtgccctgcgctttatgcacatagcggcagcgcggaaagccgcgcacgccatccggcggcggcggcaggcattcgctgccatccggttttttaatttccaggttatagcagttttccgcccattcgcccgcttcatagctcaccactttcggcggcacgccgctgcgaaagccccagcgtttggtcgcgctcggaatatcggtgctcacgccgctgccttccaggttcaggcccacgcttttcagctgatcggtgctcgccagatgatctttgcacaccagctgatcaatttcggtcacttccagggtgctgttggtcaccacgcccagcggcatgctaaacgctttctgaaacagaataatcacccacacaaaaaagctgcttttgcgaaatttatcgcgcggcagctgcagcaggctcaggccgcccatctcgaggcctccaccgcctccgcgtttatcaatcgccaccgccagcaccatatgtgccttggctttgacgtatcgcccaggcgcgggttcgattgcgcgatagcgcgcattgccatagttggcgggcgcggcgcgtttcgcgccggcctgcccggccagccatgcggtccagtccggccaccagctgccgtgatgctcgatggcgccggccagccattgctgcggcgactccggcagcgcatcgttagtccagtggctgcgcttgttcttggccggcgggttgatcacaccggcgatatggcccgacgcacccagcacgaagcgcagcttgttcgccagcagcgcggtcgaggcataggccgcggtccacggcacgatatggtcttcgcgcgagccgtagatataggtcggcacgtcgatgctggccaggtccaccggcacgccgcacacggtcagcttgcccggtaccttgagctcgttctgcaggtaggtgtggcgcaggtaccagcagtaccacggccccggcaggttggtggcgtcgccgttccagaacagcaggtcgaacggcaccggcgtgttgcccttcaggtagttgtcgaccacgtagttccacaccaggtcgttcgggcgcaagaacgagaaggtattggccagctcaaggccgcgcagcagcgcgcacggcgcgccggcgccgccgcccagcgtggcctcgcgcaactgcacatggccctcgtcgacaaagacgtcgaggatgcccgtgtcggcaaagtccagcagcgtggtcagcagcgtgacgctggcggccgggtgctcgccgcgcgcggccagcaccgccagcgcggtcgagacaatggtgccgcccacgcagaagccgagcacgttgatcttgtcctggccgctgatgtcgcgcgcgacttcgatggcgcggatggccgcgtgctcgatgtagtcgtcccaggtgctgccggccatgctggcgtccggattgcgccacgacaccagaaacaccgtatgtccctgctccaccacatggcgcaccagcgagctctccggctgcaggtccaggatgtagtacttgttgatgcacggcggcaccatcagcagcgggcgcgcgtgcaccttgtcggtcagcggcttgtactgcaacagctggaagtactcgttctcgaagaccacggcgccttcggtcaccgcgacattgcggccgacctcaaacgcgctctcgtcggtctgcgagatcttgccgcgtgtcaggtcttccatcatgttgcgcacgccggcacgcagcgattcgccgcccgactcgatcagcaggcgctgcgcctcgggattggtggcaaggaagttggcgggcgacatcgcatcgacccattgcgagatcgcgaagcggatgcgctggcgggtcttggcatcggcctcgacggcatcggccagctcggtcaaggcgcgcgcattgagcaggtagaacgcggcagcgaagcgatatgggaggttggtgcgccatgcgtcgccggcgaagcgccggtcgtgcagcggaccggtggcctcggccttgccctcggccatggcctgccacagcgctgagaagtccttcatgtagcgctgctggatatcacccagctgcgccggcgcgatcttgacgcctgccagcgcatccaggcccggaatgccggacgcggccgcgtggccgttgccttcagtgccctgccactggcgggaccattccagccatgtggctggatcgaatggccccggcgtgaccttgaatggttgggacttgccttcctgcgtggaagctgccgcgcctttgccggtcgcactagtaaacacaaatttgcaaatgcaaaacagcgcaatcaccgcaataatcacgccggtcacgccaatgcccgccggaatccactggcgccagccggtccaccagttatcgttatcgccctgatccggcagggttttatccacaaaatcatgaataatctgatcaattttatcggtaatgtttttggtccaatcatgcggttcaatgcagcaatccgggcccagaatatggcaggtgccgccccagcgctgcagcagaaaatcaatcgctttgcggttcagaatgctaaaggtgcgcagttcggtggtcgcgcgcagaaacagctgcagcgcctgggtggtttcgttcgccagctggcgcaggccgcaaatcaggccatcctggttatgcatcaggccttcaatataaatgccttccgccgccgggccaaaatacggaatccacgccaggccaatcgccgcgccttcatcctgggtggtccaataatgcaggttcgggttgcatttcggctgcgcgttcacaatcgcttcgcggcgggtgcggcggccgccggtaatcaggcccgccacgcccgcaatggtgttggtaatcaggcccagtttgccgctgctcgcgctttcttcgccggtatcctgatgatgggtgttgttgttgcccgcggtttcgctatggttctgcgggctggtggtggtcgccggatccagaaaatcggtgcttttgctggtgttggtgttttccgctttcggcgggcccgccgcggtggtcgcgctcggggtatcgctcgcggtgctatcgttatcggtgcggcgatgatgctgttccacctgggtcgcttcgctaatatccagtttatacaccggggtgttatgggtgctgttatccgggcccggtttggtggtcaggctctgcgggctggtgctaatggtcgccagggtggtcagatggctcaccgccgcttcgcggccctggctatgcacctgcaccatcgcgctgctgttttcgctcgccataattttatgatcttcggtggtggtgttggtgcccggatcgctgctggtgcgcgccgggctctggccgctaatgtttttcgcgccgttgctcaccacggtaaagctcagttcttcgctgcgaattttgcgggtcaggttttttttggtttcccaaaacgcccattcgccaatggtggtatcaatttccgggttcactttccaaatcagtttgccggtggtgttgctgcgtttgccgctggtataaatggtttcgttcagctgcagcagaaactgcggggtaaagcggctttccagctgcacataggtcaggttatccacttcaaacagatattcggtttcgttggtgccaaagccggtcgcctgatagcgaatggtggtgctataatagccgctgctcggatcttcggtcgcgttcaccggttcgcgcagcggatggctgctaaaaaaatcttttttcgcctgcggcagaatcagaaacgccaccacgccttccgcaaaggtggtgccgcgataaatcacggtgctcgccaggcgatcatacagaaaaaacgcgccttctttatgaaacgcaaaatcgcccgcgcacgggccggtgccgctcactttatgcacatagcggcagcgcggaaagccgcgaatgccatccggcgccgccggcaggcattcgctgccatccggttttttaatttccaggttatagcagttttccgcccattcgcccgcttcatagttcaccactttcggcggcacgccgctgcgaaagccccagcgtttggtcgcgctcggcacatcggtcgccacgccgttgccttccaggttcaggcccacgctgcgcagctggttggtgctgctcagtttatcgcggcacaccagtttatccacatcgctcacctgcagggtgctgttatgaatcacgcccagcggaatgctaaaggtgcgctgaaacagaataatcacccacagaaaaaagctggtgcgtttaaagcgatcgcgcggcagctgcagaatgccggtcacgcccatactagaatctcctatattctagagggaaaccgttgtggtctccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 50 - protein, 1960 aa, Artificial Sequence
  Description of Artificial Sequence Synthetic ZEBOVGP-PhaC-SEBOVGP fusion polypeptide encoded by pET-14b-ZEBOVGP-PhaC-SEBOVGP
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVFTSATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKAHMVLAVAIDKRGGGGGLEMGGLSLLQLPRDKFRKSSFFVWVIILFQKAFSMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIREAVNYTENTSSYYATSYLEYEIENFGAQHSTTLFKIDNNTFVRLDRPHTPQFLFQLNDTIHLHQQLSNTTGRLIWTLDANINADIGEWAFWENKKNLSEQLRGEELSFEALSLNETEDDDAASSRITKGRISDRATRKYSDLVPKNSPGMVPLHIPEGETTLPSQNSTEGRRVGVNTQETITETAATIIGTNGNHMQISTIGIRPSSSQIPSSSPTTAPSPEAQTPTTHTSGPSVMATEEPTTPPGSSPGPTTEAPTLTTPENITTAVKTVLPQESTSNGLITSTVTGILGSLGLRKRSRRQTNTKATGKCNPNLHYWTAQEQHNAAGIAWIPYFGPGAEGIYTEGLMHNQNALVCGLRQLANETTQALQLFLRATTELRTYTILNRKAIDFLLRRWGGTCRILGPDCCIEPHDWTKNITDKINQIIHDFIDNPLPNQDNDDNWWTGWRQWIPAGIGITGIIIAIIALLCVCKLLC

SEQ ID NO: 51 - DNA, 10468 bp, Artificial Sequence
  Description of Artificial Sequence Synthetic construct - pET-SEBOVGP-PhaC-ZEBOVGP
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatatccggatatagttcctcctttcagcaaaaaacccctcaagacccgtttagaggccccaaggggttatgctagttattgctcagcggtggcagcagccaactcagcttcctttcgggctttgttagcagccggatccttaaaacacaaatttgcaaatgcaaaacagcgcaatcaccgcaataatcacgccggtcacgccaatgcccgccggaatccactggcgccagccggtccaccagttatcgttatcgccctgatccggcagggttttatccacaaaatcatgaataatctgatcaattttatcggtaatgtttttggtccaatcatgcggttcaatgcagcaatccgggcccagaatatggcaggtgccgccccagcgctgcagcagaaaatcaatcgctttgcggttcagaatgctaaaggtgcgcagttcggtggtcgcgcgcagaaacagctgcagcgcctgggtggtttcgttcgccagctggcgcaggccgcaaatcaggccatcctggttatgcatcaggccttcaatataaatgccttccgccgccgggccaaaatacggaatccacgccaggccaatcgccgcgccttcatcctgggtggtccaataatgcaggttcgggttgcatttcggctgcgcgttcacaatcgcttcgcggcgggtgcggcggccgccggtaatcaggcccgccacgcccgcaatggtgttggtaatcaggcccagtttgccgctgctcgcgctttcttcgccggtatcctgatgatgggtgttgttgttgcccgcggtttcgctatggttctgcgggctggtggtggtcgccggatccagaaaatcggtgcttttgctggtgttggtgttttccgctttcggcgggcccgccgcggtggtcgcgctcggggtatcgctcgcggtgctatcgttatcggtgcggcgatgatgctgttccacctgggtcgcttcgctaatatccagtttatacaccggggtgttatgggtgctgttatccgggcccggtttggtggtcaggctctgcgggctggtgctaatggtcgccagggtggtcagatggctcaccgccgcttcgcggccctggctatgcacctgcaccatcgcgctgctgttttcgctcgccataattttatgatcttcggtggtggtgttggtgcccggatcgctgctggtgcgcgccgggctctggccgctaatgtttttcgcgccgttgctcaccacggtaaagctcagttcttcgctgcgaattttgcgggtcaggttttttttggtttcccaaaacgcccattcgccaatggtggtatcaatttccgggttcactttccaaatcagtttgccggtggtgttgctgcgtttgccgctggtataaatggtttcgttcagctgcagcagaaactgcggggtaaagcggctttccagctgcacataggtcaggttatccacttcaaacagatattcggtttcgttggtgccaaagccggtcgcctgatagcgaatggtggtgctataatagccgctgctcggatcttcggtcgcgttcaccggttcgcgcagcggatggctgctaaaaaaatcttttttcgcctgcggcagaatcagaaacgccaccacgccttccgcaaaggtggtgccgcgataaatcacggtgctcgccaggcgatcatacagaaaaaacgcgccttctttatgaaacgcaaaatcgcccgcgcacgggccggtgccgctcactttatgcacatagcggcagcgcggaaagccgcgaatgccatccggcgccgccggcaggcattcgctgccatccggttttttaatttccaggttatagcagttttccgcccattcgcccgcttcatagttcaccactttcggcggcacgccgctgcgaaagccccagcgtttggtcgcgctcggcacatcggtcgccacgccgttgccttccaggttcaggcccacgctgcgcagctggttggtgctgctcagtttatcgcggcacaccagtttatccacatcgctcacctgcagggtgctgttatgaatcacgcccagcggaatgctaaaggtgcgctgaaacagaataatcacccacagaaaaaagctggtgcgtttaaagcgatcgcgcggcagctgcagaatgccggtcacgcccatctcgaggcctccaccgcctccgcgtttatcaatcgccaccgccagcaccatatgtgccttggctttgacgtatcgcccaggcgcgggttcgattgcgcgatagcgcgcattgccatagttggcgggcgcggcgcgtttcgcgccggcctgcccggccagccatgcggtccagtccggccaccagctgccgtgatgctcgatggcgccggccagccattgctgcggcgactccggcagcgcatcgttagtccagtggctgcgcttgttcttggccggcgggttgatcacaccggcgatatggcccgacgcacccagcacgaagcgcagcttgttcgccagcagcgcggtcgaggcataggccgcggtccacggcacgatatggtcttcgcgcgagccgtagatataggtcggcacgtcgatgctggccaggtccaccggcacgccgcacacggtcagcttgcccggtaccttgagctcgttctgcaggtaggtgtggcgcaggtaccagcagtaccacggccccggcaggttggtggcgtcgccgttccagaacagcaggtcgaacggcaccggcgtgttgcccttcaggtagttgtcgaccacgtagttccacaccaggtcgttcgggcgcaagaacgagaaggtattggccagctcaaggccgcgcagcagcgcgcacggcgcgccggcgccgccgcccagcgtggcctcgcgcaactgcacatggccctcgtcgacaaagacgtcgaggatgcccgtgtcggcaaagtccagcagcgtggtcagcagcgtgacgctggcggccgggtgctcgccgcgcgcggccagcaccgccagcgcggtcgagacaatggtgccgcccacgcagaagccgagcacgttgatcttgtcctggccgctgatgtcgcgcgcgacttcgatggcgcggatggccgcgtgctcgatgtagtcgtcccaggtgctgccggccatgctggcgtccggattgcgccacgacaccagaaacaccgtatgtccctgctccaccacatggcgcaccagcgagctctccggctgcaggtccaggatgtagtacttgttgatgcacggcggcaccatcagcagcgggcgcgcgtgcaccttgtcggtcagcggcttgtactgcaacagctggaagtactcgttctcgaagaccacggcgccttcggtcaccgcgacattgcggccgacctcaaacgcgctctcgtcggtctgcgagatcttgccgcgtgtcaggtcttccatcatgttgcgcacgccggcacgcagcgattcgccgcccgactcgatcagcaggcgctgcgcctcgggattggtggcaaggaagttggcgggcgacatcgcatcgacccattgcgagatcgcgaagcggatgcgctggcgggtcttggcatcggcctcgacggcatcggccagctcggtcaaggcgcgcgcattgagcaggtagaacgcggcagcgaagcgatatgggaggttggtgcgccatgcgtcgccggcgaagcgccggtcgtgcagcggaccggtggcctcggccttgccctcggccatggcctgccacagcgctgagaagtccttcatgtagcgctgctggatatcacccagctgcgccggcgcgatcttgacgcctgccagcgcatccaggcccggaatgccggacgcggccgcgtggccgttgccttcagtgccctgccactggcgggaccattccagccatgtggctggatcgaatggccccggcgtgaccttgaatggttgggacttgccttcctgcgtggaagctgccgcgcctttgccggtcgcactagtgcacagcagtttgcacacgcacagcagcgcaataatcgcaataataatgccggtaatgccaatgcccgccggaatccactggcgccagccggtccaccagttatcatcgttatcctggttcggcagcgggttatcaataaaatcatgaataatctggttaattttatcggtaatgtttttggtccaatcatgcggttcaatgcagcaatccgggcccagaatgcggcaggtgccgccccagcggcgcagcagaaaatcaatcgctttgcggttcagaatggtataggtgcgcagttcggtggtcgcgcgcagaaacagctgcagcgcctgggtggtttcgttcgccagctggcgcaggccgcacaccagcgcgttctggttatgcatcaggccttcggtataaatgccttccgcgcccgggccaaaatacggaatccacgcaatgcccgccgcgttatgctgttcctgcgcggtccaataatgcaggttcgggttgcatttgccggtcgctttggtgttggtctggcggcggctgcgtttgcgcaggcccaggctgcccagaatgccggtcacggtgctggtaatcaggccgttgctggtgctttcctgcggcagcacggttttcaccgcggtggtaatgttttccggggtggtcagggtcggcgcttcggtggtcgggcccgggctgctgcccggcggggtggtcggttcttcggtcgccatcacgctcgggccgctggtatgggtggtcggggtctgcgcttccgggctcggcgcggtggtcgggctgctgctcggaatctggctgctgctcgggcgaatgccaatggtgctaatctgcatatggttgccgttggtgccaataatggtcgccgcggtttcggtaatggtttcctgggtgttcacgcccacgcggcggccttcggtgctgttctggctcggcagggtggtttcgccttccggaatatgcagcggcaccatgcccgggctgtttttcggcaccagatcgctatatttgcgggtcgcgcgatcgctaatgcggcctttggtaatgcggctgctcgccgcatcatcatcttcggtttcgttcaggctcagcgcttcaaagctcagttcttcgccgcgcagctgttcgctcaggttttttttgttttcccaaaacgcccattcgccaatatccgcgttaatgttcgcatccagggtccaaatcaggcggccggtggtgttgctcagctgctgatgcagatgaatggtatcgttcagctgaaacagaaactgcggggtatgcgggcgatccaggcgcacaaaggtgttgttatcaattttaaacagggtggtgctatgctgcgcgccaaagttttcaatttcatattccagatagctggtcgcataatagctgctggtgttttcggtatagttcaccgcttcgcgaatcggcgggctctgcagaaaggtttctttcggtttcgccagaatcagaaacgcaatcacgccttccgcaaagttcacgccgcgataaatcacggtgctcgccaggcgatcatacagaaaaaacgcgccatctttatgaaacgcataatcgcccgggcacgggccggtgccctgcgctttatgcacatagcggcagcgcggaaagccgcgcacgccatccggcggcggcggcaggcattcgctgccatccggttttttaatttccaggttatagcagttttccgcccattcgcccgcttcatagctcaccactttcggcggcacgccgctgcgaaagccccagcgtttggtcgcgctcggaatatcggtgctcacgccgctgccttccaggttcaggcccacgcttttcagctgatcggtgctcgccagatgatctttgcacaccagctgatcaatttcggtcacttccagggtgctgttggtcaccacgcccagcggcatgctaaacgctttctgaaacagaataatcacccacacaaaaaagctgcttttgcgaaatttatcgcgcggcagctgcagcaggctcaggccgcccatactagaatctcctatattctagagggaaaccgttgtggtctccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 52 - protein, 1960 aa, Artificial Sequence
  Description of Artificial Sequence Synthetic SEBOVGP-PhaC-ZEBOVGO fusion polypeptide encoded by pET-14b-SEBOVGP-PhaC-ZEBOVGO
MGGLSLLQLPRDKFRKSSFFVWVIILFQKAFSMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIREAVNYTENTSSYYATSYLEYEIENFGAQHSTTLFKIDNNTFVRLDRPHTPQFLFQLNDTIHLHQQLSNTTGRLIWTLDANINADIGEWAFWENKKNLSEQLRGEELSFEALSLNETEDDDAASSRITKGRISDRATRKYSDLVPKNSPGMVPLHIPEGETTLPSQNSTEGRRVGVNTQETITETAATIIGTNGNHMQISTIGIRPSSSQIPSSSPTTAPSPEAQTPTTHTSGPSVMATEEPTTPPGSSPGPTTEAPTLTTPENITTAVKTVLPQESTSNGLITSTVTGILGSLGLRKRSRRQTNTKATGKCNPNLHYWTAQEQHNAAGIAWIPYFGPGAEGIYTEGLMHNQNALVCGLRQLANETTQALQLFLRATTELRTYTILNRKAIDFLLRRWGGTCRILGPDCCIEPHDWTKNITDKINQIIHDFIDNPLPNQDNDDNWWTGWRQWIPAGIGITGIIIAIIALLCVCKLLCTSATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKAHMVLAVAIDKRGGGGGLEMGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 53 - DNA, 7912 bp, Artificial Sequence
  Description of Artificial Sequence Synthetic construct - pET-14b-PhaC-WNVE
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataaggagatactagtatggcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcacatatggtgctggcggtggcgattgataaacgcggaggcggtggaggcctcgagtttaactgcctgggcatgagcaaccgcgattttctggaaggcgtgagcggcgcgacctgggtggatctggtgctggaaggcgatagctgcgtgaccattatgagcaaagataaaccgaccattgatgtgaaaatgatgaacatggaagcggcgaacctggcggaagtgcgcagctattgctatctggcgaccgtgagcgatctgagcaccaaagcggcgtgcccgaccatgggcgaagcgcataacgataaacgcgcggatccggcgtttgtgtgccgccagggcgtggtggatcgcggctggggcaacggctgcggcctgtttggcaaaggcagcattgatacctgcgcgaaatttgcgtgcagcaccaaagcgattggccgcaccattctgaaagaaaacattaaatatgaagtggcgatttttgtgcatggcccgaccaccgtggaaagccatggcaactatagcacccaggtgggcgcgacccaggcgggccgcctgagcattaccccggcggcgccgagctataccctgaaactgggcgaatatggcgaagtgaccgtggattgcgaaccgcgcagcggcattgataccaacgcgtattatgtgatgaccgtgggcaccaaaacctttctggtgcatcgcgaatggtttatggatctgaacctgccgtggagcagcgcgggcagcaccgtgtggcgcaaccgcgaaaccctgatggaatttgaagaaccgcatgcgaccaaacagagcgtgattgcgctgggcagccaggaaggcgcgctgcatcaggcgctggcgggcgcgattccggtggaatttagcagcaacaccgtgaaactgaccagcggccatctgaaatgccgcgtgaaaatggaaaaactgcagctgaaaggcaccacctatggcgtgtgcagcaaagcgtttaaatttctgggcaccccggcggataccggccatggcaccgtggtgctggaactgcagtataccggcaccgatggcccgtgcaaagtgccgattagcagcgtggcgagcctgaacgatctgaccccggtgggccgcctggtgaccgtgaacccgtttgtgagcgtggcgaccgcgaacgcgaaagtgctgattgaactggaaccgccgtttggcgatagctatattgtggtgggccgcggcgaacagcagattaaccatcattggcataaaagcggcagcagcattggcaaagcgtttaccaccaccctgaaaggcgcgcagcgcctggcggcgctgggcgataccgcgtgggattttggcagcgtgggcggcgtgtttaccagcgtgggcaaagcggtgcatcaggtgtttggcggcgcgtttcgcagcctgtttggcggcatgagctggattacccagggcctgctgggcgcgctgctgctgtggatgggcattaacgcgcgcgatcgcagcattgcgctgacctttctggcggtgggcggcgtgctgctgtttctgagcgtgaacgtgcatgcgtgaggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 54 - protein, 1108 aa, Artificial Sequence
  Description of Artificial Sequence Synthetic PhaC-WNVE fusion polypeptide encoded by pET-14b-PhaC-WNVE
MATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKAHMVLAVAIDKRGGGGGLEFNCLGMSNRDFLEGVSGATWVDLVLEGDSCVTIMSKDKPTIDVKMMNMEAANLAEVRSYCYLATVSDLSTKAACPTMGEAHNDKRADPAFVCRQGVVDRGWGNGCGLFGKGSIDTCAKFACSTKAIGRTILKENIKYEVAIFVHGPTTVESHGNYSTQVGATQAGRLSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSSIGKAFTTTLKGAQRLAALGDTAWDFGSVGGVFTSVGKAVHQVFGGAFRSLFGGMSWITQGLLGALLLWMGINARDRSIALTFLAVGGVLLFLSVNVHA

SEQ ID NO: 55 - DNA, 64 bp, Artificial Sequence
  Description of Artificial Sequence Synthetic primer
cgcctttgccggtcgcacaacaacaacaacaacacatactagtatctccttatttctagaggga

SEQ ID NO: 56 - DNA, 45 bp, Artificial Sequence
  Description of Artificial Sequence Synthetic primer
gatacgtcaaagccaaggcatgtagggatccggctgctaacaaag

SEQ ID NO: 57 - protein, 6 aa, Artificial Sequence
  Description of Artificial Sequence Synthetic peptide
CCCCCC

(19) United States
(12) Patent Application Publication
(10) Pub. No.: US 2018/0015156 A1
(43) Pub. Date: Jan. 18, 2018

(54) POLYMER PARTICLES AND USES THEREOF

(71) Applicant: Polybatics Limited, Palmerston North (NZ)

(72) Inventors: Bernd Helmut Adam REHM, Palmerston North (NZ); Natalie Anne PARLANE, Palmerston North (NZ); David Neil WEDLOCK, Manawatu-Wanganui (NZ); Bryce Malcolm BUDDLE, Palmerston North (NZ)

(21) Appl. No.: 15/453,073

(22) Filed: Mar. 8, 2017

Related U.S. Application Data
(63) Continuation of application No. 14/927,321, filed on Oct. 29, 2015, now abandoned.
Application No. 14/927,321 is a continuation of application No. 13/387,739, filed on Mar. 14, 2012, now abandoned.
(60) Provisional application No. 61/229,318, filed on Jul. 29, 2009.
(60) Provisional application No. 61/229,348, filed on Jul. 29, 2009.

(51) Int. Cl. A61K 39/04; A61K 39/02; A61K 39/07; A61K 39/12; A61K 39/145; A61K 38/02; A61K 39/29; A61K 39/39; A61K 39/095; A61K 39/00

(52) U.S. Cl. CPC A61K 39/04; A61K 39/39; A61K 39/0208; A61K 39/07; A61K 39/095; A61K 39/098; A61K 39/12; A61K 39/145; A61K 38/02; A61K 39/29; A61K 2039/55555; A61K 2039/55566; A61K 2039/70; C12N 2760/14134; C12N 2760/16134; C12N 2770/24134; C12N 2770/24234

(57) ABSTRACT

The present invention relates to polymer particles and uses thereof. In particular the present invention relates to functionalised polymer particles, processes of production and uses thereof in eliciting a cell-mediated immune response and in the treatment or prevention of diseases or conditions including those caused by intracellular pathogens.

### TECHNICAL FIELD

The present invention relates to recombinant proteins and related constructs and methods, and to polymer particles and uses thereof. In particular the present invention relates to functionalised polymer particles, processes of production and uses thereof in eliciting an immune response and in the treatment or prevention of diseases or conditions including those caused by intracellular or extracellular pathogens.

### BACKGROUND

The following includes information that is useful in understanding the present invention. It is not an admission that any of the information provided herein is prior art, or relevant, to the presently described or claimed inventions, or that any publication or document that is specifically or implicitly referenced is prior art.

Pathogens including intracellular and extracellular pathogens are known to cause a number of harmful diseases in humans, including, for example, tuberculosis, hepatitis, influenza, leprosy, listeriosis, typhoid fever, dysentery, plague, pneumonia, typhus, chlamydia, anthrax disease, and meningitis, amongst others. Both the ability to generate a robust cell-mediated immune response and a humoral response, elicited by traditional vaccination strategies, are encompassed herein.

Tuberculosis (Tb), for example, is estimated to kill over 2 million people each year. Current methods for the treatment or prevention of tuberculosis are being challenged by the emergence of multi-drug resistant strains of Mycobacterium tuberculosis bacteria (Anderson, 2007; Mustafa, 2001). The treatment or prophylaxis of Tb is complicated by the inaccessibility of the intracellular bacteria to the host's immune system.

It would be desirable to develop a safe and efficient method for delivering targeted vaccinations that overcomes many of the disadvantages of conventional vaccine delivery systems. Disadvantages include increased cost and a need for repeated administration, frequently due to diminished efficacy over time. Generating an immune response, and particularly a cell-mediated immune response, has also been proposed as a method of treating a variety of other diseases and conditions, including for example, cancer. There is thus a need for vaccine compositions capable of eliciting a robust immune response, and particularly compositions capable of eliciting a cell-mediated immune response or a humoral response or both.

The properties of polyhydroxyalkyl carboxylates, in particular polyhydroxy alkanoates (PHAs) have been investigated for their application in bioplastics, in addition to their use as a matrix for the transport of drugs and other active agents in medical, pharmaceutical and food industry applications. Through bioengineering of the PHA molecule, the composition and expression of the PHA molecule can be manipulated to suit a particular function.

It is an object of the present invention to provide polymer particles for use in the treatment or prevention of various diseases and conditions, including, for example, by immunisation or vaccination, to provide methods and compositions for eliciting an effective immune response in subjects in need thereof, or to at least provide the public with a useful choice.

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or nonlimiting embodiments identified in this Brief Summary, which is included for purposes of illustration only and not restriction.

Disclosed herein are methods for producing polymer particles, the method comprising providing a host cell comprising at least one expression construct, the at least one expression construct comprising:
at least one nucleic acid sequence encoding a particle-forming protein; and either
at least one nucleic acid sequence encoding an antigen capable of eliciting an immune response; or
at least one nucleic acid sequence encoding a binding domain capable of binding an antigen capable of eliciting an immune response;
maintaining the host cell under conditions suitable for expression of the expression construct; and

separating polymer particles from host cells.

In one embodiment the method comprises providing a host cell comprising at least one expression construct, the at least one expression construct comprising:
at least one nucleic acid sequence encoding a particle-forming protein; and either
at least one nucleic acid sequence encoding an antigen capable of eliciting a cell-mediated immune response, for example; or
at least one nucleic acid sequence encoding a binding domain capable of binding an antigen capable of eliciting a cell-mediated immune response, for example;
maintaining the host cell under conditions suitable for expression of the expression construct; and

separating polymer particles from host cells.

In one embodiment the particle-forming protein is a polymer synthase.

In one embodiment the expression construct is in a high copy number vector.

In one embodiment the at least one nucleic acid sequence encoding a particle-forming protein, is operably linked to a strong promoter.

In one embodiment the strong promoter is a viral promoter or a phage promoter.

In one embodiment the promoter is a phage promoter, for example a T7 phage promoter.

In one embodiment the host cell is maintained in the presence of a substrate of a polymer synthase, preferably a substrate of a polymer synthase when present or a substrate mixture, including monomeric substrate, or a precursor substrate able to be metabolised by the host cell to form a substrate of the particle-forming protein.

In one embodiment the host cell comprises at least two different expression constructs.

In some embodiments in which the host cell comprises at least two different expression constructs, at least one of the expression constructs is selected from the group comprising:
an expression construct comprising a nucleic acid sequence encoding a particle-forming protein, and at least one antigen capable of eliciting an immune response, or
an expression construct comprising a nucleic acid sequence encoding a particle-forming protein, and a binding domain capable of binding at least one antigen capable of eliciting an immune response, including, for example, a cell-mediated immune response, or
an expression construct comprising a nucleic acid sequence encoding a particle-forming protein, and at least one antigen capable of eliciting a cell-mediated immune response, or
an expression construct comprising a nucleic acid sequence encoding a particle-forming protein, and a binding domain capable of binding at least one antigen capable of eliciting a cell-mediated immune response, or
an expression construct comprising a nucleic acid sequence encoding an adjuvant, or
an expression construct comprising a nucleic acid sequence encoding at least one antigen capable of eliciting an immune response, or
an expression construct comprising a nucleic acid sequence encoding at least one antigen capable of eliciting a cell-mediated immune response.

In other embodiments in which the host cell comprises at least two different expression constructs, one of the expression constructs is selected from the group comprising:
an expression construct comprising a nucleic acid sequence encoding a particle-forming protein, or
an expression construct comprising a nucleic acid sequence encoding a particle-size determining protein, or
an expression construct comprising a nucleic acid sequence encoding a polymer regulator.

In other embodiments in which the host cell comprises at least two different expression constructs, one of expression constructs comprises a nucleic acid sequence encoding a particle-forming protein, preferably a polymer synthase, and a binding domain capable of binding at least one antigen capable of eliciting an immune response, for example, a cell-mediated immune response, and at least one expression construct selected from the group comprising:
an expression construct comprising a nucleic acid sequence encoding a particle-forming protein, and at least one antigen capable of eliciting an immune response, or
an expression construct comprising a nucleic acid sequence encoding a particle-forming protein, and a binding domain capable of binding at least one antigen capable of eliciting an immune response, or
an expression construct comprising a nucleic acid sequence encoding a particle-forming protein, and at least one antigen capable of eliciting a cell-mediated immune response, or
an expression construct comprising a nucleic acid sequence encoding a particle-forming protein, and a binding domain capable of binding at least one antigen capable of eliciting a cell-mediated immune response, or
an expression construct comprising a nucleic acid sequence encoding an adjuvant, or
an expression construct comprising a nucleic acid sequence encoding at least one antigen capable of eliciting an immune response, or
an expression construct comprising a nucleic acid sequence encoding at least one antigen capable of eliciting a cell-mediated immune response.

In one embodiment the host cell comprises a mixed population of expression constructs wherein each expression construct comprises a nucleic acid sequence encoding a fusion polypeptide, the fusion polypeptide comprising:
at least one particle-forming protein, and either
at least one antigen capable of eliciting an immune response, or
at least one binding domain capable of binding at least one antigen capable of eliciting an immune response.

In various embodiments, the antigen is an antigen capable of eliciting a cell-mediated immune response.

Another aspect of the present invention relates to an expression construct, the expression construct comprising:
at least one nucleic acid sequence encoding a particle-forming protein; and
at least one nucleic acid sequence encoding an antigen capable of eliciting an immune response.
In one embodiment, the nucleic acid encodes an antigen capable of eliciting a cell-mediated immune response.

Another aspect of the present invention relates to an expression construct, the expression construct comprising:
at least one nucleic acid sequence encoding a particle-forming protein; and
at least one nucleic acid sequence encoding a binding domain capable of binding an antigen capable of eliciting an immune response.

In various embodiments, the antigen is capable of eliciting a cell-mediated immune response, or the binding domain is capable of binding an antigen capable of eliciting a cell-mediated immune response.

In one embodiment the expression construct encodes a fusion polypeptide comprising the particle-forming protein, and the antigen capable of eliciting an immune response.

In one embodiment the expression construct encodes a fusion polypeptide comprising the particle-forming protein, and a binding domain capable of binding an antigen capable of eliciting an immune response.

In one embodiment the at least one nucleic acid sequence encoding the particle-forming protein and the at least one nucleic acid sequence encoding the antigen capable of eliciting an immune response are present as a single open reading frame.

In one embodiment the at least one nucleic acid sequence encoding the particle-forming protein and the at least one nucleic acid sequence encoding the binding domain capable of binding an antigen capable of eliciting an immune response are present as a single open reading frame.

In one embodiment the at least one nucleic acid sequence encoding the particle-forming protein is operably linked to a strong promoter.

In one embodiment the expression construct comprises at least one nucleic acid sequence encoding an additional polypeptide.

In one embodiment, the expression construct comprises:
at least one nucleic acid sequence encoding a fusion polypeptide that comprises a particle-forming protein, and at least one a binding domain capable of binding an antigen capable of eliciting an immune response; and
at least one nucleic acid sequence encoding an additional polypeptide that binds the binding domain capable of binding an antigen capable of eliciting an immune response of the fusion polypeptide.

In one embodiment the additional polypeptide is a fusion polypeptide comprising a particle-forming protein, and at least one antigen capable of eliciting an immune response, such as an antigen capable of eliciting a cell-mediated immune response.

In one embodiment the construct additionally comprises a nucleic acid encoding
i. at least one thiolase, or
ii. at least one reductase, or
iii. both (i) and (ii).

In one embodiment the construct comprises a nucleic acid encoding
i. at least one thiolase,
ii. at least one reductase,
iii. at least one polymer synthase;
iv. at least one antigen capable of eliciting an immune response, or
v. at least one binding domain capable of binding at least one antigen capable of eliciting an immune response,
vi. a fusion protein comprising one or more of i) to v) above,
vii. any combination of i) to vi) above.

In one embodiment the expression construct comprises:
at least one nucleic acid sequence encoding a fusion polypeptide that comprises a particle-forming protein, and at least one antigen capable of eliciting an immune response, for example, a cell-mediated immune response; and
at least one nucleic acid sequence encoding an additional polypeptide that comprises a binding domain capable of binding at least one antigen capable of eliciting an immune response, for example, a cell-mediated immune response.

In one embodiment the expression construct comprises:
at least one nucleic acid sequence encoding a fusion polypeptide that comprises a particle-forming protein, and at least one antigen capable of eliciting a cell-mediated immune response; and
at least one nucleic acid sequence encoding an additional polypeptide that comprises a binding domain capable of binding at least one antigen capable of eliciting a cell-mediated immune response.

In one embodiment the additional polypeptide is a fusion polypeptide comprising a particle-forming protein, and a binding domain capable of binding at least one antigen capable of eliciting an immune response, for example a cell-mediated immune response.

Another aspect of the present invention relates to a vector comprising an expression construct of the invention.

In one embodiment the vector is a high copy number vector.

In one embodiment the vector is a low copy number vector.

Another aspect of the present invention relates to a host cell comprising an expression construct or a vector as defined above.

In one embodiment the host cell comprises an expression construct selected from the group comprising:
an expression construct comprising a nucleic acid sequence encoding a particle-forming protein, and at least one antigen capable of eliciting an immune response, or
an expression construct comprising a nucleic acid sequence encoding a particle-forming protein, and a binding domain capable of binding at least one antigen capable of eliciting an immune response, for example, a cell-mediated immune response, or
an expression construct comprising a nucleic acid sequence encoding a particle-forming protein, and at least one antigen capable of eliciting a cell-mediated immune response, or
an expression construct comprising a nucleic acid sequence encoding a particle-forming protein, and a binding domain capable of binding at least one antigen capable of eliciting a cell-mediated immune response, or
an expression construct comprising a nucleic acid sequence encoding an adjuvant, or
an expression construct comprising a nucleic acid sequence encoding at least one antigen capable of eliciting an immune response, or
an expression construct comprising a nucleic acid sequence encoding at least one antigen capable of eliciting a cell-mediated immune response.

Another aspect of the present invention relates to a polymer particle comprising one or more fusion polypeptides comprising a particle-forming protein fused to at least one antigen capable of eliciting an immune response, for example, a cell-mediated immune response.

Another aspect of the present invention relates to a polymer particle comprising one or more fusion polypeptides comprising a particle-forming protein fused to a binding domain capable of binding at least one antigen capable of eliciting an immune response, for example, a cell-mediated immune response.

In one embodiment the polymer particle comprises two or more different fusion polypeptides.

In one embodiment the polymer particle comprises two or more different fusion polypeptides on the polymer particle surface.

In one embodiment the polymer particle comprises three or more different fusion polypeptides, such as three or more different fusion polypeptides on the polymer particle surface.

In one embodiment the polymer particle comprises two or more different antigens capable of eliciting an immune response, for example a cell-mediated immune response.

In one embodiment the polymer particle comprises binding domains of at least two or more different antigens capable of eliciting an immune response, for example a cell-mediated immune response.

In one embodiment the polymer particle further comprises at least one substance bound to or incorporated into the polymer particle, or a combination thereof.

In one embodiment the substance is an antigen, or an adjuvant, or an immunostimulatory molecule.

In one embodiment the substance is bound by cross-linking.

In one embodiment the at least one polymer particle comprises at least one antigen selected from the group comprising a M. tuberculosis antigen, a hepatitis C antigen, an influenza antigen, a Francisella tularensis antigen, a Brucella abortus antigen, a Neisseria meningitidis antigen, a Bacillus anthracis antigen, a dengue virus antigen, an ebola virus antigen, a West Nile virus antigen, including one of the antigens described herein.

Another aspect of the present invention relates to a polymer particle produced according to a method defined above.

Another aspect of the present invention relates to a composition of polymer particles, wherein the polymer particles comprise one or more fusion polypeptides comprising a particle-forming protein fused to at least one antigen capable of eliciting an immune response, for example a cell-mediated immune response.

Another aspect of the present invention relates to a composition of polymer particles, wherein the polymer particles comprise one or more fusion polypeptides comprising a particle-forming protein fused to a binding domain capable of binding at least one antigen capable of eliciting an immune response, for example a cell-mediated immune response.

Another aspect of the present invention relates to a composition of polymer particles, wherein the polymer particles are produced according to a method defined above.

In various embodiments, the composition is a vaccine composition. In various embodiments the vaccine composition additionally comprises one or more adjuvants or immunostimulatory molecules.

Another aspect of the present invention relates to a diagnostic reagent comprising a composition of polymer particles as defined above.

Another aspect of the present invention relates to a diagnostic kit comprising a composition of polymer particles as defined above.

In one embodiment, the composition comprises an homogenous population of polymer particles.

In one embodiment, the composition comprises a mixed population of polymer particles.

In one embodiment, the composition additionally comprises one or more of the following:
one or more antigens capable of eliciting an immune response, for example a cell-mediated immune response,
one or more binding domains of one or more antigens capable of eliciting an immune response, for example a cell-mediated immune response,
one or more adjuvants, or
one or more immunomodulatory agents or molecules.

Another aspect of the present invention relates to a method of eliciting an immune response in a subject, wherein the method comprises administering to a subject in need thereof at least one polymer particle comprising one or more fusion polypeptides, wherein at least one of the fusion polypeptides comprises a particle-forming protein fused to at least one antigen capable of eliciting an immune response in a subject.

In one embodiment, the immune response is a cell-mediated immune response. In one embodiment, the antigen is an antigen capable of eliciting a cell-mediated immune response.

In one embodiment, the immune response is a humoral immune response. In one embodiment, the antigen is an antigen capable of eliciting a humoral immune response.

Another aspect of the present invention relates to a method of elicting an immune response in a subject, wherein the method comprises administering to a subject in need thereof at least one polymer particle comprising one or more fusion polypeptides, wherein at least one of the fusion polypeptides comprises a particle-forming protein fused to a binding domain capable of binding at least one antigen capable of eliciting an immune response in a subject, wherein the binding domain capable of binding at least one antigen capable of eliciting an immune response is bound to, the subject comprises, or the subject is administered, at least one antigen capable of eliciting an immune response.

In one embodiment, the immune response is a cell-mediated immune response. In one embodiment, the binding domain is capable of binding an antigen capable of eliciting a cell-mediated immune response.

In one embodiment the method relates to a method of immunising a subject against tuberculosis, wherein the method comprises administering to a subject in need thereof at least one polymer particle comprising one or more fusion polypeptides, wherein at least one of the fusion polypeptides comprises a particle-forming protein fused to at least one antigen capable of eliciting a cell-mediated or other immune response.

In one embodiment the method relates to a method of immunising a subject against tuberculosis, wherein the method comprises administering to a subject in need thereof at least one polymer particle comprising one or more fusion polypeptides, wherein at least one of the fusion polypeptides comprises a particle-forming protein fused to at least one binding domain capable of binding at least one antigen capable of eliciting a cell-mediated immune response, wherein the binding domain capable of binding at least one antigen capable of eliciting a cell-mediated or other immune response is bound to, the subject comprises, or the subject is administered, at least one antigen capable of eliciting a cell-mediated or other immune response.

In one embodiment the at least one polymer particle is present in a composition comprising at least one antigen capable of eliciting an immune response in a subject, such as a composition comprising at least one antigen capable of eliciting a cell-mediated or other immune response in a subject.

In one embodiment the invention relates to a method of eliciting an immune response in a subject infected with tuberculosis, wherein the method comprises administering to a subject in need thereof a polymer particle comprising a particle-forming protein, preferably a polymer synthase, for example, fused to a M. tuberculosis antigen binding domain, for example.

In one embodiment, the M. tuberculosis antigen binding domain binds to an endogenous M. tuberculosis antigen, for example.

Another aspect of the present invention relates to a polymer particle for eliciting an immune response in a subject, for example a cell-mediated immune response, wherein the polymer particle comprises one or more fusion polypeptides, wherein at least one of the fusion polypeptides comprises a particle-forming protein, preferably a polymer synthase, fused to at least one antigen capable of eliciting an immune response in a subject.

In one embodiment, the immune response is a cell-mediated immune response. In one embodiment, the antigen is an antigen capable of eliciting a cell-mediated immune response.

In one embodiment, the immune response is a humoral immune response. In one embodiment, the antigen is an antigen capable of eliciting a humoral immune response.

Another aspect of the present invention relates to a polymer particle for eliciting an immune response in a subject in need thereof, wherein the at least one polymer particle comprises one or more fusion polypeptides, wherein at least one of the fusion polypeptides comprises a particle-forming protein fused to a binding domain capable of binding at least one antigen capable of eliciting an immune response in a subject, wherein the binding domain capable of binding at least one antigen capable of eliciting an immune response is bound to, the subject comprises, or the subject is administered, at least one antigen capable of eliciting an immune response.

In one embodiment, the immune response is a cell-mediated immune response. In one embodiment, the binding domain is capable of binding at least one antigen capable of eliciting a cell-mediated immune response. In one embodiment, the immune response is a humoral immune response. In one embodiment, the antigen is an antigen capable of eliciting a humoral immune response.

In one embodiment the at least one polymer particle is present in a composition comprising at least one antigen capable of eliciting an immune response, for example a cell-mediated immune response.

In one embodiment the at least one polymer particle is present in a composition comprising at least one M. tuberculosis antigen, for example.

In one embodiment, by way of example, the at least one polymer particle is present in a composition comprising at least one antigen selected from the group comprising a M. tuberculosis antigen, a hepatitis C antigen, an influenza antigen, a Francisella tularensis antigen, a Brucella abortus antigen, a Neisseria meningitidis antigen, a Bacillus anthracis antigen, a dengue virus antigen, an ebola virus antigen, a West Nile virus antigen, including one of the antigens described herein, for example.

In one embodiment the subject is infected with an intracellular pathogen or is at risk of being infected with an intracellular pathogen, for example. In another embodiment the subject is infected or is at risk of being infected with a pathogen having a predominantly intracellular life-cycle, for example.

In various embodiments the subject is infected with hepatitis, influenza or tuberculosis.

In another embodiment the subject has been immunised against an intracellular pathogen, for example. For example, the subject has been vaccinated with Bacillus Calmette-Guerin (BCG).

In one embodiment the subject is infected with an extracellular pathogen or is at risk of being infected with an extracellular pathogen, for example. In another embodiment the subject is infected or is at risk of being infected with a pathogen having a predominantly extracellular life-cycle, for example.

Another aspect of the present invention relates to a polymer particle for eliciting an immune response in a subject infected with or immunised against an intracellular pathogen, wherein the at least one polymer particle comprises a particle-forming protein, preferably a polymer synthase, fused to a binding domain capable of binding at least one antigen capable of eliciting an immune response.

The use of a polymer particle as described above in the preparation of a medicament for immunising a subject against an intracellular pathogen, or for eliciting an immune response in a subject including a subject infected with or immunised against an intracellular pathogen, is also contemplated.

Another aspect of the present invention relates to a polymer particle for eliciting an immune response in a subject infected with or immunised against an extracellular pathogen, for example, wherein the at least one polymer particle comprises a particle-forming protein, preferably a polymer synthase, fused to a binding domain capable of binding at least one antigen capable of eliciting an immune response.

The use of a polymer particle as described above in the preparation of a medicament for immunising a subject against an extracellular pathogen, for example, or for eliciting an immune response in a subject including a subject infected with or immunised against an extracellular pathogen, for example, is also contemplated.

The invention further provides a polymer particle as described herein for vaccination of a subject in need thereof. The use of a polymer particle as described herein in the preparation of a medicament for vaccinating a subject in need thereof is thus contemplated.

Another aspect of the present invention relates to a method of diagnosing infection from a pathogen, wherein the method comprises administering to a subject at least one polymer particle of the invention and detecting a response indicative of the presence of the pathogen.

In one embodiment, the pathogen is an intracellular pathogen. In another embodiment the pathogen is an extracellular pathogen.

In one embodiment the response indicative of the presence of the pathogen, such as an intracellular pathogen, is a delayed-type hypersensitivity response.

Another aspect of the present invention relates to a method of diagnosing infection from an pathogen, wherein the method comprises contacting a sample obtained from the subject with a polymer particle of the invention and detecting a response indicative of the presence of the pathogen.

Again, in certain embodiments the pathogen is an intracellular pathogen, an extracellular pathogen, a pathogen having a predominantly intracellular life-cycle, for example, or a pathogen having a predominantly extracellular life-cycle, for example.

In one embodiment, the response indicative of the presence of the pathogen is a detecting the presence of an antibody to the pathogen in said sample.

In one embodiment, the response indicative of the presence of the pathogen is a detecting the presence of an immune cell responsive to the pathogen in said sample.

In one embodiment the detection of the presence of antibodies to the pathogen is by immunoassay.

In one embodiment the detection of the presence of antibodies to the pathogen is by ELISA, radioimmunoassay-assay, or Western Blot.

In one embodiment the response indicative of the presence of the pathogen is a detecting the presence of an immune cell responsive to the pathogen in said sample.

Another aspect of the present invention provides a method for producing polymer particles, the method comprising:
providing a host cell comprising at least one expression construct, the at least one expression construct comprising:
   at least one nucleic acid sequence encoding a particle-forming protein; and
   at least one nucleic acid sequence encoding a M. tuberculosis antigen or a M. tuberculosis antigen binding domain;
maintaining the host cell under conditions suitable for expression of the expression construct and for formation of polymer particles; and
separating the polymer particles from the host cells.

In some embodiments in which the host cell comprises at least two different expression constructs, at least one of the expression constructs is selected from the group comprising:
an expression construct comprising a nucleic acid sequence encoding a particle-forming protein and at least one M. tuberculosis antigen, or
an expression construct comprising a nucleic acid sequence encoding a particle-forming protein and at least one M. tuberculosis antigen binding domain, or
an expression construct comprising a nucleic acid sequence encoding an adjuvant, or
an expression construct comprising a nucleic acid sequence encoding at least one M. tuberculosis antigen.

In other embodiments in which the host cell comprises at least two different expression constructs, one of the expression constructs comprises a nucleic acid sequence encoding a particle-forming protein and at least one M. tuberculosis antigen binding domain, and at least one expression construct selected from the group comprising:
an expression construct comprising a nucleic acid sequence encoding a particle-forming protein and at least one M. tuberculosis antigen, or
an expression construct comprising a nucleic acid sequence encoding a particle-forming protein and at least one M. tuberculosis antigen binding domain, or
an expression construct comprising a nucleic acid sequence encoding an adjuvant, or
an expression construct comprising a nucleic acid sequence encoding at least one M. tuberculosis antigen.

In one embodiment the host cell comprises a mixed population of expression constructs wherein each expression construct comprises a nucleic acid sequence encoding a fusion polypeptide, the fusion polypeptide comprising:
at least one particle-forming protein and
at least one M. tuberculosis antigen or at least one M. tuberculosis antigen binding domain.

Another aspect of the present invention relates to an expression construct, the expression construct comprising:
at least one nucleic acid sequence encoding a particle-forming protein; and
at least one nucleic acid sequence encoding a M. tuberculosis antigen.

Another aspect of the present invention relates to an expression construct, the expression construct comprising:
at least one nucleic acid sequence encoding a particle-forming protein; and
at least one nucleic acid sequence encoding a M. tuberculosis antigen binding domain.

In one embodiment the expression construct encodes a fusion polypeptide comprising the particle-forming protein and the M. tuberculosis antigen.

In one embodiment the expression construct encodes a fusion polypeptide comprising the particle-forming protein and the M. tuberculosis antigen binding domain.

In one embodiment the at least one nucleic acid sequence encoding the particle-forming protein and the at least one nucleic acid sequence encoding the M. tuberculosis antigen are present as a single open reading frame.

In one embodiment the at least one nucleic acid sequence encoding the particle-forming protein and the at least one nucleic acid sequence encoding the M. tuberculosis antigen binding domain are present as a single open reading frame.

In one embodiment the expression construct comprises:
at least one nucleic acid sequence encoding a fusion polypeptide that comprises a particle-forming protein and at least one M. tuberculosis antigen binding domain; and
at least one nucleic acid sequence encoding an additional polypeptide that comprises at least one polypeptide that binds the M. tuberculosis antigen binding domain of the fusion polypeptide.

In one embodiment the additional polypeptide is a M. tuberculosis antigen, or comprises at least one M. tuberculosis antigen.

In one embodiment the additional polypeptide is a fusion polypeptide comprising a particle-forming protein and at least one M. tuberculosis antigen.

In one embodiment the expression construct comprises:
at least one nucleic acid sequence encoding a fusion polypeptide that comprises a particle-forming protein and at least one M. tuberculosis antigen; and
at least one nucleic acid sequence encoding an additional polypeptide that comprises at least one M. tuberculosis antigen binding domain.

In one embodiment the additional polypeptide is a fusion polypeptide comprising a particle-forming protein and at least one M. tuberculosis antigen binding domain.

In one embodiment the host cell comprises an expression construct selected from the group comprising:
an expression construct comprising a nucleic acid sequence encoding a particle-forming protein and at least one M. tuberculosis antigen, or
an expression construct comprising a nucleic acid sequence encoding a particle-forming protein and at least one M. tuberculosis antigen binding domain, or
an expression construct comprising a nucleic acid sequence encoding an adjuvant, or
an expression construct comprising a nucleic acid sequence encoding at least one M. tuberculosis antigen.

Another aspect of the present invention relates to a polymer particle comprising one or more fusion polypeptides comprising a particle-forming protein fused to at least one M. tuberculosis antigen.

Another aspect of the present invention relates to a polymer particle comprising one or more fusion polypeptides comprising a particle-forming protein fused to at least one M. tuberculosis antigen binding domain.

In one embodiment the polymer particle comprises two or more different M. tuberculosis antigens.

In one embodiment the polymer particle comprises two or more different M. tuberculosis antigen binding domains.

Another aspect of the present invention relates to a composition of polymer particles, wherein the polymer particles comprise one or more fusion polypeptides comprising a particle-forming protein fused to at least one M. tuberculosis antigen.

Another aspect of the present invention relates to a composition of polymer particles, wherein the polymer particles comprise one or more fusion polypeptides comprising a particle-forming protein fused to at least one M. tuberculosis antigen binding domain.

In one embodiment, the composition additionally comprises one or more of the following:
one or more M. tuberculosis antigens,
one or more M. tuberculosis antigen binding domains,
one or more adjuvants, or
one or more immunomodulatory agents or molecules.

Another aspect of the present invention relates to a method of immunising a subject against tuberculosis, wherein the method comprises administering to a subject in need thereof at least one polymer particle comprising one or more fusion polypeptides, wherein at least one of the fusion polypeptides comprises a particle-forming protein fused to at least one M. tuberculosis antigen.

Another aspect of the present invention relates to a method of immunising a subject against tuberculosis, wherein the method comprises administering to a subject in need thereof at least one polymer particle comprising one or more fusion polypeptides, wherein at least one of the fusion polypeptides comprises a particle-forming protein fused to at least one M. tuberculosis antigen binding domain, wherein the M. tuberculosis antigen binding domain is bound to, the subject comprises, or the subject is administered, at least one M. tuberculosis antigen.

In one embodiment the polymer particle is present in a composition comprising at least one M. tuberculosis antigen.

Another aspect of the present invention relates to a method of eliciting an immune response in a subject, wherein the method comprises administering to a subject in need thereof at least one polymer particle comprising one or more fusion polypeptides, wherein at least one of the fusion polypeptides comprises a particle-forming protein fused to at least one M. tuberculosis antigen.

Another aspect of the present invention relates to a method of eliciting an immune response in a subject, wherein the method comprises administering to a subject in need thereof at least one polymer particle comprising one or more fusion polypeptides, wherein at least one of the fusion polypeptides comprises a particle-forming protein fused to a M. tuberculosis antigen binding domain, wherein the M. tuberculosis antigen binding domain is bound to, the subject comprises, or the subject is administered, at least one M. tuberculosis antigen.

In one embodiment the at least one polymer particle is present in a composition comprising at least one M. tuberculosis antigen.

In one embodiment the subject is infected with tuberculosis.

In another embodiment the subject has been immunised against tuberculosis. In one example, the subject has been vaccinated with Bacillus Calmette-Guerin (BCG) (World Health Organisation—http://www.who.int).

Another aspect of the present invention relates to a method of eliciting an immune response in a subject infected with tuberculosis, wherein the method comprises administering to a subject in need thereof at least one polymer particle comprising a particle-forming protein fused to a M. tuberculosis antigen binding domain.

In one embodiment, the M. tuberculosis antigen binding domain binds to an endogenous M. tuberculosis antigen.

Another aspect of the present invention relates to a polymer particle for immunising a subject against tuberculosis, wherein the polymer particle comprises one or more fusion polypeptides, wherein at least one of the fusion polypeptides comprises a particle-forming protein fused to at least one M. tuberculosis antigen.

Another aspect of the present invention relates to a polymer particle for immunising a subject against tuberculosis, wherein the polymer particle comprises one or more fusion polypeptides, wherein at least one of the fusion polypeptides comprises a particle-forming protein fused to at least one M. tuberculosis antigen binding domain.

Another aspect of the present invention relates to a polymer particle for eliciting an immune response in a subject, wherein the polymer particle comprises one or more fusion polypeptides, wherein at least one of the fusion polypeptides comprises a particle-forming protein fused to at least one M. tuberculosis antigen.

Another aspect of the present invention relates to a polymer particle for eliciting an immune response in a subject, wherein the polymer particle comprises one or more fusion polypeptides, wherein at least one of the fusion polypeptides comprises a particle-forming protein fused to at least one M. tuberculosis antigen binding domain.

In one embodiment the polymer particle is present in a composition comprising at least one M. tuberculosis antigen.

In one embodiment the subject is infected with tuberculosis.

In another embodiment the subject has been immunised against tuberculosis. For example, the subject has been vaccinated with Bacillus Calmette-Guerin (BCG).

Another aspect of the present invention relates to a polymer particle for eliciting an immune response in a subject infected with or immunised against tuberculosis, wherein the polymer particle comprises a particle-forming protein fused to a M. tuberculosis antigen binding domain

The use of a polymer particle as described above in the preparation of a medicament for immunising a subject against tuberculosis, or for eliciting an immune response in a subject including a subject infected with or immunised against tuberculosis, is also contemplated.

Another aspect of the present invention relates to a method of diagnosing tuberculosis in a subject, wherein the method comprises administering to a subject at least one polymer particle of the invention and detecting a response indicative of the presence of Mycobacterium tuberculosis.

In one embodiment the response indicative of the presence of Mycobacterium tuberculosis is a delayed-type hypersensitivity response.

Another aspect of the present invention relates to a method of diagnosing tuberculosis in a subject, wherein the method comprises contacting a sample obtained from the subject with a polymer particle of the invention and detecting a response indicative of the presence of Mycobacterium tuberculosis.

In one embodiment the response indicative of the presence of Mycobacterium tuberculosis is the presence of an antibody to the Mycobacterium tuberculosis antigen in said sample.

In one embodiment the presence of antibodies to the Mycobacterium tuberculosis antigen is detected by immunoassay.

In one embodiment the detection of the presence of antibodies to the Mycobacterium tuberculosis antigen is by ELISA, radioimmunoassay-assay, or Western Blot.

In one embodiment the response indicative of the presence of the intracellular pathogen is the presence of an immune cell responsive to the Mycobacterium tuberculosis antigen in said sample.

In one embodiment the presence of an immune cell responsive to the Mycobacterium tuberculosis antigen is detected by a cell proliferation assay, a cell sorting assay including FACS, or an in situ hybridisation assay.

Another aspect of the present invention provides a method for producing polymer particles, the method comprising:
providing a host cell comprising at least one expression construct, the at least one expression construct comprising:
   at least one nucleic acid sequence encoding a particle-forming protein; and
   at least one nucleic acid sequence encoding a hepatitis antigen or a hepatitis antigen binding domain;

maintaining the host cell under conditions suitable for expression of the expression construct and for formation of polymer particles; and separating the polymer particles from the host cells.

In some embodiments in which the host cell comprises at least two different expression constructs, at least one of the expression constructs is selected from the group comprising:

an expression construct comprising a nucleic acid sequence encoding a particle-forming protein and at least one hepatitis antigen, or

an expression construct comprising a nucleic acid sequence encoding a particle-forming protein and at least one hepatitis antigen binding domain, or

an expression construct comprising a nucleic acid sequence encoding an adjuvant, or

an expression construct comprising a nucleic acid sequence encoding at least one hepatitis antigen.

In other embodiments in which the host cell comprises at least two different expression constructs, one of the expression constructs comprises a nucleic acid sequence encoding a particle-forming protein and at least one hepatitis antigen binding domain, and at least one expression construct selected from the group comprising:

an expression construct comprising a nucleic acid sequence encoding a particle-forming protein and at least one hepatitis antigen, or

an expression construct comprising a nucleic acid sequence encoding a particle-forming protein and at least one hepatitis antigen binding domain, or

an expression construct comprising a nucleic acid sequence encoding an adjuvant, or

an expression construct comprising a nucleic acid sequence encoding at least one hepatitis antigen.

In one embodiment the host cell comprises a mixed population of expression constructs wherein each expression construct comprises a nucleic acid sequence encoding a fusion polypeptide, the fusion polypeptide comprising:

at least one particle-forming protein and

at least one hepatitis antigen or at least one hepatitis antigen binding domain.

Another aspect of the present invention relates to an expression construct, the expression construct comprising:

at least one nucleic acid sequence encoding a particle-forming protein; and

at least one nucleic acid sequence encoding a hepatitis antigen.

Another aspect of the present invention relates to an expression construct, the expression construct comprising:

at least one nucleic acid sequence encoding a particle-forming protein; and

at least one nucleic acid sequence encoding a hepatitis antigen binding domain.

In one embodiment the expression construct encodes a fusion polypeptide comprising the particle-forming protein and the hepatitis antigen.

In one embodiment the expression construct encodes a fusion polypeptide comprising the particle-forming protein and the hepatitis antigen binding domain.

In one embodiment the at least one nucleic acid sequence encoding the particle-forming protein and the at least one nucleic acid sequence encoding the hepatitis antigen are present as a single open reading frame.

In one embodiment the at least one nucleic acid sequence encoding the particle-forming protein and the at least one nucleic acid sequence encoding the hepatitis antigen binding domain are present as a single open reading frame.

In one embodiment the expression construct comprises:

at least one nucleic acid sequence encoding a fusion polypeptide that comprises a particle-forming protein and at least one hepatitis antigen binding domain; and

at least one nucleic acid sequence encoding an additional polypeptide that comprises at least one polypeptide that binds the hepatitis antigen binding domain of the fusion polypeptide.

In one embodiment the additional polypeptide is a hepatitis antigen, or comprises at least one Hepatitis antigen.

In one embodiment the additional polypeptide is a fusion polypeptide comprising a particle-forming protein and at least one hepatitis antigen.

In one embodiment the expression construct comprises:

at least one nucleic acid sequence encoding a fusion polypeptide that comprises a particle-forming protein and at least one hepatitis antigen; and

at least one nucleic acid sequence encoding an additional polypeptide that comprises at least one hepatitis antigen binding domain.

In one embodiment the additional polypeptide is a fusion polypeptide comprising a particle-forming protein and at least one hepatitis antigen binding domain.

In one embodiment the host cell comprises an expression construct selected from the group comprising:
an expression construct comprising a nucleic acid sequence encoding a particle-forming protein and at least one hepatitis antigen, or
an expression construct comprising a nucleic acid sequence encoding a particle-forming protein and at least one hepatitis antigen binding domain, or
an expression construct comprising a nucleic acid sequence encoding an adjuvant, or
an expression construct comprising a nucleic acid sequence encoding at least one hepatitis antigen.

Another aspect of the present invention relates to a polymer particle comprising one or more fusion polypeptides comprising a particle-forming protein fused to at least one hepatitis antigen.

Another aspect of the present invention relates to a polymer particle comprising one or more fusion polypeptides comprising a particle-forming protein fused to at least one hepatitis antigen binding domain.

In one embodiment the polymer particle comprises two or more different hepatitis antigens.

In one embodiment the polymer particle comprises two or more different hepatitis antigen binding domains.

Another aspect of the present invention relates to a composition of polymer particles, wherein the polymer particles comprise one or more fusion polypeptides comprising a particle-forming protein fused to at least one hepatitis antigen.

Another aspect of the present invention relates to a composition of polymer particles, wherein the polymer particles comprise one or more fusion polypeptides comprising a particle-forming protein fused to at least one hepatitis antigen binding domain.

In one embodiment, the composition additionally comprises one or more of the following:

one or more hepatitis antigens,

one or more hepatitis antigen binding domains,

one or more adjuvants, or

one or more immunomodulatory agents or molecules.

Another aspect of the present invention relates to a method of immunising a subject against hepatitis, wherein the method comprises administering to a subject in need thereof at least one polymer particle comprising one or more fusion polypeptides, wherein at least one of the fusion polypeptides comprises a particle-forming protein fused to at least one hepatitis antigen.

Another aspect of the present invention relates to a method of immunising a subject against hepatitis, wherein the method comprises administering to a subject in need thereof at least one polymer particle comprising one or more fusion polypeptides, wherein at least one of the fusion polypeptides comprises a particle-forming protein fused to at least one hepatitis antigen binding domain, wherein the hepatitis antigen binding domain is bound to, the subject comprises, or the subject is administered, at least one hepatitis antigen.

In one embodiment the polymer particle is present in a composition comprising at least one Hepatitis antigen.

Another aspect of the present invention relates to a method of eliciting an immune response in a subject, wherein the method comprises administering to a subject in need thereof at least one polymer particle comprising one or more fusion polypeptides, wherein at least one of the fusion polypeptides comprises a particle-forming protein fused to at least one hepatitis antigen.

Another aspect of the present invention relates to a method of eliciting an immune response in a subject, wherein the method comprises administering to a subject in need thereof at least one polymer particle comprising one or more fusion polypeptides, wherein at least one of the fusion polypeptides comprises a particle-forming protein fused to a hepatitis antigen binding domain, wherein the hepatitis antigen binding domain is bound to, the subject comprises, or the subject is administered, at least one hepatitis antigen.

In one embodiment the at least one polymer particle is present in a composition comprising at least one hepatitis antigen.

In one embodiment the subject is infected with hepatitis.

In another embodiment the subject has been immunised against hepatitis.

Another aspect of the present invention relates to a method of eliciting an immune response in a subject infected with hepatitis, wherein the method comprises administering to a subject in need thereof at least one polymer particle comprising a particle-forming protein fused to a hepatitis antigen binding domain.

In one embodiment, the hepatitis antigen binding domain binds to an endogenous Hepatitis antigen.

Another aspect of the present invention relates to a polymer particle for immunising a subject against hepatitis, wherein the polymer particle comprises one or more fusion polypeptides, wherein at least one of the fusion polypeptides comprises a particle-forming protein fused to at least one hepatitis antigen.

Another aspect of the present invention relates to a polymer particle for immunising a subject against hepatitis, wherein the polymer particle comprises one or more fusion polypeptides, wherein at least one of the fusion polypeptides comprises a particle-forming protein fused to at least one hepatitis antigen binding domain.

Another aspect of the present invention relates to a polymer particle for eliciting an immune response in a subject, wherein the polymer particle comprises one or more fusion polypeptides, wherein at least one of the fusion polypeptides comprises a particle-forming protein fused to at least one hepatitis antigen.

Another aspect of the present invention relates to a polymer particle for eliciting an immune response in a subject, wherein the polymer particle comprises one or more fusion polypeptides, wherein at least one of the fusion polypeptides comprises a particle-forming protein fused to at least one hepatitis antigen binding domain.

In one embodiment the polymer particle is present in a composition comprising at least one hepatitis antigen.

In one embodiment the subject is infected with hepatitis.

In another embodiment the subject has been immunised against hepatitis.

Another aspect of the present invention relates to a polymer particle for eliciting an immune response in a subject infected with or immunised against hepatitis, wherein the polymer particle comprises a particle-forming protein fused to a hepatitis antigen binding domain

The use of a polymer particle as described above in the preparation of a medicament for immunising a subject against hepatitis, or for eliciting an immune response in a subject including a subject infected with or immunised against hepatitis, is also contemplated.

Another aspect of the present invention relates to a method of diagnosing hepatitis in a subject, wherein the method comprises administering to a subject at least one polymer particle of the invention and detecting a response indicative of the presence of viral hepatitis.

In one embodiment the response indicative of the presence of viral hepatitis is a delayed-type hypersensitivity response.

Another aspect of the present invention relates to a method of diagnosing hepatitis in a subject, wherein the method comprises contacting a sample obtained from the subject with a polymer particle of the invention and detecting a response indicative of the presence of viral hepatitis.

In one embodiment the response indicative of the presence of viral hepatitis is the presence of an antibody to the viral hepatitis antigen in said sample.

In one embodiment the presence of antibodies to the hepatitis antigen is detected by immunoassay.

In one embodiment the detection of the presence of antibodies to the viral hepatitis antigen is by ELISA, radioimmunoassay-assay, or Western Blot.

In one embodiment the response indicative of the presence of the intracellular pathogen is the presence of an immune cell responsive to the hepatitis antigen in said sample.

In one embodiment the presence of an immune cell responsive to the viral hepatitis antigen is detected by a cell proliferation assay, a cell sorting assay including FACS, or an in situ hybridisation assay.

Another aspect of the present invention provides a method for producing polymer particles, the method comprising:
providing a host cell comprising at least one expression construct, the at least one expression construct comprising:
   at least one nucleic acid sequence encoding a particle-forming protein; and
   at least one nucleic acid sequence encoding an influenza antigen or an influenza antigen binding domain;
maintaining the host cell under conditions suitable for expression of the expression construct and for formation of polymer particles; and separating the polymer particles from the host cells.

In some embodiments in which the host cell comprises at least two different expression constructs, at least one of the expression constructs is selected from the group comprising:
an expression construct comprising a nucleic acid sequence encoding a particle-forming protein and at least one influenza antigen, or
an expression construct comprising a nucleic acid sequence encoding a particle-forming protein and at least one influenza antigen binding domain, or
an expression construct comprising a nucleic acid sequence encoding an adjuvant, or
an expression construct comprising a nucleic acid sequence encoding at least one influenza antigen.

In other embodiments in which the host cell comprises at least two different expression constructs, one of the expression constructs comprises a nucleic acid sequence encoding a particle-forming protein and at least one influenza antigen binding domain, and at least one expression construct selected from the group comprising:
an expression construct comprising a nucleic acid sequence encoding a particle-forming protein and at least one influenza antigen, or
an expression construct comprising a nucleic acid sequence encoding a particle-forming protein and at least one influenza antigen binding domain, or
an expression construct comprising a nucleic acid sequence encoding an adjuvant, or
an expression construct comprising a nucleic acid sequence encoding at least one influenza antigen.

In one embodiment the host cell comprises a mixed population of expression constructs wherein each expression construct comprises a nucleic acid sequence encoding a fusion polypeptide, the fusion polypeptide comprising:
at least one particle-forming protein and
at least one influenza antigen or at least one influenza antigen binding domain.

Another aspect of the present invention relates to an expression construct, the expression construct comprising:
at least one nucleic acid sequence encoding a particle-forming protein; and
at least one nucleic acid sequence encoding a influenza antigen.

Another aspect of the present invention relates to an expression construct, the expression construct comprising:
at least one nucleic acid sequence encoding a particle-forming protein; and
at least one nucleic acid sequence encoding a influenza antigen binding domain.

In one embodiment the expression construct encodes a fusion polypeptide comprising the particle-forming protein and the influenza antigen.

In one embodiment the expression construct encodes a fusion polypeptide comprising the particle-forming protein and the influenza antigen binding domain.

In one embodiment the at least one nucleic acid sequence encoding the particle-forming protein and the at least one nucleic acid sequence encoding the influenza antigen are present as a single open reading frame.

In one embodiment the at least one nucleic acid sequence encoding the particle-forming protein and the at least one nucleic acid sequence encoding the influenza antigen binding domain are present as a single open reading frame.

In one embodiment the expression construct comprises:
at least one nucleic acid sequence encoding a fusion polypeptide that comprises a particle-forming protein and at least one influenza antigen binding domain; and
at least one nucleic acid sequence encoding an additional polypeptide that comprises at least one polypeptide that binds the influenza antigen binding domain of the fusion polypeptide.

In one embodiment the additional polypeptide is an influenza antigen, or comprises at least one influenza antigen.

In one embodiment the additional polypeptide is a fusion polypeptide comprising a particle-forming protein and at least one influenza antigen.

In one embodiment the expression construct comprises:
at least one nucleic acid sequence encoding a fusion polypeptide that comprises a particle-forming protein and at least one influenza antigen; and
at least one nucleic acid sequence encoding an additional polypeptide that comprises at least one influenza antigen binding domain.

In one embodiment the additional polypeptide is a fusion polypeptide comprising a particle-forming protein and at least one influenza antigen binding domain.

In one embodiment the host cell comprises an expression construct selected from the group comprising:
an expression construct comprising a nucleic acid sequence encoding a particle-forming protein and at least one influenza antigen, or
an expression construct comprising a nucleic acid sequence encoding a particle-forming protein and at least one influenza antigen binding domain, or
an expression construct comprising a nucleic acid sequence encoding an adjuvant, or
an expression construct comprising a nucleic acid sequence encoding at least one influenza antigen.

Another aspect of the present invention relates to a polymer particle comprising one or more fusion polypeptides comprising a particle-forming protein fused to at least one influenza antigen.

Another aspect of the present invention relates to a polymer particle comprising one or more fusion polypeptides comprising a particle-forming protein fused to at least one influenza antigen binding domain.

In one embodiment the polymer particle comprises two or more different influenza antigens.

In one embodiment the polymer particle comprises two or more different influenza antigen binding domains.

Another aspect of the present invention relates to a composition of polymer particles, wherein the polymer particles comprise one or more fusion polypeptides comprising a particle-forming protein fused to at least one influenza antigen.

Another aspect of the present invention relates to a composition of polymer particles, wherein the polymer particles comprise one or more fusion polypeptides comprising a particle-forming protein fused to at least one influenza antigen binding domain.

In one embodiment, the composition additionally comprises one or more of the following:
one or more influenza antigens,
one or more influenza antigen binding domains,
one or more adjuvants, or
one or more immunomodulatory agents or molecules.

Another aspect of the present invention relates to a method of immunising a subject against influenza, wherein the method comprises administering to a subject in need thereof at least one polymer particle comprising one or more fusion polypeptides, wherein at least one of the fusion polypeptides comprises a particle-forming protein fused to at least one influenza antigen.

Another aspect of the present invention relates to a method of immunising a subject against influenza, wherein the method comprises administering to a subject in need thereof at least one polymer particle comprising one or more fusion polypeptides, wherein at least one of the fusion polypeptides comprises a particle-forming protein fused to at least one influenza antigen binding domain, wherein the influenza antigen binding domain is bound to, the subject comprises, or the subject is administered, at least one influenza antigen.

In one embodiment the polymer particle is present in a composition comprising at least one influenza antigen.

Another aspect of the present invention relates to a method of eliciting an immune response in a subject, wherein the method comprises administering to a subject in need thereof at least one polymer particle comprising one or more fusion polypeptides, wherein at least one of the fusion polypeptides comprises a particle-forming protein fused to at least one influenza antigen.

Another aspect of the present invention relates to a method of eliciting an immune response in a subject, wherein the method comprises administering to a subject in need thereof at least one polymer particle comprising one or more fusion polypeptides, wherein at least one of the fusion polypeptides comprises a particle-forming protein fused to an influenza antigen binding domain, wherein the influenza antigen binding domain is bound to, the subject comprises, or the subject is administered, at least one influenza antigen.

In one embodiment the at least one polymer particle is present in a composition comprising at least one influenza antigen.

In one embodiment the subject is infected with influenza.

In another embodiment the subject has been immunised against influenza.

Another aspect of the present invention relates to a method of eliciting an immune response in a subject infected with influenza, wherein the method comprises administering to a subject in need thereof at least one polymer particle comprising a particle-forming protein fused to a Influenza antigen binding domain.

In one embodiment, the influenza antigen binding domain binds to an endogenous Influenza antigen.

Another aspect of the present invention relates to a polymer particle for immunising a subject against influenza, wherein the polymer particle comprises one or more fusion polypeptides, wherein at least one of the fusion polypeptides comprises a particle-forming protein fused to at least one influenza antigen.

Another aspect of the present invention relates to a polymer particle for immunising a subject against influenza, wherein the polymer particle comprises one or more fusion polypeptides, wherein at least one of the fusion polypeptides comprises a particle-forming protein fused to at least one influenza antigen binding domain.

Another aspect of the present invention relates to a polymer particle for eliciting an immune response in a subject, wherein the polymer particle comprises one or more fusion polypeptides, wherein at least one of the fusion polypeptides comprises a particle-forming protein fused to at least one influenza antigen.

Another aspect of the present invention relates to a polymer particle for eliciting an immune response in a subject, wherein the polymer particle comprises one or more fusion polypeptides, wherein at least one of the fusion polypeptides comprises a particle-forming protein fused to at least one influenza antigen binding domain.

In one embodiment the polymer particle is present in a composition comprising at least one influenza antigen.

In one embodiment the subject is infected with influenza.

In another embodiment the subject has been immunised against influenza.

Another aspect of the present invention relates to a polymer particle for eliciting an immune response in a subject infected with or immunised against influenza, wherein the polymer particle comprises a particle-forming protein fused to an influenza antigen binding domain

The use of a polymer particle as described above in the preparation of a medicament for immunising a subject against influenza, or for eliciting an immune response in a subject including a subject infected with or immunised against influenza, is also contemplated.

Another aspect of the present invention relates to a method of diagnosing influenza in a subject, wherein the method comprises administering to a subject at least one polymer particle of the invention and detecting a response indicative of the presence of influenza virus.

In one embodiment the response indicative of the presence of influenza virus is a delayed-type hypersensitivity response.

Another aspect of the present invention relates to a method of diagnosing influenza in a subject, wherein the method comprises contacting a sample obtained from the subject with a polymer particle of the invention and detecting a response indicative of the presence of influenza virus.

In one embodiment the response indicative of the presence of influenza virus is the presence of an antibody to the influenza antigen in said sample.

In one embodiment the presence of antibodies to the influenza antigen is detected by immunoassay.

In one embodiment the detection of the presence of antibodies to the influenza antigen is by ELISA, radioimmunoassay-assay, or Western Blot.

In one embodiment the response indicative of the presence of the intracellular pathogen is the presence of an immune cell responsive to the influenza antigen in said sample.

In one embodiment the presence of an immune cell responsive to the influenza antigen is detected by a cell proliferation assay, a cell sorting assay including FACS, or an in situ hybridisation assay.

The following embodiments may relate to any of the above aspects.

In various embodiments the particle-forming protein is a polymer synthase.

In various embodiments the polymer particle comprises a polymer selected from poly-beta-amino acids, polylactates, polythioesters and polyesters. Most preferably the polymer comprises polyhydroxyalkanoate (PHA), preferably poly(3-hydroxybutyrate) (PHB).

In various embodiments the polymer particle comprises a polymer particle encapsulated by a phospholipid monolayer.

In various embodiments the polymer particle comprises two or more different fusion polypeptides.

In various embodiments the polymer particle comprises two or more different fusion polypeptides on the polymer particle surface.

In various embodiments the polymer particle comprises three or more different fusion polypeptides, such as three or more different fusion polypeptides on the polymer particle surface.

In various embodiments the polymer particle further comprises at least one substance bound to or incorporated into the polymer particle, or a combination thereof.

In various embodiments the substance is an antigen, adjuvant or immunostimulatory molecule.

In various embodiments the substance is bound to the polymer particle by cross-linking.

In various embodiments the polymer synthase is bound to the polymer particle or to the phospholipid monolayer or is bound to both.

In various embodiments the polymer synthase is covalently or non-covalently bound to the polymer particle it forms.

In various embodiments the polymer synthase is a PHA synthase from the class 1 genera Acinetobacter, Vibrio, Aeromonas, Chromobacterium, Pseudomonas, Zoogloea, Alcaligenes, Delftia, Burkholderia, Ralstonia, Rhodococcus, Gordonia, Rhodobacter, Paracoccus, Rickettsia, Caulobacter, Methylobacterium, Azorhizobium, Agrobacterium, Rhizobium, Sinorhizobium, Rickettsia, Crenarchaeota, Synechocystis, Ectothiorhodospira, Thiocapsa, Thyocystis and Allochromatium, the class 2 genera Burkholderia and Pseudomonas, or the class 4 genera Bacillus, more preferably from the group comprising class 1 Acinetobacter sp. RA3849, Vibrio cholerae, Vibrio parahaemolyticus, Aeromonas punctata FA440, Aeromonas hydrophila, Chromobacterium violaceum, Pseudomonas sp. 61-3, Zoogloea ramigera, Alcaligenes latus, Alcaligenes sp. SH-69, Delftia acidovorans, Burkholderia sp. DSMZ9242, Ralstonia eutrophia H16, Burkholderia cepacia, Rhodococcus rubber PP2, Gordonia rubripertinctus, Rickettsia prowazekii, Synechocystis sp. PCC6803, Ectothiorhodospira shaposhnikovii N1, Thiocapsa pfennigii 9111, Allochromatium vinosum D, Thyocystis violacea 2311, Rhodobacter sphaeroides, Paracoccus denitrificans, Rhodobacter capsulatus, Caulobacter crescentus, Methylobacterium extorquens, Azorhizobium caulinodans, Agrobacterium tumefaciens, Sinorhizobium meliloti 41, Rhodospirillum rubrum HA, and Rhodospirillum rubrum ATCC25903, class 2 Burkholderia caryophylli, Pseudomonas chloraphis, Pseudomonas sp. 61-3, Pseudomonas putida U, Pseudomonas oleovorans, Pseudomonas aeruginosa, Pseudomonas resinovorans, Pseudomonas stutzeri, Pseudomonas mendocina, Pseudomonas pseudolcaligenes, Pseudomonas putida BM01, Pseudomonas nitroreducins, Pseudomonas chloraphis, and class 4 Bacillus megaterium and Bacillus sp. INT005.

In other embodiments the polymer synthase is a PHA polymer synthase from Gram-negative and Gram-positive eubacteria, or from archaea.

In various examples, the polymer synthase may comprise a PHA polymer synthase from C. necator, P. aeruginosa, A. vinosum, B. megaterium, H. marismortui, P. aureofaciens, or P. putida, which have Accession Nos. AY836680, AE004091, AB205104, AF109909, YP137339, AB049413 and AF150670, respectively.

Other polymer synthases amenable to use in the present invention include polymer synthases, each identified by it accession number, from the following organisms: R. eutropha (A34341), T. pfennigii (X93599), A. punctata (032472), Pseudomonas sp. 61-3 (AB014757 and AB014758), R. sphaeroides (AAA72004), C. violaceum (AAC69615), A. borkumensis SK2 (CAL17662), A. borkumensis SK2 (CAL16866), R. sphaeroides KD131 (ACM01571 and YP002526072), R. opacus B4 (BAH51880 and YP002780825), B. multivorans ATCC 17616 (YP001946215 and BAG43679), A. borkumensis SK2(YP693934 and YP693138), R. rubrum (AAD53179), gamma proteobacterium HTCC5015 (ZP05061661 and EDY86606), Azoarcus sp. BH72 (YP932525), C. violaceum ATCC 12472 (NP902459), Limnobacter sp. MED105 (ZP01915838 and EDM82867), M. algicola DG893 (ZP01895922 and EDM46004), R. sphaeroides (CAA65833), C. violaceum ATCC 12472 (AAQ60457), A. latus (AAD10274, AAD01209 and AAC83658), S. maltophilia K279a (CAQ46418 and YP001972712), R. solanacearum IP01609 (CAQ59975 and YP002258080), B. multivorans ATCC 17616 (YP001941448 and BAG47458), Pseudomonas sp. g113 (ACJ02400), Pseudomonas sp. g106 (ACJ02399), Pseudomonas sp. gl01 (ACJ02398), R. sp. g132 (ACJ02397), R. leguminosarum bv. viciae 3841 (CAK10329 and YP770390), Azoarcus sp. BH72 (CAL93638), Pseudomonas sp. LDC-5 (AAV36510), L. nitroferrum 2002 (ZP03698179), Thauera sp. MZ1T (YP002890098 and ACR01721), M. radiotolerans JCM 2831 (YP001755078 and ACB24395), Methylobacterium sp. 4-46 (YP001767769 and ACA15335), L. nitroferrum 2002 (EEG08921), P. denitrificans (BAA77257), M. gryphiswaldense (ABG23018), Pseudomonas sp. USM4-55 (ABX64435 and ABX64434), A. hydrophila (AAT77261 and AAT77258), Bacillus sp. INT005 (BAC45232 and BAC45230), P. putida (AAM63409 and AAM63407), G. rubripertinctus (AAB94058), B. megaterium (AAD05260), D. acidovorans (BAA33155), P. seriniphilus (ACM68662), Pseudomonas sp. 14-3 (CAK18904), Pseudomonas sp. LDC-5 (AAX18690), Pseudomonas sp. PC17 (ABV25706), Pseudomonas sp. 3Y2 (AAV35431, AAV35429 and AAV35426), P. mendocina (AAM10546 and AAM10544), P. nitroreducens (AAK19608), P. pseudoalcaligenes (AAK19605), P. resinovorans (AAD26367 and AAD26365), Pseudomonas sp. USM7-7 (ACM90523 and ACM90522), P. fluorescens (AAP58480) and other uncultured bacterium (BAE02881, BAE02880, BAE02879, BAE02878, BAE02877, BAE02876, BAE02875, BAE02874, BAE02873, BAE02872, BAE02871, BAE02870, BAE02869, BAE02868, BAE02867, BAE0286, BAE02865, BAE02864, BAE02863, BAE02862, BAE02861, BAE02860, BAE02859, BAE02858, BAE02857, BAE07146, BAE07145, BAE07144, BAE07143, BAE07142, BAE07141, BAE07140, BAE07139, BAE07138, BAE07137, BAE07136, BAE07135, BAE07134, BAE07133, BAE07132, BAE07131, BAE07130, BAE07129, BAE07128, BAE07127, BAE07126, BAE07125, BAE07124, BAE07123, BAE07122, BAE07121, BAE07120, BAE07119, BAE07118, BAE07117, BAE07116, BAE7115, BAE07114, BAE07113, BAE7112, BAE07111, BAE7110, BAE07109, BAE7108, BAE07107, BAE07106, BAE7105, BAE7104, BAE7103, BAE07102, BAE07101, BAE07100, BAE07099, BAE07098, BAE07097, BAE07096, BAE07095, BAE07094, BAE07093, BAE07092, BAE07091, BAE07090, BAE07089, BAE07088, BAE07053, BAE07052, BAE07051, BAE07050, BAE07049, BAE07048, BAE07047, BAE07046, BAE07045, BAE07044, BAE07043, BAE07042, BAE07041, BAE07040, BAE07039, BAE07038, BAE07037, BAE07036, BAE07035, BAE07034, BAE07033, BAE07032, BAE07031, BAE07030, BAE07029, BAE07028, BAE07027, BAE07026, BAE07025, BAE07024, BAE07023, BAE07022, BAE07021, BAE07020, BAE07019, BAE07018, BAE07017, BAE7016, BAE715, BAE7014, BAE7013, BAE07012, BAE7011, BAE07010, BAE07009, BAE7008, BAE7007, BAE7006, BAE7005, BAE7004, BAE7003, BAE7002, BAE7001, BAE7000, BAE06999, BAE06998, BAE06997, BAE06996, BAE06995, BAE06994, BAE06993, BAE06992, BAE06991, BAE06990, BAE06989, BAE06988, BAE06987, BAE06986, BAE06985, BAE06984, BAE06983, BAE06982, BAE06981, BAE06980, BAE06979, BAE06978, BAE06977, BAE06976, BAE06975, BAE06974, BAE06973, BAE06972, BAE06971, BAE06970, BAE06969, BAE06968, BAE06967, BAE06966, BAE06965, BAE06964, BAE06963, BAE06962, BAE06961, BAE06960, BAE06959, BAE06958, BAE06957, BAE06956, BAE06955, BAE06954, BAE06953, BAE06952, BAE06951, BAE06950, BAE06949, BAE06948, BAE06947, BAE06946, BAE06945, BAE06944, BAE06943, BAE06942, BAE06941, BAE06940, BAE06939, BAE06938, BAE06937, BAE06936, BAE06935, BAE06934, BAE06933, BAE06932, BAE06931, BAE06930, BAE06929, BAE06928, BAE06927, BAE06926, BAE06925, BAE06924, BAE06923, BAE06922, BAE06921, BAE06920, BAE06919, BAE06918, BAE06917, BAE06916, BAE06915, BAE06914, BAE06913, BAE06912, BAE06911, BAE06910, BAE06909, BAE06908, BAE06907, BAE06906, BAE06905, BAE06904, BAE06903, BAE06902, BAE06901, BAE06900, BAE06899, BAE06898, BAE06897, BAE06896, BAE06895, BAE06894, BAE06893, BAE06892, BAE06891, BAE06890, BAE06889, BAE06888, BAE06887, BAE06886, BAE06885, BAE06884, BAE06883, BAE06882, BAE06881, BAE06880, BAE06879, BAE06878, BAE06877, BAE06876, BAE06875, BAE06874, BAE06873, BAE06872, BAE06871, BAE06870, BAE06869, BAE06868, BAE06867, BAE06866, BAE06865, BAE06864, BAE06863, BAE06862, BAE06861, BAE06860, BAE06859, BAE06858, BAE06857, BAE06856, BAE06855, BAE06854, BAE06853 and BAE06852).

In various embodiments the polymer synthase can be used for the in vitro production of polymer particles by polymerising or facilitating the polymerisation of the substrates (R)-Hydroxyacyl-CoA or other CoA thioester or derivatives thereof.

In various embodiments the substrate or the substrate mixture comprises at least one optionally substituted amino acid, lactate, ester or saturated or unsaturated fatty acid, preferably acetyl-CoA.

In various embodiments the expression construct is in a high copy number vector.

In various embodiments the expression construct comprises at least one nucleic acid sequence encoding an additional polypeptide.

In various embodiments the construct additionally comprises a nucleic acid encoding
i. at least one thiolase, or
ii. at least one reductase, or
iii. both (i) and (ii).

In various embodiments the construct comprises a nucleic acid encoding
i. at least one thiolase,
ii. at least one reductase,
iii. at least one polymer synthase;
iv. at least one antigen capable of eliciting an immune response, or
v. at least one binding domain capable of binding at least one antigen capable of eliciting an immune response,
vi. a fusion protein comprising one or more of i) to v) above,
vii. any combination of i) to vi) above.

In various embodiments the construct comprises a nucleic acid encoding
i. at least one thiolase,
ii. at least one reductase,
iii. at least one polymer synthase;
iv. at least one antigen capable of eliciting a cell-mediated immune response, or
v. at least one binding domain capable of binding at least one antigen capable of eliciting a cell-mediated immune response,
vi. a fusion protein comprising one or more of i) to v) above,
vii. any combination of i) to vi) above.

In various embodiments the at least one nucleic acid sequence encoding a particle-forming protein, is operably linked to a strong promoter.

In various embodiments the strong promoter is a viral promoter or a phage promoter.

In various embodiments the promoter is a phage promoter, for example a T7 phage promoter.

In various embodiments the host cell is maintained in the presence of a substrate of the particle-forming protein, preferably a substrate of polymer synthase when present, or a substrate mixture, including monomeric substrate, or a precursor substrate able to be metabolised by the host cell to form a substrate of the particle-forming protein.

In various embodiments the host cell comprises at least two different expression constructs.

In various embodiments in which the host cell comprises at least two different expression constructs, one of the expression constructs is selected from the group comprising:

an expression construct comprising a nucleic acid sequence encoding a particle-forming protein, or

an expression construct comprising a nucleic acid sequence encoding a particle-size determining protein, or

an expression construct comprising a nucleic acid sequence encoding a polymer regulator.

In various embodiments the nucleic acid sequence that codes for a fusion polypeptide comprises:

a nucleic acid sequence that codes for an antigen capable of eliciting a cell-mediated response in a subject, or a binding domain capable of binding an antigen capable of eliciting a cell-mediated response in a subject, contiguous with the 5′ or 3′ end of the nucleic acid sequence that codes for a particle-forming protein, preferably a polymer synthase, or

a nucleic acid sequence that codes for an antigen capable of eliciting a cell-mediated response in a subject or a binding domain capable of binding an antigen capable of eliciting a cell-mediated response in a subject indirectly fused with the 5′ or 3′ end of the nucleic acid sequence that codes for a particle-forming protein, preferably a polymer synthase, through a polynucleotide linker or spacer sequence of a desired length; or

a nucleic acid sequence that codes for an antigen capable of eliciting a cell-mediated response in a subject or a binding domain capable of binding an antigen capable of eliciting a cell-mediated response in a subject that is inserted into the nucleic acid sequence that codes for a particle-forming protein, preferably a polymer synthase, optionally through a polynucleotide linker or spacer sequence of a desired length; or

a nucleic acid sequence that codes for a protease cleavage site spaced between the nucleic acid sequence that codes for an antigen capable of eliciting a cell-mediated response in a subject or a binding domain capable of binding an antigen capable of eliciting a cell-mediated response in a subject and the nucleic acid sequence that codes for a particle-forming protein, preferably a polymer synthase; or

a nucleic acid sequence that codes for a self-splicing element spaced between the nucleic acid sequence that codes for an antigen capable of eliciting a cell-mediated response or a binding domain capable of binding an antigen capable of eliciting a cell-mediated response and the nucleic acid sequence that codes for a particle-forming protein, preferably a polymer synthase; or

any combination of two or more thereof.

In various embodiments the at least one fusion polypeptide comprises:

an amino acid sequence that comprises an antigen capable of eliciting a cell-mediated response or that comprises a binding domain capable of binding an antigen capable of eliciting a cell-mediated response contiguous with the N- or C-terminal end of the amino acid sequence that comprises a particle-forming protein, preferably a polymer synthase; or

an amino acid sequence that comprises a an antigen capable of eliciting a cell-mediated response or a binding domain capable of binding an antigen capable of eliciting a cell-mediated response indirectly fused with the N- or C-terminal of the amino acid sequence that comprises a particle-forming protein, preferably a polymer synthase, through a peptide linker or spacer sequence of a desired length; or

an amino acid sequence that comprises an antigen capable of eliciting a cell-mediated response or a binding domain capable of binding an antigen capable of eliciting a cell-mediated response that is inserted into the amino acid sequence that comprises a particle-forming protein, preferably a polymer synthase, through a peptide linker or spacer sequence of a desired length; or

an amino acid sequence that comprises a protease cleavage site spaced between the amino acid sequence that comprises an antigen capable of eliciting a cell-mediated response or a binding domain capable of binding an antigen capable of eliciting a cell-mediated response and the amino acid sequence that codes for a particle-forming protein, preferably a polymer synthase; or

an amino acid sequence that comprises a self-splicing element spaced between the amino acid sequence that comprises an antigen capable of eliciting a cell-mediated response or a binding domain capable of binding an antigen capable of eliciting a cell-mediated response and the amino acid sequence that codes for a particle-forming protein, preferably a polymer synthase; or

any combination of two or more thereof.

In various embodiments the nucleic acid sequence that codes for a fusion polypeptide comprises:

a nucleic acid sequence that codes for a M. tuberculosis antigen or a M. tuberculosis antigen binding domain contiguous with the 5′ or 3′ end of the nucleic acid sequence that codes for a particle-forming protein or

a nucleic acid sequence that codes for a M. tuberculosis antigen or a M. tuberculosis antigen binding domain indirectly fused with the 5′ or 3′ end of the nucleic acid sequence that codes for a particle-forming protein through a polynucleotide linker or spacer sequence of a desired length; or

a nucleic acid sequence that codes for a M. tuberculosis antigen or a M. tuberculosis antigen binding domain that is inserted into the nucleic acid sequence that codes for a particle-forming protein optionally through a polynucleotide linker or spacer sequence of a desired length; or

a nucleic acid sequence that codes for a protease cleavage site spaced between the nucleic acid sequence that codes for a M. tuberculosis antigen or a M. tuberculosis antigen binding domain and the nucleic acid sequence that codes for a particle-forming protein; or

a nucleic acid sequence that codes for a self-splicing element spaced between the nucleic acid sequence that codes for a M. tuberculosis antigen or a M. tuberculosis antigen binding domain and the nucleic acid sequence that codes for a particle-forming protein; or

any combination of two or more thereof.

In various embodiments the at least one fusion polypeptide comprises:

an amino acid sequence that comprises a M. tuberculosis antigen or that comprises a M. tuberculosis antigen binding domain contiguous with the N- or C-terminal end of the amino acid sequence that comprises a particle-forming protein; or

an amino acid sequence that comprises a M. tuberculosis antigen or a M. tuberculosis antigen binding domain indirectly fused with the N- or C-terminal of the amino acid sequence that comprises a particle-forming protein through a peptide linker or spacer sequence of a desired length; or

an amino acid sequence that comprises a M. tuberculosis antigen or a M. tuberculosis antigen binding domain that is inserted into the amino acid sequence that comprises a particle-forming protein through a peptide linker or spacer sequence of a desired length; or

an amino acid sequence that comprises a protease cleavage site spaced between the amino acid sequence that comprises a M. tuberculosis antigen or a M. tuberculosis antigen binding domain and the amino acid sequence that codes for a particle-forming protein; or

an amino acid sequence that comprises a self-splicing element spaced between the amino acid sequence that comprises a M. tuberculosis antigen or a M. tuberculosis antigen binding domain and the amino acid sequence that codes for a particle-forming protein; or

any combination of two or more thereof.

In various embodiments the expression construct comprises a constitutive or regulatable promoter system.

In various embodiments the regulatable promoter system is an inducible or repressible promoter system.

In various embodiments the regulatable promoter system is selected from LacI, Trp, phage γ and phage RNA polymerase.

In one embodiment the promoter is any strong promoter known to those skilled in the art.

Suitable strong promoters comprise adenoviral promoters, such as the adenoviral major late promoter; or heterologous promoters, such as the cytomegalovirus (CMV) promoter; the respiratory syncytial virus (RSV) promoter; the simian virus 40 (SV40) promoter; inducible promoters, such as the MMT promoter, the metallothionein promoter; heat shock promoters; the albumin promoter; the ApoAI promoter; human globin promoters; viral thymidine kinase promoters, such as the Herpes simplex thymidine kinase promoter; retroviral LTRs; the b-actin promoter; human growth hormone promoters; phage promoters such as the T7, SP6 and T3 RNA polymerase promoters and the cauliflower mosaic 35S (CaMV 35S) promoter.

In various embodiments the promoter is a T7 RNA polymerase promoter, such as a T7 RNA polymerase promoter as described in PCT/NZ2006/000251, published as WO 2007/037706.

In various embodiments the cell comprises two or more different expression constructs that each encode a different fusion polypeptide.

In various embodiments the antigen capable of eliciting a cell-mediated immune response is an antigen derived from an intracellular pathogen.

In various embodiments the antigen capable of eliciting a cell-mediated immune response is selected from an antigen derived from the group of pathogens comprising Mycobacterium (e.g. M. bovis, M. tuberculosis, M. leprae, M. kansasii, M. avium, M. avium paratuberculosis, Mycobacterium sp.), Listeria (e.g. L. monocytogenes, Listeria sp.), Salmonella (e.g. S. typhi), Yersinia (e.g Y. pestis, Y. enterocolitica, Y. pseudotuberculosis), Bacillus anthracis, Legionella (e.g. L. pneumophila, L. longbeachae, L. bozemanii, Legionella sp.), Rickettsia (e.g. R. rickettsii, R. akari, R. conorii, R. siberica, R. australis, R. japonica, R. africae, R. prowazekii, R. typhi, Rickettsia sp.), Chlamydia (e.g. C. pneumoniae, C. trachomatis, Chlamydia sp.), Clamydophila (e.g. C. psittaci, C. abortus), Streptococcus (e.g. S. pneumoniae, S. pyogenes, S. agalactiae), Staphylococcus (S. aureus) including Methicillin resistant Staphylococcus aureus (MRSA), Ehrlichia (e.g. E. chaffeensis, Ehrlichia phagocytophila geno group, Ehrlichia sp.), Coxiella burnetii, Leishmania sp., Toxpolasma gondii, Trypanosoma cruzi, Histoplasma sp., Francisella tularensis, and viruses including Hepatitis C, Adenoviruses, Picornaviruses including coxsackievirus, hepatitis A virus, poliovirus, Herpesviruses including epstein-barr virus, herpes simplex type 1, herpes simplex type 2, human cytomegalovirus, human herpesvirus type 8, varicella-zoster virus, Hepadnaviruses including hepatitis B virus, Flaviviruses including hepatitis C virus, yellow fever virus, dengue virus, West Nile virus, Retroviruses including human immunodeficiency virus (HIV), Orthomyxoviruses including influenza virus, Paramyxoviruses including measles virus, mumps virus, parainfluenza virus, respiratory syncytial virus, Papillomaviruses including papillomavirus, Rhabdoviruses including rabies virus, Togaviruses including Rubella virus, and other viruses including vaccinia, avipox, adeno-associated virus, modified Vaccinia Strain Ankara, Semliki Forest virus, poxvirus, and coronaviruses, or at least one antigenic portion or T-cell epitope of any of the above mentioned antigens.

In various embodiments the M. tuberculosis antigen is selected from the group comprising early secretary antigen target (ESAT)-6, Ag85A, Ag85B (MPT59), Ag85B, Ag85C, MPT32, MPT51, MPT59, MPT63, MPT64, MPT83, MPB5, MPB59, MPB64, MTC28, Mtb2, Mtb8.4, Mtb9.9, Mtb32A, Mtb39, Mtb41, TB10.4, TB10C, TB11B, TB12.5, TB13A, TB14, TB15, TB15A, TB16, TB16A, TB17, TB18, TB21, TB20.6, TB24, TB27B, TB32, TB32A, TB33, TB38, TB40.8, TB51, TB54, TB64, CFP6, CFP7, CFP7A, CFP7B, CFP8A, CFP8B, CFP9, CFP10, CFP1, CFP16, CFP17, CFP19, CFP19A, CFP19B, CFP20, CFP21, CFP22, CFP22A, CFP23, CFP23A, CFP23B, CFP25, CFP25A, CFP27, CFP28, CFP28B, CFP29, CFP30A, CFP30B, CFP50, CWP32, hspX (alpha-crystalline), APA, Tuberculin purified protein derivative (PPD), ST-CF, PPE68, LppX, PstS-1, PstS-2, PstS-3, HBHA, GroEL, GroEL2, GrpES, LHP, 19 kDa lipoprotein, 71 kDa, RD1-ORF2, RD1-ORF3, RD1-ORF4, RD1-ORF5, RD1-ORF8, RD1-ORF9A, RD1-ORF9B, Rv1984c, Rv0577, Rv1827, BfrB, Tpx. Rv1352, Rv1810, PpiA, Cut2, FbpB, FbpA, FbpC, DnaK, FecB, Ssb, Rp1L, FixA, FixB, AhpC2, Rv2626c, Rv1211, Mdh, Rv1626, Adk, ClpP, SucD (Belisle et al, 2005; U.S. Pat. No. 7,037,510; US 2004/0057963; US 2008/0199493; US 2008/0267990), or at least one antigenic portion or T-cell epitope of any of the above mentioned antigens.

In one example, the M. tuberculosis antigen is early secretary antigen target (ESAT)-6, Ag85A, at least one antigenic portion of ESAT-6, at least one antigenic portion of Ag85A, or any combination of two or more thereof, such as, for example, both ESAT-6 and Ag85A.

In various embodiments the binding domain capable of binding the antigen capable of eliciting an immune response, such as a binding domain capable of binding an antigen capable of eliciting a cell-mediated immune response is selected from a protein, a protein fragment, a binding domain, a target-binding domain, a binding protein, a binding protein fragment, an antibody, an antibody fragment, an antibody heavy chain, an antibody light chain, a single chain antibody, a single-domain antibody (a VHH for example), a Fab antibody fragment, an Fc antibody fragment, an Fv antibody fragment, a F(ab′)2 antibody fragment, a Fab′ antibody fragment, a single-chain Fv (scFv) antibody fragment, a T-cell receptor, a MHC Class I molecule, MHC Class II molecule, or a combination thereof.

For example, in various embodiments the M. tuberculosis antigen binding domain is selected from a protein, a protein fragment, a binding domain, a target-binding domain, a binding protein, a binding protein fragment, an antibody, an antibody fragment, an antibody heavy chain, an antibody light chain, a single chain antibody, a single-domain antibody (a VHH for example), a Fab antibody fragment, an Fc antibody fragment, an Fv antibody fragment, a F(ab′)2 antibody fragment, a Fab′ antibody fragment, a single-chain Fv (scFv) antibody fragment, a T-cell receptor, a MHC Class I molecule, MHC Class II molecule, or a combination thereof.

In various embodiments, the composition comprises an homogenous population of polymer particles.

In various embodiments, the composition comprises a mixed population of polymer particles.

The immune response are a cell-mediated immune response, or are a humoral immune response, or are a combination of both a cell-mediated immune response and a humoral immune response.

For example, the immune response are a cell-mediated immune response without significant humoral response. For example, the immune response are a cell-mediated immune response, such as that indicated by an IFN-γ response, in the absence of a significant IgA response, or in the absence of a significant IgE response, or in the absence of a significant IgG response, including the absence of a significant IgG1 response, or the absence of a significant IgG2 response, or in the absence of a significant IgM response.

In another example, the immune response is a humoral response without significant cell-mediated response.

It will be appreciated that the focus of the invention is to elicit an immune response so as to be effective in the treatment or prevention of the diseases or conditions described herein. It will similarly be appreciated that, given the nature of the immune response, eliciting a cell-mediated immune response may also elicit a humoral response, such that the subject's response to the methods of the invention may in fact be a combination of both a cell-mediated immune response and a humoral immune response.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Further aspects and advantages of the present invention will become apparent from the ensuing description which is given by way of example only.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### BRIEF DESCRIPTION OF THE DRAWINGS

Further aspects of the present invention will become apparent from the following description which is given by way of example only and with reference to the accompanying drawings.

FIG. 1 shows the binding of anti-Hep C antibody to Hep C polymer particles. See Example 4 herein.

FIG. 2 shows the IgG1 antibody response in mice immunised with various polymer particle vaccines against Hepatitis C. EC50 refers to the reciprocal serum titre which gives half-maximal optical density. Level of detection is 25. * indicates significant difference to other groups. (p<0.05). Bars indicate SEM. See Example 4 herein.

FIG. 3 shows the IgG2c antibody response in mice immunised with various polymer particle vaccines against Hepatitis C. EC50 refers to the reciprocal serum titre which gives half-maximal optical density. Level of detection is 25. * indicates significant difference to other groups. (p<0.05). Bars indicate SEM. See Example 4 herein.

FIG. 4 shows the IFN-γ responses in mice immunised with various polymer particle vaccines against Hepatitis. * indicates a significant difference to other groups (p<0.05). Bars indicate SEM. See Example 4 herein.

FIG. 5 shows the antibody responses in mice immunised 3 times with 0-90 μg polymer particles displaying Ag85A-ESAT-6 or 30 μg recombinant Ag85A-ESAT-6. * indicates a significantly greater response than the PBS immunised control group (p<0.01). ** indicates a significantly greater response than all the other vaccine groups (p<0.01). See Example 5 herein.

FIG. 6 shows the antibody responses in mice immunised 3 times with 30 μg of wild-type polymer particles, Ag85A-ESAT-6 polymer particles, Ag85A-ESAT-6 polymer particles with Emulsigen or non-immunised. * indicates a significantly greater response than the PBS immunised control group (p<0.01). ** indicates a significantly greater response than all the other vaccine groups (p<0.01). See Example 5 herein.

FIG. 7 shows the IFN-γ responses in mice immunised 3 times with 0-90 μg polymer particles displaying Ag85A-ESAT-6 or 30 μg recombinant Ag85A-ESAT-6. * indicates a significantly greater response than the PBS immunised control group (p<0.01). ** indicates a significantly greater response than all the other vaccine groups (p<0.01). See Example 5 herein.

FIG. 8 shows the IFN-γ responses in mice immunised 3 times with 30 μg of wild-type polymer particles, Ag85A-ESAT-6 polymer particles, Ag85A-ESAT-6 polymer particles with Emulsigen or non-immunised. * indicates a significantly greater response than the PBS immunised control group (p<0.01). ** indicates a significantly greater response than all the other vaccine groups (p<0.01). See Example 6 herein.

FIG. 9 shows the binding of anti-ESAT-6 antibody to Ag85a-ESAT-6 polymer particles. See Example 5 herein.

FIG. 10 shows the lung culture results following vaccination of mice with various polymer particle vaccines and then challenged with M. bovis. * indicates statistical difference to the non-vaccinated group (p<0.05). Bars indicate SEM. See Example 6 herein.

FIG. 11 shows the spleen culture results following vaccination of mice with various polymer particle vaccines. * indicates statistical difference to the non-vaccinated group (p<0.05). Bars indicate SEM. See Example 6 herein.

FIG. 12 shows the IgG1 antibody response in mice immunised with various polymer particle vaccines and then challenged with M. bovis. EC50 refers to the reciprocal serum titre which gives half-maximal optical density. Level of detection is 25. * indicates significant difference to other groups. (p<0.05). Bars indicate SEM. See Example 6 herein.

FIG. 13 shows the IgG2c antibody response in mice immunised with various polymer particle vaccines and then challenged with M. bovis. EC50 refers to the reciprocal serum titre which gives half-maximal optical density. Level of detection is 25. * indicates significant difference to other groups. (p<0.05). Bars indicate SEM. See Example 6 herein.

### DETAILED DESCRIPTION

The present invention relates to polymer particles and uses thereof. In particular the present invention relates to functionalised polymer particles, for example, processes of production of functionalised polymer particles, and uses thereof in the treatment or prevention of various diseases and conditions, including those caused by or associated with pathogens including those identified or described herein.

Functionalised polymer particles of the present invention may comprise one or more surface-bound fusion polypeptides, and may also comprise one or more substances incorporated or adsorbed into the polymer particle core, one or more substances bound to surface bound fusion polypeptides, or a combination thereof.

### 1. Definitions

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct, a “coding sequence” is capable of being expressed when it is operably linked to promoter and terminator sequences.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

The term “coupling reagent” as used herein refers to an inorganic or organic compound that is suitable for binding at least one substance or a further coupling reagent that is suitable for binding a coupling reagent on one side and at least one substance on the other side. Examples of suitable coupling reagents, as well as exemplary methods for their use including methods suitable for the chemical modification of particles or fusion proteins of the present invention, are presented in PCT/DE2003/002799, published as WO 2004/020623 (Bernd Rehm), herein incorporated by reference in its entirety.

The term “expression construct” refers to a genetic construct that includes elements that permit transcribing the insert polynucleotide molecule, and, optionally, translating the transcript into a polypeptide. An expression construct typically comprises in a 5′ to 3′ direction:
(1) a promoter, functional in the host cell into which the construct will be introduced,
(2) the polynucleotide to be expressed, and
(3) a terminator functional in the host cell into which the construct will be introduced.

Expression constructs of the invention are inserted into a replicable vector for cloning or for expression, or are incorporated into the host genome.

Examples of expression constructs amenable for adaptation for use in the present invention are provided in PCT/DE2003/002799 published as WO 2004/020623 (Bernd Rehm) and PCT/NZ2006/000251 published as WO 2007/037706 (Bernd Rehm) which are each herein incorporated by reference in their entirety.

The terms “form a polymer particle” and “formation of polymer particles”, as used herein, refer to the activity of a particle-forming protein as discussed herein.

A “fragment” of a polypeptide is a subsequence of the polypeptide that performs a function that is required for the enzymatic or binding activity and/or provides three dimensional structure of the polypeptide.

The term “fusion polypeptide”, as used herein, refers to a polypeptide comprising two or amino acid sequences, for example two or more polypeptide domains, fused through respective amino and carboxyl residues by a peptide linkage to form a single continuous polypeptide. It should be understood that the two or more amino acid sequences can either be directly fused or indirectly fused through their respective amino and carboxyl terimini through a linker or spacer or an additional polypeptide.

In one embodiment, one of the amino acid sequences comprising the fusion polypeptide comprises a particle-forming protein.

In one embodiment, one of the amino acid sequences comprising the fusion polypeptide comprises a M. tuberculosis antigen, or a M. tuberculosis antigen binding domain, or a fusion partner.

The term “fusion partner” as used herein refers to a polypeptide such as a protein, a protein fragment, a binding domain, a target-binding domain, a binding protein, a binding protein fragment, an antibody, an antibody fragment, an antibody heavy chain, an antibody light chain, a single chain antibody, a single-domain antibody (a VHH for example), a Fab antibody fragment, an Fc antibody fragment, an Fv antibody fragment, a F(ab′)2 antibody fragment, a Fab′ antibody fragment, a single-chain Fv (scFv) antibody fragment, an antibody binding domain (a ZZ domain for example), an antigen, an antigenic determinant, an epitope, a hapten, an immunogen, an immunogen fragment, biotin, a biotin derivative, an avidin, a streptavidin, a substrate, an enzyme, an abzyme, a co-factor, a receptor, a receptor fragment, a receptor subunit, a receptor subunit fragment, a ligand, an inhibitor, a hormone, a lectin, a polyhistidine, a coupling domain, a DNA binding domain, a FLAG epitope, a cysteine residue, a library peptide, a reporter peptide, an affinity purification peptide, or any combination of any two or more thereof.

It should be understood that two or more polypeptides listed above can form the fusion partner.

In one embodiment the amino acid sequences of the fusion polypeptide are indirectly fused through a linker or spacer, the amino acid sequences of said fusion polypeptide arranged in the order of polymer synthase-linker-antigen capable of eliciting an immune response, or antigen capable of eliciting an immune response-linker-polymer synthase, or polymer synthase-linker-binding domain of an antigen capable of eliciting an immune response, or binding domain of antigen capable of eliciting an immune response-linker-polymer synthase, for example. In other embodiments the amino acid sequences of the fusion polypeptide are indirectly fused through or comprise an additional polypeptide arranged in the order of polymer synthase-additional polypeptide-antigen capable of eliciting an immune response or polymer synthase-additional polypeptide-binding domain of an antigen capable of eliciting an immune response, or polymer synthase-linker-antigen capable of eliciting an immune response-additional polypeptide or polymer synthase-linker-binding domain of an antigen capable of eliciting an immune response—additional polypeptide. Again, N-terminal extensions of the polymer synthase are expressly contemplated herein.

Immune responses include cell-mediated and humoral immune responses.

In one embodiment the amino acid sequences of the fusion polypeptide are indirectly fused through a linker or spacer, the amino acid sequences of said fusion polypeptide arranged in the order of polymer synthase-linker-M. tuberculosis antigen or M. tuberculosis antigen-linker-polymer synthase, or polymer synthase-linker-M. tuberculosis antigen binding domain or M. tuberculosis antigen binding domain-linker-polymer synthase, for example. In other embodiments the amino acid sequences of the fusion polypeptide are indirectly fused through or comprise an additional polypeptide arranged in the order of polymer synthase-additional polypeptide-M. tuberculosis antigen or polymer synthase-additional polypeptide-M. tuberculosis antigen binding domain, or polymer synthase-linker-M. tuberculosis antigen-additional polypeptide or polymer synthase-linker-M. tuberculosis antigen binding domain-additional polypeptide. Again, N-terminal extensions of the polymer synthase are expressly contemplated herein.

A fusion polypeptide according to the invention may also comprise one or more polypeptide sequences inserted within the sequence of another polypeptide. For example, a polypeptide sequence such as a protease recognition sequence are inserted into a variable region of a protein comprising a particle binding domain.

Conveniently, a fusion polypeptide of the invention are encoded by a single nucleic acid sequence, wherein the nucleic acid sequence comprises at least two subsequences each encoding a polypeptide or a polypeptide domain. In certain embodiments, the at least two subsequences will be present “in frame” so as comprise a single open reading frame and thus will encode a fusion polypeptide as contemplated herein. In other embodiments, the at least two subsequences are present “out of frame”, and are separated by a ribosomal frame-shifting site or other sequence that promotes a shift in reading frame such that, on translation, a fusion polypeptide is formed. In certain embodiments, the at least two subsequences are contiguous. In other embodiments, such as those discussed above where the at least two polypeptides or polypeptide domains are indirectly fused through an additional polypeptide, the at least two subsequences are not contiguous.

Reference to a “binding domain” or a “domain capable of binding” is intended to mean one half of a complementary binding pair and may include binding pairs from the list above. For example, antibody-antigen, antibody-antibody binding domain, biotin-streptavidin, receptor-ligand, enzyme-inhibitor pairs. A target-binding domain will bind a target molecule in a sample, and are an antibody or antibody fragment, for example. A polypeptide-binding domain will bind a polypeptide, and are an antibody or antibody fragment, or a binding domain from a receptor or signalling protein, for example.

Examples of substances that are bound by a binding domain include a protein, a protein fragment, a peptide, a polypeptide, a polypeptide fragment, an antibody, an antibody fragment, an antibody binding domain, an antigen, an antigen fragment, an antigenic determinant, an epitope, a hapten, an immunogen, an immunogen fragment, a pharmaceutically active agent, a biologically active agent, an adjuvant or any combination of any two or more thereof. Such substances are “target components” in a sample that is analysed according to a method of the invention.

Accordingly, a “domain capable of binding an antigen capable of eliciting an immune response” and grammatical equivalents will be understood to refer to one component in a complementary binding pair, wherein the other component is the antigen capable of eliciting an immune response.

Likewise, a “domain capable of binding an antigen capable of eliciting a cell-mediated immune response” and grammatical equivalents will be understood to refer to one component in a complementary binding pair, wherein the other component is the antigen capable of eliciting a cell-mediated response. For example, a domain capable of binding a M. tuberculosis antigen, which may also be referred to as a M. tuberculosis antigen binding domain, is a domain that is able to bind one or more M. tuberculosis antigens.

Accordingly, a “M. tuberculosis antigen binding domain” is a domain that is able to bind one or more M. tuberculosis antigens.

A “M. tuberculosis antigen” as used herein is an antigen derived from M. tuberculosis. Likewise, other antigens are identified by the organism from which they are derived.

The phrase “antigen capable of eliciting an immune response” refers to an antigen that, when contacted with one or more agents of the immune system, such as one or more antibodies or one or more cells, is able to elicit or upregulate the responsiveness of the immune system, such as, for example, an upregulation in one or more T cell populations, such as for example increased CD8+ T-cell or CD4+ T cell activity or number, or an upregulation in one or more B cell populations, such as one or more B cell populations capable of producing antibodies specific to the antigen or capable of binding the antigen, or an increase in the amount or activity of one or more populations of antibodies.

The phrase “antigen capable of eliciting a cell-mediated response” refers to an antigen that, when contacted with one or more cells of the immune system, is able to elicit or upregulate the responsiveness of the immune system, such as, for example, an upregulation in one or more T cell populations, such as for example increased CD8+ T-cell or CD4+ T cell activity or number.

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain the necessary elements that permit transcribing the insert polynucleotide molecule, and, optionally, translating the transcript into a polypeptide. The insert polynucleotide molecule are derived from the host cell, or are derived from a different cell or organism and/or are a recombinant polynucleotide. Once inside the host cell the genetic construct becomes integrated in the host chromosomal DNA. In one example the genetic construct is linked to a vector.

The term “host cell” refers to a bacterial cell, a fungi cell, yeast cell, a plant cell, an insect cell or an animal cell such as a mammalian host cell that is either 1) a natural PHA particle producing host cell, or 2) a host cell carrying an expression construct comprising nucleic acid sequences encoding at least a thiolase and a reductase and optionally a phasin. Which genes are required to augment what the host cell lacks for polymer particle formation will be dependent on the genetic makeup of the host cell and which substrates are provided in the culture medium.

The term “linker or spacer” as used herein relates to an amino acid or nucleotide sequence that indirectly fuses two or more polypeptides or two or more nucleic acid sequences encoding two or more polypeptides. In some embodiments the linker or spacer is about 1, 5, 10, 15, 20, 25, 30, 35, 40, 45, 50, 55, 60, 65, 70, 75, 80, 85, 90, 95 or about 100 amino acids or nucleotides in length. In other embodiments the linker or spacer is about 100, 125, 150, 175, 200, 225, 250, 275, 300, 325, 350, 375, 400, 450, 500, 550, 600, 650, 700, 750, 800, 850, 900, 950 or about 1000 amino acids or nucleotides in length. In still other embodiments the linker or spacer is from about 1 to about 1000 amino acids or nucleotides in length, from about 10 to about 1000, from about 50 to about 1000, from about 100 to about 1000, from about 200 to about 1000, from about 300 to about 1000, from about 400 to about 1000, from about 500 to about 1000, from about 600 to about 1000, from about 700 to about 1000, from about 800 to about 1000, or from about 900 to about 1000 amino acids or nucleotides in length.

In one embodiment the linker or spacer may comprise a restriction enzyme recognition site. In another embodiment the linker or spacer may comprise a protease cleavage recognition sequence such as enterokinase, thrombin or Factor Xa recognition sequence, or a self-splicing element such as an intein. In another embodiment the linker or spacer facilitates independent folding of the fusion polypeptides.

The term “mixed population”, as used herein, refers to two or more populations of entities, each population of entities within the mixed population differing in some respect from another population of entities within the mixed population. For example, when used in reference to a mixed population of expression constructs, this refers to two or more populations of expression constructs where each population of expression construct differs in respect of the fusion polypeptide encoded by the members of that population, or in respect of some other aspect of the construct, such as for example the identity of the promoter present in the construct. Alternatively, when used in reference to a mixed population of fusion polypeptides, this refers to two or more populations of fusion polypeptides where each population of fusion polypeptides differs in respect of the polypepetides, such as polymer synthase, the antigen capable of eliciting a cell-mediated immune response, or the binding domain capable of binding an antigen capable of eliciting a cell-mediated immune response, the members that population contains. For example, in the context of use in the treatment of tuberculosis, a mixed population of fusion polypeptides refers to two or more populations of fusion polypeptides where each population of fusion polypeptides differs in respect of the polypepetides, such as polymer synthase, the M. tuberculosis antigen, or the M. tuberculosis antigen binding domain, the members that population contains. Similarly, in the context of hepatitis or influenza a mixed population of fusion polypeptides refers to two or more populations of fusion polypeptides where each population of fusion polypeptides differs in respect of the polypepetides, such as polymer synthase, the hepatitis antigen, the hepatitis antigen binding domain, the influenza antigen or the influenza antigen binding domain the members that population contains. Still further, when used in reference to a mixed population of polymer particles, this refers to two or more populations of polymer particles where each population of polymer particles differs in respect of the fusion polypeptide or fusion polypeptides the members of that population carry.

The term “nucleic acid” as used herein refers to a single- or double-stranded polymer of deoxyribonucleotide, ribonucleotide bases or known analogues of natural nucleotides, or mixtures thereof. The term includes reference to a specified sequence as well as to a sequence complimentary thereto, unless otherwise indicated. The terms “nucleic acid” and “polynucleotide” are used herein interchangeably.

“Operably-linked” means that the sequenced to be expressed is placed under the control of regulatory elements that include promoters, tissue-specific regulatory elements, temporal regulatory elements, enhancers, repressors and terminators.

The term “over-expression” generally refers to the production of a gene product in a host cell that exceeds levels of production in normal or non-transformed host cells. The term “overexpression” when used in relation to levels of messenger RNA preferably indicates a level of expression at least about 3-fold higher than that typically observed in a host cell in a control or non-transformed cell. More preferably the level of expression is at least about 5-fold higher, about 10-fold higher, about 15-fold higher, about 20-fold higher, about 25-fold higher, about 30-fold higher, about 35-fold higher, about 40-fold higher, about 45-fold higher, about 50-fold higher, about 55-fold higher, about 60-fold higher, about 65-fold higher, about 70-fold higher, about 75-fold higher, about 80-fold higher, about 85-fold higher, about 90-fold higher, about 95-fold higher, or about 100-fold higher or above, than typically observed in a control host cell or non-transformed cell.

Levels of mRNA are measured using any of a number of techniques known to those skilled in the art including, but not limited to, Northern blot analysis and RT-PCR, including quantitative RT-PCR.

The term “particle-forming protein”, as used herein, refers to proteins involved in the formation of the particle. It may, for example, be selected from the group of proteins which comprises a polymer depolymerase, a polymer regulator, a polymer synthase and a particle size-determining protein. Preferably the particle-forming protein is selected from the group comprising a thiolase, a reductase, a polymer synthase and a phasin. A particle-forming protein such as a synthase may catalyse the formation of a polymer particle by polymerising a substrate or a derivative of a substrate to form a polymer particle. Alternatively, a particle-forming protein such as a thiolase, a reductase or a phasin may facilitate the formation of a polymer particle by facilitating polymerisation. For example, a thiolase or reductase may catalyse production of suitable substrates for a polymerase. A phasin may control the size of the polymer particle formed. Preferably the particle-forming protein comprises a particle binding domain and a particle forming domain.

As used herein, the term “particle-forming reaction mixture” refers to at least a polymer synthase substrate if the host cell or expression construct comprises a synthase catalytic domain or a polymer synthase and its substrate if the host cell or expression construct comprises another particle-forming protein or a particle binding domain that is not a polymer synthase catalytic domain.

A “particle size-determining protein” refers to a protein that controls the size of the polymer particles. It may for example be derived from the family of phasin-like proteins, preferably selected from the those from the genera Ralstonia, Alcaligenes and Pseudomonas, more preferably the phasin gene phaP from Ralstonia eutropha and the phasin gene phaF from Pseudomonas oleovorans. Phasins are amphiphilic proteins with a molecular weight of 14 to 28 kDa which bind tightly to the hydrophobic surface of the polymer particles. It may also comprise other host cell proteins that bind particles and influence particle size.

The term “pathogen” or “intracellular pathogen” or “microbe” refers to any organism that exists within a host cell, either in the cytoplasm or within a vacuole, for at least part of its reproductive or life cycle. Intracellular pathogens include viruses (e.g. CMV, HIV), bacteria (Mycobacterium, Listeria, Salmonella, Shigella, Yersinia, Brucella, Bacillus, Legionella, Rickettsiae, Clamydia, Clamydophilia, Streptococcus, Staphylococcus, Ehrlichia, Francisella, enteropathogenic Escherichia coli, enterohaemorrhagic Escherichia coli), protozoa (e.g. Taxoplasma), fungi, and intracellular parasites (e.g. Plasmodium).

It will be appreciated that pathogens are typically host-specific. Accordingly, the methods and compositions of the invention are amenable to modification (use) in immunising a particular host species against a particular pathogen, including against a species-specific pathogen. For example, humans are immunised against pathogens, including human-specific pathogens, such as for example Mycobacterium (e.g M. bovis, M. tuberculosis, M. leprae, M. kansasii, M. avium, M. avium paratuberculosis, Mycobacterium sp.), Listeria (e.g. L. monocytogenes, Listeria sp.), Salmonella (e.g. S. typhi), Yersinia (e.g. Y. pestis, Y. enterocolitica, Y. pseudotuberculosis), Bacillus anthracis, Legionella (e.g. L. pneumophila, L. longbeachae, L. bozemanii, Legionella sp.), Rickettsia (e.g. R. rickettsii, R. akari, R. conorii, R. siberica, R. australis, R. japonica, R. africae, R. prowazekii, R. typhi, Rickettsia sp.), Chlamydia (e.g. C. pneumoniae, C. trachomatis, Chlamydia sp.), Clamydophila (e.g. C. psittaci, C. abortus), Streptococcus (e.g. S. pneumoniae, S. pyogenes, S. agalactiae), Staphylococcus (e.g. S. aureus), Ehrlichia (e.g. E. chaffeensis, Ehrlichia phagocytophila geno group, Ehrlichia sp.), Coxiella burnetii, Leishmania sp., Toxpolasma gondii, Trypanosoma cruzi, Histoplasma sp., Francisella tularensis, and adenovirus, vaccinia, avipox, adeno-associated virus, modified Vaccinia Strain Ankara, Semliki Forest virus, poxvirus, and herpes viruses.

Other genres of intracellular pathogens have wide host specificity, and include for example the Brucella species. Brucella is a genus of Gram-negative non-motile, non-encapsulated coccobacilli. Brucella is the cause of brucellosis. Examples of different Brucella species include B. melitensis, B. abortus, B. suis, B. ovis, B. pinnipediae, and B. neotomae.

In other examples, non-human subjects are immunised against pathogens, including species-specific pathogens. For example, bovine, corvine and ovine subjects are immunised against Mycobacterium spp., including for example e.g M. bovis, M. tuberculosis, M. leprae, M. kansasii, M. avium, M. avium paratuberculosis, and other Mycobacterium spp.

Accordingly, a “subject” is an animal, such as a mammal, including a mammalian companion animal or a human. Representative companion animals include feline, equine, and canine. Representative agricultural animals include bovine, ovine, cervine, and porcine. In one embodiment the human is an adult, a child, or an infant, including an immunocompromised adult, child, or infant, or an adult, a child or an infant vaccinated against, infected with, exposed to or at risk of infection or exposure to a pathogen.

The term “treat” and its derivatives (including “treatment”) should be interpreted in their broadest possible context. The term should not be taken to imply that a subject is treated until total recovery. Accordingly, “treat” broadly includes amelioration and/or prevention of the onset of the symptoms or severity of a particular condition.

A “polymer regulator” as used herein refers to a protein which regulates the transcription of the genes phaA, phaB and phaC involved in the formation of the polymer particles. It is withdrawn from transcription regulation by binding to the particle surface. One example of such a regulator is the phasin repressor (phaR) from R. eutropha YP_725943, which binds to the promoter of a phasin-like gene, the expression product of which regulates the size of polymer particles formed, and prevents the gene from being read. Because the phasin repressor is bound on the surface of the polymer particles formed, this site on the promoter is released and transcription of the underlying gene can begin. A “polymer synthase” as used herein refers to a protein which is capable of catalysing the formation of a polymer particle by polymerising a substrate or a derivative of a substrate to form a polymer particle. The nucleotide sequences of 88 PHA synthase genes from >45 different bacteria have been obtained, differing in primary structure, substrate specificity and subunit composition (Rehm, 2007).

A polymer synthase comprises at least the synthase catalytic domain at the C-terminus of the synthase protein that mediates polymerisation of the polymer and attachment of the synthase protein to the particle core. Polymer synthases for use in the present invention are described in detail in Rehm, 2003, which is herein incorporated by reference in its entirety. For example, the polymer synthase is a PHA synthase from the class 1 genera Acinetobacter, Vibrio, Aeromonas, Chromobacterium, Pseudomonas, Zoogloea, Alcaligenes, Delftia, Burkholderia, Ralstonia, Rhodococcus, Gordonia, Rhodobacter, Paracoccus, Rickettsia, Caulobacter, Methylobacterium, Azorhizobium, Agrobacterium, Rhizobium, Sinorhizobium, Rickettsia, Crenarchaeota, Synechocystis, Ectothiorhodospira, Thiocapsa, Thyocystis and Allochromatium, the class 2 genera Burkholderia and Pseudomonas, or the class 4 genera Bacillus, more preferably from the group comprising class 1 Acinetobacter sp. RA3849, Vibrio cholerae, Vibrio parahaemolyticus, Aeromonas punctata FA440, Aeromonas hydrophila, Chromobacterium violaceum, Pseudomonas sp. 61-3, Zoogloea ramigera, Alcaligenes latus, Alcaligenes sp. SH-69, Delftia acidovorans, Burkholderia sp. DSMZ9242, Ralstonia eutrophia H16, Burkholderia cepacia, Rhodococcus rubber PP2, Gordonia rubripertinctus, Rickettsia prowazekii, Synechocystis sp. PCC6803, Ectothiorhodospira shaposhnikovii N1, Thiocapsa pfennigii 9111, Allochromatium vinosum D, Thyocystis violacea 2311, Rhodobacter sphaeroides, Paracoccus denitrificans, Rhodobacter capsulatus, Caulobacter crescentus, Methylobacterium extorquens, Azorhizobium caulinodans, Agrobacterium tumefaciens, Sinorhizobium meliloti 41, Rhodospirillum rubrum HA, and Rhodospirillum rubrum ATCC25903, class 2 Burkholderia caryophylli, Pseudomonas chloraphis, Pseudomonas sp. 61-3, Pseudomonas putida U, Pseudomonas oleovorans, Pseudomonas aeruginosa, Pseudomonas resinovorans, Pseudomonas stutzeri, Pseudomonas mendocina, Pseudomonas pseudolcaligenes, Pseudomonas putida BM01, Pseudomonas nitroreducins, Pseudomonas chloraphis, and class 4 Bacillus megaterium and Bacillus sp. INT005.

Other polymer synthases amenable to use in the present invention include polymer synthases, each identified by it accession number, from the following organisms: C. necator (AY836680), P. aeruginosa (AE004091), A. vinosum (AB205104), B. megaterium (AF109909), H. marismortui (YP137339), P. aureofaciens (AB049413), P. putida (AF150670), R. eutropha (A34341), T. pfennigii (X93599), A. punctata (032472), Pseudomonas sp. 61-3 (AB014757 and AB014758), R. sphaeroides (AAA72004, C. violaceum (AAC69615), A. borkumensis SK2 (CAL17662), A. borkumensis SK2 (CAL16866), R. sphaeroides KD131 (ACM01571 AND YP002526072), R. opacus B4 (BAH51880 and YP002780825), B. multivorans ATCC 17616 (YP001946215 and BAG43679), A. borkumensis SK2(YP693934 and YP693138), R. rubrum (AAD53179), gamma proteobacterium HTCC5015 (ZP05061661 and EDY86606), Azoarcus sp. BH72 (YP932525), C. violaceum ATCC 12472 (NP902459), Limnobacter sp. MED105 (ZP01915838 and EDM82867), M. algicola DG893 (ZP01895922 and EDM46004), R. sphaeroides (CAA65833), C. violaceum ATCC 12472 (AAQ60457), A. latus (AAD10274, AAD01209 and AAC83658), S. maltophilia K279a (CAQ46418 and YP001972712), R. solanacearum IP01609 (CAQ59975 and YP002258080), B. multivorans ATCC 17616 (YP001941448 and BAG47458), Pseudomonas sp. g113 (ACJ02400), Pseudomonas sp. g106 (ACJ02399), Pseudomonas sp. gl01 (ACJ02398), R. sp. g132 (ACJ02397), R. leguminosarum bv. viciae 3841 (CAK10329 and YP770390), Azoarcus sp. BH72 (CAL93638), Pseudomonas sp. LDC-5 (AAV36510), L. nitroferrum 2002 (ZP03698179), Thauera sp. MZ1T (YP002890098 and ACR01721), M. radiotolerans JCM 2831 (YP001755078 and ACB24395), Methylobacterium sp. 4-46 (YP001767769 and ACA15335), L. nitroferrum 2002 (EEG08921), P. denitrificans (BAA77257), M. gryphiswaldense (ABG23018), Pseudomonas sp. USM4-55 (ABX64435 and ABX64434), A. hydrophila (AAT77261 and AAT77258), Bacillus sp. INT005 (BAC45232 and BAC45230), P. putida (AAM63409 and AAM63407), G. rubripertinctus (AAB94058), B. megaterium (AAD05260), D. acidovorans (BAA33155), P. seriniphilus (ACM68662), Pseudomonas sp. 14-3 (CAK18904), Pseudomonas sp. LDC-5 (AAX18690), Pseudomonas sp. PC17 (ABV25706), Pseudomonas sp. 3Y2 (AAV35431, AAV35429 and AAV35426), P. mendocina (AAM10546 and AAM10544), P. nitroreducens (AAK19608), P. pseudoalcaligenes (AAK19605), P. resinovorans (AAD26367 and AAD26365), Pseudomonas sp. USM7-7 (ACM90523 and ACM90522), P. fluorescens (AAP58480) and other uncultured bacterium (BAE02881, BAE02880, BAE02879, BAE02878, BAE02877, BAE02876, BAE02875, BAE02874, BAE02873, BAE02872, BAE02871, BAE02870, BAE02869, BAE02868, BAE02867, BAE0286, BAE02865, BAE02864, BAE02863, BAE02862, BAE02861, BAE02860, BAE02859, BAE02858, BAE02857, BAE07146, BAE07145, BAE07144, BAE07143, BAE07142, BAE07141, BAE07140, BAE07139, BAE07138, BAE07137, BAE07136, BAE07135, BAE07134, BAE07133, BAE07132, BAE07131, BAE07130, BAE07129, BAE07128, BAE07127, BAE07126, BAE07125, BAE07124, BAE07123, BAE07122, BAE07121, BAE07120, BAE07119, BAE07118, BAE07117, BAE07116, BAE07115, BAE07114, BAE07113, BAE07112, BAE07111, BAE07110, BAE07109, BAE07108, BAE07107, BAE07106, BAE07105, BAE07104, BAE07103, BAE07102, BAE07101, BAE07100, BAE07099, BAE07098, BAE07097, BAE07096, BAE07095, BAE07094, BAE07093, BAE07092, BAE07091, BAE07090, BAE07089, BAE07088, BAE07053, BAE07052, BAE07051, BAE07050, BAE07049, BAE07048, BAE07047, BAE07046, BAE07045, BAE07044, BAE07043, BAE07042, BAE07041, BAE07040, BAE07039, BAE07038, BAE07037, BAE07036, BAE07035, BAE07034, BAE07033, BAE07032, BAE07031, BAE07030, BAE07029, BAE07028, BAE07027, BAE07026, BAE07025, BAE07024, BAE07023, BAE07022, BAE07021, BAE07020, BAE07019, BAE07018, BAE07017, BAE07016, BAE07015, BAE07014, BAE07013, BAE07012, BAE07011, BAE07010, BAE07009, BAE07008, BAE07007, BAE07006, BAE07005, BAE07004, BAE07003, BAE07002, BAE07001, BAE07000, BAE06999, BAE06998, BAE06997, BAE06996, BAE06995, BAE06994, BAE06993, BAE06992, BAE06991, BAE06990, BAE06989, BAE06988, BAE06987, BAE06986, BAE06985, BAE06984, BAE06983, BAE06982, BAE06981, BAE06980, BAE06979, BAE06978, BAE06977, BAE06976, BAE06975, BAE06974, BAE06973, BAE06972, BAE06971, BAE06970, BAE06969, BAE06968, BAE06967, BAE06966, BAE06965, BAE06964, BAE06963, BAE06962, BAE06961, BAE06960, BAE06959, BAE06958, BAE06957, BAE06956, BAE06955, BAE06954, BAE06953, BAE06952, BAE06951, BAE06950, BAE06949, BAE06948, BAE06947, BAE06946, BAE06945, BAE06944, BAE06943, BAE06942, BAE06941, BAE06940, BAE06939, BAE06938, BAE06937, BAE06936, BAE06935, BAE06934, BAE06933, BAE06932, BAE06931, BAE06930, BAE06929, BAE06928, BAE06927, BAE06926, BAE06925, BAE06924, BAE06923, BAE06922, BAE06921, BAE06920, BAE06919, BAE06918, BAE06917, BAE06916, BAE06915, BAE06914, BAE06913, BAE06912, BAE06911, BAE06910, BAE06909, BAE06908, BAE06907, BAE06906, BAE06905, BAE06904, BAE06903, BAE06902, BAE06901, BAE06900, BAE06899, BAE06898, BAE06897, BAE06896, BAE06895, BAE06894, BAE06893, BAE06892, BAE06891, BAE06890, BAE06889, BAE06888, BAE06887, BAE06886, BAE06885, BAE06884, BAE06883, BAE06882, BAE06881, BAE06880, BAE06879, BAE06878, BAE06877, BAE06876, BAE06875, BAE06874, BAE06873, BAE06872, BAE06871, BAE06870, BAE06869, BAE06868, BAE06867, BAE06866, BAE06865, BAE06864, BAE06863, BAE06862, BAE06861, BAE06860, BAE06859, BAE06858, BAE06857, BAE06856, BAE06855, BAE06854, BAE06853 and BAE06852).

The N-terminal fragment of PHA synthase protein (about amino acids 1 to 200, or 1 to 150, or 1 to 100) is highly variable and in some examples is deleted or replaced by an antigen, an antigen binding domain, or another fusion partner without inactivating the enzyme or preventing covalent attachment of the synthase via the polymer particle binding domain (i.e. the C-terminal fragment) to the polymer core. The polymer particle a binding domain capable of binding the synthase comprises at least the catalytic domain of the synthase protein that mediates polymerisation of the polymer core and formation of the polymer particles.

In some embodiments the C-terminal fragment of PHA synthase protein is modified, partially deleted or partially replaced by an antigen capable of eliciting an immune response, a binding domain capable of binding an antigen capable of eliciting an immune response, or another fusion partner without inactivating the enzyme or preventing covalent attachment of the synthase to the polymer particle.

In certain cases, the antigen capable of eliciting an immune response, the binding domain capable of binding an antigen capable of binding an immune response, or another fusion partner are fused to the N-terminus or to the C-terminus of PHA synthase protein without inactivating the enzyme or preventing covalent attachment of the synthase to the polymer particle. Similarly, in other cases the antigen capable of eliciting an immune response, the binding domain capable of binding an antigen capable of eliciting an immune response, or another fusion partner are inserted within the PHA synthase protein, or indeed within the particle-forming protein. Examples of PhaC fusions are known in the art and presented herein.

In one example, the N-terminal fragment of PHA synthase protein (about amino acids 1 to 200, or 1 to 150, or 1 to 100) is highly variable and is deleted or replaced by a M. tuberculosis antigen, a M. tuberculosis antigen binding domain, a hepatitis antigen, a hepatitis antigen binding domain, an influenza antigen or an influenza antigen binding domain or another fusion partner without inactivating the enzyme or preventing covalent attachment (covalent attachment occurs through the active site from which the nascent polyester protrudes) of the synthase via the polymer particle binding domain (i.e. the C-terminal fragment (this domain binds via hydrophobic interaction)) to the polymer particle. The polymer particle binding domain of the synthase comprises at least the catalytic domain of the synthase protein that mediates polymerisation of the polymer particle and formation of the polymer particles.

The C-terminal fragment of PHA synthase protein may also be modified, partially deleted or partially replaced, for example by a M. tuberculosis antigen, a M. tuberculosis antigen binding domain, a hepatitis antigen, a hepatitis antigen binding domain, an influenza antigen or an influenza antigen binding or another fusion partner without inactivating the enzyme or preventing covalent attachment of the synthase to the polymer particle.

In certain cases, the M. tuberculosis antigen, the M. tuberculosis antigen binding domain, a hepatitis antigen, a hepatitis antigen binding domain, an influenza antigen or an influenza antigen binding or another fusion partner are fused to the N-terminus or to the C-terminus of PHA synthase protein without inactivating the enzyme or preventing covalent attachment of the synthase to the polymer particle. Similarly, in other cases the M. tuberculosis antigen, a M. tuberculosis antigen binding domain, a hepatitis antigen, a hepatitis antigen binding domain, an influenza antigen or an influenza antigen binding or another fusion partner are inserted within the PHA synthase protein, or indeed within the particle-forming protein. Examples of PhaC fusions are known in the art and presented herein.

A “polymer depolymerase” as used herein refers to a protein which is capable of hydrolysing existing polymer, such as that found in a polymer particle, into water soluble monomers and oligomers. Examples of polymer depolymerases occur in a wide variety of PHA-degrading bacteria and fungi, and include the PhaZ1-PhaZ7 extracellular depolymerases from Paucimonas lemoignei, the PhaZ depolymerases from Acidovorax sp., A. faecalis (strains AE122 and T1), Delftia (Comamonas) acidovorans strain YM1069, Comamonas testosteroni, Comamonas sp., Leptothrix sp. strain HS, Pseudomonas sp. strain GM101 (accession no. AF293347), P. fluorescens strain GK13, P. stutzeri, R. pickettii (strains A1 and K1, accession no. JO4223, D25315), S. exfoliatus K10 and Streptomyces hygroscopicus (see Jendrossek D., and Handrick, R., Microbial Degredation of Polyhydroxyalkanoates, Annual Review of Microbiology, 2002, 56:403-32).

The term “polypeptide”, as used herein, encompasses amino acid chains of any length but preferably at least 5 amino acids, including full-length proteins, in which amino acid residues are linked by covalent peptide bonds. Polypeptides of the present invention are purified natural products, or are produced partially or wholly using recombinant or synthetic techniques. The term may refer to a polypeptide, an aggregate of a polypeptide such as a dimer or other multimer, a fusion polypeptide, a polypeptide variant, or derivative thereof.

The term “promoter” refers to non transcribed cis-regulatory elements upstream of the coding region that regulate gene transcription. Promoters comprise cis-initiator elements which specify the transcription initiation site and conserved boxes such as the TATA box, and motifs that are bound by transcription factors.

The term “terminator” refers to sequences that terminate transcription, which are found in the 3′ untranslated ends of genes downstream of the translated sequence. Terminators are important determinants of mRNA stability and in some cases have been found to have spatial regulatory functions.

The term “substance” when referred to in relation to being bound to or absorbed into or incorporated within a polymer particle is intended to mean a substance that is bound by a fusion partner or a substance that is able to be absorbed into or incorporated within a polymer particle.

The term “variant” as used herein refers to polynucleotide or polypeptide sequences different from the specifically identified sequences, wherein one or more nucleotides or amino acid residues is deleted, substituted, or added. Variants are naturally-occurring allelic variants, or non-naturally occurring variants. Variants are from the same or from other species and may encompass homologues, paralogues and orthologues. In certain embodiments, variants of the polynucleotides and polypeptides possess biological activities that are the same or similar to those of the wild type polynucleotides or polypeptides. The term “variant” with reference to polynucleotides and polypeptides encompasses all forms of polynucleotides and polypeptides as defined herein.

Polynucleotide and Polypeptide Variants

The term “polynucleotide(s),” as used herein, means a single or double-stranded deoxyribonucleotide or ribonucleotide polymer of any length but preferably at least 15 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments. A number of nucleic acid analogues are well known in the art and are also contemplated.

A “fragment” of a polynucleotide sequence provided herein is a subsequence of contiguous nucleotides that is preferably at least 15 nucleotides in length. The fragments of the invention preferably comprises at least 20 nucleotides, more preferably at least 30 nucleotides, more preferably at least 40 nucleotides, more preferably at least 50 nucleotides and most preferably at least 60 contiguous nucleotides of a polynucleotide of the invention. A fragment of a polynucleotide sequence can be used in antisense, gene silencing, triple helix or ribozyme technology, or as a primer, a probe, included in a microarray, or used in polynucleotide-based selection methods.

The term “fragment” in relation to promoter polynucleotide sequences is intended to include sequences comprising cis-elements and regions of the promoter polynucleotide sequence capable of regulating expression of a polynucleotide sequence to which the fragment is operably linked.

Preferably fragments of promoter polynucleotide sequences of the invention comprise at least 20, more preferably at least 30, more preferably at least 40, more preferably at least 50, more preferably at least 100, more preferably at least 200, more preferably at least 300, more preferably at least 400, more preferably at least 500, more preferably at least 600, more preferably at least 700, more preferably at least 800, more preferably at least 900 and most preferably at least 1000 contiguous nucleotides of a promoter polynucleotide of the invention.

The term “primer” refers to a short polynucleotide, usually having a free 3′OH group, that is hybridized to a template and used for priming polymerization of a polynucleotide complementary to the template. Such a primer is preferably at least 5, more preferably at least 6, more preferably at least 7, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20 nucleotides in length.

The term “probe” refers to a short polynucleotide that is used to detect a polynucleotide sequence that is complementary to the probe, in a hybridization-based assay. The probe may consist of a “fragment” of a polynucleotide as defined herein. Preferably such a probe is at least 5, more preferably at least 10, more preferably at least 20, more preferably at least 30, more preferably at least 40, more preferably at least 50, more preferably at least 100, more preferably at least 200, more preferably at least 300, more preferably at least 400 and most preferably at least 500 nucleotides in length.

The term “variant” as used herein refers to polynucleotide or polypeptide sequences different from the specifically identified sequences, wherein one or more nucleotides or amino acid residues is deleted, substituted, or added. Variants are naturally-occurring allelic variants, or non-naturally occurring variants. Variants are from the same or from other species and may encompass homologues, paralogues and orthologues. In certain embodiments, variants of the polynucleotides and polypeptides possess biological activities that are the same or similar to those of the wild type polynucleotides or polypeptides. The term “variant” with reference to polynucleotides and polypeptides encompasses all forms of polynucleotides and polypeptides as defined herein.

Polynucleotide Variants

Variant polynucleotide sequences preferably exhibit at least 50%, more preferably at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least %, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a specified polynucleotide sequence. Identity is found over a comparison window of at least 20 nucleotide positions, preferably at least 50 nucleotide positions, at least 100 nucleotide positions, or over the entire length of the specified polynucleotide sequence.

Polynucleotide sequence identity can be determined in the following manner. The subject polynucleotide sequence is compared to a candidate polynucleotide sequence using BLASTN (from the BLAST suite of programs, version 2.2.10 [October 2004]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). The default parameters of bl2seq are utilized except that filtering of low complexity parts should be turned off.

The identity of polynucleotide sequences can be examined using the following unix command line parameters:
bl2seq -i nucleotideseq1 -j nucleotideseq2 -F F -p blastn

The parameter -F F turns off filtering of low complexity sections. The parameter -p selects the appropriate algorithm for the pair of sequences. The bl2seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program can be used which computes an optimal global alignment of two sequences without penalizing terminal gaps. GAP is described in the following paper: Huang, X. (1994) On Global Sequence Alignment. Computer Applications in the Biosciences 10, 227-235.

Polynucleotide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides determined using the publicly available bl2seq program from the BLAST suite of programs (version 2.2.10 [October 2004]) from NCBI (ftp://ftp.ncbi.nih.gov/blast/).

The similarity of polynucleotide sequences can be examined using the following unix command line parameters:

bl2seq -i nucleotideseq1 -j nucleotideseq2 -F F -p tblastx

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. The size of this database is set by default in the bl2seq program. For small E values, much less than one, the E value is approximately the probability of such a random match.

Variant polynucleotide sequences preferably exhibit an E value of less than 1×10-10, more preferably less than 1×10-20, less than 1×10-30, less than 1×10-40, less than 1×10-50, less than 1×10-60, less than 1×10-70, less than 1×10-80, less than 1×10-90, less than 1×10-100, less than 1×10-110, less than 1×10-120 or less than 1×10-123 when compared with any one of the specifically identified sequences.

Alternatively, variant polynucleotides of the present invention hybridize to a specified polynucleotide sequence, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration.

The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing,). Tm for polynucleotide molecules greater than about 100 bases can be calculated by the formula Tm=81. 5+0. 41% (G+C-log (Na+). (Sambrook et al., Eds, 1987, Molecular Cloning, A Laboratory Manual, 2nd Ed. Cold Spring Harbor Press; Bolton and, McCarthy, 1962, PNAS 84:1390). Typical stringent conditions for polynucleotide of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length)° C.

With respect to the DNA mimics known as peptide nucleic acids (PNAs) (Nielsen et al., Science. 1991 Dec. 6; 254(5037):1497-500) Tm values are higher than those for DNA-DNA or DNA-RNA hybrids, and can be calculated using the formula described in Giesen et al., Nucleic Acids Res. 1998 Nov. 1; 26(21):5004-6. Exemplary stringent hybridization conditions for a DNA-PNA hybrid having a length less than 100 bases are 5 to 10° C. below the Tm.

Variant polynucleotides of the present invention also encompasses polynucleotides that differ from the sequences of the invention but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), in some examples other codons for the same amino acid are changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence can be determined using the publicly available bl2seq program from the BLAST suite of programs (version 2.2.10 [October 2004]) from NCBI (ftp://ftp.ncbi.nih.gov/blast/) via the tblastx algorithm as previously described.

Polypeptide Variants

The term “variant” with reference to polypeptides encompasses naturally occurring, recombinantly and synthetically produced polypeptides. Variant polypeptide sequences preferably exhibit at least 50%, more preferably at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least %, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequences of the present invention. Identity is found over a comparison window of at least 20 amino acid positions, preferably at least 50 amino acid positions, at least 100 amino acid positions, or over the entire length of a polypeptide of the invention.

Polypeptide sequence identity can be determined in the following manner. The subject polypeptide sequence is compared to a candidate polypeptide sequence using BLASTP (from the BLAST suite of programs, version 2.2.10 [October 2004]) in bl2seq, which is publicly available from NCBI (ftp://ftp.ncbi.nih.gov/blast/). The default parameters of bl2seq are utilized except that filtering of low complexity regions should be turned off.

Polypeptide sequence identity may also be calculated over the entire length of the overlap between a candidate and subject polynucleotide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

Polypeptide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides can be determined using the publicly available bl2seq program from the BLAST suite of programs (version 2.2.10 [October 2004]) from NCBI (ftp://ftp.ncbi.nih.gov/blast/). The similarity of polypeptide sequences can be examined using the following unix command line parameters:

bl2seq -i peptideseq1 -j peptideseq2 -F F -p blastp

Variant polypeptide sequences preferably exhibit an E value of less than 1×10-10, more preferably less than 1×10-20, less than 1×10-30, less than 1×10-40, less than 1×10-50, less than 1×10-60, less than 1×10-70, less than 1×10-80, less than 1×10-90, less than 1×10-100, less than 1×10-110, less than 1×10-120 or less than 1×10-123 when compared with any one of the specifically identified sequences.

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Conservative substitutions of one or several amino acids of a described polypeptide sequence without significantly altering its biological activity are also included in the invention.

A skilled artisan will be aware of methods for making phenotypically silent amino acid substitutions (see, e.g., Bowie et al., 1990, Science 247, 1306).

A polypeptide variant of the present invention also encompasses that which is produced from the nucleic acid encoding a polypeptide, but differs from the wild type polypeptide in that it is processed differently such that it has an altered amino acid sequence. For example, a variant is produced by an alternative splicing pattern of the primary RNA transcript to that which produces a wild type polypeptide.

The term “vector” refers to a polynucleotide molecule, usually double stranded DNA, which is used to transport the genetic construct into a host cell. In certain examples the vector is capable of replication in at least one additional host system, such as E. coli.

### 2. Pathogens

It will be appreciated that the polymer particles, methods and compositions of the present invention are in part directed to the prevention or treatment of diseases caused by pathogens, including intracellular pathogens. Accordingly, antigens derived from an intracellular pathogen are amenable for use in the present invention and can be selected by persons skilled in the art. Representative intracellular pathogens are described in more detail below, but those skilled in the art will appreciate that the invention has application in the treatment or prevention of any disease or condition associated with an intracellular pathogen in accordance with the methods described herein, for example, by selecting one or more antigens from the target intracellular pathogen or one or more binding domains capable of binding an antigen from the target intracellular pathogen.

Mycobacterium is a genus of Actinobacteria. The genus includes pathogens known to cause serious diseases in mammals, including tuberculosis and leprosy. Examples of pathogen species include M. tuberculosis, M. bovis, M. africanum, M. microti; M. leprae (leprosy), M. avium paratuberculosis (associated with Crohn's disease in humans and Johne's disease in sheep).

Listeria species are Gram-positive bacilli. The most known pathogen in this genus is L. monocytogenes, the causative agent of literiosis. Listeria ivanovii is a pathogen of ruminants and is only rarely the cause of human disease.

Shigella is a genus of Gram-negative, non-spore forming rod-shaped bacteria closely related to Escherichia coli and Salmonella. Shigella is the causative agent of human shigellosis (dysentery), infecting only primates but not other mammals.

Yersinia is a Gram-negative rod shaped bacteria. Specific human pathogens include Y. enterocolitica, causing Yersiniosis, Y. pestis, the causative agent of plague and the least common pathogen Y. pseudotuberculosis. Yersinia is implicated as one of the pathogenic causes of Reactive Arthritis.

Brucella is a genus of Gram-negative non-motile, non-encapsulated coccobacilli. Brucella is the cause of brucellosis. Examples of different Brucella species include B. melitensis and B. ovis which infect ovine species, B. abortus which infects cattle, B. suis which infects swine species, B. pinnipediae isolated from marine mammals and B. neotomae. Humans typically become infected through contact with fluids from infected animals (sheep, cattle or pigs) or derived food products such as unpasteurized milk and cheese.

Legionella is a Gram-negative bacterium. The most notable species, L. pneumophila causes legionellosis or Legionnaires' disease.

Rickettsia is a genus of motile, Gram-negative, non-spore forming bacteria. Rickettsia species are carried as parasites by many ticks, fleas, and lice, causing diseases such as Rocky Mountain spotted fever (R. rickettsii), Rickettsialpox (R. akari), Boutonneuse fever (R. conorii), Siberian tick typhus (R. siberica), Australian tick typhus (R. australis), Oriental spotted fever (R. japonica), African tick bite fever (R. africae), Epidemic typhus (R. prowazekii), and Endemic typhus (R. typhi)

Salmonella is a genus of rod-shaped, Gram-negative, non-spore forming, motile enterobateria that cause illnesses in humans and many animals, including typhoid fever, paratyphoid fever, and the salmonellosis.

Chlamydia refers to a genus of bacteria, which includes the human pathogen Chlamydia trachomatis. Chlamydophila is a related bacterium, which includes the human pathogens Chlamydophila pneumoniae, causing pnemonia, Chlamydophila psittaci, causing respiratory psittacosis, and Chlamydophila abortus, which is associated with abortion in humans.

Streptococcus is a genus of spherical Gram-positive bacteria known to cause a number of human diseases including meningitis, bacterial pneumonia (S. pneumoniae), endocarditis, erysipelas and necrotizing fasciitis (S. pyogenes).

Staphylococcus is a genus of Gram-positive bacteria and is a common cause of food poisoning.

Plasmodium is a genus of parasitic protozoa. Infection with these parasites is known to cause malaria (P. falciparum).

### 2.1 Tuberculosis

Tuberculosis is a severe global health concern, resulting in over 2 million human deaths worldwide per year. The disease is caused by the bacterium M. tuberculosis. The bacterium commonly invades the lungs, through inhalation, causing infection in the lung, which can ultimately spread to other parts of the body, including the central nervous system, the lymphatic system, the circulatory system, the genitourinary system, the gastrointestinal systems, bones, joints and the skin (Dietrich, 2006; Mustafa, 2001). Various forms of tuberculosis in agricultural animals, such as bovine tuberculosis and Johne's disease, also have a significant negative effect on production.

The spread of infection by M. tuberculosis is limited by the immune system. Many individuals show few symptoms other than a cough and fever. However, approximately 30% of individuals are not able to sufficiently control the infection and develop a primary disease. Despite this, the disease is capable of sitting dormant in individuals, infecting them again years or even decades later. For this reason, M. tuberculosis is unique among infectious bacteria, as it can evade the immune response and survive in a refractory non- or slow-replicating phase for long periods of time.

Tuberculosis infection expresses itself in three phases. The first acute stage is identified by a proliferation of bacteria in the body's organs. An immune response quickly follows, controlling the infection and eventually resulting in a decline in bacterial load. Following the acute phase, the second latent phase is established. During this second stage, bacterial load is maintained at a stable and low level. M. tuberculosis change from an active multiplication state in the acute phase to a dormant state in the latent phase. A third reactivation phase may occur whereby the bacteria begin replicating again. The factors that influence this third stage are still largely unknown (Barnes and Cave, 2003).

It thought that changes in antigen specificity of the immune response occur throughout the different stages of infection, as the bacterium is capable of modulating gene expression during transition from active replication to dormancy.

### 2.2 Hepatitis

Hepatitis is a collective name for diseases commonly caused by various Hepatitis viruses. Other contributory causes of hepatitis include alcohol, toxins, drugs and autoimmune disease. Hepatitis is an inflammation of the liver, with symptoms including malaise, muscle and joint aches, loss of appetite, and jaundice and eventual liver failure in some cases. Hepatitis can be both acute and chronic, with cirrhosis observed in chronic sufferers of the disease.

### 2.3 Influenza

Influenza (more commonly referred to as the ‘flu’) is caused by RNA viruses of the Orthomyxoviridae family. Influenza results in the deaths of between 250,000 and 500,000 people a year. Common symptoms include chills, fever, sore throat, muscle aches and pains, headaches, coughing, weakness and fatigue. In severe cases, influenza can lead to pneumonia, a potentially fatal condition in the young and elderly. Influenza can be transmitted through the air, or through direct contact with infected bird droppings or nasal secretions.

Three classes of influenza virus exist (A, B and C), all sharing similar structure. Two large glycoproteins, hemagglutinin and neuraminidase, are displayed on the surface of the viral particle and are involved in the binding of the virus to target cells, transfer of the viral genome into the target cell and release of viral progeny from infected cells. There are 16 known subtypes of hemagglutinin (H1 to H16) and 9 subtypes of neuraminidase (N1 to N9).

### 2.4 Current Treatment Strategies

Current treatment strategies for protection against intracellular pathogens include specific vaccines against known antigens, or antibiotic treatment in patients infected with intracellular bacterial pathogens.

The lack of suitable vaccines for protecting against reactivation of intracellular pathogens, either prophylactically prior to infection, or therapeutically after onset of infection, has prompted the need for new and improved treatment strategies against intracellular pathogens.

For example, the only currently available vaccine for tuberculosis is Bacille Calmette-Geurin (BCG), which contains live attenuated strains of Mycobacterium bovis. The efficacy of BCG in controlling tuberculosis infection is limited. Although the vaccine appears to protect children against the primary disease, its protective efficacy against the adult form of the disease (reactivation after latency) is reduced (World Health Organisation—http://www.who.int). It has also been reported that efficacy of BCG is limited in many Third World countries where tuberculosis is prevalent. In addition, as the BCG vaccine is a live vaccine it is not suitable for administration to patients who are immuno-compromised. While the BCG vaccine reportedly reduces dissemination of M. tuberculosis to the spleen (and other organs), it does not prevent bacterial growth in the lungs.

The lack of a suitable vaccine for protecting against reactivation, either prophylactically prior to infection, or therapeutically after onset of infection, together with the other problems associated with live vaccines, has prompted the need for new and improved treatment strategies against intracellular pathogens including tuberculosis, hepatitis or influenza.

### 3. Immune Response

### 3.1. Cell-Mediated Response

Cell-mediated immunity is primarily mediated by T-lymphocytes. Pathogenic antigens are expressed on the surface of antigen presenting cells (such as macrophages, B-lymphocytes, and dendritic cells), bound to either major histocompatibility MHC Class I or MHC Class II molecules. Presentation of pathogenic antigen coupled to MHC Class II activates a helper (CD4+) T-cell response. Upon binding of the T-cell to the antigen-MHC II complex, CD4+ T-cells proliferate, releasing cytokines, including interferon-gamma (IFN-γ) and interleukin 2 (IL-2), IL-4, IL-7, and IL-12.

Presentation of pathogenic antigens bound to MHC Class I molecules activates a cytotoxic (CD8+) T-cell response. Upon binding of the T-cell to the antigen-MHC I complex, CD8+ cells secrete perforin, resulting in pathogen cell lysis, swelling and death. Alternatively, CD8+ cells induce programmed cell death or apoptosis. Activation of CD8+ T-cells is amplified by the release of specific cytokines by CD4+ T-cells.

A cell-mediated immune response is believed to be central to the immunity against various pathogens, including intracellular pathogens such as M. tuberculosis.

Methods to assess and monitor the onset or progression of a cell-mediated response in a subject are well known in the art. Convenient exemplary methods include those in which the presence of or the level of one or more cytokines associated with a cell-mediated response, such as those identified herein, is assessed. Similarly, cell-based methods to assess or monitor the onset and progression of a cell-mediated response are amenable to use in the present invention, and may include cell proliferation or activation assays, including assays targeted at identifying activation or expansion of one or more populations of immune cells, such as T-lymphocytes.

In certain embodiments, methods of the invention that elicit both a cell-mediated immune response and a humoral response are preferred.

In other embodiments, methods of the invention that elicit predominantly a cell-mediated response are preferred. Such methods may include those that elicit a cell-mediated immune response without a significant humoral response, or without any detectable humoral response. In one example, the immune response is a cell-mediated immune response, such as that indicated by an IFN-γ response, in the absence of a significant IgA response, or in the absence of a significant IgE response, or in the absence of a significant IgG response, including the absence of a significant IgG1 response, or the absence of a significant IgG2 response, or in the absence of a significant IgM response.

### 3.2. Humoral Response

The humoral immune response is mediated by secreted antibodies produced by B cells. The secreted antibodies bind to antigens presented on the surface of invading pathogens, flagging them for distruction.

It has been suggested that a combined cell-mediated and humoral response (such as that as a consequence of an initiated cell-mediated response) would be beneficial to achieve a more highly sensitive immune response to or enhance the level of protection against intracellular pathogens.

Again, methods to assess and monitor the onset or progression of a humoral response are well known in the art. These include antibody binding assays, ELISA, skink-prick tests and the like.

### 4. Antigens

It will be appreciated that a great many antigens from various pathogenic organisms have been characterised and are suitable for use in the present invention. All antigens, whether or not presently characterized, that are capable of eliciting an immune response are contemplated.

### 4.1 Tuberculosis Antigensantigens

It will be appreciated that a great many M. tuberculosis antigens have been characterised and are suitable for use in the present invention. All M. tuberculosis antigens, whether or not presently characterized, that are capable of eliciting an immune response are contemplated.

Exemplary M. tuberculosis antigens suitable for use in the present invention include early secretary antigen target (ESAT)-6, Ag85A, Ag85B (MPT59), Ag85B, Ag85C, MPT32, MPT51, MPT59, MPT63, MPT64, MPT83, MPB5, MPB59, MPB64, MTC28, Mtb2, Mtb8.4, Mtb9.9, Mtb32A, Mtb39, Mtb41, TB10.4, TB10C, TB11B, TB12.5, TB13A, TB14, TB15, TB15A, TB16, TB16A, TB17, TB18, TB21, TB20.6, TB24, TB27B, TB32, TB32A, TB33, TB38, TB40.8, TB51, TB54, TB64, CFP6, CFP7, CFP7A, CFP7B, CFP8A, CFP8B, CFP9, CFP10, CFP11, CFP16, CFP17, CFP19, CFP19A, CFP19B, CFP20, CFP21, CFP22, CFP22A, CFP23, CFP23A, CFP23B, CFP25, CFP25A, CFP27, CFP28, CFP28B, CFP29, CFP30A, CFP30B, CFP50, CWP32, hspX (alpha-crystalline), APA, Tuberculin purified protein derivative (PPD), ST-CF, PPE68, LppX, PstS-1, PstS-2, PstS-3, HBHA, GroEL, GroEL2, GrpES, LHP, 19 kDa lipoprotein, 71 kDa, RD1-ORF2, RD1-ORF3, RD1-ORF4, RD1-ORF5, RD1-ORF8, RD1-ORF9A, RD1-ORF9B, Rv1984c, Rv0577, Rv1827, BfrB, Tpx. Rv1352, Rv1810, PpiA, Cut2, FbpB, FbpA, FbpC, DnaK, FecB, Ssb, Rp1L, FixA, FixB, AhpC2, Rv2626c, Rv1211, Mdh, Rv1626, Adk, ClpP, SucD (Belisle et al, 2005; U.S. Pat. No. 7,037,510; US 2004/0057963; US 2008/0199493; US 2008/0267990), or at least one antigenic portion or T-cell epitope of any of the above mentioned antigens.

The present invention contemplates the use of a single M. tuberculosis antigen. However, embodiments reliant on the use of two or more M. tuberculosis antigens are also specifically contemplated.

In various examples, the two or more antigens are produced as fusion proteins comprising two or more M. tuberculosis antigens, including two or more M. tuberculosis antigens selected from above mentioned antigens.

### 4.2 Hepatitis Antigens

A number of hepatitis antigens have been characterised and are suitable for use in the present invention. Exemplary hepatitis C antigens include C—p22, E1—gp35, E2—gp70, NS1—p7, NS2—p23, NS3—p70, NS4A—p8, NS4B—p27, NS5A—p56/58, and NS5B—p68, and each (whether alone or in combination) are suitable for application in the present invention. All hepatitis antigens, whether or not presently characterized, that are capable of eliciting an immune response are contemplated.

### 4.3 Influenza Antigens

A great many influenza antigens have been characterised and are suitable for use in the present invention. Exemplary influenza antigens suitable for use in the present invention include PB, PB2, PA, any of the hemagglutinin (HA) or neuramimidase (NA) proteins, NP, M, and NS, and each (whether alone or in combination) are suitable for application in the present invention.

All influenza antigens, whether or not presently characterized, that are capable of eliciting an immune response are contemplated.

### 4.4 Anthrax Antigens

A number of B. anthracis antigens have been identified as potential candidates for vaccine development and are useful in the present invention. For example, PA83 is one such antigen for vaccine development. Currently, only one FDA licensed vaccine for anthrax is available called “Anthrax Vaccine Adsorbed” (AVA) or BioThrax®. This vaccine is derived from the cell-free supernatant of a non-encapsulated strain of B. anthracis adsorbed to aluminum adjuvant. PA is the primary immunogen in AVA. Other exemplary anthrax antigens suitable for use in the present invention include Protective antigen (PA or PA63), LF and EF (proteins), poly-gamma-(D-glutamate) capsule, spore antigen (endospore specific components), BclA (exosporium specific protein), BxpB (spore-associated protein), and secreted proteins. All anthrax antigens, whether or not presently characterized, that are capable of eliciting an immune response are contemplated.

### 4.5 Tularemia Antigens

A number of F. tularensis antigens have been identified as potential candidates for vaccine development and are useful in the present invention. For example, AcpA and IglC are antigens suitable for vaccine development. Other exemplary Tularemia antigens suitable for use in the present invention include O-antigen, CPS, outer membrane proteins (e.g. FopA), lipoproteins (e.g. Tu14), secreted proteins and lipopolysaccharide. All tularemia antigens, whether or not presently characterized, that are capable of eliciting an immune response are contemplated.

### 4.6 Brucellosis Antigens

A number of B. abortusis antigens have been identified as potential candidates for vaccine development and are useful in the present invention. For example, Omp16 is one such antigen for vaccine development. Other exemplary Brucellosis antigens suitable for use in the present invention include O-antigen, lipopolysaccharide, outer membrane proteins (e.g. Omp16), secreted proteins, ribosomal proteins (e.g. L7 and L12), bacterioferritin, p39 (a putative periplasmic binding protein), groEL (heat-shock protein), lumazine synthase, BCSP31 surface protein, PAL16.5 OM lipoprotein, catalase, 26 kDa periplasmic protein, 31 kDa Omp31, 28 kDa Omp, 25 kDa Omp, and 10 kDA Om lipoprotein. All brucellosis antigens, whether or not presently characterized, that are capable of eliciting an immune response are contemplated.

### 4.7 Meningitis Antigens

A number of N. meningitidis antigens have been identified as potential candidates for vaccine development and are useful in the present invention. For example, Cys6, PorA, PorB, FetA, and ZnuD are antigens suitable for vaccine development. Other exemplary Meningitis antigens suitable for use in the present invention include O-antigen, factor H binding protein (fHbp), TbpB, NspA, NadA, outer membrane proteins, group B CPS, secreted proteins and lipopolysaccharide. All meningitis antigens, whether or not presently characterized, that are capable of eliciting an immune response are contemplated.

### 4.8 Dengue Antigens

A number of Flavivirus antigens have been identified as potential candidates for vaccine development to treat dengue fever and are useful in the present invention. For example, dengue virus envelope proteins E1-E4 and the membrane proteins M1-M4 are antigens suitable for vaccine development. Other exemplary dengue antigens suitable for use in the present invention include C, preM, 1, 2A, 2B, 3, 4A, 4B and 5. All dengue antigens, whether or not presently characterized, that are capable of eliciting an immune response are contemplated.

### 4.9 Ebola Antigens

A number of ebola virus antigens have been identified as potential candidates for vaccine development to treat ebola infection and are useful in the present invention. For example, Filoviridae Zaire ebolavirus and Sudan ebolavirus virion spike glycoprotein precursor antigens ZEBOV-GP, and SEBOV-GP, respectively, are suitable for vaccine development. Other exemplary ebola antigens suitable for use in the present invention include NP, vp35, vp40, GP, vp30, vp24 and L. All ebola antigens, whether or not presently characterized, that are capable of eliciting an immune response are contemplated.

### 4.10 West Nile Antigens

A number of West Nile virus antigens have been identified as potential candidates for vaccine development to treat infection and are useful in the present invention. For example, Flavivirus envelope antigen (E) from West Nile virus (WNV) is a non-toxic protein expressed on the surface of WNV virions (WNVE) and are suitable for vaccine development. Other exemplary WNV antigens suitable for use in the present invention include Cp, Prm, NS1, NS2A, NS2B, NS3, NS4A, NS4B and NS5. All West Nile antigens, whether or not presently characterized, that are capable of eliciting an immune response are contemplated.

The above-listed or referenced antigens are exemplary, not limiting, of the present inventions.

### 5. Expression Constructs

Processes for producing and using expression constructs for expression of fusion polypeptides in microorganisms, plant cells or animal cells (cellular expression systems) or in cell free expression systems, and host cells comprising expression constructs useful for forming polymer particles for use in the invention are well known in the art (e.g. Sambrook et al., 1987; Ausubel et al., 1987).

Expression constructs for use in methods of the invention are in one embodiment inserted into a replicable vector for cloning or for expression, or in another embodiment are incorporated into the host genome. Various vectors are publicly available. The vector is, for example, in the form of a plasmid, cosmid, viral particle, or phage. The appropriate nucleic acid sequence can be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more selectable marker genes, an enhancer element, a promoter, and a transcription termination sequence. Construction of suitable vectors containing one or more of these components employs standard ligation techniques known in the art.

Both expression and cloning vectors contain a nucleic acid sequence that enables the vector to replicate in one or more selected host cells. Such sequences are well known for a variety of bacteria, yeast, and viruses.

In one embodiment the expression construct is present on a high copy number vector.

In one embodiment the high copy number vector is selected from those that are present at 20 to 3000 copies per host cell.

In one embodiment the high copy number vector contain a high copy number origin of replication (ori), such as ColE1 or a ColE1-derived origin of replication. For example, the ColE-1 derived origin of replication may comprise the pUC19 origin of replication.

Numerous high copy number origins of replication suitable for use in the vectors of the present invention are known to those skilled in the art. These include the ColE1-derived origin of replication from pBR322 and its derivatives as well as other high copy number origins of replication, such as M13 FR ori or p15A ori. The 2μ plasmid origin is suitable for yeast, and various viral origins (SV40, polyoma, adenovirus, VSV or BPV) are useful for cloning vectors in mammalian cells.

Preferably, the high copy number origin of replication comprises the ColE1-derived pUC19 origin of replication.

The restriction site is positioned in the origin of replication such that cloning of an insert into the restriction site will inactivate the origin, rendering it incapable of directing replication of the vector. Alternatively, the at least one restriction site is positioned within the origin such that cloning of an insert into the restriction site will render it capable of supporting only low or single copy number replication of the vector.

Expression and cloning vectors will typically contain a selection gene, also termed a selectable marker to detect the presence of the vector in the transformed host cell. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

Selectable markers commonly used in plant transformation include the neomycin phophotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

Examples of suitable selectable markers for mammalian cells are those that enable the identification of cells competent to take up expression constructs, such as DHFR or thymidine kinase. An appropriate host cell when wild-type DHFR is employed is the CHO cell line deficient in DHFR activity, prepared and propagated as described by Urlaub et al., 1980. A suitable selection gene for use in yeast is the trp1 gene present in the yeast plasmid YRp7 (Stinchcomb et al., 1979; Kingsman et al., 1979; Tschemper et al., 1980). The trp1 gene provides a selection marker for a mutant strain of yeast lacking the ability to grow in tryptophan, for example, ATCC No. 44076 or PEP4-1 [Jones, Genetics, 85:12 (1977)].

An expression construct useful for forming polymer particles preferably includes a promoter which controls expression of at least one nucleic acid encoding a polymer synthase, particle-forming protein or fusion polypeptide.

Promoters recognized by a variety of potential host cells are well known. Promoters suitable for use with prokaryotic hosts include the β-lactamase and lactose promoter systems [Chang et al., 1978; Goeddel et al., 1979), alkaline phosphatase, a tryptophan (trp) promoter system [Goeddel, Nucleic Acids Res., 8:4057 (1980); EP 36,776], and hybrid promoters such as the tac promoter [deBoer et al., 1983). Promoters for use in bacterial systems also will contain a Shine-Dalgarno (S.D.) sequence operably linked to the nucleic acid encoding a polymer synthase, particle-forming protein or fusion polypeptide.

Examples of suitable promoting sequences for use with yeast hosts include the promoters for 3-phosphoglycerate kinase [Hitzeman et al., 1980) or other glycolytic enzymes [Hess et al., 1968; Holland, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization.

Examples of suitable promoters for use in plant host cells, including tissue or organ of a monocot or dicot plant include cell-, tissue- and organ-specific promoters, cell cycle specific promoters, temporal promoters, inducible promoters, constitutive promoters that are active in most plant tissues, and recombinant promoters. Choice of promoter will depend upon the temporal and spatial expression of the cloned polynucleotide, so desired. The promoters are those from the host cell, or promoters which are derived from genes of other plants, viruses, and plant pathogenic bacteria and fungi. Those skilled in the art will, without undue experimentation, be able to select promoters that are suitable for use in modifying and modulating expression constructs using genetic constructs comprising the polynucleotide sequences of the invention. Examples of constitutive plant promoters include the CaMV 35S promoter, the nopaline synthase promoter and the octopine synthase promoter, and the Ubi 1 promoter from maize. Plant promoters which are active in specific tissues, respond to internal developmental signals or external abiotic or biotic stresses are described in the scientific literature. Exemplary promoters are described, e.g., in WO 02/00894, which is herein incorporated by reference.

Examples of suitable promoters for use in mammalian host cells comprise those obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of an expression construct by higher eukaryotes is in some examples increased by inserting an enhancer sequence into the vector. Enhancers are cis-acting elements of DNA, usually about from 10 to 300 bp that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. Typically, the enhancer is spliced into the vector at a position 5′ or 3′ to the polymer synthase, particle-forming protein or fusion polypeptide coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the polymer synthase, particle-forming protein or fusion polypeptide.

In one embodiment the expression construct comprises an upstream inducible promoter, such as a BAD promoter, which is induced by arabinose.

In one embodiment the expression construct comprises a constitutive or regulatable promoter system.

In one embodiment the regulatable promoter system is an inducible or repressible promoter system.

While it is desirable to use strong promoters in the production of recombinant proteins, regulation of these promoters is essential since constitutive overproduction of heterologous proteins leads to decreases in growth rate, plasmid stability and culture viability.

A number of promoters are regulated by the interaction of a repressor protein with the operator (a region downstream from the promoter). The most well known operators are those from the lac operon and from bacteriophage A. An overview of regulated promoters in E. coli is provided in Table 1 of Friehs & Reardon, 1991.

A major difference between standard bacterial cultivations and those involving recombinant E. coli is the separation of the growth and production or induction phases. Recombinant protein production often takes advantage of regulated promoters to achieve high cell densities in the growth phase (when the promoter is “off” and the metabolic burden on the host cell is slight) and then high rates of heterologous protein production in the induction phase (following induction to turn the promoter “on”).

In one embodiment the regulatable promoter system is selected from LacI, Trp, phage γ and phage RNA polymerase.

In one embodiment the promoter system is selected from the lac or Ptac promoter and the lacI repressor, or the trp promoter and the TrpR repressor.

In one embodiment the LacI repressor is inactivated by addition of isopropyl-β-D-thiogalactopyranoside (IPTG) which binds to the active repressor causes dissociation from the operator, allowing expression.

In one embodiment the trp promoter system uses a synthetic media with a defined tryptophan concentration, such that when the concentration falls below a threshold level the system becomes self-inducible. In one embodiment 3-β-indole-acrylic acid is added to inactivate the TrpR repressor.

In one embodiment the promoter system may make use of the bacteriophage γ repressor cI. This repressor makes use of the γ prophage and prevent expression of all the lytic genes by interacting with two operators termed OL and OR. These operators overlap with two strong promoters PL and PR respectively. In the presence of the cI repressor, binding of RNA polymerase is prevented. The cI repressor can be inactivated by UV-irradiation or treatment of the cells with mitomycin C. A more convenient way to allow expression of the recombinant polypeptide is the application of a temperature-sensitive version of the cI repressor cI857. Host cells carrying a γ-based expression system can be grown to mid-exponential phase at low temperature and then transferred to high temperature to induce expression of the recombinant polypeptide.

A widely used expression system makes use of the phage T7 RNA polymerase which recognises only promoters found on the T7 DNA, and not promoters present on the host cell chromosome. Therefore, the expression construct may contain one of the T7 promoters (normally the promoter present in front of gene 10) to which the recombinant gene will be fused. The gene coding for the T7 RNA polymerase is either present on the expression construct, on a second compatible expression construct or integrated into the host cell chromosome. In all three cases, the gene is fused to an inducible promoter allowing its transcription and translation during the expression phase.

The E. coli strains BL21 (DE3) and BL21 (DE3) pLysS (Invitrogen, CA) are examples of host cells carrying the T7 RNA polymerase gene (there are a few more very suitable and commercially available E. coli strains harbouring the T7RNA polymerase gene such as e.g. KRX and XJ (autolysing)). Other cell strains carrying the T7 RNA polymerase gene are known in the art, such as Pseudomonas aeruginosa ADD1976 harboring the T7 RNA polymerase gene integrated into the genome (Brunschwig & Darzins, 1992) and Cupriavidus necator (formerly Ralstonia eutropha) harboring the T7 RNA polymerase gene integrated into the genome under phaP promoter control (Barnard et al., 2004).

The T7 RNA polymerase offers three advantages over the host cell enzymes: First, it consists of only one subunit, second it exerts a higher processivity, and third it is insensitive towards rifampicin. The latter characteristic can be used especially to enhance the amount of fusion polypeptide by adding this antibiotic about 10 min after induction of the gene coding for the T7 RNA polymerase. During that time, enough polymerase has been synthesised to allow high-level expression of the fusion polypeptide, and inhibition of the host cell enzymes prevents further expression of all the other genes present on both the plasmid and the chromosome. Other antibiotics which inhibit the bacterial RNA polymerase but not the T7 RNA polymerase are known in the art, such as streptolydigin and streptovaricin.

Since all promoter systems are leaky, low-level expression of the gene coding for T7 RNA polymerase may be deleterious to the cell in those cases where the recombinant polypeptide encodes a toxic protein. These polymerase molecules present during the growth phase can be inhibited by expressing the T7-encoded gene for lysozyme. This enzyme is a bifunctional protein that cuts a bond in the cell wall of the host cell and selectively inhibits the T7 RNA polymerase by binding to it, a feed-back mechanism that ensures a controlled burst of transcription during T7 infection. The E. coli strain BL21 (DE3) pLysS is an example of a host cell that carries the plasmid pLysS that constitutively expresses T7 lysozyme.

In one embodiment the promoter system makes use of promoters such as API or APR which are induced or “switched on” to initiate the induction cycle by a temperature shift, such as by elevating the temperature from about 30-37° C. to 42° C. to initiate the induction cycle.

A strong promoter may enhance fusion polypeptide density at the surface of the particle during in-vivo production.

Preferred fusion polypeptides comprise:

a polymer synthase, and a fusion partner comprising
(i) at least one antigen capable of eliciting an immune response, or
(ii) a binding domain capable of binding at least one antigen capable of eliciting an immune response, or
(iii) both (i) and (ii).

A nucleic acid sequence encoding both (i) and (ii) for use herein comprises a nucleic acid encoding a polymer synthase and a nucleic acid encoding an antigen capable of eliciting a cell-mediated immune response, or a nucleic acid sequence encoding polymer synthase and a nucleic acid encoding a binding domain capable of binding an antigen capable of eliciting a cell-mediated immune response. Once expressed, the fusion polypeptide is able to form or facilitate formation of a polymer particle.

In one embodiment the nucleic acid sequence encoding at least polymer synthase is indirectly fused with the nucleic acid sequence encoding a particle-forming protein and a nucleic acid encoding an antigen capable of eliciting a cell-mediated immune response or a particle-forming protein, preferably a polymer synthase, and a nucleic acid encoding a binding domain capable of binding an antigen capable of eliciting a cell-mediated immune response, through a polynucleotide linker or spacer sequence of a desired length.

In one embodiment the amino acid sequence of the fusion polypeptide encoding at least one antigen capable of eliciting a cell-mediated immune response or a binding domain capable of binding at least one antigen capable of eliciting a cell-mediated immune response is contiguous with the C-terminus of the amino acid sequence comprising a polymer synthase.

In one embodiment the amino acid sequence of the fusion protein comprising at least one antigen capable of eliciting a cell-mediated immune response or a binding domain capable of binding an antigen capable of eliciting a cell-mediated immune response is indirectly fused with the N-terminus of the amino acid sequence comprising a polymer synthase fragment through a peptide linker or spacer of a desired length that facilitates independent folding of the fusion polypeptides.

In one embodiment the amino acid sequence of the fusion polypeptide encoding at least one antigen capable of eliciting a cell-mediated immune response or a binding domain capable of binding an antigen capable of eliciting a cell-mediated immune response is contiguous with the N-terminus of the amino acid sequence comprising a particle-forming protein, preferably a polymer synthase, or a C-terminal synthase fragment.

In one embodiment the amino acid sequence of the fusion protein encoding at least one antigen capable of eliciting a cell-mediated immune response or a binding domain capable of binding an antigen capable of eliciting a cell-mediated immune response is indirectly fused with the C-terminus of the amino acid sequence comprising a particle-forming protein, preferably a polymer synthase, or a N-terminal polymer synthase fragment through a peptide linker or spacer of a desired length to facilitate independent folding of the fusion polypeptides.

In one embodiment the amino acid sequence of the fusion polypeptide encoding at least one antigen capable of eliciting a cell-mediated immune response or a binding domain capable of binding at least one antigen capable of eliciting a cell-mediated immune response is contiguous with the N-terminus of the amino acid sequence encoding a depolymerase, or a C-terminal depolymerase fragment.

In various embodiments directed to the treatment or prevention of tuberculosis, exemplary fusion polypeptides comprise:

a polymer synthase, and a fusion partner comprising
(i) at least one M. tuberculosis antigen, or
(ii) at least one M. tuberculosis antigen binding domain, or
(iii) both (i) and (ii).

A nucleic acid sequence encoding both (i) and (ii) for use herein comprises a nucleic acid encoding a polymer synthase and a nucleic acid encoding a M. tuberculosis antigen, or a nucleic acid sequence encoding polymer synthase and a nucleic acid encoding a M. tuberculosis antigen binding domain. Once expressed, the fusion polypeptide is able to form or facilitate formation of a polymer particle.

In one embodiment the nucleic acid sequence encoding at least polymer synthase is indirectly fused with the nucleic acid sequence encoding a particle-forming protein and a nucleic acid encoding a M. tuberculosis antigen or a particle-forming protein and a nucleic acid encoding a M. tuberculosis antigen binding domain, through a polynucleotide linker or spacer sequence of a desired length.

In one embodiment the amino acid sequence of the fusion polypeptide encoding at least one M. tuberculosis antigen or at least one M. tuberculosis antigen binding domain is contiguous with the C-terminus of the amino acid sequence comprising a polymer synthase.

In one embodiment the amino acid sequence of the fusion protein comprising at least one M. tuberculosis antigen or at least one M. tuberculosis antigen binding domain is indirectly fused with the N-terminus of the amino acid sequence comprising a polymer synthase fragment through a peptide linker or spacer of a desired length that facilitates independent folding of the fusion polypeptides.

In one embodiment the amino acid sequence of the fusion polypeptide encoding at least one M. tuberculosis antigen or at least one M. tuberculosis antigen binding domain is contiguous with the N-terminus of the amino acid sequence comprising a particle-forming protein or a C-terminal synthase fragment.

In one embodiment the amino acid sequence of the fusion protein encoding at least one M. tuberculosis antigen or at least one M. tuberculosis antigen binding domain is indirectly fused with the C-terminus of the amino acid sequence comprising a particle-forming protein or a N-terminal polymer synthase fragment through a peptide linker or spacer of a desired length to facilitate independent folding of the fusion polypeptides.

In one embodiment the amino acid sequence of the fusion polypeptide encoding at least one M. tuberculosis antigen or at least one M. tuberculosis antigen binding domain is contiguous with the N-terminus of the amino acid sequence encoding a depolymerase, or a C-terminal depolymerase fragment.

In various embodiments directed to the treatment or prevention of hepatitis, exemplary fusion polypeptides comprise:

a polymer synthase, and a fusion partner comprising
(i) at least one hepatitis antigen, or
(ii) at least one hepatitis antigen binding domain, or
(iii) both (i) and (ii).

A nucleic acid sequence encoding both (i) and (ii) for use herein comprises a nucleic acid encoding a polymer synthase and a nucleic acid encoding an hepatitis antigen, or a nucleic acid sequence encoding polymer synthase and a nucleic acid encoding an hepatitis antigen binding domain. Once expressed, the fusion polypeptide is able to form or facilitate formation of a polymer particle.

In one embodiment the nucleic acid sequence encoding at least polymer synthase is indirectly fused with the nucleic acid sequence encoding a particle-forming protein and a nucleic acid encoding an hepatitis antigen or a particle-forming protein and a nucleic acid encoding an hepatitis antigen binding domain, through a polynucleotide linker or spacer sequence of a desired length.

In one embodiment the amino acid sequence of the fusion polypeptide encoding at least one hepatitis antigen or at least one hepatitis antigen binding domain is contiguous with the C-terminus of the amino acid sequence comprising a polymer synthase.

In one embodiment the amino acid sequence of the fusion protein comprising at least one hepatitis antigen or at least one hepatitis antigen binding domain is indirectly fused with the N-terminus of the amino acid sequence comprising a polymer synthase fragment through a peptide linker or spacer of a desired length that facilitates independent folding of the fusion polypeptides.

In one embodiment the amino acid sequence of the fusion polypeptide encoding at least one hepatitis antigen or at least one hepatitis antigen binding domain is contiguous with the N-terminus of the amino acid sequence comprising a particle-forming protein or a C-terminal synthase fragment.

In one embodiment the amino acid sequence of the fusion protein encoding at least one hepatitis antigen or at least one hepatitis antigen binding domain is indirectly fused with the C-terminus of the amino acid sequence comprising a particle-forming protein or a N-terminal polymer synthase fragment through a peptide linker or spacer of a desired length to facilitate independent folding of the fusion polypeptides.

In one embodiment the amino acid sequence of the fusion polypeptide encoding at least one hepatitis antigen or at least one hepatitis antigen binding domain is contiguous with the N-terminus of the amino acid sequence encoding a depolymerase, or a C-terminal depolymerase fragment.

In various embodiments directed to the treatment or prevention of influenza, exemplary fusion polypeptides comprise:

a polymer synthase, and a fusion partner comprising
(i) at least one influenza antigen, or
(ii) at least one influenza antigen binding domain, or
(iii) both (i) and (ii).

A nucleic acid sequence encoding both (i) and (ii) for use herein comprises a nucleic acid encoding a polymer synthase and a nucleic acid encoding an influenza antigen, or a nucleic acid sequence encoding polymer synthase and a nucleic acid encoding an influenza antigen binding domain. Once expressed, the fusion polypeptide is able to form or facilitate formation of a polymer particle.

In one embodiment the nucleic acid sequence encoding at least polymer synthase is indirectly fused with the nucleic acid sequence encoding a particle-forming protein and a nucleic acid encoding an influenza antigen or a particle-forming protein and a nucleic acid encoding an influenza antigen binding domain, through a polynucleotide linker or spacer sequence of a desired length.

In one embodiment the amino acid sequence of the fusion polypeptide encoding at least one influenza antigen or at least one influenza antigen binding domain is contiguous with the C-terminus of the amino acid sequence comprising a polymer synthase.

In one embodiment the amino acid sequence of the fusion protein comprising at least one influenza antigen or at least one influenza antigen binding domain is indirectly fused with the N-terminus of the amino acid sequence comprising a polymer synthase fragment through a peptide linker or spacer of a desired length that facilitates independent folding of the fusion polypeptides.

In one embodiment the amino acid sequence of the fusion polypeptide encoding at least one influenza antigen or at least one influenza antigen binding domain is contiguous with the N-terminus of the amino acid sequence comprising a particle-forming protein or a C-terminal synthase fragment.

In one embodiment the amino acid sequence of the fusion protein encoding at least one influenza antigen or at least one influenza antigen binding domain is indirectly fused with the C-terminus of the amino acid sequence comprising a particle-forming protein or a N-terminal polymer synthase fragment through a peptide linker or spacer of a desired length to facilitate independent folding of the fusion polypeptides.

In one embodiment the amino acid sequence of the fusion polypeptide encoding at least one influenza antigen or at least one influenza antigen binding domain is contiguous with the N-terminus of the amino acid sequence encoding a depolymerase, or a C-terminal depolymerase fragment.

One advantage of the fusion polypeptides according to the present invention is that the modification of the proteins binding to the surface of the polymer particles does not affect the functionality of the proteins involved in the formation of the polymer particles. For example, the functionality of the polymer synthase is retained if a recombinant polypeptide is fused with the N-terminal end thereof, resulting in the production of recombinant polypeptide on the surface of the particle. Should the functionality of a protein nevertheless be impaired by the fusion, this shortcoming is offset by the presence of an additional particle-forming protein which performs the same function and is present in an active state.

In this manner, it is possible to ensure a stable bond of the recombinant polypeptide bound to the polymer particles via the particle-forming proteins.

It should be appreciated that the arrangement of the proteins in the fusion polypeptide is dependent on the order of gene sequences in the nucleic acid contained in the plasmid.

For example, it may be desired to produce a fusion polypeptide wherein the antigen capable of eliciting a cell-mediated immune response or a binding domain capable of binding at least one antigen capable of eliciting a cell-mediated immune response is indirectly fused to the polymer synthase. The term “indirectly fused” refers to a fusion polypeptide comprising a particle-forming protein, preferably a polymer synthase, and at least one antigen capable of eliciting a cell-mediated immune response or a binding domain capable of binding at least one antigen capable of eliciting a cell-mediated immune response that are separated by an additional protein which may be any protein that is desired to be expressed in the fusion polypeptide.

When used in the context of particles for use in the treatment of tuberculosis, it may be desired to produce a fusion polypeptide wherein the M. tuberculosis antigen or at least one M. tuberculosis antigen binding domain is indirectly fused to the polymer synthase. Similarly, when used in the treatment of hepatitis or influenza, it may be desired to produce a fusion polypeptide wherein the hepatitis antigen or the influenza antigen or at least one hepatitis antigen binding domain or at least one influenza antigen binding domain is indirectly fused to the polymer synthase. The term “indirectly fused” refers to a fusion polypeptide comprising a particle-forming protein and at least a M. tuberculosis antigen or at least one M. tuberculosis antigen binding domain that are separated by an additional protein which may be any protein that is desired to be expressed in the fusion polypeptide. Similarly, the term can refer to a fusion polypeptide comprising a particle-forming protein and at least one hepatitis antigen or at least one hepatitis antigen binding domain that are separated by an additional protein which may be any protein that is desired to be expressed in the fusion polypeptide. Alternatively, the term can refer to a fusion polypeptide comprising a particle-forming protein and at least one influenza antigen or at least one influenza antigen binding domain that are separated by an additional protein which may be any protein that is desired to be expressed in the fusion polypeptide.

In one embodiment the additional protein is selected from a particle-forming protein or a fusion polypeptide, or a linker or spacer to facilitate independent folding of the fusion polypeptides, as discussed above. In this embodiment it would be necessary to order the sequence of genes in the plasmid to reflect the desired arrangement of the fusion polypeptide.

In one embodiment the antigen capable of eliciting a cell-mediated immune response or a binding domain capable of binding at least one antigen capable of eliciting a cell-mediated immune response may bear directly fused to the polymer synthase. The term “directly fused” is used herein to indicate where two or more peptides are linked via peptide bonds.

In various embodiments directed to the treatment or prevention of tuberculosis, for example, the M. tuberculosis antigen or at least one M. tuberculosis antigen binding domain may be directly fused to the polymer synthase.

The term “directly fused” is used herein to indicate where two or more peptides are linked via peptide bonds.

In various embodiments directed to the treatment or prevention of hepatitis, the hepatitis antigen or at least one hepatitis antigen binding domain may be directly fused to the polymer synthase.

In various embodiments directed to the treatment or prevention of influenza, the influenza antigen or at least one influenza antigen binding domain may be directly fused to the polymer synthase.

The term “directly fused” is used herein to indicate where two or more peptides are linked via peptide bonds.

It may also be possible to form a particle wherein the particle comprises at least two distinct fusion polypeptides that are bound to the polymer particle. For example, a first fusion polypeptide comprising an antigen capable of eliciting a cell-mediated immune response or a binding domain capable of binding at least one antigen capable of eliciting a cell-mediated immune response fused to a polymer synthase could be bound to the polymer particle. When used in the context of particles for use in the treatment of tuberculosis, the particle comprises a first fusion polypeptide comprising a M. tuberculosis antigen, for example, or at least one M. tuberculosis antigen binding domain fused to a polymer synthase could be bound to the polymer particle. When used in the context of particles for use in the treatment of hepatitis, the particle comprises a first fusion polypeptide comprising a hepatitis antigen or at least one hepatitis antigen binding domain fused to a polymer synthase could be bound to the polymer particle. When used in the context of particles for use in the treatment of influenza, the particle comprises a first fusion polypeptide comprising an influenza antigen or at least one influenza antigen binding domain fused to a polymer synthase could be bound to the polymer particle.

In one embodiment the expression construct is expressed in vivo. Preferably the expression construct is a plasmid which is expressed in a microorganism, preferably Escherichia coli.

In one embodiment the expression construct is expressed in vitro. Preferably the expression construct is expressed in vitro using a cell free expression system.

In one embodiment one or more genes can be inserted into a single expression construct, or one or more genes can be integrated into the host cell genome. In all cases expression can be controlled through promoters as described above.

In one embodiment the expression construct further encodes at least one additional fusion polypeptide comprising an antigen capable of eliciting a cell-mediated immune response or a binding domain capable of binding at least one antigen capable of eliciting a cell-mediated immune response and a particle-forming protein, preferably a polymer synthase, as discussed above.

In one embodiment the expression construct further encodes at least one additional fusion polypeptide comprising a M. tuberculosis antigen or at least one M. tuberculosis antigen binding domain and a particle-forming protein as discussed above.

In one embodiment the expression construct further encodes at least one additional fusion polypeptide comprising a hepatitis antigen or at least one hepatitis antigen binding domain and a particle-forming protein as discussed above.

In one embodiment the expression construct further encodes at least one additional fusion polypeptide comprising a influenza antigen or at least one influenza antigen binding domain and a particle-forming protein as discussed above.

Plasmids useful herein are shown in the examples and are described in detail in PCT/DE2003/002799 published as WO 2004/020623 (Bernd Rehm) and PCT/NZ2006/000251 published as WO 2007/037706 (Bernd Rehm) which are each herein incorporated by reference in their entirety.

It will be appreciated that the binding domains of the antigens capable of eliciting a cell-mediated immune response are able to bind at least one antigen capable of eliciting a cell-mediated immune response, for example an antigen capable of eliciting a cell-mediated immune response present in the subject to which the binding domain capable of binding the antigen capable of eliciting a cell-mediated immune response is administered or in which the immune response is to be elicited.

In the context of use for the treatment of tuberculosis, it will be appreciated that the M. tuberculosis antigen binding domains are able to bind at least one M. tuberculosis antigen, for example a M. tuberculosis antigen present in the subject to which the M. tuberculosis antigen binding domain is administered or in which the immune response is to be elicited. Similarly, in the use for the treatment of hepatitis, it will be appreciated that the hepatitis antigen binding domains are able to bind at least one hepatitis antigen, for example a hepatitis antigen present in the subject to which the hepatitis antigen binding domain is administered or in which the immune response is to be elicited. In use for the treatment of influenza, it will be appreciated that the influenza antigen binding domains are able to bind at least one influenza antigen, for example an influenza antigen present in the subject to which the influenza antigen binding domain is administered or in which the immune response is to be elicited.

### 6. Hosts for Particle Production

The particles of the present invention are conveniently produced in a host cell, using one or more expression constructs as herein described. Polymer particles of the invention can be produced by enabling the host cell to express the expression construct. This can be achieved by first introducing the expression construct into the host cell or a progenitor of the host cell, for example by transforming or transfecting a host cell or a progenitor of the host cell with the expression construct, or by otherwise ensuring the expression construct is present in the host cell.

Following transformation, the transformed host cell is maintained under conditions suitable for expression of the fusion polypeptides from the expression constructs and for formation of polymer particles. Such conditions comprise those suitable for expression of the chosen expression construct, such as a plasmid in a suitable organism, as are known in the art. For example, and particularly when high yield or overexpression is desired, provision of a suitable substrate in the culture media allows the particle-forming protein component of a fusion polypeptide to form a polymer particle.

Accordingly, the present invention provides a method for producing polymer particles, the method comprising:
providing a host cell comprising at least one expression construct, the expression construct comprising:
   at least one nucleic acid sequence encoding a particle-forming protein, preferably a polymer synthase; and
   at least one nucleic acid sequence encoding an antigen capable of eliciting a cell-mediated immune response or a binding domain capable of binding an antigen capable of eliciting a cell-mediated immune response;
maintaining the host cell under conditions suitable for expression of the expression construct and for formation of polymer particles; and
separating the polymer particles from the host cells.

In one embodiment, the present invention provides a method for producing polymer particles, the method comprising:
providing a host cell comprising at least one expression construct, the expression construct comprising:
   at least one nucleic acid sequence encoding a particle-forming protein; and
   at least one nucleic acid sequence encoding a M. tuberculosis antigen or a M. tuberculosis antigen binding domain, for example;
maintaining the host cell under conditions suitable for expression of the expression construct and for formation of polymer particles by the polymer synthase; and
separating the polymer particles from the host cells to produce a composition comprising polymer particles.

In one embodiment, the present invention provides a method for producing polymer particles, the method comprising:
providing a host cell comprising at least one expression construct, the expression construct comprising:
   at least one nucleic acid sequence encoding a particle-forming protein; and
   at least one nucleic acid sequence encoding an hepatitis antigen or an hepatitis antigen binding domain or an influenza antigen or an influenza-antigen binding domain;
maintaining the host cell under conditions suitable for expression of the expression construct and for formation of polymer particles by the polymer synthase; and
separating the polymer particles from the host cells to produce a composition comprising polymer particles.

Preferably the host cell is, for example, a bacterial cell, a fungi cell, yeast cell, a plant cell, an insect cell or an animal cell, preferably an isolated or non-human host cell. Host cells useful in methods well known in the art (e.g. Sambrook et al., 1987; Ausubel et al., 1987) for the production of recombinant polymer particles are frequently suitable for use in the methods of the present invention, bearing in mind the considerations discussed herein.

Suitable prokaryote host cells comprise, for example, eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include other Enterobacteriaceae such as Escherichia spp., Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Actinomycetes such as Streptomyces, Rhodococcus, Corynebacterium and Mycobaterium.

In some embodiments, for example, E. coli strain W3110 may be used because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kanr; E. coli W3110 strain 37D6, which has the complete genotype tonA ptr3 phoA E15 (argF-lac)169 degP ompT rbs7 ilvG kanr; E. coli W3110 strain 40B4, which is strain 37D6 with a non-kanamycin resistant degP deletion mutation.

In some preferred embodiments, for example, Lactococcus lactis strains that do not produce lipopolysaccharide endotoxins may be used. Examples of Lactococcus lactis strains include MG1363 and Lactococcus lactis subspecies cremoris NZ9000.

In addition to prokaryotes, eukaryotic microbes such as filamentous fungi or yeast are suitable cloning or expression hosts for use in the methods of the invention, for example. Examples include Saccharomyces cerevisiae, a commonly used lower eukaryotic host microorganism. Other examples include Schizosaccharomyces pombe (Beach and Nurse, 1981; EP 139,383), Kluyveromyces hosts (U.S. Pat. No. 4,943,529; Fleer et al., 1991) such as, e.g., K. lactis (MW98-8C, CBS683, CBS4574; Louvencourt et al., 1983), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al, 1990), K. thermotolerans, and K. marxianus; yarrowia (EP 402,226); Pichia pastoris (EP 183,070; Sreekrishna et al., 1988); Candida; Trichoderma reesia (EP 244,234); Neurospora crassa (Case et al., 1979); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., 1983; Tilburn et al., 1983; Yelton et al., 1984) and A. niger (Kelly and Hynes, 1985). Methylotropic yeasts are suitable herein and comprise yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in Anthony, 1982.

Examples of invertebrate host cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., 1980); mouse sertoli cells (TM4, Mather, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., 1982); MRC 5 cells; FS4 cells; and a human hepatoma line (Hep G2).

Eukaryotic cell lines, and particularly mammalian cell lines, will be preferred when, for example, the antigen capable of eliciting a cell-mediated immune response or the binding domain capable of binding the antigen capable of eliciting a cell-mediated immune response or the M. tuberculosis antigen or the M. tuberculosis antigen binding domain or the hepatitis antigen or the hepatitis antigen binding domain or the influenza antigen or the influenza antigen binding domain requires one or more post-translational modifications, such as, for example, glycation. For example, one or more antigens capable of eliciting a cell-mediated immune response may require post-translational modification to be immunogenic or optimally immunogenic, and may thus be usefully expressed in an expression host capable of such post-translational modifications.

In one embodiment the host cell is a cell with an oxidising cytosol, for example the E. coli Origami strain (Novagen).

In another embodiment the host cell is a cell with a reducing cytosol, preferably E. coli.

The host cell, for example, may be selected from the genera comprising Ralstonia, Acaligenes, Pseudomonas and Halobiforma. Preferably the microorganism used is selected from the group comprising, for example, Ralstonia eutropha, Alcaligenes latus, Escherichia coli, Pseudomonas fragi, Pseudomonas putida, Pseudomonas oleovorans, Pseudomonas aeruginosa, Pseudomonas fluorescens, and Halobiforma haloterrestris. This group comprises both microorganisms which are naturally capable of producing biocompatible, biodegradable particles and microorganisms, such as for example E. coli, which, due to their genetic makeup, are not capable of so doing. The genes required to enable the latter-stated microorganisms to produce the particles are introduced as described above.

Extremely halophilic archaea produce polymer particles with lower levels of unspecific binding of protein, allowing easier isolation and purification of the particles from the cells.

In principle, any culturable host cell may be used for the production of polymer particles by means of the above-described process, even if the host cell cannot produce the substrates required to form the polymer particles due to a different metabolism. In such cases, the necessary substrates are added to the culture medium and are then converted into polymer particle by the proteins which have been expressed by the genes which have been introduced into the cell.

Genes utilized to enable the latter-stated host cells to produce the polymer particles include, for example, a thiolase, a reductase or a polymer synthase, such as phaA thiolase, phaB ketoacyl reductase or phaC synthase from Ralstonia eutropha. Which genes are used to augment what the host cell lacks for polymer particle formation will be dependent on the genetic makeup of the host cell and which substrates are provided in the culture medium.

The genes and proteins involved in the formation of polyhydroxyalkanoate (PHA) particles, and general considerations for particle formation are reported in Madison, et al, 1999; published PCT International Application WO 2004/020623 (Bernd Rehm); and Rehm, 2003; Brockelbank J A. et al., 2006; Peters and Rehm, 2006; Bäckström et al, (2006) and Rehm, (2006), all of which are herein incorporated by reference.

A polymer synthase alone can be used in any host cell with (R)-Hydroxyacyl-CoA or other CoA thioester or derivatives thereof as a substrate.

The polymer particle can also be formed in vitro. Preferably, for example, a cell free expression system is used. In such systems a polymer synthase is provided. Purified polymer synthase, such as that obtainable from recombinant production, or in cell free systems capable of protein translation, that obtainable in the cell free system itself by way of introduction of an expression construct encoding a polymer synthase, will be preferred. In order to produce an environment to allow particle formation in vitro the necessary substrates for polymer particle formation should be included in the media.

The polymer synthase can be used for the in vitro production of functionalised polymer particles using (R)-Hydroxyacyl-CoA or other CoA thioester as a substrate, for example.

The fusion polypeptides can be purified from lysed cells using a cell sorter, centrifugation, filtration or affinity chromatography prior to use in in vitro polymer particle production.

In vitro polymer particle formation enables optimum control of surface composition, including the level of fusion polypeptide coverage, phospholipid composition and so forth.

It will be appreciated that the characteristics of the polymer particle may be influenced or controlled by controlling the conditions in which the polymer particle is produced. This may include, for example, the genetic make up of the host cell, eg cell division mutants that produce large granules, as discussed in Peters and Rehm, 2005. The conditions in which a host cell is maintained, for example temperature, the presence of substrate, the presence of one or more particle-forming proteins such as a particle size-determining protein, the presence of a polymer regulator, and the like.

In one embodiment, a desirable characteristic of the polymer particle is that it is persistent. The term “persistent” refers to the ability of the polymer particle to resist degradation in a selected environment. An additional desirable characteristic of the polymer particle is that it is formed from the polymer synthase or particle-forming protein and binds to the C- or N-terminal of the polymer synthase or particle-forming protein during particle assembly.

In some embodiments of the invention it is desirable to achieve overexpression of the expression constructs in the host cell. Mechanisms for overexpression a particular expression construct are well known in the art, and will depend on the construct itself, the host in which it is to be expressed, and other factors including the degree of overexpression desired or required. For example, overexpression can be achieved by i) use of a strong promoter system, for example the T7 RNA polymerase promoter systemin prokaryotic hosts; ii) use of a high copy number plasmid, for example a plasmid containing the colE1 origin of replication or iii) stabilisation of the messenger RNA, for example through use of fusion sequences, or iv) optimization of translation through, for example, optimization of codon usage, of ribosomal binding sites, or termination sites, and the like. The benefits of overexpression may allow the production of smaller particles where desired and the production of a higher number of polymer particles.

The composition of the polymers forming the polymer particles may affect the mechanical or physiochemical properties of the polymer particles. For example, polymer particles differing in their polymer composition may differ in half-life or may release biologically active substances, in particular pharmaceutical active ingredients, at different rates. For example, polymer particles composed of C6-C14 3-hydroxy fatty acids exhibit a higher rate of polymer degradation due to the low crystallinity of the polymer. An increase in the molar ratio of polymer constituents with relatively large side chains on the polymer backbone usually reduces crystallinity and results in more pronounced elastomeric properties. By controlling polymer composition in accordance with the process described in the invention, it is accordingly possible to influence the biodegradability of the polymer particles and thus affect the duration the polymer particles (and when present the one or more antigens capable of eliciting a cell-mediated immune response or the binding domains of the antigens capable of eliciting a cell-mediated immune response on the particle or the one or more M. tuberculosis antigens or M. tuberculosis antigen binding domains on the particle, or the hepatitis antigen or the hepatitis antigen binding domain or the influenza antigen or the influenza antigen binding domain are maintained in, for example, a subject to whom they are administered, or to affect the release rate for biologically active substances present on or in the polymer particles, in particular pharmaceutically active agents or skin-care ingredients.

At least one fatty acid with functional side groups is preferably introduced into the culture medium as a substrate for the formation of the polymer particles, with at least one hydroxy fatty acid and/or at least one mercapto fatty acid and/or at least one β-amino fatty acid particularly preferably being introduced. “Fatty acids with functional side groups” should be taken to mean saturated or unsaturated fatty acids. These also include fatty acids containing functional side groups which are selected from the group comprising methyl groups, alkyl groups, hydroxyl groups, phenyl groups, sulfhydryl groups, primary, secondary and tertiary amino groups, aldehyde groups, keto groups, ether groups, carboxyl groups, O-ester groups, thioester groups, carboxylic acid amide groups, hemiacetal groups, acetal groups, phosphate monoester groups and phosphate diester groups. Use of the substrates is determined by the desired composition and the desired properties of the polymer particle.

The substrate or the substrate mixture may comprise at least one optionally substituted amino acid, lactate, ester or saturated or unsaturated fatty acid, preferably acetyl-CoA.

In one embodiment an adjuvant, an immunomodulatory agent or molecule, such as an immunostimulatory agent or molecule, or other compound useful in the preparation of vaccines is provided in the substrate mixture and is incorporated into the polymer particle during polymer particle formation, or is allowed to diffuse into the polymer particle.

The polymer particle may comprise a polymer selected from poly-beta-amino acids, polylactates, polythioesters and polyesters, for example. Most preferably the polymer comprises polyhydroxyalkanoate (PHA), preferably poly(3-hydroxybutyrate) (PHB).

The polymer synthase or polymer particle preferably comprises a phospholipid monolayer that encapsulates the polymer particle. Preferably said particle-forming protein spans said lipid monolayer.

The polymer synthase or particle-forming protein is preferably bound to the polymer particle or to the phospholipid monolayer or is bound to both.

The particle-forming protein is preferably covalently or non-covalently bound to the polymer particle it forms.

Preferably at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or 100% of the surface area of the polymer particle is covered by fusion polypeptides.

In certain circumstances it may be desirable to control the size of the particles produced in the methods of the invention, for example to produce particles particularly suited to a given application. For example, it may be desirable to produce polymer particles comprising one or more antigens capable of eliciting a cell-mediated immune response of a relatively large size, for example to elicit a robust cell-mediated immune response. For example, in the context of particles for use in the treatment of tuberculosis, it may be desirable to produce polymer particles comprising one or more M. tuberculosis antigens of a relatively large size, for example to elicit a robust cell-mediated immune response. Similar conditions may be applicable for the treatment of hepatitis or influenza, where is may be desirable to produce polymer particles comprising one or more the hepatitis antigens or one or more influenza antigens of a relatively large size, for example to elicit a robust cell-mediated immune response. Methods to control the size of polymer particles are described in PCT/DE2003/002799 published as WO 2004/020623, and PCT/NZ2006/000251 published as WO 2007/037706.

In some embodiments, particle size is controlled by controlling the expression of the particle-forming protein, or by controlling the expression of a particle size-determining protein if present, for example.

In other embodiments of the present invention, for example, particle size control may be achieved by controlling the availability of a substrate, for example the availability of a substrate in the culture medium. In certain examples, the substrate may be added to the culture medium in a quantity such that it is sufficient to ensure control of the size of the polymer particle.

It will be appreciated that a combination of such methods may be used, allowing the possibility for exerting still more effective control over particle size.

In various embodiments, for example, particle size may be controlled to produce particles having a diameter of from about 10 nm to 3 μm, preferably from about 10 nm to about 900 nm, from about 10 nm to about 800 nm, from about 10 nm to about 700 nm, from about 10 nm to about 600 nm, from about 10 nm to about 500 nm, from about 10 nm to about 400 nm, from about 10 nm to about 300 nm, from about 10 nm to about 200 nm, and particularly preferably of from about 10 nm to about 100 nm.

In other embodiments, for example, particle size may be controlled to produce particles having a diameter of from about 10 nm to about 90 nm, from about 10 nm to about 80 nm, from about 10 nm to about 70 nm, from about 10 nm to about 60 nm, from about 10 nm to about 50 nm, from about 10 nm to about 40 nm, from about 10 nm to about 30 nm, or from about 10 nm to about 20 nm.

Other ranges of average polymer size, for example, including ranges within the above recited ranges, are specifically contemplated, for example polymer particles having a diameter of from about 50 to about 500 nm, from about 150 to about 250 nm, or from about 100 to about 500 nm, etc.

In various embodiments, for example, 90% of the particles produced have a diameter of about 200 nm or below, 80% have a diameter about 150 nm or below, 60% have a diameter about 100 nm or below, 45% have a diameter about 80 nm or below, 40% have a diameter about 60 nm or below, 25% have a diameter about 50 nm or below, and 5% have a diameter about 35 nm or below

In various embodiments, for example, the method produces polymer particles with an average diameter less than about 200 nm, less than about 150 nm, or less than about 110 nm.

### 7. Compositions and Formulations

The polymer particles of the invention can be formulated as compositions suitable for use in the methods of the invention for a number of different applications, for example, formulated for administration via a particular route or formulated for storage, can be stably maintained as particles outside the host cell that produced them, and that these particles can be designed to suit a number of applications.

In one embodiment, for example, the compositions useful herein are formulated to allow for administration to a subject by any chosen route, including but not limited to oral or parenteral (including topical, subcutaneous, intramuscular and intravenous) administration.

Thus, for example, a pharmaceutical composition useful according to the invention may be formulated with an appropriate pharmaceutically acceptable carrier (including excipients, diluents, auxiliaries, and combinations thereof) selected with regard to the intended route of administration and standard pharmaceutical practice. For example, pharmaceutical compositions intended for vaccination can contain one or more adjuvants or immunostimulants, as are well known in the art. For example, a composition useful according to the invention can be administered orally as a powder, liquid, tablet or capsule, or topically as an ointment, cream or lotion. Suitable formulations may contain additional agents as required, including emulsifying, antioxidant, flavouring or colouring agents, and may be adapted for immediate-, delayed-, modified-, sustained-, pulsed- or controlled-release.

Thus, the invention also is directed to doses, dosage forms, formulations, compositions and/or devices comprising one or more polymer particles of the invention including those disclosed herein, useful for the therapy of diseases, disorders, and/or conditions in humans and other mammals and other disorders as disclosed herein. The use of these dosage forms, formulations compositions and/or devices comprising one or more polymer particles of the invention enables effective treatment of these conditions. The invention provides, for example, dosage forms, formulations, devices and/or compositions containing one or more comprising one or more polymer particles of the invention, such as one or more polymer particles comprising a Tb antigen. The dosage forms, formulations, devices and/or compositions of the invention may be formulated to optimize bioavailability, immunogenicity, or to maintain plasma, blood, or tissue concentrations within the immunogenic or therapeutic range, including for extended periods. Controlled delivery preparations may also be used to optimize the antigen concentration at the site of action, for example.

The dosage forms, formulations, devices and/or compositions of the invention may be formulated for periodic administration, for example to provide continued exposure to the one or more polymer particles of the invention. Strategies to elicit a beneficial immunological response, for example those that employ one or more “booster” vaccinations, are well known in the art, and such strategies may be adopted in the practise of the present invention.

Pharmaceutical compositions and dosage forms can be administered via the parenteral route, and this route will be preferred for certain embodiments of methods of eliciting an immune response, such as those described herein. Examples of parenteral dosage forms include aqueous solutions, isotonic saline or 5% glucose of the active agent, or other well-known pharmaceutically acceptable excipients. Cyclodextrins, for example, or other solubilising agents well-known to those familiar with the art, can be utilized as pharmaceutical excipients for delivery of the therapeutic agent.

Examples of dosage forms suitable for oral administration include, but are not limited to tablets, capsules, lozenges, or like forms, or any liquid forms such as syrups, aqueous solutions, emulsions and the like, capable of providing a therapeutically effective amount of a polymer particle of the invention. Capsules can contain any standard pharmaceutically acceptable materials such as gelatin or cellulose. Tablets can be formulated in accordance with conventional procedures by compressing mixtures of the active ingredients with a solid carrier and a lubricant. Examples of solid carriers include starch and sugar bentonite. Active ingredients can also be administered in a form of a hard shell tablet or a capsule containing a binder, e.g., lactose or mannitol, a conventional filler, and a tabletting agent.

Examples of dosage forms suitable for transdermal administration include, but are not limited, to transdermal patches, transdermal bandages, and the like. Examples of dosage forms suitable for topical administration of the compositions and formulations of the invention are any lotion, stick, spray, ointment, paste, cream, gel, etc., whether applied directly to the skin or via an intermediary such as a pad, patch or the like.

Examples of dosage forms suitable for suppository administration of the compositions and formulations of the invention include any solid dosage form inserted into a bodily orifice particularly those inserted rectally, vaginally and urethrally.

Examples of dosage of forms suitable for injection of the compositions and formulations of the invention include delivery via bolus such as single or multiple administrations by intravenous injection, subcutaneous, subdermal, and intramuscular administration or oral administration.

Examples of dosage forms suitable for depot administration of the compositions and formulations of the invention include pellets or small cylinders of polymer particles of the invention or solid forms wherein the polymer particles of the invention are entrapped in a matrix of biodegradable polymers, microemulsions, liposomes or are microencapsulated.

Examples of infusion devices for compositions and formulations of the invention include infusion pumps containing one or more polymer particles of the invention at a desired amount for a desired number of doses or steady state administration, and include implantable drug pumps.

Examples of implantable infusion devices for compositions, and formulations of the invention include any solid form in which the polymer particles of the invention are encapsulated within or dispersed throughout a biodegradable polymer or synthetic, polymer such as silicone, silicone rubber, silastic or similar polymer.

Examples of dosage forms suitable for transmucosal delivery of the compositions and formulations of the invention include depositories solutions for enemas, pessaries, tampons, creams, gels, pastes, foams, nebulised solutions, powders and similar formulations containing in addition to the active ingredients such carriers as are known in the art to be appropriate.

Specifically contemplated are dosage forms suitable for inhalation or insufflation of the compositions and formulations of the invention, including compositions comprising solutions and/or suspensions in pharmaceutically acceptable, aqueous, or organic solvents, or mixture thereof and/or powders. Transmucosal administration of the compositions and formulations of the invention may utilize any mucosal membrane but commonly utilizes the nasal, buccal, vaginal and rectal tissues. Formulations suitable for nasal administration of the compositions and formulations of the invention may be administered in a liquid form, for example, nasal spray, nasal drops, or by aerosol administration by nebulizer, including aqueous or oily solutions of the polymer particles. Formulations for nasal administration, wherein the carrier is a solid, include a coarse powder having a particle size, for example, of less than about 100 microns, preferably less, most preferably less than about 50 microns, which is administered in the manner in which snuff is taken, i.e., by rapid inhalation through the nasal passage from a container of the powder held close up to the nose. Formulations of the invention may be prepared as aqueous solutions for example in saline, solutions employing benzyl alcohol or other suitable preservatives, absorption promoters to enhance bio-availability, fluorocarbons, and/or other solubilising or dispersing agents known in the art.

Examples of dosage forms suitable for buccal administration of the compositions and formulations of the invention include lozenges, tablets and the like, compositions comprising solutions and/or suspensions in pharmaceutically acceptable, aqueous, or organic solvents, or mixtures thereof and/or powders.

Examples of dosage forms suitable for sublingual administration of the compositions and formulations of the invention include lozenges, tablets and the like, compositions comprising solutions and/or suspensions in pharmaceutically acceptable, aqueous, or organic solvents, or mixtures thereof and/or powders.

Examples of dosage forms suitable for opthalmic administration of the compositions and formulations of the invention include inserts and/or compositions comprising solutions and/or suspensions in pharmaceutically acceptable, aqueous, or organic solvents.

Examples of formulations of compositions, including vaccines and controlled drug formulations, useful for delivery of the compositions and formulations of the invention are found in, for example, Sweetman, S. C. (Ed.). Martindale. The Complete Drug Reference, 33rd Edition, Pharmaceutical Press, Chicago, 2002, 2483 pp.; Aulton, M. E. (Ed.) Pharmaceutics. The Science of Dosage Form Design. Churchill Livingstone, Edinburgh, 2000, 734 pp.; and, Ansel, H. C., Allen, L. V. and Popovich, N. G. Pharmaceutical Dosage Forms and Drug Delivery Systems, 7th Ed., Lippincott 1999, 676 pp. Excipients employed in the manufacture of drug delivery systems are described in various publications known to those skilled in the art including, for example, Kibbe, E. H. Handbook of Pharmaceutical Excipients, 3rd Ed., American Pharmaceutical Association, Washington, 2000, 665 pp. The USP also provides examples of modified-release oral dosage forms, including those formulated as tablets or capsules. See, for example, The United States Pharmacopeia 23/National Formulary 18, The United States Pharmacopeial Convention, Inc., Rockville Md., 1995 (hereinafter “the USP”), which also describes specific tests to determine the drug release capabilities of extended-release and delayed-release tablets and capsules. The USP test for drug release for extended-release and delayed-release articles is based on drug dissolution from the dosage unit against elapsed test time. Descriptions of various test apparatus and procedures may be found in the USP. Further guidance concerning the analysis of extended release dosage forms has been provided by the F.D.A. (See Guidance for Industry. Extended release oral dosage forms: development, evaluation, and application of in vitro/in vivo correlations. Rockville, Md.: Center for Drug Evaluation and Research, Food and Drug Administration, 1997).

Further examples of dosage forms of the invention include, but are not limited to modified-release (MR) dosage forms including delayed-release (DR) forms; prolonged-action (PA) forms; controlled-release (CR) forms; extended-release (ER) forms; timed-release (TR) forms; and long-acting (LA) forms. For the most part, these terms are used to describe orally administered dosage forms, however these terms may be applicable to any of the dosage forms, formulations, compositions and/or devices described herein. These formulations effect delayed total drug release for some time after drug administration, and/or drug release in small aliquots intermittently after administration, and/or drug release slowly at a controlled rate governed by the delivery system, and/or drug release at a constant rate that does not vary, and/or drug release for a significantly longer period than usual formulations.

In certain embodiments, a therapeutically effective amount of one or more polymer particles of the invention or of one or more antigens comprising one or more polymer particles of the invention is from about 1 ug/kg to about 1 μg/kg. Exemplary therapeutically effective dose ranges include, for example, from about 1 μg/kg to about 500 mg/kg, from about 1 μg/kg to about 400 mg/kg, from about 1 μg/kg to about 300 mg/kg, from about 1 μg/kg to about 200 mg/kg, from about 1 μg/kg to about 100 mg/kg, from about 1 μg/kg to about 90 mg/kg, from about 1 μg/kg to about 80 mg/kg, from about 1 μg/kg to about 70 mg/kg, from about 1 μg/kg to about 60 mg/kg, from about 1 μg/kg to about 50 mg/kg, from about 5 μg/kg to about 50 mg/kg, from about 10 μg/kg to about 50 mg/kg, from about 50 μg/kg to about 50 mg/kg, from about 100 μg/kg to about 50 mg/kg, from about 200 μg/kg to about 50 mg/kg, from about 300 μg/kg to about 50 mg/kg, from about 400 μg/kg to about 50 mg/kg, from about 500 μg/kg to about 50 mg/kg, from about 600 μg/kg to about 50 mg/kg, from about 700 μg/kg to about 50 mg/kg, from about 800 μg/kg to about 50 mg/kg, from about 900 μg/kg to about 50 mg/kg, about 1 mg/kg to about 50 mg/kg, about 5 mg/kg to about 50 mg/kg, about 10 mg/kg to about 50 mg/kg, about 15 mg/kg to about 50 mg/kg, about 20 mg/kg to about 50 mg/kg, about 25 mg/kg to about 50 mg/kg, about 30 mg/kg to about 50 mg/kg, about 35 mg/kg to about 50 mg/kg, about 40 mg/kg to about 50 mg/kg, or about 45 mg/kg to about 50 mg/kg.

Other therapeutically effective dose ranges include, for example, from about 1 mg/kg to about 1 μg/kg, from about 1.5 mg/kg to about 950 mg/kg, about 2 mg/kg to about 900 mg/kg, about 3 mg/kg to about 850 mg/kg, about 4 mg/kg to about 800 mg/kg, about 5 mg/kg to about 750 mg/kg, about 5 mg/kg to about 700 mg/kg, 5 mg/kg to about 600 mg/kg, about 5 mg/kg to about 500 mg/kg, about 10 mg/kg to about 400 mg/kg, about 10 mg/kg to about 300 mg/kg, about 10 mg/kg to about 200 mg/kg, about 10 mg/kg to about 250 mg/kg, about 10 mg/kg to about 200 mg/kg, about 10 mg/kg to about 200 mg/kg, about 10 mg/kg to about 150 mg/kg, about 10 mg/kg to about 100 mg/kg, about 10 mg/kg to about 75 mg/kg, about 10 mg/kg to about 50 mg/kg, or about 15 mg/kg to about 35 mg/kg.

In some embodiments of the invention targeting human subjects, a therapeutically effective amount of one or more polymer particles of the invention or of one or more antigens comprising one or more polymer particles of the invention is, for example, from about 10 mg to about 10 g per dose. Other therapeutically effective dose ranges include, for example, from about 20 mg to about 9 g, from about 30 mg to about 8 g, from about 40 mg to about 7 g, from about 50 mg to about 6 g, from about 60 mg to about 5 g, from about 70 mg to about 4 g, about 80 mg to about 3 g, about 100 mg to about 2 g, about 100 mg to about 1.5 g, about 200 mg to about 1400 mg, about 200 mg to about 1300 mg, about 200 mg to about 1200 mg, about 200 mg to about 1100 mg, about 200 mg to about 1000 mg, about 300 mg to about 900 mg, about 300 mg to about 800, about 300 mg to about 700 mg or about 300 mg to about 600 mg per dose.

The invention also in part provides low dose compositions, formulations and devices comprising one or more one or more polymer particles of the invention. For example, low dose compositions, formulations and the like, are administered in an amount sufficient to provide, for example, dosages from about 0.001 mg/kg to about 5 mg/kg, about 0.01 mg/kg to about 4.5 mg/kg, about 0.02 mg/kg to about 4 mg/kg, about 0.02 to about 3.5 mg/kg, about 0.02 mg/kg to about 3 mg/kg, about 0.05 mg/kg to about 2.5 mg/kg, about 0.05 mg/kg to about 2 mg/kg, about 0.05-0.1 mg/kg to about 5 mg/kg, about 0.05-0.1 mg/kg to about 4 mg/kg, about 0.05-0.1 mg/kg to about 3 mg/kg, about 0.05-0.1 mg/kg to about 2 mg/kg, about 0.05-0.1 mg/kg to about 1 mg/kg, and/or any other doses or dose ranges within the ranges set forth herein, of one or more one or more polymer particles of the invention or of one or more antigens comprising one or more polymer particles of the invention.

The doses described herein, may be administered in a single dose or multiple doses or divided doses. For example, doses may be administered, once, twice, three, four or more times over a treatment regime, as is well known in the immunological arts.

The efficacy of a composition useful according to the invention can be evaluated both in vitro and in vivo. See, e.g., the examples below. Briefly, the composition can be tested in vitro or in vivo for its ability to induce a cell-mediated immune response. For in vivo studies, the composition can be fed to or injected into an animal (e.g., a mouse) and its effects on eliciting an immune response are then assessed. Based on the results, an appropriate dosage range and administration route can be determined.

In some embodiments of the invention, a therapeutically effective amount is an amount effective to elicit an immunological response, such as, for example, a concentration of IFN-gamma in the blood of from about 0.5 ng/mL to about 20 ng/mL, about 0.5 ng/mL to about 15 ng/mL, about 0.5 ng/mL to about 10 ng/mL, about 0.5 ng/mL to about 9 ng/mL, about 1 ng/mL to about 8 ng/mL, about 2 ng/mL to about 7 ng/mL or about 3 ng/mL to about 6 ng/mL.

In some circumstances, including post infection or during prolonged infection, elevated IFN-gamma blood concentrations are observed, and such elevated concentrations should be accounted for in assessing a baseline against which elicitation of an effective immunological response by the polymer particles of the invention is to be assessed.

### 8. Treatment with Polymer Particles

It has been discovered that the polymer particles, e.g., polyhydroxyalkyl polymer particles, can be stably maintained as particles outside the host cell that produced them, and that these particles can be designed to suit a number of applications.

Functionalised polymer particles may comprise one or more surface-bound antigens capable of eliciting a cell-mediated or other immune response, one or more substances bound to binding domains of an antigen capable of eliciting a cell-mediated or other immune response, or a combination thereof.

In one embodiment, for example, a substance is immobilised on the particle surface during particle formation, bound to a binding domain capable of binding an antigen capable of eliciting a cell-mediated immune response, or integrated into the particle by loading, diffusion or incorporation.

In the context of use in the treatment of tuberculosis, for example, the polymer particles may comprise one or more surface-bound M. tuberculosis antigens, one or more substances bound to M. tuberculosis antigen binding domains, or a combination thereof.

In one embodiment a substance may be immobilised on the particle surface during particle formation, bound to, for example, a M. tuberculosis antigen binding domain, or integrated into the particle by loading, diffusion or incorporation. Covalent linking to the surface of the polymer particle, for example, by cross-linking, is also specifically contemplated.

In one embodiment the substance is selected from the list comprising, for example, a protein or protein fragment, a peptide, a polypeptide, an antibody or antibody fragment, an antibody binding domain, an antigen, an antigenic determinant, an epitope, an immunogen or fragment thereof, or any combination of any two or more thereof.

In one embodiment DNA from an intracellular pathogen can be fragmented and inserted into expression constructs encoding fusion polypeptides that comprise a polymer synthase. In this way, polymer particles displaying intracellular pathogen antigenic determinants can be produced and screened using serum from infected patients and antigen-presenting particles identified, isolated and reproduced using well-known and scalable bacterial production systems.

In one embodiment multiple antigens capable of eliciting a cell-mediated (or other) immune response are immobilised on the surface of the polymer particles.

In one embodiment DNA from a M. tuberculosis bacterium, for example, can be fragmented and inserted into expression constructs encoding fusion polypeptides that comprise a polymer synthase. In this way, polymer particles displaying M. tuberculosis antigenic determinants, for example, can be produced and screened using serum from infected patients and antigen-presenting particles identified, isolated and reproduced using well-known and scalable bacterial production systems.

In one embodiment, for example, multiple M. tuberculosis or other antigens are immobilised on the surface of the polymer particles.

Similarly, in various embodiments DNA from a hepatitis virus or from an influenza virus, for example, can be fragmented and inserted into expression constructs encoding fusion polypeptides that comprise a polymer synthase. In this way, polymer particles displaying hepatitis antigenic determinants or influenza antigenic determinants can be produced and screened using serum from infected patients and antigen-presenting particles identified, isolated and reproduced using well-known and scalable bacterial production systems.

In one embodiment multiple hepatitis or influenza antigens, for example, are immobilised on the surface of the polymer particles.

One aspect of the invention relates to the ability of the polymer particles carrying one or more antigens to elicit an immune response. In one embodiment, the polymer particles comprise at least one antigen capable of eliciting a cell-mediated or other immune response fused to the polymer bead. The polymer polymer particles display at least one antigens capable of eliciting a cell-mediated or other immune response on their surface to stimulate an optimal immune response to the antigenic moieties.

In one embodiment, the polymer particles carrying one or more antigens elicit an immune response. In one embodiment, the polymer particles comprise at least one M. tuberculosis antigen, for example, fused to the polymer bead. The polymer polymer particles display at least one M. tuberculosis antigen, for example, on their surface to stimulate an optimal immune response to the antigenic moieties.

In one embodiment, the polymer particles carrying one or more antigens elicit an immune response to hepatitis. In one embodiment, the polymer particles comprise at least one hepatitis antigen, for example, fused to the polymer bead. The polymer polymer particles display at least one hepatitis antigen, for example, on their surface to stimulate an optimal immune response to the antigenic moieties. In one embodiment, the polymer particles comprise at least one influenza antigen, for example, fused to the polymer bead. The polymer polymer particles display at least one influenza antigen, for example, on their surface to stimulate an optimal immune response to the antigenic moieties. Other antigens are contemplated, as noted herein.

In one embodiment, for example, more than one antigen or a combination of antigen and adjuvant or other immunomodulatory agent or molecule, such as an immunostimulatory agent or molecule, are present in or on the particle or present in a composition. Typically, the presence of the combination of antigens, adjuvants, or other immunomodulatory agents or molecules will be to further enhance the immune response.

In one embodiment, the invention provided a multiphase vaccine composition, for example. This hybrid vaccine displays different antigens specific to various stages of tuberculosis infection. For example, an early stage antigen is co-expressed with a latent stage antigen. Antigens specific to the various antigens, including intracellular antigens, are well known in the art and representative antigens for exemplary pathogens are described herein.

The present invention also relates to a method of eliciting a cell-mediated (and/or other) immune response in a subject, wherein the method comprises administering to a subject in need thereof a polymer particle comprising a particle-forming protein, preferably a polymer synthase, for example, fused to a binding domain capable of binding an antigen capable of eliciting a cell-mediated immune response.

In this embodiment, on administration to the subject the binding domain capable of binding an antigen capable of eliciting a cell-mediated immune response may bind to an endogenous antigen capable of eliciting a cell-mediated immune response. It will be appreciated that binding of a polymer particle comprising a binding domain capable of binding an antigen capable of eliciting a cell-mediated immune response to endogenous antigens capable of eliciting a cell-mediated immune response is able to elicit or enhance the subject's immune response.

For example, antigens capable of eliciting a cell-mediated immune response that is present in the subject prior to administration of the particle comprising at least one M. tuberculosis antigen binding domain, for example, but is unable to elicit an effective immune response in the subject, is on binding to the particle able to elicit an effective immune response or is effective to enhance the subject's immune response.

In one embodiment, the invention provides a method of eliciting an immune response in a subject infected with tuberculosis, for example, or previously immunised against tuberculosis, for example, wherein the method comprises administering to a subject in need thereof a polymer particle comprising a particle-forming protein fused to a M. tuberculosis antigen binding domain, for example.

In this embodiment, for example, on administration to the subject the M. tuberculosis antigen binding domain may bind to an endogenous M. tuberculosis antigen. It will be appreciated that binding of a polymer particle comprising a M. tuberculosis antigen binding domain to endogenous M. tuberculosis antigen, for example, is able to elicit or enhance the subject's immune response.

For example, M. tuberculosis antigen that is present in the subject prior to administration of the particle comprising at least one M. tuberculosis antigen binding domain, but is unable to elicit an effective immune response in the subject, is on binding to the particle able to elicit an effective immune response or is effective to enhance the subject's immune response.

In one embodiment, the invention provides a method of eliciting an immune response in a subject infected with hepatitis or previously immunised against hepatitis, for example, wherein the method comprises administering to a subject in need thereof a polymer particle comprising a particle-forming protein fused to a hepatitis antigen binding domain.

In this embodiment, on administration to the subject the hepatitis antigen binding domain may bind to an endogenous hepatitis antigen, for example. It will be appreciated that binding of a polymer particle comprising a hepatitis antigen binding domain to endogenous hepatitis antigen is able to elicit or enhance the subject's immune response.

For example, hepatitis antigen that is present in the subject prior to administration of the particle comprising at least one hepatitis antigen binding domain, but is unable to elicit an effective immune response in the subject, is on binding to the particle able to elicit an effective immune response or is effective to enhance the subject's immune response.

In one embodiment, for example, the invention provides a method of eliciting an immune response in a subject infected with hepatitis or previously immunised against influenza, wherein the method comprises administering to a subject in need thereof a polymer particle comprising a particle-forming protein fused to a hepatitis antigen binding domain.

In this embodiment, for example, on administration to the subject the influenza antigen binding domain may bind to an endogenous influenza antigen. It will be appreciated that binding of a polymer particle comprising a influenza antigen binding domain to endogenous influenza antigen is able to elicit or enhance the subject's immune response.

For example, influenza antigen that is present in the subject prior to administration of the particle comprising at least one hepatitis antigen binding domain, but is unable to elicit an effective immune response in the subject, is on binding to the particle able to elicit an effective immune response or is effective to enhance the subject's immune response.

It will be appreciated that the present invention provides particles, compositions and methods that elicit an immune response in subjects to whom they are administered. Preferably, the magnitude of the immune response elicited in response to one or more antigens presented to a subject using the particles, compositions and methods of the invention is greater than that elicited in response to the antigen alone (that is, in the absence of a particle or composition of the invention or presented by a method other than those provided herein). Methods to quantify the magnitude of an immune response, and particularly a cell-mediated immune response, are well known in the art.

### 9. Modulators of an Immune Response

In certain circumstances it will be desirable to produce polymer particles displaying a fusion protein comprising at least one antigen capable of eliciting a cell-mediated immune response. Alternatively, a fusion protein comprising at least one or more antigens capable of eliciting a cell-mediated immune response with an adjuvant or other modulator of an immune response is desirable for eliciting an immune response.

In certain circumstances it will be desirable to produce polymer particles displaying a fusion protein comprising at least one antigen capable of eliciting a humoral immune response. Alternatively, a fusion protein comprising at least one or more antigens capable of eliciting a humoral immune response with an adjuvant or other modulator of an immune response is desirable for eliciting an immune response.

For example, in the treatment of tuberculosis, it would be desirable to produce polymer particles displaying a fusion protein comprising at least one M. tuberculosis antigen, where the polymer particle is administered together with one or more adjuvants or other modulators of the immune system. Alternatively, a polymer particle comprising a fusion protein comprising one or more M. tuberculosis antigens, for example, and an adjuvant or other modulator of an immune response may be desirable for eliciting an immune response. In the treatment of hepatitis, it would be desirable to produce polymer particles displaying a fusion protein comprising at least one hepatitis antigen, where the polymer particle is administered together with one or more adjuvants or other modulators of the immune system. Alternatively, a polymer particle comprising a fusion protein comprising one or more hepatitis antigens and an adjuvant or other modulator of an immune response may be desirable for eliciting an immune response. In the treatment of influenza, it would be desirable to produce polymer particles displaying a fusion protein comprising at least one influenza antigen, where the polymer particle is administered together with one or more adjuvants or other modulators of the immune system. Alternatively, a polymer particle comprising a fusion protein comprising one or more influenza antigens and an adjuvant or other modulator of an immune response may be desirable for eliciting an immune response.

In one example, a polymer particle of the invention may comprise one or more antigens together with one or more toll-like receptors, including one or more toll-like receptors able to bind one or more of the group of ligands comprising LPS, lipoproteins, lipopeptides, flagellin, double-stranded RNA, unmethylated CpG islands, or bacterial or viral DNA or RNA. Similarly, a composition of the invention may comprise a population of polymer particles comprising one or more Tb antigens, and a population of polymer particles comprising one or more immunomodulatory molecules, such as one or more toll-like receptors.

The presence of one or more immunomodulatory molecules may be useful in eliciting a humoral-specific immune response, or a cell-mediated-specific immune response, or in eliciting an immune response comprising a combination of both humoral and cell-mediated responses.

Specific antigens may be selected from any known M. tuberculosis antigens, including those described above and in the documents referred to herein. Antigens may be selected so as to produce a vaccine suitable for treating or immunising against early stage infection. Alternatively, a multi-phase vaccine comprising antigens from early and latent stages of infection is provided. For example, a vaccine delivery system comprising a polymer particle displaying an Ag85A-ESAT-6 fusion protein is provided. A second example may include a polymer particle expressing Ag85A antigen with a known adjuvant suitable for stimulating an immune response against tuberculosis.

Specific antigens may be selected from any known antigens capable of eliciting a cell-mediated immune response, including those described above and in the documents referred to herein. Antigens may be selected so as to produce a vaccine suitable for treating or immunising against early stage infection. Alternatively, a multi-phase vaccine comprising antigens from early and latent stages of infection is provided.

The invention consists in the foregoing and also envisages constructions of which the following gives examples only.

### EXAMPLES

### Example 1—Construction of Plasmids and Production of PHA Polymer Particles in E. coli

This example describes the construction of plasmids for the production in E. coli of polymer particles displaying the tuberculosis antigens Ag-85A and ESAT-6, the Hepatitis C core antigen, and the H1 subtype of the influenza hemagglutinin (HA) antigen together with an analysis of the immunogenecity of the polymer particles.

### Materials and Methods

1. Growth of Escherichia coli Strains

Escherichia coli DH5α (Invitrogen) was grown in Difco™ Luria Broth (see Table 1) supplemented with 1% (w/w) glucose and 75 μg/mL ampicillin. Escherichia coli BL21 (Invitrogen) was grown in Difco™ Luria Broth supplemented with 1% (w/w) glucose, 75 μg/mL ampicillin, and 30 μg/mL chloramphenicol.

TABLE 1
Difco ™ Luria Broth

| | |
|---|---|
| Pancreatic Digest of Casein | 10 g |
| Yeast Extract | 5 g |
| Sodium Chloride | 0.5 g |

Disolved in 1000 mL water

### 2. Construction of Plasmids

All plasmids and oligonucleotides used in this example are listed in Table 2.

The PhaA and PhaB enzymes were encoded by plasmid pMCS69. For tuberculosis antigen polymer particles, the plasmid DK1.2-Ag85A-ESAT-6 contained a hybrid gene comprised of the coding region (without the secretory signal sequence) of Ag85A (N-terminal component) and the coding region of ESAT-6 (C-terminal component). A DNA fragment encoding the Ag85A-ESAT-6 fusion protein and including a translation initiation site and start codon was isolated from this plasmid by PCR using primers Ag85A-SpeI [SEQ ID No. 3] and ESAT-6-SpeI [SEQ ID No. 4] and ligated into XbaI, ClaI—endonucleased pHAS vector to generate the plasmid pHAS-Ag85A-ESAT-6.

The coding sequence from the 3′OH terminal fragment of the Ag85A-ESAT6 fusion is shown as SEQ ID No. 1, with the derived amino acid sequence shown as SEQ ID No. 2.

For Hepatitis C antigen polymer particles, Hep C DNA synthesized by DNA 2.0 as an SpeI/NotI fragment was subcloned into the pET-14b-scFv-PhaC vector, resulting in the formation of pET-14b Hep-PhaC.

The coding sequence from the 3′OH terminal fragment of the HepC-PhaC fusion is shown as SEQ ID No. 7, with the derived amino acid sequence shown as SEQ ID No. 8.

For HA antigen polymer particles, a full length hemagglutinin sequence was synthesized by GenScript, as an SpeI/NotI fragment. This fragment was subcloned into the pET-14b-scFv-PhaC vector, resulting in the formation of pET-14b hemagglutinin-PhaC. To create the shorter H1 part of the hemagglutinin antigen, the H1 sequence was amplified using pET-14b hemagglutinin-PhaC as a template with primers as described in Table 2. The SpeI/SunI fragment was subcloned into pET-14b hemagglutinin-PhaC, resulting in the formation of pET-14b HA1 of H3-PhaC. The XhoI/BamHI fragment was subcloned into pET-14b PhaC-linker-MalE, resulting in the formation of pET-14b PhaC-linker-HA1 of H3.

The coding sequence from the 3′OH terminal fragment of the HA1 of H3-PhaC fusion is shown as SEQ ID No. 11 with the derived amino acid sequence shown as SEQ ID No. 12.

TABLE 2
Plasmids and Oligonucleotides

| | Description |
|---|---|
| Plasmids | |
| pET-14b | Ap^{r}, T7 promoter |
| pHAS | pET14b derivative containing the |
| | NdeI/BamHI inserted phaC gene |
| | from C. necator |
| pMCS69 | pBBR1MCS derivative containing |
| | genes phaA and phaB from C. necator |
| pCWE | pBluescript SK(−) derivated |
| SpeI | containing the PHA synthase |
| | gene from C. necator |
| DK1.2- | pBluescript II SK(+) containing |
| Ag85A- | fusion between Ag85A and ESAT-6 |
| ESAT-6 | |
| pCWE | pCWE derivative containing Ag85A- |
| SpeI- | ESAT-6 hybrid gene inserted into |
| Ag85AESAT- | SpeI site |
| 6 | |
| pHAS- | pHAS containing Ag85A-ESAT-6 hybrid |
| Ag85A- | gene upstream of phaC |
| ESAT-6 | |
| Oligonu- | |
| cleotides | |
| Ag85A- | 5′-gctactagtaataaggagatatacatatgtttt |
| SpeI | cccggccgggcttgc-3′ |
| | [SEQ ID No. 5] |
| ESAT-6- | 5′-tgcactagttgcgaacatcccagtgacgtt-3′ |
| SpeI | [SEQ ID No. 6] |
| HA1 of | 5′-agatactagtatgcagaaactgccgggtaacga |
| H3-SpeI | taatagtacc-3′ |
| | [SEQ ID No 13] |
| HA1 of | 5′-gatgcgtacgggtctgtttttccggcacattgc |
| H3-SunI | gcatgcc-3′ |
| | [SEQ ID No. 14] |
| HA1 of | 5′-agatctcgagcagaaactgccgggtaacgataat |
| H3-XhoI | agtacc-3′ |
| | [SEQ ID No. 15] |
| HA1 of | 5′-gatgggatcctcaggtctgtttttccggcacatt |
| H3-BamHI | gcgcatgcc-3′ |
| | [SEQ ID No. 16] |

### 3. Production of Ag85A-ESAT-6 Displaying Polymer Particles

Plasmids pHAS-Ag85A-ESAT-6 and pHAS were introduced into E. coli BL21 (DE3) cells harbouring plasmid pMCS69. The transformants were cultured in conditions suitable for the production of biopolyester polymer particles, as described above. The ability to produce Ag85A-ESAT-6 polymer particles, or wild-type polymer particles, respectively, was then assessed as described below.

### 4. Gas Chromatography Mass Spectroscopy (GC-MS)

The polyester content of bacterial cells harboring the various plasmids corresponds to the activity of the PhaC synthase in vivo. The amount of accumulated polyester was assessed by gas chromatography-mass spectroscopy (GC-MS) analysis to determine phaC synthase activity, and particularly to assess whether the PhaC-Tb antigen fusion still catalyses polyester synthesis and mediates intracellular granule formation. Polyester content was quantitatively determined by GC-MS after conversion of the polyester into 3-hydroxymethyl ester by acid-catalysed methanolysis.

### Results

GC-MS analysis of cells carrying pHAS-Ag85A-ESAT-6 and pMCS69, or pHAS and pMCS69, confirmed the presence of the polyester polyhydroxybutyrate. The presence of intracellular polyester inclusions was further confirmed by fluorescent microscopy using Nile Red staining.

### Discussion

The presence of polyhydroxybutyrate in cells carrying pHAS-Ag85A-ESAT-6 and pMCS69 indicated that the phaC polyester synthase domain retained polymer synthase activity when present as a tripartite fusion protein.

### Example 2—Construction of Plasmids and Production of PHA Polymer Particles in L. lactis

This example describes the construction of plasmids for the production in L. lactis of polymer polymer particles displaying the tuberculosis antigens Ag-85A and ESAT-6.

### Materials and Methods

### 1. Construction of Plasmids

All plasmids and strains of L. lactis used in this example are listed in Table 3. The gene encoding the antigen(s) Ag85A/ESAT6 was synthesized by GeneScript Corporation (Piscataway, N.J.). Codon usage was adapted to the codon usage bias of L. lactis.

A fragment of pUC57-ZZ comprising part of the nisA promoter (P_{nisA}) was obtained by NdeI digest of pUC57-ZZ and ligated with NdeI-digested pUC57-ESAT6 to obtain pUC57-nisESAT6. A BstBI-BamHI fragment of pUC57-nisESAT6 containing part of P_{nisA} and the Ag85A/ESAT6 gene was then inserted upstream of phaB at the corresponding sites of pNZ-AB, resulting in pNZ-ESAT6-B. To introduce the phaC and phaA comprising fragment of pNZ-CAB into pNZ-ESAT6-B, both plasmids were hydrolyzed with NheI and BamHI and the phaCA fragment of pNZ-CAB was inserted into pNZ-ESAT6-B, resulting in pNZ-ESAT6-CAB.

The coding sequence from the 3′OH terminal fragment of the nisA promoter (P_{nisA}) is shown as SEQ ID No. 3, with the derived amino acid sequence shown as SEQ ID No. 4.

For Hepatitis C antigen polymer particles, the Hep C DNA sequence was codon optimised for expression in L. lactis and synthesized by GenScript as an NcoI/NheI fragment. The fragment was subcloned into the pNZ-CAB plasmid as described in Table 3, resulting in the formation of pNZ-HepC-PhaCAB.

The coding sequence from the 3′OH terminal fragment of the HepC-PhaC (pNZ) fusion is shown as SEQ ID No. 9, with the derived amino acid sequence shown as SEQ ID No. 10.

TABLE 3
Plasmids and Oligonucleotides

| | Description |
|---|---|
| L. lactis strain | |
| MG1363 | NCDO 712 derivative, plasmid and phage free strain |
| NZ9000 | MG1363 derivative, pepN::nisRK |
| Plasmids | |
| pUC57 | Cloning vector, ColE1 origin, Amp^{r} |
| pUC57-ESAT6 | Codon-optimised gene for Ag85A/ESAT6 in EcoRV |
| | site of pUC57 |
| pUC57-ZZ | Codon-optimised gene for ZZ domain in EcoRV |
| | site of pUC57 |
| pUC-nisESAT6 | pUC57 derivative, P_{nisA}-Ag85A/ESAT6 |
| pNZ8148 | Cm^{r}, pSH71 origin, P_{nisA} |
| pNZ-AB | pUC8148 derivative, P_{nisA}-phaAB |
| pNZ-CAB | pUC8148 derivative, P_{nisA}-phaCAB |
| pNZ-ESAT6-B | pUC8148 derivative, P_{nisA}-Ag85A/ESAT6-phaB |
| pNZ-ESAT6-CAB | pUC8148 derivative, P_{nisA}-Ag85A/ESAT6-phaC- |
| | phaAB |
| pNZ-HepC-PhaCAB | pUC8148 derivative, P_{nisA}-HepC-phaC-phaAB |

### Example 3—Isolation of Polyester Polymer Particles and Characterization of the Fusion Protein

This example describes the characterization of biopolyester polymer particles displaying Ag85A-ESAT-6 at their surface.

### Materials and Methods

### 1. Isolation of Polyester Polymer Particles

Polyester granules were isolated by disrupting the bacteria and whole cell lysates were centrifuged at 4000 g for 15 minutes at 4° C. to sediment the polyester polymer particles. The polymer particles were purified via glycerol gradient ultracentrifugation

### 2. Protein Concentration Determination

The concentration of protein attached to polymer particles was determined using the Bio-Rad Protein Assay according to the manufacturer's instructions (Bio-Rad). Following concentration determination, the proteins were separated by SDS-PAGE and stained with SimplyBlue Safe Stain (Invitrogen).

The amount of Ag85A-ESAT-6 PhaC fusion protein relative to the amount of total protein attached to the polymer particles was detected using a Gel Doc™ XR and analysed using Quantity One software (version 4.6.2, Bio-Rad Laboratories). Proteins of interest were excised from the gel and subjected to tryptic peptide fingerprinting using matrix-assisted laser desorption/ionization time-of-flight spectrometry (MALDI-TOF-MS).

### 3. ELISA

Maxisorb plates (Nunc) were coated overnight at 4° C. with purified Ag85A-ESAT-6 polymer particles or wild-type polymer particles, diluted in carbonate-bicarbonate coating buffer (pH 9.6) (Sigma-Aldrich). Serial dilutions of the buffer were used, ranging from 1 mg/ml to 0.015 mg/ml protein concentration. Plates were washed and blocked (see Table 4) for 2 h at 25° C.

Plates were then washed in PBS-Tween 20, incubated with mouse antibody to ESAT-6 (Abcam), washed and further incubated for 1 hour at room temperature with anti-mouse IgG:horse radish peroxidase conjugate (Sigma-Aldrich) diluted in 1% (w/v) BSA in PBS. After further washing, o-phenylenediamine (OPD) substrate (Sigma-Aldrich) was added and the plates were incubated for 30 minutes at room temperature.

The reaction was stopped with 0.5 M H2SO4 and absorbance recorded at 495 nm.

### 4. Flow Cytometry

Twenty-five micrograms of purified Ag85A-ESAT-6 polymer particles or wild-type polymer particles were washed twice in ice-cold flow cytometry buffer (see Table 4) and incubated with mouse anti-ESAT-6 antibodies (Abcam). After washing, polymer particles were stained with rat anti-mouse Fluorescein isothiocyanate (FITC)-labelled antibody (BD Pharmingen, CA, USA), incubated for 30 minutes on ice in the dark and washed again. A BD FACScalibur (BD Biosciences, CA, USA) was used to collect at least 10,000 events for each sample and analysed using CellQuest software.

TABLE 4
Buffers

| ELISA wash buffer | | ELISA block buffer | | Flow Cytometry buffer | |
|---|---|---|---|---|---|
| PBS | | PBS | | PBS | |
| Tween 20 | 0.05% | Bovine Serum | 3% | Foetal Calf Serum | 1% |
| | | Albumin | | Sodium Azide | 0.1% |

### Results

The polymer particles displayed high levels of protein as determined by a prominent protein band with an apparent molecular weight of 102 kDa and 63 kDa for Ag85A-ESAT-6-PhaC, and PhaC, respectively. The identity of these proteins was confirmed by tryptic peptide fingerprinting using MALDI-TOF-MS. ELISA indicated that Ag85A-ESAT-6 polymer particles bound to the anti-ESAT-6 antibody in a dose-dependent manner, while wild-type polymer particles did not bind to the antibody. Flow cytometry showed that >98% of Ag85A-ESAT-6 polymer particles bound anti-ESAT-6 antibodies.

### Discussion

The results of this example indicated that the expression in recombinant E. coli of a hybrid gene encoding a tripartite fusion protein Ag85A-ESAT-6-PhaC was successful, leading to the overproduction of polyester polymer particles displaying the fusion protein at their surface.

### Example 4—Immunogenicity of Influenza Polymer Particle Vaccines

This example describes the construction of plasmids for the production in transformed hosts, in this case, E. coli, of polymer particles simultaneously displaying the influenza antigens neuraminidase, M1 influenza coat protein and hemagglutinin, together with an analysis of the immunogenecity of the polymer particles. Particles with these antigens are useful as prophylactic and therapeutic vaccines against influenza.

### Materials and Methods

All animal experiments were approved by the AgResearch Grasslands Animal Ethics Committee (Palmerston North, New Zealand).

1. Growth of Escherichia coli Strains

Escherichia coli DH5α (Invitrogen) is grown in Difco™ Luria Broth as detailed in Table 1 of Example 1 supplemented with 1% (w/w) glucose and 75 μg/mL ampicillin. Escherichia coli BL21 (Invitrogen) is grown in Difco™ Luria Broth or a defined medium supplemented with 1% (w/w) glucose, 75 μg/mL ampicillin, and 30 μg/mL chloramphenicol.

### 2. Construction of Plasmids

All plasmids and oligonucleotides in this example are listed in Table 5. The PhaA and PhaB enzymes are encoded by plasmid pMCS69.

To produce polymer particles displaying the neuraminidase antigen, the gene encoding neuraminidase was codon optimised and synthesised by GenScript Inc as SpeI/SunI and XhoI/BamHI fragments. The SpeI/SunI fragment was inserted into the pET-14b HA1 of H3-PhaC plasmid, yielding plasmid pET-14b-NA-PhaC. The XhoI/BamHI fragment was subcloned into pET-14b-PhaC-linker-MalE, resulting in plasmid pET-14b-PhaC-linker-NA.

To produce polymer particles displaying the M1 influenza coat protein, the M1 gene sequence was codon optimised and synthesised by GenScript as SpeI/SunI and XhoI/BamHI fragments. The SpeI/SunI fragment was inserted into the pET-14b HA1 of H3-PhaC plasmid, yielding plasmid pET-14b-M1-PhaC. The XhoI/BamHI fragment was subcloned into pET-14b-PhaC-linker-MalE, resulting in plasmid pET-14b-PhaC-linker-M1.

To produce polymer particles simultaneously displaying all three influenza antigens, the XbaI/NotI fragment from plasmid pET-14b-NA-PhaC is subcloned into plasmid pET-14b-PhaC-linker-M1, yielding plasmid pET-14b-NA-PhaC-linker-M1. Hemagglutinin-PhaC is PCR amplified using the BamH1 H3 primer as described in Table 2 of Example 1. The respective BamH1/BlpI fragment is subcloned into plasmid pET-14b-NA-PhaC-linker-M1, resulting in plasmid pET-14b-NA-PhaC-linker-M1/hemagglutinin-PhaC.

The construct for the NA-PhaC fusion and PhaC-linker-NA fusion are shown as SEQ ID No. 17 and 19, respectively, with the derived amino acid sequences shown as SEQ ID No. 18 and 20, respectively. The construct for the M1-PhaC fusion and PhaC-linker-M1 fusion are shown as SEQ ID No. 21 and 23, respectively, with the derived amino acid sequences shown as SEQ ID No. 22 and 24, respectively. The construct for the NA-PhaC-linker-M1 fusion is shown as SEQ ID No. 25, with the derived amino acid sequence shown as SEQ ID No. 26. The construct for the hemagglutinin-PhaC fusion is shown as SEQ ID No. 27, with the derived amino acid sequence shown as SEQ ID No. 28.

TABLE 5
Plasmids and Oligonucleotides

| Plasmids | Description |
|---|---|
| pHAS | pET14b derivative containing the NdeI/BamHI |
| | inserted phaC gene from C. necator |
| pMCS69 | pBBR1MCS derivative containing genes phaA and |
| | phaB from C. necator |
| pET-14b M-PhaC- | pET-14b PhaC-linker-MalE derivative |
| linker-MalE | containing the mpl sequence fused to the |
| | 5′ end of phaC |
| pET-14b-PhaC- | pET-14b PhaC-linker-MalE derivative |
| linker-NA | containing the NA sequence fused to the |
| | 3′ end of phaC |
| pET-14b-PhaC- | pET-14b PhaC-linker-MalE derivative |
| linker-M1 | containing the M1 sequence fused to the |
| | 3′ end of phaC |
| pET-14b-NA-PhaC- | pET-14b PhaC-linker-MalE derivative |
| linker-M1/ | containing the NA sequence fused to the 5′ end of |
| hemagglutinin- | phaC and the M1/hemagglutinin sequence |
| PhaC | fused to the 3′ end of phaC |

### 3. Production of AcpA-IglC Displaying Particles

Plasmids pET-14b-PhaC-linker-NA, pET-14b-PhaC-linker-M1, pET-14b-NA-PhaC-linker-M1/hemagglutinin-PhaC and pHAS are introduced into E. coli BL21 (DE3) cells harbouring plasmid pMCS69. The transformants are cultured in conditions suitable for the production of biopolyester particles, as described in Example 1. The ability to produce NA, M1 or NA-M1-Hemagglutinin particles or wild-type particles, respectively, is assessed as described below.

### 4. Gas Chromatography Mass Spectroscopy (GC-MS)

The polyester content of bacterial cells harbouring the various plasmids corresponds to the activity of the PhaC synthase in vivo. The amount of accumulated polyester is assessed by gas chromatography-mass spectroscopy (GC-MS) analysis to determine phaC synthase activity, and particularly to confirm that the PhaC-NA, Pha-M1 and PhaC-NA-M1-HA fusions catalyse polyester synthesis and mediate intracellular granule formation. Polyester content is quantitatively determined by GC-MS after conversion of the polyester into 3-hydroxymethyl ester by acid-catalysed methanolysis.

### 5. Isolation of Polyester Particles

Polyester granules are isolated by disrupting the bacteria and whole cell lysates are centrifuged at 4000 g for 15 minutes at 4° C. to sediment the polyester particles. The particles are purified via glycerol gradient ultracentrifugation

### 6. Protein Concentration Determination

The concentration of protein attached to particles is determined using the Bio-Rad Protein Assay according to the manufacturer's instructions (Bio-Rad). Following concentration determination, the proteins are separated by SDS-PAGE and stained with SimplyBlue Safe Stain (Invitrogen).

The amount of PhaC-NA, PhaC-M1 and PhaC-NA-M1-HA fusion protein relative to the amount of total protein attached to the particles is detected using a Gel Doc™ XR and analysed using Quantity One software (version 4.6.2, Bio-Rad Laboratories). Proteins of interest are excised from the gel and subjected to tryptic peptide fingerprinting using matrix-assisted laser desorption/ionization time-of-flight spectrometry (MALDI-TOF-MS), which allows identification of the fusion protein domains.

### 7. ELISA

Maxisorb plates (Nunc) are coated overnight at 4° C. with purified PorA-C-PorB particles or HA, M1, NA-M1-HA particles or wild-type particles, diluted in carbonate-bicarbonate coating buffer (pH 9.6) (Sigma-Aldrich). Serial dilutions of the buffer are used, ranging from 1 mg/ml to 0.015 mg/ml protein concentration. Plates are washed and blocked for 2 h at 25° C. (see Table 4).

Plates are then washed in PBS-Tween 20, incubated with mouse antibodies raised against the various antigens, washed and further incubated for 1 hour at room temperature with anti-mouse IgG:horse radish peroxidase conjugate (Sigma-Aldrich) diluted in 1% (w/v) BSA in PBS. After further ishing, o-phenylenediamine (OPD) substrate (Sigma-Aldrich) is added and the plates are incubated for 30 minutes at room temperature.

The reaction is stopped with 0.5 M H2SO4 and absorbance recorded at 495 nm.

### 8. Flow Cytometry

Twenty-five micrograms of various purified antigen-displaying particles or wild-type particles are washed twice in ice-cold flow cytometry buffer as described in Table 4 of Example 3 and incubated with mouse anti-antigen antibodies. After washing, particles are stained with rat anti-mouse Fluorescein isothiocyanate (FITC)-labelled antibody (BD Pharmingen, CA, USA), incubated for 30 minutes on ice in the dark and washed again. A BD FACScalibur (BD Biosciences, CA, USA) is used to collect at least 10,000 events for each sample and analysed using CellQuest software.

### 9. Immunisation of Mice

Female C57BL/6 mice (Malaghan Institute, Wellington, NZ) aged 6-8 weeks are intramuscularly immunized three times at 2 week intervals. The three treatment groups are as follows:
a) individuals immunised with wild-type particles (i.e., particles prepared from bacterial cells carrying pHAS and pMCS69);
b) individuals immunised with antigen particles alone (i.e., particles prepared from bacterial cells carrying plasmids encoding the various antigen-PhaC fusion proteins and pMCS69);
c) individuals immunised with the various antigen particles mixed with 20% Emulsigen™ adjuvant (MVP Laboratories).

Non-vaccinated control animals are included for each set of experiments.

### 10. Immunological Assay

The mice are anaesthetised three weeks after the last immunisation and blood is collected, centrifuged, and the serum collected and frozen at −20° C. until assayed.

The mice are then euthanized, their spleens removed and a single cell suspension is prepared by passage through an 80 gauge wire mesh sieve. Spleen red blood cells (RBCs) are lysed using a solution of 17 mM TRIS-HCl and 140 mM NH4Cl. After washing, the RBCs are cultured in Dulbecco's Modified Eagle media (DMEM) supplemented with 2 mM glutamine (Invitrogen), 100 U/mL penicillin (Invitrogen), 100 μg/mL streptomycin (Invitrogen), 5×10-5 M 2-mercaptoethanol (Sigma) and 5% (w/w) Foetal Calf Serum (Invitrogen).

The cells are incubated at 37° C. in 10% CO2 in medium alone, or in medium containing the respective antigens.

### 11. Quantification of IFN-γ

Culture supernatants are removed after 4 days incubation and frozen at −20° C. until assayed. Levels of IFN-γ in the supernatants are measured by ELISA (BD Biosciences) according to manufacturer's instructions using commercially available antibodies and standards (BD Pharmingen).

### 12. Quantification of Serum Antibody

Serum antibody is measured by ELISA using immobilized antigen displaying particles for antibody capture.

### 13. Statistical Analysis

Analysis of IFN-γ and antibody responses is performed by Kruskal-Wallis one-way analysis of variance (ANOVA).

### Results

Expression in recombinant E. coli of the respective hybrid genes encoding the various antigen-PhaC fusion proteins allows production of polyester particles displaying the fusion protein at their surface.

GC-MS analysis of cells carrying plasmids pET-14b-PhaC-linker-NA, pET-14b-PhaC-linker-M1, pET-14b-NA-PhaC-linker-M1/hemagglutinin-PhaC and pHAS all in the presence of pMCS69, will confirm the presence of the polyester polyhydroxybutyrate. The presence of intracellular polyester inclusions may be further confirmed by fluorescent microscopy using Nile Red staining.

The presence of polyhydroxybutyrate in cells carrying plasmids pET-14b-PhaC-linker-NA, pET-14b-PhaC-linker-M1, pET-14b-NA-PhaC-linker-M1/hemagglutinin-PhaC and pHAS (wildtype control) all in the presence of pMCS69 indicates that the phaC polyester synthase domain retains polymer synthase activity when present as a single or tripartite fusion protein.

High level protein display by polymer particles is determined by a prominent protein band with an apparent molecular weight directly aligning with molecular weight deduced from the fusion protein sequence. The identity of these proteins is confirmed by tryptic peptide fingerprinting using MALDI-TOF-MS. ELISA results indicate that the various antigen displaying particles bind to the respective anti-antigen antibody in a dose-dependent manner, while wild-type particles show significantly less binding of antibody. Flow cytometry results preferably show that >98% of antigen particles bind anti-antigen antibodies.

Preferably, no overt toxicity is observed in any of the animals after immunization, and mouse weights does not differ significantly between groups during the time-course of the experiment, and mice in all groups gained weight. Mice immunised with polyester particles will develop small lumps (2.5 mm in diameter) at the immunisation sites but generally without abscesses or suppuration, and are typically healthy throughout the trial with normal behaviour and good quality fur.

A dose of 10-100 μg of antigen particles is optimal at generating a significant antibody response in mice. This dose induces significantly higher antibody titres when compared to a 10-100 μg dose of wildtype particles alone. Other doses may also be tested and used. In a second experiment which includes non-immunised control mice compare bead formulations with and without an adjuvant, and evaluated for significantly higher antigen-specific serum antibody responses for both vaccine groups given antigen particles compared to non-vaccinated mice. The highest antibody responses may be observed in mice immunised with antigen particles in Emulsigen. Antibody responses for the IgG1 isotype will typically be stronger than responses for IgG2 in both experiments.

The cell-mediated response to antigens of mice immunised with 10-100 μg antigen particles is also significantly enhanced compared to that of mice immunised with wildtype particles alone, or with PBS alone, and there should typically be no significant difference in the cell-mediated responses of mice immunised with wildtype particles alone compared to PBS-immunised control mice.

The IFN-γ response to either antigen in mice immunised 3 times with 10-100 μg of wild-type particles (no influenza antigen) will typically not differ significantly from that of PBS-immunised control mice. In contrast, a significantly greater IFN-γ response to each antigen may be observed in mice immunised 3 times with antigen particles, and in mice immunised 3 times with antigen particles and Emulsigen. Expected is a significantly greater IFN-γ response to each antigen observed in mice immunised 3 times with antigen particles and Emulsigen than all the other vaccine groups.

The engineered polyester particles which display neuroaminidase, M1 coat protein or hemagglutinin antigens are capable of producing an antigen-specific cell-mediated response, as well as significantly increasing the production of IgG1 and IgG2 antibodies.

In addition to generation of both humoral and cell-mediated immune responses, the lack of adverse side effects such as weight loss, and absence of abscesses and suppuration at the injection site indicate that the polyester particles are well tolerated, safe, and non-toxic.

### Example 5—Immunogenicity of Francisella tularensis Polymer Particle Vaccines

This example describes the construction of plasmids for the production in transformed hosts, in this case, E. coli, of polymer particles simulataneously displaying the Francisella tularensis antigens AcpA and IglC, together with an analysis of the immunogenecity of the polymer particles. Particles with these antigens are useful as prophylactic and therapeutic vaccines against Tularemia.

### Materials and Methods

All animal experiments were approved by the AgResearch Grasslands Animal Ethics Committee (Palmerston North, New Zealand).

1. Construction of Plasmids and Production of PHA Particles in E. coli

All plasmids and oligonucleotides in this example are listed in Table 6. The PhaA and PhaB enzymes are encoded by plasmid pMCS69.

To produce polymer particles simultaneously displaying two F. tularensis antigens, genes encoding the antigens AcpA and IglC are codon optimized and synthesized by Genscript Inc. to allow subcloning into pET-14b M-PhaC-linker-MalE XbaI-SpeI site for an N-terminal fusion and into XhoI-BamHI sites for a C-terminal fusion to the PhaC polymer bead forming enzyme. The AcpA encoding gene is inserted into the XbaI-SpeI sites and on the same plasmid the IglC encoding gene is inserted into the XhoI-BamHI sites. Both gene insertions are in frame with the M and MalE encoding regions of the original plasmid replaced, yielding plasmid pET14B-AcpA-C-IglC.

The construct for the AcpA-C-IglC fusion is shown as SEQ ID No. 29, with the derived amino acid sequence shown as SEQ ID No. 30.

TABLE 6
Plasmids and Oligonucleotides

| Plasmids | Description |
|---|---|
| pHAS | pET14b derivative containing the NdeI/BamHI inserted |
| | phaC gene from C. necator |
| pMCS69 | pBBR1MCS derivative containing genes phaA and phaB |
| | from C. necator |
| pET-14b M- | pET-14b PhaC-linker-MalE derivative |
| PhaC-linker- | containing the mpl sequence fused to the |
| MalE | 5′ end of phaC |
| pET14B- | pET-14b M-PhaC-linker-MalE derivative |
| AcpA-C-IglC | containing the acpA sequence fused to the |
| | 5′ end and iglC fused to the 3′ end of phaC |

Plasmid pET14B-AcpA-C-IglC and pHAS are introduced into E. coli BL21 (DE3) cells harbouring plasmid pMCS69. The transformants are cultured in conditions suitable for the production of biopolyester particles, as described in Example 1. The ability to produce AcpA-IglC particles or wild-type particles, respectively, is assessed as described below.

### 2. Isolation of Polyester Particles

Polyester granules are isolated by disrupting the bacteria and whole cell lysates are centrifuged at 4000 g for 15 minutes at 4° C. to sediment the polyester particles. The particles are purified via glycerol gradient ultracentrifugation

The concentration of protein attached to particles is determined using the Bio-Rad Protein Assay as described in Example 3.

The amount of AcpA-PhaC-IglC fusion protein relative to the amount of total protein attached to the particles is detected using a Gel Doc™ XR, analysed using Quantity One software (version 4.6.2, Bio-Rad Laboratories) and the proteins of interest identified as described in Example 3.

### 3. ELISA

Immuno-reactivity of the F. tularensis polymer particles is determined by enzyme-linked immunosorbent assay (ELISA) as described in Example 3. Briefly, maxisorb plates (Nunc) are coated overnight at 4° C. with purified PorA-C-PorB particles or AcpA-IglC particles or wild-type particles, diluted in carbonate-bicarbonate coating buffer (pH 9.6) (Sigma-Aldrich). Serial dilutions of the buffer are used, ranging from 1 mg/ml to 0.015 mg/ml protein concentration. Plates are washed and blocked for 2 h at 25° C.

Plates are then washed in PBS-Tween 20, incubated with mouse antibodies raised against the various antigens, washed and further incubated for 1 hour at room temperature with anti-mouse IgG:horse radish peroxidase conjugate (Sigma-Aldrich) diluted in 1% (w/v) BSA in PBS.

After further ishing, o-phenylenediamine (OPD) substrate (Sigma-Aldrich) is added and the plates are incubated for 30 minutes at room temperature.

The reaction is stopped with 0.5 M H2SO4 and absorbance recorded at 495 nm.

### 4. Immunisation of Mice

Female C57BL/6 mice (Malaghan Institute, Wellington, NZ) aged 6-8 weeks are intramuscularly immunized three times at 2 week intervals. The three treatment groups are as follows:
a) individuals immunised with wild-type particles (i.e., particles prepared from bacterial cells carrying pHAS and pMCS69);
b) individuals immunised with antigen particles alone (i.e., particles prepared from bacterial cells carrying plasmids encoding the various antigen-PhaC fusion proteins and pMCS69);
c) individuals immunised with the various antigen particles mixed with 20% Emulsigen™ adjuvant (MVP Laboratories).

Non-vaccinated control animals are included for each set of experiments.

### 5. Immunological Assay

The mice are anaesthetised three weeks after the last immunisation and blood is collected, centrifuged, and the serum collected and frozen at −20° C. until assayed.

The mice are then euthanized, their spleens removed and a single cell suspension is prepared by passage through an 80 gauge wire mesh sieve. Spleen red blood cells (RBCs) are processed as described in Example 4.

### 6. Quantification of IFN-γ

Culture supernatants are removed after 4 days incubation and frozen at −20° C. until assayed.

Levels of IFN-γ in the supernatants are measured by ELISA (BD Biosciences) according to manufacturer's instructions using commercially available antibodies and standards (BD Pharmingen).

### 7. Quantification of Serum Antibody

Serum antibody is measured by ELISA using immobilized antigen displaying particles for antibody capture.

### 8. Statistical Analysis

Analysis of IFN-γ and antibody responses is performed by Kruskal-Wallis one-way analysis of variance (ANOVA).

### Results

GC-MS analysis of cells carrying plasmids pET14B-AcpA-C-IglC and pHAS all in the presence of pMCS69, will confirm the presence of the polyester polyhydroxybutyrate. The presence of intracellular polyester inclusions may be further confirmed by fluorescent microscopy using Nile Red staining.

The presence of polyhydroxybutyrate in cells carrying plasmids pET14B-AcpA-C-IglC and pHAS (wildtype control) all in the presence of pMCS69 indicates that the phaC polyester synthase domain retains polymer synthase activity when present as a single or tripartite fusion protein.

High level protein display by particles is determined by a prominent protein band with an apparent molecular weight directly aligning with molecular weight deduced from the fusion protein sequence. The identity of these proteins is confirmed by tryptic peptide fingerprinting using MALDI-TOF-MS. ELISA results indicate that the various antigen displaying particles bind to the respective anti-antigen antibody in a dose-dependent manner, while wild-type particles show significantly less binding of antibody. Flow cytometry results preferably show that >98% of antigen particles bind anti-antigen antibodies.

Expression in recombinant E. coli of the respective hybrid genes encoding the various antigen-PhaC fusion proteins allow production of polyester particles displaying the fusion protein at their surface.

Preferably, no overt toxicity is observed in any of the animals after immunization, and mouse weights does not differ significantly between groups during the time-course of the experiment, and mice in all groups gained weight. Mice immunised with polyester particles will develop small lumps (2.5 mm in diameter) at the immunisation sites but generally without abscesses or suppuration, and are typically healthy throughout the trial with normal behaviour and good quality fur.

A dose of 10-100 μg of antigen particles is optimal at generating a significant antibody response in mice. This dose induces significantly higher antibody titres when compared to a 10-100 μg dose of wildtype particles alone. Other doses may also be tested and used. In a second experiment which includes non-immunised control mice compare bead formulations with and without an adjuvant, and evaluated for significantly higher antigen-specific serum antibody responses for both vaccine groups given antigen particles compared to non-vaccinated mice. The highest antibody responses may be observed in mice immunised with antigen particles in Emulsigen. Antibody responses for the IgG1 isotype will typically be stronger than responses for IgG2 in both experiments.

The cell-mediated response to antigens of mice immunised with 10-100 μg antigen particles is also significantly enhanced compared to that of mice immunised with wildtype particles alone, or with PBS alone, and there should typically be no significant difference in the cell-mediated responses of mice immunised with wildtype particles alone compared to PBS-immunised control mice.

The IFN-γ response to either antigen in mice immunised 3 times with 10-100 μg of wild-type particles (no F. tularensis antigen) will typically not differ significantly from that of PBS-immunised control mice. In contrast, a significantly greater IFN-γ response to each antigen may be observed in mice immunised 3 times with antigen particles, and in mice immunised 3 times with antigen particles and Emulsigen. Expected is a significantly greater IFN-γ response to each antigen observed in mice immunised 3 times with antigen particles and Emulsigen than all the other vaccine groups.

The engineered polyester particles which simultaneously display antigens AcpA and IglC are capable of producing an antigen-specific cell-mediated response, as well as significantly increasing the production of IgG1 and IgG2 antibodies.

In addition to generation of both humoral and cell-mediated immune responses, the lack of adverse side effects such as weight loss, and absence of abscesses and suppuration at the injection site indicate that the polyester particles are well tolerated, safe, and non-toxic.

### Example 6—Immunogenicity of Brucella abortus Polymer Particle Vaccines

This example describes the construction of plasmids for the production in transformed hosts, in this case, E. coli, of polymer particles displaying the Brucella abortus antigen Omp16, an immunogenic outer membrane protein, together with an analysis of the immunogenecity of the polymer particles. Polymer particles displaying this antigen as produced in this example are useful as prophylactic and therapeutic vaccines against brucellosis.

### Materials and Methods

All animal experiments were approved by the AgResearch Grasslands Animal Ethics Committee (Palmerston North, New Zealand).

### 1. Overexpression Plasmid Construction

All plasmids and oligonucleotides in this example are listed in Table 7. The beta-ketothiolase and acetoacetyl-Coenzyme A reductase are encoded by plasmid pMCS69 and provide substrate for the polymer synthase by catalysing conversion of acetyl CoA to 3-hydroxybutyryl-Coenzyme A.

To produce B. abortus Omp16 displaying polymer particles, a gene encoding the antigen Omp16 is codon-optimized and synthesized by Genscript Inc. to allow subcloning into pET-14b PhaC-linker-GFP XhoI-BamHI sites for a C-terminal fusion to the PhaC polymer bead forming enzyme. The omp16 encoding gene is inserted into the XhoI-BamHI site. This gene insertion is in frame with GFP encoding region of the original plasmid replaced, yielding plasmid pET14B-C-omp16.

The construct for the PhaC-omp16 fusion and is shown as SEQ ID No. 31, with the derived amino acid sequence shown as SEQ ID No. 32.

TABLE 7
Plasmids and Oligonucleotides

| Plasmids | Description |
|---|---|
| pHAS | pET14b derivative containing the NdeI/BamHI inserted |
| | phaC gene from C. necator |
| pMCS69 | pBBR1MCS derivative containing genes phaA and phaB |
| | from C. necator |
| pET-14b PhaC- | pET-14b derivative containing the GFP encoding DNA |
| linker-GFP | sequence fused to the 3′ end of phaC |
| pET14B-C- | pET-14b PhaC-linker-GFP derivative |
| omp16 | containing the Omp16 encoding DNA sequence fused to |
| | the 3′ end of phaC |

### 2. Production of Omp16 Displaying Particles

Plasmid pET14B-C-omp16 and pHAS are introduced into E. coli KRX cells harbouring plasmid pMCS69. The transformants are cultured in conditions suitable for the production of biopolyester particles, as described in Example 1.

### 3. Isolation of Polyester Particles

Polyester granules are isolated as described in Example 3. The concentration of protein attached to particles is determined using the Bio-Rad Protein Assay as described in Example 3 and the proteins of interest identified using matrix-assisted laser desorption/ionization time-of-flight spectrometry (MALDI-TOF-MS) as described in Example 3.

### 4. ELISA

Immuno-reactivity of the B. abortus polymer particles is determined by enzyme-linked immunosorbent assay (ELISA) as described in Example 3 using mouse antibodies raised against the various antigens.

### 5. Immunisation of Mice

Female C57BL/6 mice (Malaghan Institute, Wellington, NZ) aged 6-8 weeks are intraperitoneally (i.p.) immunized two times at 2 week intervals. The three treatment groups are as follows:
a) individuals immunised with wild-type particles (i.e., particles prepared from bacterial cells carrying pHAS and pMCS69);
b) individuals immunised with antigen particles alone (i.e., particles prepared from bacterial cells carrying plasmids encoding the various antigen-PhaC fusion proteins and pMCS69);
c) individuals immunised with the various antigen particles mixed with 20% Emulsigen™ adjuvant (MVP Laboratories).

Non-vaccinated control animals are included for each set of experiments.

### 6. Immunological Assay

The mice are anaesthetised three weeks after the last immunisation and blood is collected, centrifuged, and the serum collected and frozen at −20° C. until assayed. The mice are then euthanized, their spleens removed and a single cell suspension is prepared by passage through an 80 gauge wire mesh sieve. Spleen red blood cells (RBCs) are lysed using a solution of 17 mM TRIS-HCl and 140 mM NH4Cl. After washing, the RBCs are cultured in Dulbecco's Modified Eagle media (DMEM) supplemented with 2 mM glutamine (Invitrogen), 100 U/mL penicillin (Invitrogen), 100 μg/mL streptomycin (Invitrogen), 5×10-5 M2-mercaptoethanol (Sigma) and 5% (w/w) Foetal Calf Serum (Invitrogen). The cells are incubated at 37° C. in 10% CO2 in medium alone, or in medium containing the respective antigens.

### 7. Quantification of IFN-γ

Culture supernatants are removed after 4 days incubation and frozen at −20° C. until assayed.

Levels of IFN-γ in the supernatants are measured by ELISA (BD Biosciences) according to manufacturer's instructions using commercially available antibodies and standards (BD Pharmingen).

### 8. Quantification of Serum Antibody

Serum antibody is measured by ELISA using immobilized antigen displaying particles for antibody capture.

### 9. Statistical Analysis

Analysis of the IFN-γ and antibody responses is performed by Kruskal-Wallis one-way analysis of variance (ANOVA).

### Results

GC-MS analysis of cells carrying plasmids pET14B-C-omp16 and pHAS all in the presence of pMCS69, will confirm the presence of the polyester polyhydroxybutyrate. The presence of intracellular polyester inclusions may be further confirmed by fluorescent microscopy using Nile Red staining.

The presence of polyhydroxybutyrate in cells carrying plasmids pET14B-C-omp16 and pHAS (wildtype control) all in the presence of pMCS69 indicates that the PhaC polyester synthase domain retained polymer synthase activity when present as a single or tripartite fusion protein.

High level protein display by particles is determined by a prominent protein band with an apparent molecular weight directly aligning with molecular weight deduced from the fusion protein sequence, respectively. The identity of these proteins is confirmed by tryptic peptide fingerprinting using MALDI-TOF-MS. ELISA results indicate that the various antigen displaying particles bind to the respective anti-antigen antibody in a dose-dependent manner, while wild-type particles show significantly less binding to the antibody. Flow cytometry results preferably show that >95% of antigen particles bind anti-antigen antibodies. Expression in recombinant E. coli of the respective hybrid gene encoding the PhaC-antigen fusion protein allow production of polyester particles displaying the fusion protein at their surface.

No overt toxicity is observed, preferably, in any of the animals after immunization, and mouse weights do not differ significantly between groups during the time-course of the experiment, and mice in all groups gained weight (data not shown). Mice immunised with polyester particles will be typically healthy throughout the trial with normal behaviour and good quality fur.

A dose range of about 10-50 μg of antigen particles is generating a significant antibody response in mice. This dose induces significantly higher antibody titres when compared to a 10-50 μg dose of wildtype particles alone. Other doses may also be tested and used, for example 50-500 μg. In a second experiment which includes non-immunised control mice and compare bead formulations with and without an adjuvant, and evaluated for significantly higher antigen-specific serum antibody responses for both vaccine groups given antigen particles compared to non-vaccinated mice. The highest antibody responses may be observed in mice immunised with antigen particles in Emulsigen. Antibody responses for the IgG1 isotype will typically be stronger than responses for IgG2 in both experiments.

The cell-mediated response to antigens of mice immunised with 10-50 μg antigen particles is also significantly enhanced compared to that of mice immunised with wildtype particles alone, or with PBS alone and there should typically be no significant difference in the cell-mediated responses of mice immunised with wildtype particles alone compared to PBS-immunised control mice.

The IFN-γ response to the antigen in mice immunised 2 times with 10-50 μg of wild-type particles (no B. abortus antigen) will typically not differ significantly from that of PBS-immunised control mice. In contrast, a significantly greater IFN-γ response to each antigen is observed in mice immunised 2 times with antigen particles, and in mice immunised 2 times with antigen particles and Emulsigen. Expected is a significantly greater IFN-γ response to each antigen is observed in mice immunised 2 times with antigen particles and Emulsigen than all the other vaccine groups.

The engineered polyester particles which display antigen Omp16 are capable of producing an antigen-specific cell-mediated response, as well as significantly increasing the production of IgG1 and IgG2 antibodies.

In addition to generation of both humoral and cell-mediated immune responses, the lack of adverse side effects such as weight loss, and absence of abscesses and suppuration at the injection site indicate that the polyester particles are well tolerated, safe, and non-toxic.

### Example 7—Immunogenicity of Neisseria meningitidis Polymer Particle Vaccines

This example describes the construction of plasmids for the production in transformed hosts, in this case, E. coli, of polymer particles displaying the Neisseria meningitidis antigens PorA, PorB, FetA, ZnuD, as well as chemically cross-linked or non-covalently bound Neisseria meningitidis B capsular polysaccharide (CPS), together with an analysis of the immunogenecity of the polymer particles. Particles with these antigens are useful as prophylactic and therapeutic vaccines against meningitis.

Materials and Methods

All animal experiments were approved by the AgResearch Grasslands Animal Ethics Committee (Palmerston North, New Zealand).

### 1. Construction of Plasmids

All plasmids and oligonucleotides for this example are listed in Table 8.

The PhaA and PhaB enzymes are encoded by plasmid pMCS69. A DNA fragment encoding the six-cysteine-PhaC fusion protein and including a translation initiation site and start codon is obtained from genomic DNA isolated from Ralstonia eutropha H16 by PCR using primers Cys6-XbaI [SEQ ID No. 55] and PhaC-C-BamHI [SEQ ID No. 56] and as template. The PCR product is ligated into XbaI, BamHI—endonucleased pET14B vector to generate the plasmid pET-14b-Cys6-PhaC.

To produce polymer particles simultaneously displaying two Neisseria meningitidis antigens, genes encoding the antigens PorA, PorB, FetA, ZnuD are codon optimized and synthesized by Genscript Inc. to allow subcloning into pET-14b M-PhaC-linker-MalE XbaI-SpeI site for an N-terminal fusion and into XhoI-BamHI sites for a C-terminal fusion to the PhaC polymer bead forming enzyme. The PorA encoding gene is inserted into the XbaI-SpeI sites and on the same plasmid the PorB encoding gene is inserted into the XhoI-BamHI sites. Both gene insertions are in frame with the M and MalE encoding regions of the original plasmid replaced, yielding plasmid pET14B-PorA-C-PorB.

The FetA encoding gene is inserted into the XbaI-SpeI sites and on the same plasmid the ZnuD encoding gene is inserted into the XhoI-BamHI sites. Both gene insertions are in frame with the M and MalE encoding regions of the original plasmid replaced, yielding plasmid pET14B-FetA-C-ZnuD.

The construct for the Cys6-PhaC fusion is shown as SEQ ID No. 33, with the derived amino acid sequence shown as SEQ ID No. 34. The construct for the PorA-C-PorB fusion is shown as SEQ ID No. 35, with the derived amino acid sequence shown as SEQ ID No. 36. The construct for of the FetA-C-ZnuD fusion is shown as SEQ ID No. 37, with the derived amino acid sequence shown as SEQ ID No. 38.

TABLE 8
Plasmids and Oligonucleotides

| Plasmids | Description |
|---|---|
| pET-14b | Ap^{r}, T7 promoter |
| pHAS | pET14b derivative containing the |
| | NdeI/BamHI inserted phaC gene from |
| | C. necator |
| pET-14b- | pET14b derivative containing the |
| Cys6- | NdeI/BamHI inserted phaC gene from |
| PhaC | C. necator with a 5′ extension |
| | encoding six N-terminally inserted |
| | cysteine residues |
| pMCS69 | pBBR1MCS derivative containing genes |
| | phaA and phaB from C. necator |
| pET-14b | pET-14b PhaC-linker-MalE derivative |
| M-PhaC- | containing the mpl sequence fused |
| linker- | to the 5′ end of phaC |
| MalE | |
| pET14B- | pET-14b M-PhaC-linker-MalE derivative |
| PorA-C- | containing the porA sequence fused |
| PorB | to the 5′ end and porB fused to the |
| | 3′ end of phaC |
| pET14B- | pET-14b M-PhaC-linker-MalE derivative |
| FetA-C- | containing the fetA sequence fused |
| ZnuD | to the 5′ end and znuD fused to the |
| | 3′ end of phaC |
| Cys6- | 5′- |
| XbaI | cgcctttgccggtcgcacaacaacaacaacaacacat |
| | actagtatctccttatttctagaggga-3′ |
| | [SEQ ID No. 55] |
| PhaC-C- | 5′-gatacgtcaaagccaaggcatgtagggatccggc |
| BamHI | tgctaacaaag-3′ |
| | [SEQ ID No. 56] |

### 2. Production of Cys-6, PorA/B and FetA/ZnuD Displaying Particles

Plasmids pET-14b-Cys6-PhaC, pET14B-PorA-C-PorB, pET14B-FetA-C-ZnuD and pHAS are introduced into E. coli BL21 (DE3) cells harbouring plasmid pMCS69. The transformants are cultured in conditions suitable for the production of biopolyester particles, as described in Example 1.

### 3. Isolation of Polyester Particles

Polyester granules are isolated by disrupting the bacteria and whole cell lysates are centrifuged at 4000 g for 15 minutes at 4° C. to sediment the polyester particles. The particles are purified via glycerol gradient ultracentrifugation

The concentration of protein attached to particles is determined using the Bio-Rad Protein Assay as described in Example 3. Following concentration determination, the proteins are separated by SDS-PAGE and stained with SimplyBlue Safe Stain (Invitrogen).

The amount of Cys6-C, PorA-C-PorB or FetA-C-ZnuD fusion protein, respectively, relative to the amount of total protein attached to the particles is detected using a Gel Doc™ XR and analysed using Quantity One software (version 4.6.2, Bio-Rad Laboratories). Proteins of are identified using matrix-assisted laser desorption/ionization time-of-flight spectrometry (MALDI-TOF-MS). In case of Cys6-C N-terminal sequencing is used to confirm the presence of six cysteine residues in the N-terminus of PhaC.

4. Chemical Cross-Linking of N. meningitidis CPS to Cys6 Polyester Particles

Chemical cross-linking of the capsular polysaccharide (CPS) to the Cys6 particles is achieved by using purified N. meningitidis CPS and the chemical cross-linker PMPI (N-[p-Maleimidophenyl]isocyanate) as previously described by Annunziato et al. PMPI is a heterobifunctional linker for hydroxyl to thiol coupling which allows covalent coupling of N. meningitidis CPS to polymer particles which display six cysteine residues which are engineered into the N terminus of the polymer particle forming enzyme, the PHA synthase from Ralstonia eutropha.

5. Non-Covalent Binding of N. meningitidis CPS to Specific Antibody Displaying Polyester Particles

CPS specific antibodies are raised by immunizing rabbits. Monospecific polyclonal sera are subjected to protein A affinity purification. The resulting purified IgG's are bound to ZZ domain displaying polyester particles. These particles are then incubated for 30 min with N. meningitidis CPS using a ratio of 1:1 on dry weight basis. This allows specific but noncovalent binding of CPS to polyester particles

### 6. ELISA

Immuno-reactivity of the N. meningitidis polymer particles is determined by enzyme-linked immunosorbent assay (ELISA) as described in Example 3 using mouse antibodies raised against the various antigens.

### 7. Immunisation of Mice

Female C57BL/6 mice (Malaghan Institute, Wellington, NZ) aged 6-8 weeks are intramuscularly immunized three times at 2 week intervals. The three treatment groups are as follows:
a) individuals immunised with wild-type particles (i.e., particles prepared from bacterial cells carrying pHAS and pMCS69);
b) individuals immunised with antigen particles alone (i.e., particles prepared from bacterial cells carrying plasmids encoding the various antigen-PhaC fusion proteins and pMCS69);
c) individuals immunised with the various antigen particles mixed with 20% Emulsigen™ adjuvant (MVP Laboratories).

Non-vaccinated control animals are included for each set of experiments.

### 8. Immunological Assay

The mice are anaesthetised three weeks after the last immunisation and blood is collected, centrifuged, and the serum collected and frozen at −20° C. until assayed.

The mice are then euthanized, their spleens removed and a single cell suspension is prepared by passage through an 80 gauge wire mesh sieve. Spleen red blood cells (RBCs) are processed as described in Example 4.

### 9. Quantification of IFN-γ

Culture supernatants are removed after 4 days incubation and frozen at −20° C. until assayed.

Levels of IFN-γ in the supernatants are measured by ELISA (BD Biosciences) as described in Example 4.

### 10. Quantification of Serum Antibody

Serum antibody is measured by ELISA using immobilized antigen displaying particles for antibody capture.

### 11. Statistical Analysis

Analysis of IFN-γ and antibody responses is performed by Kruskal-Wallis one-way analysis of variance (ANOVA).

### Results

GC-MS analysis of cells carrying plasmids pET-14b-Cys6-PhaC, pET14B-PorA-C-PorB, pET14B-FetA-C-ZnuD and pHAS all in the presence of pMCS69, will confirm the presence of the polyester polyhydroxybutyrate. The presence of intracellular polyester inclusions may be further confirmed by fluorescent microscopy using Nile Red staining.

The presence of polyhydroxybutyrate in cells carrying plasmids pET-14b-Cys6-PhaC, pET14B-PorA-C-PorB, pET14B-FetA-C-ZnuD and pHAS (wildtype control) all in the presence of pMCS69 indicates that the phaC polyester synthase domain retains polymer synthase activity when present as a single or tripartite fusion protein.

High level protein display by particles is determined by a prominent protein band with an apparent molecular weight directly aligning with molecular weight deduced from the fusion protein sequence, respectively. The identity of these proteins is confirmed by tryptic peptide fingerprinting using MALDI-TOF-MS. ELISA results indicate that the various antigen displaying particles bind to the respective anti-antigen antibody in a dose-dependent manner, while wild-type particles show significantly less binding of antibody. Flow cytometry results preferably show that >98% of antigen particles bind anti-antigen antibodies.

Expression in recombinant E. coli of the respective hybrid genes encoding the various antigen-PhaC fusion proteins allow production of polyester particles displaying the fusion protein at their surface.

Preferably, no overt toxicity is observed in any of the animals after immunization, mouse weights do not differ significantly between groups during the time-course of the experiment, and mice in all groups gained weight. Mice immunised with polyester particles will typically develop small lumps (2.5 mm in diameter) at the immunization sites but generally without abscesses or suppuration, and all mice are typically healthy throughout the trial with normal behaviour and good quality fur.

A dose of 5-50 μg of antigen particles is generating a significant antibody response in mice. This dose induces significantly higher antibody titres when compared to a 5-50 μg dose of wildtype particles alone. Other doses may also be tested and used. In a second experiment which includes non-immunised control mice and compares bead formulations with and without an adjuvant, and evaluated for significantly higher antigen-specific serum antibody responses for both vaccine groups given antigen particles compared to non-vaccinated mice. The highest antibody responses may be observed in mice immunised with antigen particles in Emulsigen. Antibody responses for the IgG1 isotype will typically be stronger than responses for IgG2 in both experiments.

The cell-mediated response to antigens of mice immunised with 5-50 μg antigen particles is also significantly enhanced compared to that of mice immunised with wildtype particles alone, or with PBS alone, and there should typically be no significant difference in the cell-mediated responses of mice immunised with wildtype particles alone compared to PBS-immunised control mice.

The IFN-γ response to either antigen in mice immunised 3 times with 40 μg of wild-type particles (no N. meningitidis antigen) should not differ significantly from that of PBS-immunised control mice. In contrast, a significantly greater IFN-γ response to each antigen should be observed in mice immunised 3 times with antigen particles, and in mice immunised 3 times with antigen particles and Emulsigen. Expected is a significantly greater IFN-γ response to each antigen in mice immunised 3 times with antigen particles and Emulsigen than all the other vaccine groups.

The engineered polyester particles displaying antigens PorA, PorB, FetA, ZnuD and the CPS are capable of producing an antigen-specific cell-mediated response, as well as significantly increasing the production of IgG1 and IgG2 antibodies.

In addition to generation of both humoral and cell-mediated immune responses, the lack of adverse side effects such as weight loss, and absence of abscesses and suppuration at the injection site indicate that the polyester particles are well tolerated, safe, and non-toxic.

### Example 8—Immunogenicity of Bacillus anthracis Polymer Particle Vaccines

This example describes the construction of plasmids for the production in transformed hosts, in this case, E. coli, of polymer particles displaying the Bacillus anthracis antigen PA83, a non-toxic subunit of the anthrax toxin, together with an analysis of the immunogenecity of the polymer particles. Polymer particles displaying this antigen as produced in this example are useful as prophylactic and therapeutic vaccines against Anthrax.

### Materials and Methods

All animal experiments were approved by the AgResearch Grasslands Animal Ethics Committee (Palmerston North, New Zealand).

### 1. Construction of Plasmids

All plasmids and oligonucleotides in this example are listed in Table 9. The PhaA and PhaB enzymes are encoded by plasmid pMCS69.

To produce polymer particles displaying the B. anthracis PA83 antigen, a truncated variant of the non-toxic subunit PA of the anthrax toxin, a gene encoding the antigen PA83 is codon-optimized and synthesized by Genscript Inc. to allow subcloning into pET-14b PhaC-linker-GFP XhoI-BamHI sites for a C-terminal fusion to the PhaC polymer bead forming enzyme. The PA83 encoding gene is inserted into the XhoI-BamHI site. This gene insertion is in frame with GFP encoding region of the original plasmid replaced, yielding plasmid pET14B-PhaC-PA83.

The construct for the PhaC-PA83 fusion is shown as SEQ ID No. 39, with the derived amino acid sequence shown as SEQ ID No. 40.

TABLE 9
Plasmids and Oligonucleotides

| Plasmids | Description |
|---|---|
| pHAS | pET14b derivative containing the NdeI/BamHI inserted |
| | phaC gene from C. necator |
| pMCS69 | pBBR1MCS derivative containing genes phaA and phaB |
| | from C. necator |
| pET-14b PhaC- | pET-14b derivative containing the GFP encoding DNA |
| linker-GFP | sequence fused to the 3′ end of phaC |
| pET14B-C- | pET-14b PhaC-linker-GFP derivative |
| PA83 | containing the PA83 encoding DNA sequence fused to |
| | the 3′ end of phaC |

### 2. Production of PA83 Displaying Particles

Plasmid pET14B-C-PA83 and pHAS are introduced into E. coli BL21 (DE3) cells harbouring plasmid pMCS69. The transformants are cultured in conditions suitable for the production of biopolyester particles, as described in Example 1.

### 3. Gas Chromatography Mass Spectroscopy (GC-MS)

The polyester content of bacterial cells harboring the various plasmids corresponds to the activity of the PhaC synthase in vivo. The amount of accumulated polyester is assessed by gas chromatography-mass spectroscopy (GC-MS) analysis to determine phaC synthase activity, and particularly to catalysis by PhaC-B. anthracis antigen fusion of polyester synthesis and mediation of intracellular granule formation. Polyester content is quantitatively determined by GC-MS after conversion of the polyester into 3-hydroxymethyl ester by acid-catalysed methanolysis.

### 4. Isolation of Polyester Particles

Polyester granules are isolated as described in Example 3 and the concentration of protein attached to particles is determined using the Bio-Rad Protein Assay as described in Example 3.

### 5. ELISA

Immuno-reactivity of the B. anthracis polymer particles is determined by enzyme-linked immunosorbent assay (ELISA) as described in Example 3 using mouse antibodies raised against the various antigens.

### 6. Flow Cytometry

Twenty-five micrograms of various purified antigen-displaying particles or wild-type particles are washed twice in ice-cold flow cytometry buffer as detailed in Table 4 of Example 3 and incubated with mouse anti-antigen antibodies. After washing, particles are stained with rat anti-mouse Fluorescein isothiocyanate (FITC)-labelled antibody (BD Pharmingen, CA, USA), incubated for 30 minutes on ice in the dark and washed again. A BD FACScalibur (BD Biosciences, CA, USA) is used to collect at least 10,000 events for each sample and analysed using CellQuest software.

### 7. Immunisation of Mice

Female C57BL/6 mice (Malaghan Institute, Wellington, NZ) aged 6-8 weeks are intramuscularly immunized three times at 2 week intervals. The three treatment groups are as follows:
a) individuals immunised with wild-type particles (i.e., particles prepared from bacterial cells carrying pHAS and pMCS69);
b) individuals immunised with antigen particles alone (i.e., particles prepared from bacterial cells carrying plasmids encoding the various antigen-PhaC fusion proteins and pMCS69);
c) individuals immunised with the various antigen particles mixed with 20% Emulsigen™ adjuvant (MVP Laboratories).

Non-vaccinated control animals are included for each set of experiments.

### 8. Immunological Assay

The mice are anaesthetised three weeks after the last immunisation and blood is collected, centrifuged, and the serum collected and frozen at −20° C. until assayed.

The mice are then euthanized, their spleens removed and a single cell suspension is prepared by passage through an 80 gauge wire mesh sieve. Spleen red blood cells (RBCs) are lysed using a solution of 17 mM TRIS-HCl and 140 mM NH4Cl. After washing, the RBCs are cultured in Dulbecco's Modified Eagle media (DMEM) supplemented with 2 mM glutamine (Invitrogen), 100 U/mL penicillin (Invitrogen), 100 μg/mL streptomycin (Invitrogen), 5×10-5 M 2-mercaptoethanol (Sigma) and 5% (w/w) Foetal Calf Serum (Invitrogen).

### 9. Quantification of IFN-γ

Culture supernatants are removed after 4 days incubation and frozen at −20° C. until assayed. Levels of IFN-γ in the supernatants are measured by ELISA (BD Biosciences) according to manufacturer's instructions using commercially available antibodies and standards (BD Pharmingen).

### 10. Quantification of Serum Antibody

Serum antibody is measured by ELISA using immobilized antigen displaying particles for antibody capture.

### 11. Statistical Analysis

Analysis of the IFN-γ and antibody responses is performed by Kruskal-Wallis one-way analysis of variance (ANOVA).

### Results

GC-MS analysis of cells carrying plasmids pET14B-C-PA83 and pHAS all in the presence of pMCS69 will confirm the presence of the polyester polyhydroxybutyrate. The presence of intracellular polyester inclusions further confirmed by fluorescent microscopy using Nile Red staining.

The presence of polyhydroxybutyrate in cells carrying plasmids pET14B-C-PA83 and pHAS (wildtype control) all in the presence of pMCS69 indicates that the PhaC polyester synthase domain retained polymer synthase activity when present as a single or tripartite fusion protein.

High level protein display by particles is determined by a prominent protein band with an apparent molecular weight directly aligning with molecular weight deduced from the fusion protein sequence, respectively. The identity of these proteins is confirmed by tryptic peptide fingerprinting using MALDI-TOF-MS. ELISA results indicates that the various antigen displaying particles bind to the respective anti-antigen antibody in a dose-dependent manner, while wild-type particles show significantly less binding of antibody. Flow cytometry results preferably show that >96% of antigen particles bind anti-antigen antibodies.

Expression in recombinant E. coli of the respective hybrid gene encoding the PhaC-antigen fusion protein allows production of polyester particles displaying the fusion protein at their surface.

Preferably, no overt toxicity is observed in any of the animals after immunization, and mouse weights do not differ significantly between groups during the time-course of the experiment, and mice in all groups gained weight (data not shown). Mice immunised with polyester particles will typically develop small lumps (2.5 mm in diameter) at the immunisation sites but generally without abscesses or suppuration and are typically healthy throughout the trial with normal behaviour and good quality fur.

A dose of 40 μg of antigen particles is sufficient to generate a significant antibody response in mice. This dose induces significantly higher antibody titres when compared to a 40 μg dose of wildtype particles alone. Other doses may also be tested and used. In a second experiment which includes non-immunised control mice and compare bead formulations with and without an adjuvant, and evaluated for significantly higher antigen-specific serum antibody responses for both vaccine groups given antigen particles compared to non-vaccinated mice. The highest antibody responses may be observed in mice immunised with antigen particles in Emulsigen. Antibody responses for the IgG1 isotype will typically stronger than responses for IgG2 in both experiments.

The cell-mediated response to antigens of mice immunised with 10 μg or with 40 μg antigen particles is also significantly enhanced compared to that of mice immunised with wildtype particles alone, or with PBS alone and there should typically be no significant difference in the cell-mediated responses of mice immunised with wildtype particles alone compared to PBS-immunised control mice.

The IFN-γ response to either antigen in mice immunised 3 times with 40 μg of wild-type particles (no B. anthracis antigen) will typically not differ significantly from that of PBS-immunised control mice. In contrast, a significantly greater IFN-γ response to each antigen is observed in mice immunised 3 times with antigen particles, and in mice immunised 3 times with antigen particles and Emulsigen. Expected is a significantly greater IFN-γ response to each antigen is observed in mice immunised 3 times with antigen particles and Emulsigen than all the other vaccine groups.

The engineered polyester particles which display antigen PA83 are capable of producing an antigen-specific cell-mediated response, as well as significantly increasing the production of IgG1 and IgG2 antibodies.

In addition to generation of both humoral and cell-mediated immune responses, the lack of adverse side effects such as weight loss, and absence of abscesses and suppuration at the injection site indicate that the polyester particles are well tolerated, safe, and non-toxic.

### Example 9—Immunogenicity of Hepatitis C Polymer Particle Vaccines In Vivo in Mice

This example describes the immunisation of a mammalian model with polymer particles comprising Hep-C antigens.

Materials and Methods

All animal experiments were approved by the AgResearch Grasslands Animal Ethics Committee (Palmerston North, New Zealand).

### 1. Construction of Plasmids and Isolation of Polyester Polymer Particles

Plasmids were constructed for the production of polymer particles displaying the Hepatitis C core antigen using E. coli as the host as described in Example 1.

Polyester granules were isolated by disrupting the bacteria and whole cell lysates were centrifuged at 6000 g for 15 minutes at 4° C. to sediment the polymer particles. The particles were purified via glycerol gradient ultracentrifugation. Protein concentration was determined using the Bio-Rad Protein Assay according to the manufacturer's instructions (Bio-Rad). The amount of Hep C:PhaC fusion protein relative to the amount of total protein attached to the polymer particles was detected using a Gel Doc™ XR and analysed using Quantity One software (version 4.6.2, Bio-Rad). The Hep C antigen accounted for approximately 6.7% of the total protein of the polymer particle in E. coli and 25% of the total protein of the polymer particle in L. lactis. Identification of the protein of interest was confirmed using matrix-assisted laser desorption/ionisation time-of flight mass spectrometry (MALDI-TOF-MS).

### 2. ELISA

Immuno-reactivity of the Hep C polymer particles was determined by enzyme-linked immunosorbent assay (ELISA) as described in Example 3. After washing, plates were incubated with mouse antibody to Hep C (Devatal, USA), washed with PBST, then incubated for 1 hour at room temperature with biotinylated anti-mouse IgG (Sigma-Aldrich) diluted in 1% (w/v) BSA in PBS. After further incubation for 1 hour at room temperature, plates were washed with PBST and streptavidin-HRP conjugate was added and incubated for a further 1 hour. After further washing, o-phenylenediamine (OPD) substrate (Sigma-Aldrich) was added and the plates were incubated for 30 minutes at room temperature.

Absorbance was recorded at 490 nm on a VERSAax microplate reader.

### 3. Immunisation of Mice

Female C57BL/6 mice (Malaghan Institute, Wellington, NZ) aged 6-8 weeks were sub-cutaneously immunized three times at weekly intervals, with the exception of the commercial recombinant Hep C antigen treatment group. The commercial recombinant Hep C antigen (E. coli derived) was obtained from Devatal Inc. (USA) and contained the nucleocapsid immunodominant regions of the Hepatitis C virus. The antigen was >95% pure as determined by 10% PAGE (Coomassie staining) indicated by the supplier.

The six treatment groups (n=6 per group) were as follows:
a) individuals immunised with commercial Hep C antigen (30 μg) in Complete Freund's adjuvant (CFA)—vaccinated once only.
b) individuals immunised with commercial Hep C antigen (30 μg) and Emulsigen™ adjuvant (MVP Laboratories)—vaccinated once only.
c) individuals immunised with PBS and 20% Emulsigen™ adjuvant (MVP Laboratories).
d) individuals immunised with Hep C polymer particles (10 μg) mixed with 20% Emulsigen™ adjuvant (MVP Laboratories).
e) individuals immunised with Hep C polymer particles (30 g) mixed with 20% Emulsigen™ adjuvant (MVP Laboratories).
f) individuals immunised with wild-type polymer particles (E. coli host) mixed with 20% Emulsigen™ adjuvant (MVP Laboratories).

Non-vaccinated control animals were included for each set of experiments.

### 4. Immunological Assay

The mice were anaesthetised intraperitoneally three weeks after the last immunisation using 87 μg ketamine (Parnell Laboratories, Australia) and 2.6 μg xylazine hydrochloride (Bayer, Germany) per gram of body weight. Blood was collected, centrifuged, and the serum collected and frozen at −20° C. until assayed.

The mice were then euthanased, their spleens removed and a single cell suspension was prepared by passage through a 80 gauge wire mesh sieve. Spleen red blood cells were processed as described in Example 4. The cells were incubated at 37° C. in 10% CO₂ in medium alone, or in medium containing 5 μg/mL recombinant Hep C antigen.

### 5. Quantification of IFN-γ

Culture supernatants were removed after 4 days incubation and frozen at −20° C. until assayed. Levels of IFN-γ in the supernatants were measured by ELISA (BD Biosciences) according to manufacturer's instructions using commercially available antibodies and standards (BD Pharmingen).

### 6. Quantification of Serum Antibody

Serum antibody was measured by ELISA according to manufacturer's recommendations using monoclonal anti-Hep C antibody (Devatal). Briefly, Maxisorb (Nunc) plates were coated overnight with 3 μg/mL of recombinant Hep C, blocked with 1% BSA and washed in PBST. Dilutions of serum (from 1:50 to 1:156250) were added and incubated. Following washing, anti-mouse IgG1:HRP or IgG2c:HRP (ICL, USA) was added and the plates incubated. Plates were washed and TMB used as a substrate prior to reading at 450 nm on a VERSAmax microplate reader.

Monoclonal anti-Hep C antibodies were titrated and included as a positive control for the IgG1 plates. Results were expressed as optical density at 450 nm for sera diluted 1:50.

### 7. Statistical Analysis

Analysis of the IFN-γ and antibody responses was performed by Fisher's one-way analysis of variance (ANOVA), with a level of significance of P<0.05.

### Results

Reactivity of Hep C polymer particles showed a dose-dependent response to Hep C antibody as shown in FIG. 1.

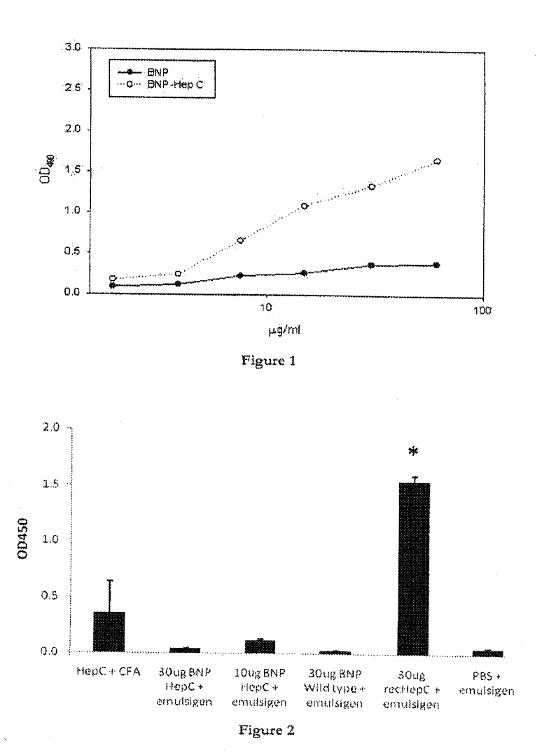

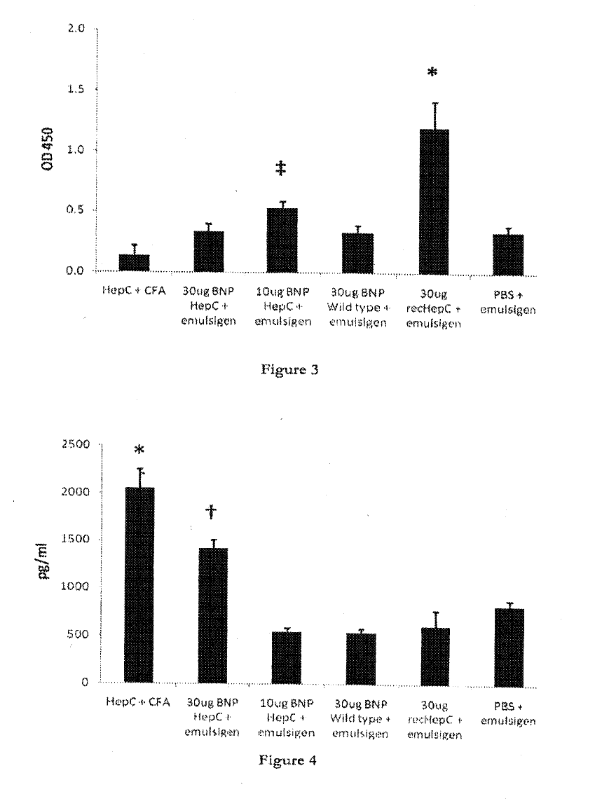

A dose of 10 μg/mL Hep C polymer particles elicited a greater IgG1 antibody response and a greater IgG2 antibody response compared to 30 μg/mL Hep C polymer particles (see FIGS. 2 and 3, respectively). Both doses of Hep C polymer particles elicited a significantly diminished IgG1 and IgG2 antibody response compared to recombinant Hep C antigen alone (see FIGS. 2 and 3, respectively).

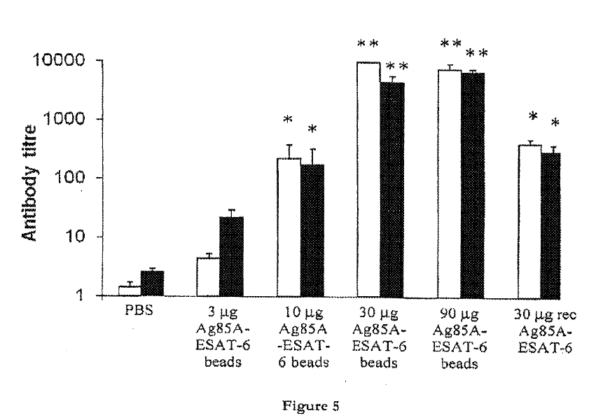

As shown in FIG. 4, the cell-mediated response to Hep C core antigen of mice immunised with 30 μg Hep C polymer particles was significantly enhanced compared to that of mice immunised with wild type polymer particles (P<0.05), with recombinant Hep C antigen alone (P<0.05), or with PBS alone (p<0.05). Indeed, there was no significant difference in the cell-mediated responses of mice immunised with antigen alone compared to PBS-immunised control mice.

### Discussion

The engineered polymer particles displaying Hep C core antigen produced in E. coli were capable of producing a targeted cell-mediated response to Hep C antigen challenge. Notably, immunisation with antigen alone (i.e., antigen not comprising a polymer particle of the present invention) was ineffective in eliciting a cell-mediated response, despite being capable of eliciting a strong humoral response.

The Hep C polymer particles of the invention were able to elicit a stronger IgG2 humoral response compared to the IgG1 response. IgG2 antibodies have been implicated in the stimulation of antibody-dependent, cell-mediated cytotoxicity (ADCC), and these data support the idea that the Hep C polymer particles can effectively stimulate, both directly and indirectly, complementary aspects of the cell-mediated response.

These results demonstrated the versatility and potential of this vaccine-delivery system to elicit different facets of the immune response, whereby a cell-mediated immune response was effectively elicited, with less stimulation of an ineffective humoral response.

The lack of adverse side effects such as weight loss, and absence of abscesses and suppuration at the injection site demonstrated that the polyester polymer particles were well tolerated, safe, and non-toxic.

### Example 10—Immunogenicity of Dengue Virus Polymer Particle Vaccines

This example describes the construction of plasmids for the production in transformed hosts, in this case, E. coli, of polymer particles displaying both the Dengue virus envelope protein (E) and the membrane protein (M), both immunogenic proteins expressed on the surface of the virion, together with an analysis of the immunogenecity of the polymer particles. Polymer particles displaying this antigen as produced in this example are useful as prophylactic and therapeutic vaccines against Dengue virus.

### Materials and Methods

All animal experiments were approved by the AgResearch Grasslands Animal Ethics Committee (Palmerston North, New Zealand).

### 1. Overexpression Plasmid Construction

All plasmids and oligonucleotides in this example are listed in Table 10. The beta-ketothiolase and acetoacetyl-Coenzyme A reductase are encoded by plasmid pMCS69 and provide substrate for the polymer synthase by catalysing conversion of acetyl CoA to 3-hydroxybutyryl-Coenzyme A.

To produce Dengue virus serotypes 1-4 E and M displaying polymer particles, genes encoding the antigens E and M are codon-optimized and synthesized by Genscript Inc. to allow subcloning into pET-14b M-PhaC-linker-MalE XbaI-SpeI sites for an N-terminal fusion and into XhoI-BamHI sites for a C-terminal fusion to the PhaC polymer bead forming enzyme. The omp16 encoding gene is inserted into the XhoI-BamHI site. This gene insertion is in frame with GFP encoding region of the original plasmid replaced, yielding plasmid pET14B-C-omp16.

The construct for the E1-PhaC-M1 fusion is shown as SEQ ID No. 41, with the derived amino acid sequence shown as SEQ ID No. 42. The construct for the E2-PhaC-M2 fusion is shown as SEQ ID No. 43, with the derived amino acid sequence shown as SEQ ID No. 44. The coding sequence of the E3-PhaC-M3 fusion is shown as SEQ ID No. 45, with the derived amino acid sequence shown as SEQ ID No. 46. The construct for the E4-PhaC-M1 fusion is shown as SEQ ID No. 47, with the derived amino acid sequence shown as SEQ ID No. 48.

TABLE 10
Plasmids and Oligonucleotides

| Plasmids | Description |
|---|---|
| pHAS | pET14b derivative containing the NdeI/BamHI inserted |
| | phaC gene from C. necator |
| pMCS69 | pBBR1MCS derivative containing genes phaA and phaB |
| | from C. necator |
| pET-14b M- | pET-14b PhaC-linker-MalE derivative |
| PhaC-linker- | containing the mpl sequence fused to the |
| MalE | 5′ end of phaC |
| pET14B-E1- | pET-14b M-PhaC-linker-MalE derivative containing the |
| C-M1 | Dengue serotype 1 E sequence fused to the 5′ end and |
| | Dengue serotype 1 M fused to the 3′ end of phaC |
| pET14B-E2- | pET-14b M-PhaC-linker-MalE derivative containing the |
| C-E2 | Dengue serotype 2 E sequence fused to the 5′ end and |
| | Dengue serotype 2 M fused to the 3′ end of phaC |
| pET14B-E3- | pET-14b M-PhaC-linker-MalE derivative containing the |
| C-M3 | Dengue serotype 3 E sequence fused to the 5′ end and |
| | Dengue serotype 3 M fused to the 3′ end of phaC |
| pET14B-E4- | pET-14b M-PhaC-linker-MalE derivative containing the |
| C-E4 | Dengue serotype 4 E sequence fused to the 5′ end and |
| | Dengue serotype 4 M fused to the 3′ end of phaC |

### 2. Production of Dengue Virus Serotypes 1-4 E and M Displaying Particles

The plasmids pET14B-E1-C-M1, pET14B-E2-C-M2, pET14B-E3-C-M3 or pET14B-E4-C-M4 and pHAS are introduced into E. coli BL21 (DE3) cells harbouring plasmid pMCS69. The transformants are cultured in conditions suitable for the production of biopolyester particles, as described in Example 1. Production of Dengue virus E-PhaC-M particles or wild-type particles, respectively, is assessed as described below.

### 3. Gas Chromatography Mass Spectroscopy (GC-MS)

The polyester content of bacterial cells harboring the various plasmids corresponds to the activity of the PhaC synthase in vivo. The amount of accumulated polyester is assessed by gas chromatography-mass spectroscopy (GC-MS) analysis to determine phaC synthase activity, and to confirm that the PhaC-Dengue virus serotype 1-4 E and M antigen fusion catalyses polyester synthesis and mediates intracellular granule formation. Polyester content is quantitatively determined by GC-MS after conversion of the polyester into 3-hydroxymethyl ester by acid-catalysed methanolysis.

### 4. Isolation of Polyester Particles

Polyester granules are isolated by disrupting the bacteria and whole cell lysates are centrifuged at 4000 g for 15 minutes at 4° C. to sediment the polyester particles. The particles are purified via glycerol gradient ultracentrifugation.

The concentration of protein attached to particles is determined using the Bio-Rad Protein Assay as described in Example 3. Following concentration determination, the proteins are separated by SDS-PAGE and stained with SimplyBlue Safe Stain (Invitrogen). The amount of E-PhaC-M fusion protein relative to the amount of total protein attached to the particles is detected using a Gel Doc™ XR and analysed using Quantity One software (version 4.6.2, Bio-Rad Laboratories). Proteins of interest are excised from the gel and subjected to tryptic peptide fingerprinting using matrix-assisted laser desorption/ionization time-of-flight spectrometry (MALDI-TOF-MS), which allows identification of the fusion protein domains.

### 5. ELISA

Immuno-reactivity of the Dengue virus polymer particles was determined by enzyme-linked immunosorbent assay (ELISA) as described in Example 3. Briefly, maxisorb plates (Nunc) are coated overnight at 4° C. with purified E-PhaC-M particles or wild-type particles, diluted in carbonate-bicarbonate coating buffer (pH 9.6) (Sigma-Aldrich). Serial dilutions of the buffer are used, ranging from 1 mg/ml to 0.015 mg/ml protein concentration. Plates are washed and blocked for 2 h at 25° C. (see Table 4). Plates are then washed in PBS-Tween 20, incubated with mouse antibodies raised against the various antigens, washed and further incubated for 1 hour at room temperature with anti-mouse IgG:horse radish peroxidase conjugate (Sigma-Aldrich) diluted in 1% (w/v) BSA in PBS. After further washing, o-phenylenediamine (OPD) substrate (Sigma-Aldrich) is added and the plates are incubated for 30 minutes at room temperature. The reaction is stopped with 0.5 M H2SO4 and absorbance recorded at 495 nm.

### 6. Flow Cytometry

Thirty micrograms of various purified antigen-displaying particles or wild-type particles are washed twice in ice-cold flow cytometry buffer as described in Table 4 of Example 3 and incubated with mouse anti-antigen antibodies. After washing, particles are stained with rat anti-mouse Fluorescein isothiocyanate (FITC)-labelled antibody (BD Pharmingen, CA, USA), incubated for 30 minutes on ice in the dark and washed again. A BD FACScalibur (BD Biosciences, CA, USA) is used to collect at least 15,000 events for each sample and analysed using CellQuest software.

### 7. Immunisation of Mice

Female C57BL/6 mice (Malaghan Institute, Wellington, NZ) aged 6-8 weeks are intraperitoneally (i.p.) immunized two times at 2 week intervals. The three treatment groups are as follows:

a) individuals immunised with wild-type particles (i.e., particles prepared from bacterial cells carrying pHAS and pMCS69);

b) individuals immunised with antigen particles alone (i.e., particles prepared from bacterial cells carrying plasmids encoding the various antigen-PhaC fusion proteins and pMCS69); and

c) individuals immunised with the various antigen particles mixed with 20% Emulsigen™ adjuvant (MVP Laboratories). Non-vaccinated control animals are included for each set of experiments.

### 8. Immunological Assay

The mice are anaesthetised three weeks after the last immunisation and blood is collected, centrifuged, and the serum collected and frozen at −20° C. until assayed. The mice are then euthanized, their spleens removed and a single cell suspension is prepared by passage through an 80 gauge wire mesh sieve. Spleen red blood cells (RBCs) are processed as described in Example 4.

### 9. Plaque Reduction Neutralization Assay

Sera from immunized mice are examined for the presence of Dengue virus neutralizing antibodies by a plaque reduction neutralization test. Serially diluted sera are heat-inactivated, mixed with 100 plaque forming units of both a homologous and heterologous serotype virus then incubated for 1 h at 37° C. The sera virus mixture is incubated with Vero cell monolayers for 1 h then overlayed with agarose containing medium. Virus plaques are stained on day 5 of the assay. The highest dilution in which there is an 80% reduction in plaque number is the Plaque reduction neutralization 80 (PRNT₈₀).

### 10. Quantification of Cytokines and Chemokines

Culture supernatants are removed after 4 days incubation and frozen at −20° C. until assayed. Levels of cytokines and chemokines in the supernatants are measured by ELISA and/or FACS (EBioscience) according to manufacturer's instructions using commercially available antibodies and standards (EBiosciene).

### 11. Mouse Virus Protection Assay

A mouse challenge model is used to ascertain the efficacy of the formulations of Dengue virus E and M antigen presenting particles with and without adjuvant. Thirteen day-old weanling mice are immunized as stated above in section 1 of Material and Methods, using 1, 5 and 10 μg dosing. Following immunization, mice are challenged intracranially (IC) with 100 LD₅₀ of mouse-adapted Dengue virus. Morbidity and mortality is monitored for 21 days post-challenge.

### 12. Quantification of Serum Antibody

Serum antibody is measured by ELISA using immobilized antigen displaying particles for antibody capture.

### 13. Statistical Analysis

Analysis of the cytokine, chemokine and antibody responses is performed by Kruskal-Wallis one-way analysis of variance (ANOVA).

### Results

GC-MS analysis of cells carrying plasmids pET14B-E1-C-M1, pET14B-E2-C-M2, pET14B-E3-C-M3 or pET14B-E4-C-M4 and pHAS all in the presence of pMCS69, will confirm the presence of the polyester polyhydroxybutyrate. The presence of intracellular polyester inclusions may be further confirmed by fluorescent microscopy using Nile Red staining. The presence of polyhydroxybutyrate in cells carrying plasmids pET14B-E1-C-M1, pET14B-E2-C-M2, pET14B-E3-C-M3 or pET14B-E4-C-M4 and pHAS (wildtype control) all in the presence of pMCS69 indicates that the PhaC polyester synthase domain retained polymer synthase activity when present as a single or tripartite fusion protein.

High level protein display by particles is determined by a prominent protein band with an apparent molecular weight directly aligning with molecular weight deduced from the fusion protein sequence, respectively. The identity of these proteins is confirmed by tryptic peptide fingerprinting using MALDI-TOF-MS. ELISA results indicate that the various antigen displaying particles bind to the respective anti-antigen antibody in a dose-dependent manner, while wild-type particles show significantly less binding to the antibody. Flow cytometry results preferably show that >97% of antigen particles bind anti-antigen antibodies. Expression in recombinant E. coli of the respective hybrid gene encoding the PhaC-antigen fusion protein allow production of polyester particles displaying the fusion protein at their surface.

No overt toxicity is observed, preferably, in any of the animals after immunization, and mouse weights do not differ significantly between groups during the time-course of the experiment, and mice in all groups gained weight (data not shown). Mice immunised with polyester particles will be typically healthy throughout the trial with normal behaviour and good quality fur.

A dose range of about 10 to about 50 μg of antigen particles is generating a significant antibody response in mice. This dose induces significantly higher antibody titres when compared to a 10-50 μg dose of wildtype particles alone. Other doses may also be tested and used, for example 50-100 μg of each antigen displaying bead (E1-C-M1, E2-C-M2, E3-C-M3 and E4-C-M4). In a second experiment which includes non-immunised control mice and compares bead formulations with and without an adjuvant, antigen-specific serum antibody responses are significantly higher for both vaccine groups given antigen particles compared to non-vaccinated mice. The highest antibody responses are observed in mice immunised with antigen particles in Emulsigen. Antibody responses for the IgG1 isotype are stronger than responses for IgG2 in both experiments.

The cell-mediated response to antigens of mice immunised with 10-50 μg antigen particles is also significantly enhanced compared to that of mice immunised with wildtype particles alone, or with PBS alone and there should typically be no significant difference in the cell-mediated responses of mice immunised with wildtype particles alone compared to PBS-immunised control mice.

The sera from mice immunized with wild-type particles will typically not differ significantly from that of PBS-immunised control mice in the plaque reduction neutralization assay. The neutralization titer of sera from mice immunized with a formulation containing a 1:1:1:1 mixture of Dengue virus serotype 1-4 E-M particles in the plaque reduction neutralization assay will be significantly higher than compared to sera of mice immunized with wild-type particles alone. The neutralization titer of sera from mice immunized with a formulation containing a 1:1:1:1 mixture of Dengue virus serotype 1-4 E-M particles in the plaque reduction neutralization assay will be significantly higher for heterologous Dengue virus serotypes than a formulation containing only one Dengue virus serotype E and M presenting bead.

The chemokine and cytokine response to the antigen in mice immunised 2 times with 10-50 μg of wild-type particles will typically not differ significantly from that of PBS-immunised control mice. In contrast, a significantly greater chemokine and cytokine response to each antigen is observed in mice immunised 2 times with antigen particles, and in mice immunised 2 times with antigen particles and Emulsigen. Expected is a significantly greater cytokine and chemokine response to each antigen is observed in mice immunised 2 times with antigen particles and Emulsigen than all the other vaccine groups. The engineered polyester particles which display antigen Dengue virus serotype 1-4 E and M proteins are capable of producing an antigen-specific cell-mediated response, as well as significantly increasing the production of IgG1 and IgG2 antibodies.

Mice immunized with either PBS or wild-type particles are expected to die upon viral challenge without any significant difference between the two groups. The mice immunized with Dengue virus serotype 1-4 E and M presenting particles with and without adjuvant are expected to be protected, with better protection derived from the formulation containing adjuvant.

In addition to generation of both humoral and cell-mediated immune responses, the lack of adverse side effects such as weight loss, and absence of abscesses and suppuration at the injection site indicate that the polyester particles are well tolerated, safe, and non-toxic.

### Example 11—Immunogenicity of Ebola Virus Polymer Particle Vaccines

This example describes the construction of plasmids for the production in E. coli of polymer particles displaying the Filoviridae Zaire ebolavirus and Sudan ebolavirus virion spike glycoprotein precursor antigens (ZEBOV-GP and SEBOV-GP, respectively) either separately or simultaneously together with an analysis of the immunogenecity of the polymer particles. Both antigens are useful for vaccine development.

### Materials and Methods

All animal experiments were approved by the AgResearch Grasslands Animal Ethics Committee (Palmerston North, New Zealand).

### 1. Construction of Plasmids Mediating Fusion Protein Overproduction and Polymer Bead Formation

All plasmids and oligonucleotides used in this example are listed in Table 11.

The polyhydroxybutyrate biosynthesis enzymes, beta-ketothiolase and the R-specific acetoacetyl-Coenzyme reductase are encoded by plasmid pMCS69. To produce polymer particles simultaneously displaying two Ebola virion spike glycoprotein precursor antigens, genes encoding the virion spike glycoprotein precursor antigens from Zaire Ebola virus and Sudan Ebola virus are codon optimized and synthesized by Genscript Inc. to allow subcloning into pET-14b M-PhaC-linker-MalE XbaI-SpeI site for an N-terminal fusion and into XhoI-BamHI sites for a C-terminal fusion to the PhaC polymer bead forming enzyme. The ZEBOV-GP encoding gene is inserted into the XbaI-SpeI sites and on the same plasmid the SEBOV-GP encoding gene is inserted into the XhoI-BamHI sites. Both gene insertion are in frame and require replacement of the M and MalE encoding regions of the original plasmid. This results in plasmid pET14B-ZEBOVGP-C-SEBOVGP. Alternatively, the SEBOV-GP encoding gene can be inserted into the XbaI-SpeI sites while the ZEBOV-GP encoding gene can be inserted into the XhoI-BamHI sites on the same plasmid, generating the plasmid pET14B-SEBOVGP-C-ZEBOVGP.

The construct for the ZEBOVGP-C-SEBOVGP fusion is shown as SEQ ID No. 49, with the derived amino acid sequence shown as SEQ ID No. 50. The construct for the SEBOVGP-C—ZEBOVGP fusion is shown as SEQ ID No. 51, with the derived amino acid sequence shown as SEQ ID No. 52.

TABLE 11
Plasmids and Oligonucleotides

| Plasmids | Description |
|---|---|
| pHAS | pET14b derivative containing the NdeI/BamHI inserted |
| | phaC gene from C. necator |
| pMCS69 | pBBR1MCS derivative containing genes phaA and phaB |
| | from C. necator |
| pET-14b M- | pET-14b PhaC-linker-MalE derivative |
| PhaC-linker- | containing the mpl sequence fused to the |
| MalE | 5′ end of phaC |
| pET14B- | pET-14b M-PhaC-linker-MalE derivative |
| ZEBOVGP-C- | containing the ZEBOV-GP sequence fused to the |
| SEBOVGP | 5′ end and SEBOV-GP fused to the 3′ end of phaC |
| pET14B- | pET-14b M-PhaC-linker-MalE derivative |
| SEBOVGP-C- | containing the SEBOV-GP sequence fused to the |
| ZEBOVGP | 5′ end and ZEBOV-GP fused to the 3′ end of phaC |

### 2. Production of ZEBOVGP—SEBOVGP Displaying Particles

Either plasmid pET14B-ZEBOVGP-C-SEBOVGP or pET14B-ZEBOVGP-C-SEBOVGP and pHAS are introduced into E. coli KRX cells harbouring plasmid pMCS69. The transformants are cultured in conditions suitable for the production of biopolyester particles, as described in Example 1. The ability to produce ZEBOVGP-SEBOVGP particles or wild-type particles, respectively, is then assessed as described below.

### 3. Isolation of Polyester Particles

Polyester granules are isolated as described in Example 3. The concentration of protein attached to particles is determined using the Bio-Rad Protein Assay as described in Example 3. Following concentration determination, the proteins are separated by SDS-PAGE and stained with SimplyBlue Safe Stain (Invitrogen). The amount of ZEBOVGP-PhaC-SEBOVGP or SEBOVGP-PhaC-ZEBOVGP fusion protein, respectively, relative to the amount of total protein attached to the particles is detected using a Gel Doc™ XR and analysed using Quantity One software (version 4.6.2, Bio-Rad Laboratories).

Proteins of interest are identified using matrix-assisted laser desorption/ionization time-of-flight spectrometry (MALDI-TOF-MS), which allows identification of the fusion protein domains.

### 4. ELISA

Immuno-reactivity of the Ebola virus polymer particles was determined by enzyme-linked immunosorbent assay (ELISA) as described in Example 3. Briefly, maxisorb plates (Nunc) are coated overnight at 4° C. with purified ZEBOVGP-PhaC-SEBOVGP particles, SEBOVGP-PhaC-ZEBOVGP particles or wild-type particles, diluted in carbonate-bicarbonate coating buffer (pH 9.6) (Sigma-Aldrich). Serial dilutions of the buffer are used, ranging from 1 mg/ml to 0.015 mg/ml protein concentration. Plates are washed and blocked for 2 h at 25° C. (see Table 4). Plates are then washed in PBS-Tween 20, incubated with mouse antibodies raised against the various antigens, washed and further incubated for 1 hour at room temperature with anti-mouse IgG:horse radish peroxidase conjugate (Sigma-Aldrich) diluted in 1% (w/v) BSA in PBS. After further washing, o-phenylenediamine (OPD) substrate (Sigma-Aldrich) is added and the plates are incubated for 30 minutes at room temperature. The reaction is stopped with 0.5 M H2SO4 and absorbance recorded at 495 nm.

### 5. Immunisation of Mice

Female C57BL/6 mice (Malaghan Institute, Wellington, NZ) aged 6-8 weeks are intramuscularly immunized three times at 2 week intervals. The three treatment groups are as follows:
a) individuals immunised with wild-type particles (ie., particles prepared from bacterial cells carrying pHAS and pMCS69);
b) individuals immunised with antigen particles alone (i.e., particles prepared from bacterial cells carrying plasmids encoding the various antigen-PhaC fusion proteins and pMCS69); and
c) individuals immunised with the various antigen particles mixed with 20% Emulsigen™ adjuvant (MVP Laboratories).

Non-vaccinated control animals are included for each set of experiments.

### 6. Immunological Assay

The mice are anaesthetised three weeks after the last immunisation and blood is collected, centrifuged, and the serum collected and frozen at −20° C. until assayed. The mice are then euthanased, their spleens removed and a single cell suspension is prepared by passage through an 80 gauge wire mesh sieve. Spleen red blood cells (RBCs) are lysed using a solution of 17 mM TRIS-HCl and 140 mM NH4Cl. After washing, the RBCs are cultured in Dulbecco's Modified Eagle media (DMEM) supplemented with 2 mM glutamine (Invitrogen), 100 U/mL penicillin (Invitrogen), 100 μg/mL streptomycin (Invitrogen), 5×10-5 M 2-mercaptoethanol (Sigma) and 5% (w/w) Foetal Calf Serum (Invitrogen).

### 7. Plaque Reduction Neutralization Assay

Sera from immunized mice are examined for the presence of Ebola virus neutralizing antibodies by a plaque reduction neutralization test. Serially diluted sera are heat-inactivated, mixed with 100 plaque forming units of both a homologous and heterologous virus then incubated for 1 h at 37° C. The sera virus mixture is incubated with Vero cell monolayers for 1 h then overlayed with agarose containing medium. Virus plaques are stained on day 10-12 of the assay. The highest dilution in which there is an 80% reduction in plaque number is the Plaque reduction neutralization 80 (PRNT₈₀).

### 8. Quantification of Cytokines and Chemokines

Culture supernatants are removed after 4 days incubation and frozen at −20° C. until assayed. Levels of cytokines and chemokines in the supernatants are measured by ELISA and/or FACS (EBioscience) according to manufacturer's instructions using commercially available antibodies and standards (EBiosciene).

### 9. Mouse Virus Protection Assay

A mouse challenge model is used to ascertain the efficacy of the formulations of ZEBOVGP and SEBOVGP antigen presenting particles with and without adjuvant. B10.BR mice (MHE H-2^{K}), The Jackson Laboratory, ME)⁵ are immunized as stated above in section 1 of Material and Methods, using 1, 5 and 10 μg dosing. Following immunization, mice are challenged by intraperitoneal injection (IP) with 1000×LD₅₀ of mouse-adapted ZEBOV. Morbidity and mortality is monitored for 12-16 days post-challenge.

Efficacy of the formulations of ZEBOVGP and SEBOVGP antigen presenting particles with and without adjuvant is ascertained via administration of the vaccine formulations 30 minutes post IP injection of 1000×LD₅₀. Morbidity and mortality is monitored for 12-16 days post-challenge.

### 10. Quantification of Serum Antibody

Serum antibody is measured by ELISA using immobilized antigen displaying particles for antibody capture.

### 11. Statistical Analysis

Analysis of the cytokine, chemokine and of the antibody responses is performed by Kruskal-Wallis one-way analysis of variance (ANOVA).

### Results

GC-MS analysis of cells carrying plasmids pET14B-ZEBOVGP-C-SEBOVGP or pET14B-SEBOVGP-C-ZEBOVGP and pHAS all in the presence of pMCS69, confirmed the presence of the polyester polyhydroxybutyrate. The presence of intracellular polyester inclusions is further confirmed by fluorescent microscopy using Nile Red staining. The presence of polyhydroxybutyrate in cells carrying plasmids pET14B-ZEBOVGP-C-SEBOVGP or pET14B-SEBOVGP-C-ZEBOVGP and pHAS (wildtype control) all in the presence of pMCS69 indicates that the PhaC polyester synthase domain retained polymer synthase activity when present as a single or tripartite fusion protein.

The particles display high levels of protein as shown by a prominent protein band with an apparent molecular weight directly aligning with molecular weight deduced from the fusion protein sequence, respectively. The identity of these proteins is confirmed by tryptic peptide fingerprinting using MALDI-TOF-MS. ELISA indicates that the various antigen displaying particles bind to the respective anti-antigen antibody in a dose-dependent manner, while wild-type particles bind significantly less to the antibody. Flow cytometry shows that >98% of antigen particles bind anti-antigen antibodies. Results will indicate that the expression in recombinant E. coli of the respective hybrid genes encoding the various antigen-PhaC fusion proteins leads to the production of polyester particles displaying the fusion protein at their surface.

No overt toxicity is observed in any of the animals after immunisation. Mouse weight does not differ significantly between groups during the time-course of the experiment, and mice in all groups gained weight. Mice immunised with polyester particles will typically develop small lumps (2.5 mm in diameter) at the immunisation sites but no abscesses or suppuration will be observed. All mice are typically healthy throughout the trial with normal behaviour and good quality fur.

A dose of 5-100 μg of antigen particles is optimal at generating a significant antibody response in mice. This dose induces significantly higher antibody titres when compared to a 5-100 dose of wildtype particles alone. In a second experiment which includes non-immunised control mice and compares bead formulations with and without an adjuvant, antigen-specific serum antibody responses are significantly higher for both vaccine groups given antigen particles compared to non-vaccinated mice. The highest antibody responses will typically be observed in mice immunised with antigen particles in Emulsigen. Antibody responses for the IgG1 isotype are stronger than responses for IgG2.

The cell-mediated response to antigens of mice immunised with 5-100 μg antigen particles is significantly enhanced compared to that of mice immunised with wildtype particles alone, or with PBS alone. There is no significant difference in the cell-mediated responses of mice immunised with wildtype particles alone compared to PBS-immunised control mice. The chemokine and cytokine response to the antigen in mice immunised 2 times with 10-50 μg of wild-type particles will typically not differ significantly from that of PBS-immunised control mice. In contrast, a significantly greater chemokine and cytokine response to each antigen is observed in mice immunised 2 times with antigen particles, and in mice immunised 2 times with antigen particles and Emulsigen. Expected is a significantly greater cytokine and chemokine response to each antigen is observed in mice immunised 2 times with antigen particles and Emulsigen than all the other vaccine groups. The engineered polyester particles which display antigen ZEBOVGP and SEBOVGP proteins are capable of producing an antigen-specific cell-mediated response, as well as significantly increasing the production of IgG1 and IgG2 antibodies.

The sera from mice immunized with wild-type particles will typically not differ significantly from that of PBS-immunised control mice in the plaque reduction neutralization assay. The neutralization titer of sera from mice immunized with a formulation ZEBOVGP and SEBOVGP presenting particles in the plaque reduction neutralization assay will be significantly higher than compared to sera of mice immunized with wild-type particles alone. The neutralization titer of sera from mice immunized with a formulation containing the ZEBOVGP and SEBOVGP particles in the plaque reduction neutralization assay will be similar for homologous and heterologous virus.

Mice immunized with either PBS or wild-type particles are expected to die upon viral challenge without any significant difference between the two groups irrespective of immunization time and order. The mice immunized with ZEBOVGP and SEBOVGP presenting particles with and without adjuvant prior to virus innoculation are expected to be protected; with better protection derived from the formulation containing adjuvant. Further, mice immunized with ZEBOVGP and SEBOVGP presenting particles with and without adjuvant are expected to be protected.

The engineered polyester particles simultaneously displaying the ZEBOV-GP and SEBOV-GP antigens are capable of producing an antigen-specific cell-mediated response, as well as significantly increasing the production of IgG1 and IgG2 antibodies The lack of adverse side effects such as weight loss, and absence of abscesses and suppuration at the injection site indicate that the polyester particles are well tolerated, safe, and non-toxic.

### Example 12—Immunogenicity of West Nile Virus Polymer Particle Vaccines

This example describes the construction of plasmids for the production in transformed hosts, in this case, E. coli, of polymer particles displaying the Flavivirus envelope antigen (E) from West Nile virus (WNV), a non-toxic protein expressed on the surface of WNV virions (WNVE), together with an analysis of the immunogenecity of the polymer particles. This antigen is considered a leading candidate for vaccine development. While several vaccine formulations are currently being examined, there is no approved WNV vaccine. Polymer particles displaying this antigen as produced in this example are useful as prophylactic and therapeutic vaccines against WNV.

### Materials and Methods

All animal experiments were approved by the AgResearch Grasslands Animal Ethics Committee (Palmerston North, New Zealand).

### 1. Construction of Plasmids

All plasmids and oligonucleotides used in this example are listed in Table 12.

Enzymes mediating the synthesis of 3-hydroxybutyryl-Coenzyme A are encoded by plasmid pMCS69.

To produce polymer particles displaying the WNVE antigen, a gene encoding the envelope (E) is codon optimized, harmonized and synthesized by Genscript Inc. to allow subcloning into pET-14b PhaC-linker-GFP XhoI-BamHI sites for a C-terminal fusion to the PhaC polymer bead forming enzyme. The E encoding gene is inserted into the XhoI-BamHI site. This gene insertion is in frame with GFP encoding region of the original plasmid replaced, yielding plasmid pET14B-C-WNVE.

The construct for the PhaC-WNVE fusion is shown as SEQ ID No. 53, with the derived amino acid sequence shown as SEQ ID No. 54.

TABLE 12
Plasmids and Oligonucleotides

| Plasmids | Description |
|---|---|
| pHAS | pET14b derivative containing the NdeI/BamHI inserted |
| | phaC gene from C. necator |
| pMCS69 | pBBR1MCS derivative containing genes phaA and phaB |
| | from C. necator |
| pET-14b PhaC- | pET-14b derivative containing the GFP encoding DNA |
| linker-GFP | sequence fused to the 3′ end of phaC |
| pET14B-C- | pET-14b PhaC-linker-GFP derivative |
| WNVE | containing the WNVE encoding DNA sequence fused to |
| | the 3′ end of phaC |

### 2. Production of WNVE Displaying Particles

Plasmid pET14B-C-WNVE and pHAS are introduced into E. coli BL21 Star (DE3) cells harbouring plasmid pMCS69. The transformants are cultured in conditions suitable for the production of biopolyester particles, as described in Example 1.

### 3. Gas Chromatography Mass Spectroscopy (GC-MS)

The polyester content of bacterial cells harbouring the various plasmids corresponds to the activity of the PhaC synthase in vivo. The amount of accumulated polyester is assessed by gas chromatography-mass spectroscopy (GC-MS) analysis to determine PhaC synthase activity, and particularly to assess whether the PhaC-WNVE antigen fusion still catalyses polyester synthesis and mediates intracellular granule formation. Polyester content is quantitatively determined by GC-MS after conversion of the polyester into 3-hydroxymethyl ester by acid-catalysed methanolysis.

### 4. Isolation of Polyester Particles

Polyester granules are isolated as described in Example 3.

### 5. Protein Concentration Determination

The concentration of protein attached to particles is determined using the Bio-Rad Protein Assay as described in Example 3.

### 6. ELISA

Immuno-reactivity of the West Nile virus polymer particles was determined by enzyme-linked immunosorbent assay (ELISA) as described in Example 3. Maxisorb plates (Nunc) are coated overnight at 4° C. with purified PhaC-WNVE particles or wild-type particles, diluted in carbonate-bicarbonate coating buffer (pH 9.6) (Sigma-Aldrich). Serial dilutions of the buffer are used, ranging from 1 mg/ml to 0.015 mg/ml protein concentration. Plates are washed and blocked for 2 h at 25° C.

Plates are then washed in PBS-Tween 20, incubated with mouse antibodies raised against the various antigens, washed and further incubated for 1 hour at room temperature with anti-mouse IgG:horse radish peroxidase conjugate (Sigma-Aldrich) diluted in 1% (w/v) BSA in PBS. After further washing, o-phenylenediamine (OPD) substrate (Sigma-Aldrich) is added and the plates are incubated for 30 minutes at room temperature.

The reaction is stopped with 0.5 M H2SO4 and absorbance recorded at 495 nm.

### 7. Immunisation of Mice

Female C57BL/6 mice (Malaghan Institute, Wellington, NZ) aged 6-8 weeks are intramuscularly immunized three times at 2 week intervals. The three treatment groups are as follows:
a) individuals immunised with wild-type particles (i.e., particles prepared from bacterial cells carrying pHAS and pMCS69);
b) individuals immunised with antigen particles alone (i.e., particles prepared from bacterial cells carrying plasmids encoding the various antigen-PhaC fusion proteins and pMCS69);
c) individuals immunised with the various antigen particles mixed with 20% Emulsigen™ adjuvant (MVP Laboratories).

Non-vaccinated control animals are included for each set of experiments.

### 8. Immunological Assay

The mice are anaesthetised three weeks after the last immunisation and blood is collected, centrifuged, and the serum collected and frozen at −20° C. until assayed.

The mice are then euthanized, their spleens removed and a single cell suspension is prepared by passage through an 80 gauge wire mesh sieve. Spleen red blood cells (RBCs) are processed as described in Example 4.

### 9. Plaque Reduction Neutralization Assay

Sera from immunized mice are examined for the presence of West Nile virus neutralizing antibodies by a plaque reduction neutralization test. Serially diluted sera are heat-inactivated, mixed with 100 plaque forming units (PFU) of both a homologous and heterologous serotype virus then incubated for 1 h at 37° C. The sera-virus mixture is incubated with Vero cell monolayers for 1 h then overlayed with agarose containing medium. Virus plaques are stained on day 5 of the assay. The highest dilution in which there is an 80% reduction in plaque number is the Plaque reduction neutralization 80 (PRNT₈₀).

10. Quantification of Cytokines and Chemokines Culture supernatants are removed after 4 days incubation and frozen at −20° C. until assayed.

Levels of cytokines and chemokines in the supernatants are measured by ELISA and/or FACS (EBioscience) according to manufacturer's instructions using commercially available antibodies and standards (EBiosciene).

### 11. Mouse Virus Protection Assay

A mouse challenge model is used to ascertain the efficacy of the formulations of West Nile E antigen presenting particles with and without adjuvant. Thirteen day-old weanling mice are immunized as stated above in section 1 of Material and Methods, using 1, 5 and 10 μg dosing. Following immunization, mice are challenged intracranially (IC) with 100 LD₅₀ of mouse-adapted West Nile virus. Morbidity and mortality is monitored for 21 days post-challenge.

### 12. Quantification of Serum Antibody

Serum antibody is measured by ELISA using immobilized antigen displaying particles for antibody capture.

### 13. Statistical Analysis

Analysis of the cytokine, chemokine and antibody responses is performed by Kruskal-Wallis one-way analysis of variance (ANOVA).

### Results

GC-MS analysis of cells carrying plasmids pET14B-C-WNVE and pHAS all in the presence of pMCS69, will confirm the presence of the polyester polyhydroxybutyrate. The presence of intracellular polyester inclusions may be further confirmed by fluorescent microscopy using Nile Red staining.

The presence of polyhydroxybutyrate in cells carrying plasmids pET14B-C-WNVE and pHAS (wildtype control) all in the presence of pMCS69 indicates that the PhaC polyester synthase domain retained polymer synthase activity when present as a single or tripartite fusion protein.

High level protein display by particles is determined by a prominent protein band with an apparent molecular weight directly aligning with molecular weight deduced from the fusion protein sequence, respectively. The identity of these proteins is confirmed by tryptic peptide fingerprinting using MALDI-TOF-MS. ELISA results indicates that the various antigen displaying particles bind to the respective anti-antigen antibody in a dose-dependent manner, while wild-type particles bind significantly less to the antibody. Flow cytometry results preferably show that >97% of antigen particles bind anti-antigen antibodies.

Expression in recombinant E. coli of the respective hybrid gene encoding the PhaC-antigen fusion protein allows production of polyester particles displaying the fusion protein at their surface.

Preferably, no overt toxicity is observed in any of the animals after immunization, and mouse weights do not differ significantly between groups during the time-course of the experiment, and mice in all groups gained weight (data not shown). Mice immunised with polyester particles will develop small lumps (2.5 mm in diameter) at the immunisation sites but generally without abscesses or suppuration and are typically healthy throughout the trial with normal behaviour and good quality fur. A dose of 5-100 μg of antigen particles is generating a significant antibody response in mice. This dose induces significantly higher antibody titres when compared to a 5-100 μg dose of wild-type particles alone. Other doses may also be tested and used. In a second experiment, which includes non-immunized mice (control group), mice immunized with both control wild-type particles (bead control groups) and WNVE presenting particles (test groups) formulated with and without an adjuvant. Mice are evaluated for significantly higher antigen-specific serum antibody responses for both mouse groups given antigen presenting particles in comparison to non-vaccinated or wild-type bead immunized mice. The highest antibody responses may be observed in mice immunised with antigen particles formulated in Emulsigen. Antibody responses for the IgG1 isotype will be stronger than responses for IgG2 in both experiments.

The cell-mediated response to antigens of mice immunised with 5-100 μg antigen particles is also significantly enhanced compared to that of mice immunised with either wildtype particles or with PBS alone. There should typically be no significant difference in the cell-mediated responses of mice immunised with wildtype particles alone compared to PBS-immunised control mice.

The sera from mice immunized with wild-type particles will typically not differ significantly from that of PBS-immunised control mice in the plaque reduction neutralization assay. The neutralization titer of sera from mice immunized with a formulation containing WNVE particles in the plaque reduction neutralization assay will be significantly higher than compared to sera of mice immunized with wild-type particles alone. Preferably, the neutralization titer of sera from mice immunized with a formulation containing the WNVE particles will be similar between homologous and heterologous West Nile virus.

The chemokine and cytokine response to the antigen in mice immunised 2 times with 5-100 μg of wild-type particles will typically not differ significantly from that of PBS-immunised control mice. In contrast, a significantly greater chemokine and cytokine response is observed in mice immunised 2 times with antigen particles, and in mice immunised 2 times with antigen particles and Emulsigen. Expected is a significantly greater cytokine and chemokine response to each antigen is observed in mice immunised 2 times with antigen particles and Emulsigen than all the other vaccine groups. The engineered polyester particles which display WNVE antigen are capable of producing an antigen-specific cell-mediated response, as well as significantly increasing the production of IgG1 and IgG2 antibodies.

Mice immunized with either PBS or wild-type particles are expected to die upon viral challenge without any significant difference between the two groups. The mice immunized with WNVE presenting particles with and without adjuvant are expected to be protected, with better protection derived from the formulation containing adjuvant.

The engineered polyester particles which display WNVE are capable of producing an antigen-specific cell-mediated response, as well as significantly increasing the production of IgG1 and IgG2 antibodies. In addition to generation of both humoral and cell-mediated immune responses, the lack of adverse side effects such as weight loss, and absence of abscesses and suppuration at the injection site indicate that the polyester particles are well tolerated, safe, and non-toxic.

### Example 13—Immunological Studies In Vivo in Mice

This example describes the immunisation of a mammalian model organism with Ag85A-ESAT-6 polymer particles.

### Materials and Methods

All animal experiments were approved by the AgResearch Grasslands Animal Ethics Committee (Palmerston North, New Zealand).

1. Construction of Plasmids and Production of Polymer Particles in E. coli and L. lactis

Plasmids were constructed for the production of polymer particles displaying the tuberculosis antigens Ag-85A and ESAT-6 in L. lactis and E. coli as described in Examples 1 and 2.

Polymer granules were isolated by disrupting the bacteria and whole cell lysates were centrifuged at 6000 g for 15 minutes at 4° C. to sediment the polymer particles. The particles were purified via glycerol gradient ultracentrifugation. Protein concentration was determined using the Bio-Rad Protein Assay according to the manufacturer's instructions (Bio-Rad). The amount of Ag85A-ESAT-6:PhaC fusion protein relative to the amount of total protein attached to the polymer particles was detected using a Gel Doc™ XR and analysed using Quantity One software (version 4.6.2, Bio-Rad). The Tb antigen accounted for approximately 20% of the total protein of the polymer particle. Identification of the protein of interest was confirmed using matrix-assisted laser desorption/ionisation time-of flight mass spectrometry (MALDI-TOF-MS).

2. ELISA Activity of the polymer particles was determined by enzyme-linked immunosorbent assay (ELISA) as described in Example 3. Absorbance was recorded at 490 nm on a VERSAax microplate reader.

### 3. Immunisation of Mice

Female C57BL/6 mice (Malaghan Institute, Wellington, NZ) aged 6-8 weeks were sub-cutaneously immunized three times at 2 week intervals with tuberculosis polymer particle vaccines constructed and isolated as described in Examples 1, 2 and 3. The three treatment groups were as follows:
a) individuals immunised with wild-type polymer particles (ie., polymer particles prepared from bacterial cells carrying pHAS and pMCS69);
b) individuals immunised with Ag85A-ESAT-6 polymer particles alone (ie., polymer particles prepared from bacterial cells carrying pHAS-Ag85A-ESAT-6 and pMCS69);
c) individuals immunised with Ag85A-ESAT-6 polymer particles mixed with 20% Emulsigen™ adjuvant (MVP Laboratories).

Non-vaccinated control animals were included for each set of experiments.

### 4. Immunological Assay

The mice were anaesthetised three weeks after the last immunisation and blood was collected, centrifuged, and the serum collected and frozen at −20° C. until assayed.

The mice were then euthanased, their spleens removed and a single cell suspension was prepared by passage through an 80 gauge wire mesh sieve. Spleen red blood cells (RBCs) were lysed using a solution of 17 mM TRIS-HCl and 140 mM NH4Cl. After washing, the RBCs were cultured in Dulbecco's Modified Eagle media (DMEM) supplemented with 2 mM glutamine (Invitrogen), 100 U/mL penicillin (Invitrogen), 100 μg/mL streptomycin (Invitrogen), 5×10-5 M 2-mercaptoethanol (Sigma) and 5% (w/w) Foetal Calf Serum (Invitrogen).

The cells were incubated at 37° C. in 10% CO2 in medium alone, or in medium containing either: Ag85A, ESAT-6, or a combination of both antigens.

### 5. Quantification of IFN-γ

Culture supernatants were removed after 4 days incubation and frozen at −20° C. until assayed. Levels of IFN-γ in the supernatants were measured by ELISA (BD Biosciences) according to manufacturer's instructions using commercially available antibodies and standards (BD Pharmingen).

### 6. Quantification of Serum Antibody

Serum antibody was measured by ELISA according to manufacturer's recommendations using monoclonal anti-ESAT-6 or anti-Ag85A antibodies (Abcam).

### 7. Statistical Analysis

Analysis of the IFN-γ responses and of the antibody responses was performed by Kruskal-Wallis one-way analysis of variance (ANOVA).

### Results

No overt toxicity was observed in any of the animals after immunisation. Mouse weights did not differ significantly between groups during the time-course of the experiment, and mice in all groups gained weight (data not shown). Mice immunised with polyester polymer particles developed small lumps (2.5 mm in diameter) at the immunisation sites but no abscesses or suppuration was observed. All mice were healthy throughout the trial with normal behaviour and good quality fur (data not shown).

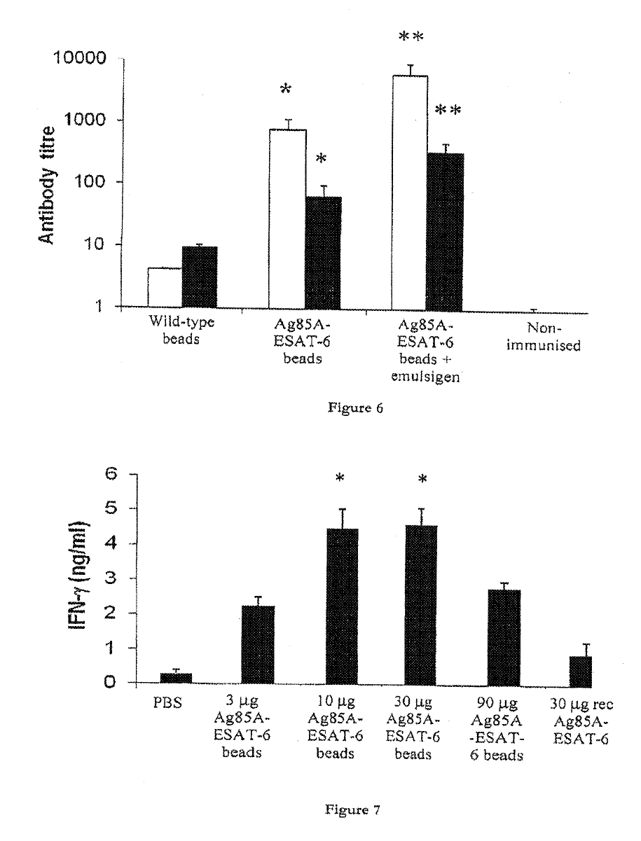

A dose of 30 μg of Ag85A-ESAT-6 polymer particles was shown to be optimal at generating a significant antibody response in mice (see FIG. 5). This dose induced significantly higher antibody titres when compared to a 30 μg dose of recombinant Ag85A-ESAT-6 protein alone (P<0.01). In a second experiment which included non-immunised control mice and compared bead formulations with and without an adjuvant, antigen-specific serum antibody responses were significantly higher for both vaccine groups given Ag85A-ESAT-6 polymer particles compared to non-vaccinated mice (P<0.01, see FIG. 6). The highest antibody responses were observed in mice immunised with Ag85A-ESAT-6 polymer particles in Emulsigen. Antibody responses for the IgG1 isotype were stronger than responses for IgG2 in both experiments.

As shown in FIG. 7, the cell-mediated response to ESAT-6 and Ag85A of mice immunised with 10 μg or with 30 μg Ag85A-ESAT-6 polymer particles was significantly enhanced compared to that of mice immunised with recombinant ESAT-6-Ag85A antigen alone (P<0.01), or with PBS alone (p<0.01). There was no significant difference in the cell-mediated responses of mice immunised with antigen alone compared to PBS-immunised control mice.

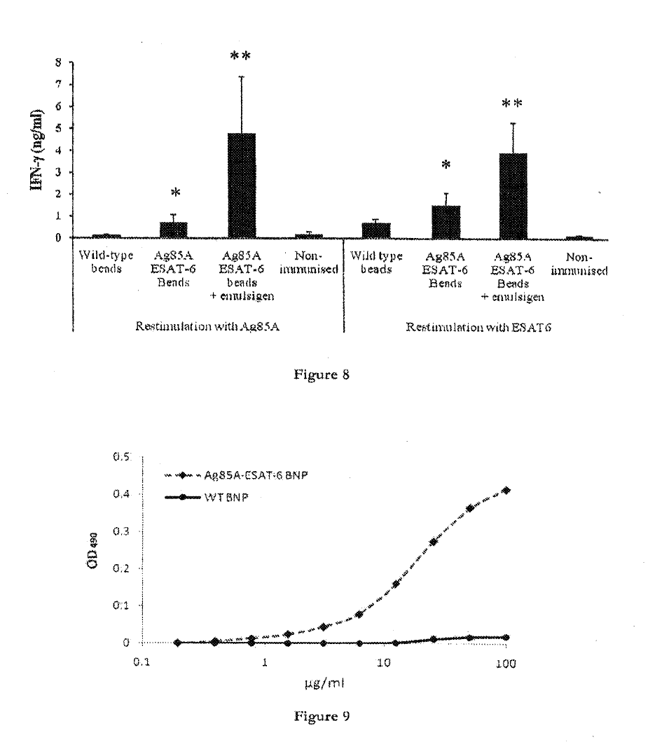

As shown in FIG. 8, the IFN-γ response to either ESAT-6 or Ag85A antigen in mice immunised 3 times with 30 μg of wild-type polymer particles (no Tb antigen) did not differ significantly from that of PBS-immunised control mice. In contrast, a significantly greater IFN-γ response to each antigen was observed in mice immunised 3 times with Ag85A-ESAT-6 polymer particles (p<0.01), and in mice immunised 3 times with Ag85A-ESAT-6 polymer particles and Emulsigen (p<0.01). Indeed, a significantly greater IFN-γ response to each antigen was observed in mice immunised 3 times with Ag85A-ESAT-6 polymer particles and Emulsigen than all the other vaccine groups (p<0.01, **).

### Discussion

The engineered polyester polymer particles displaying an Ag85A-ESAT-6 antigen fusion were capable of producing an antigen-specific cell-mediated response, as well as significantly increasing the production of IgG and IgG2 antibodies. Notably, immunisation with antigen alone (i.e., antigen not comprising a polymer particle of the present invention) was ineffective in eliciting a cell-mediated response.

These results also demonstrated the versatility and potential of this vaccine-delivery system to elicit complementary facets of the immune response, whereby both humoral and cell-mediated immune responses were elicited.

The lack of adverse side effects such as weight loss, and absence of abscesses and suppuration at the injection site demonstrated that the polyester polymer particles were well tolerated, safe, and non-toxic.

### Example 14—Pathogenic Challenge in Immunised Mice In Vivo

This example describes the efficacy of immunisation of a mammalian model with Ag85A-ESAT-6 polymer particles exposed to pathogenic challenge with M. bovis.

### Materials and Methods

All animal experiments were approved by the AgResearch Grasslands Animal Ethics Committee (Palmerston North, New Zealand).

### 1. Construction of Plasmids and Isolation of Polyester Polymer Particles

Plasmids were constructed for the production of polymer particles displaying the tuberculosis antigens Ag-85A and ESAT-6 in L. lactis and E. coli as described in Examples 1 and 2.

Polymer granules were isolated by disrupting the bacteria and whole cell lysates were centrifuged at 6000 g for 15 minutes at 4° C. to sediment the polymer particles. The particles were purified via glycerol gradient ultracentrifugation. Protein concentration was determined using the Bio-Rad Protein Assay according to the manufacturer's instructions (Bio-Rad). The amount of Ag85A-ESAT-6:PhaC fusion protein relative to the amount of total protein attached to the polymer particles was detected using a Gel Doc™ XR and analysed using Quantity One software (version 4.6.2, Bio-Rad). The Tb antigens accounted for approximately 20% of the total protein of the polymer particle. Identification of the protein of interest was confirmed using matrix-assisted laser desorption/ionisation time-of flight mass spectrometry (MALDI-TOF-MS).

### 2. ELISA

Activity of the polymer particles was determined by enzyme-linked immunosorbent assay (ELISA) as described in Example 3. Absorbance was recorded at 490 nm on a VERSAax microplate reader.

### 3. Immunisation of Mice

Female C57BL/6 mice (Malaghan Institute, Wellington, NZ) aged 6-8 weeks were sub-cutaneously immunized three times at weekly intervals. Seven treatment groups (n=6 per group) were as follows:
a) individuals immunised with PBS and Emulsigen™ adjuvant (MVP Laboratories).
b) individuals immunised with Ag85A-ESAT-6 polymer particles (E. coli host) mixed with 20% Emulsigen™ adjuvant (MVP Laboratories).
c) individuals immunised with wild-type polymer particles (E. coli host) mixed with 20% Emulsigen™ adjuvant (MVP Laboratories).
d) individuals immunised with Ag85A-ESAT-6 polymer particles (L. lactis host) mixed with 20% Emulsigen™ adjuvant (MVP Laboratories).
e) individuals immunised with wild-type polymer particles (L. lactis host) mixed with 20% Emulsigen™ adjuvant (MVP Laboratories).
f) individuals immunised with recombinant Ag85A-ESAT-6 antigen mixed with 20% Emulsigen™ adjuvant (MVP Laboratories).
g) individuals immunised with BCG 10⁶ CFU dose

Non-vaccinated control animals were included for each set of experiments.

### 4. Pathogenic Challenge

Fifteen weeks after the first vaccination, all mice were challenged with Mycobacterium bovis. M. bovis was grown from a low-passage seed lot in tween albumin broth (Tween 80, Dubos broth base and oleic acid-albumin-dextrose, Difco) to early mid-log phase. Aliquots of cultures were frozen at −70° C. until required.

To infect the mice by low-dose aerosol exposure, diluted M. bovis stock was administered using a Madison chamber aerosol generation device calibrated to deliver approximately 50 bacteria into the lungs of each mouse.

### 5. Immunological Assay

The mice were anaesthetised intraperitoneally five weeks after the pathogenic challenge using 87 μg ketamine (Parnell Laboratories, Australia) and 2.6 μg xylazine hydrochloride (Bayer, Germany) per gram of body weight. Blood was collected, centrifuged, and the serum collected and frozen at −20° C. until assayed.

The mice were then euthanased, their spleens and lungs removed. The apical lung lobe was removed from the lung and preserved in 10% buffered formalin, for subsequent histological processing. Sections were stained with the Ziehl-Neelson and haematoxylin and eosin stains.

The spleen and remaining lung samples were mechanically homogenised in 3 mL PBS with 0.5% Tween 80 using a Seward Stomacher® 80 (Seward, UK) and plated in tenfold dilutions on selectibe Middlebrook 7H11 agar supplemented with 10% oleic acid-albumin-dextrose-catalase enrichment (BD). Plates were incubated at 37° C. in humidified air for 3 weeks before counting.

### 6. Quantification of Serum Antibody

Serum antibody was measured by ELISA according to manufacturer's recommendations using monoclonal anti-ESAT-6 antibody (Abcam). Briefly, Microlon high-binding plates (Greiner) were coated overnight with 5 μg/mL of recAg85A-ESAT-6, blocked with 1% BSA and washed in PBST. Five-fold dilutions of serum (from 1:50 to 1:6250) were added and incubated. Following washing, anti-mouse IgG1:HRP or IgG2c:HRP (ICL, USA) was added and the plates incubated. Plates were washed and TMB used as a substrate prior to reading at 450 nm on a VERSAmax microplate reader.

Monoclonal anti-ESAT6 antibodies were titrated and included as a positive control for the IgG1 plates.

### 7. Statistical Analysis

Analysis of the bacterial counts from the M. bovis pathogenic challenge and antibody responses was performed by Fisher's one-way analysis of variance (ANOVA), with a level of significance of P<0.05.

### Results

Reactivity of Ag85A-ESAT-6 polymer particles produced in L. lactis showed a dose-dependent response to ESAT-6 antibody, while no antibody binding was observed for wild type polymer particles (FIG. 9).

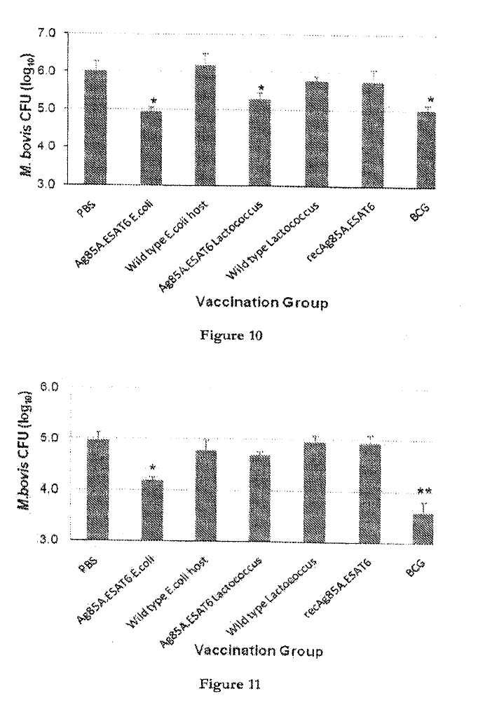

In the lung cultures, vaccination with Ag85A-ESAT-6 polymer particles provided a significantly improved resistance to infection compared to the PBS-immunised negative control group (FIG. 10, *=p<0.05). This improved resistance was conferred by particles synthesised in either E. coli or in L. lactis hosts. Also, vaccination with Ag85A-ESAT-6 polymer particles synthesised in E. coli hosts provided significantly better protection compared to that conferred by antigen alone. Indeed, Ag85A-ESAT-6 polymer particles showed comparable protection to the gold standard BCG vaccine (FIG. 10).

Importantly, vaccination with recombinant Ag85A-ESAT-6 antigen alone (i.e., antigen not comprising a polymer particle of the present invention) did not confer improved resistance to infection compared to the PBS-immunised control group.

In spleen cultures, vaccination with Ag85A-ESAT-6 polymer particles provided a significantly improved resistance to infection compared to the PBS-immunised negative control group (FIG. 11, *=p<0.05). Also, vaccination with Ag85A-ESAT-6 polymer particles synthesised in E. coli hosts provided significantly better protection compared to that conferred by antigen alone. Neither immunisation with wild type polymer particle (i.e., polymer particles with no Tb antigen), nor with recombinant Ag85A-ESAT-6 antigen alone, conferred a protective response.

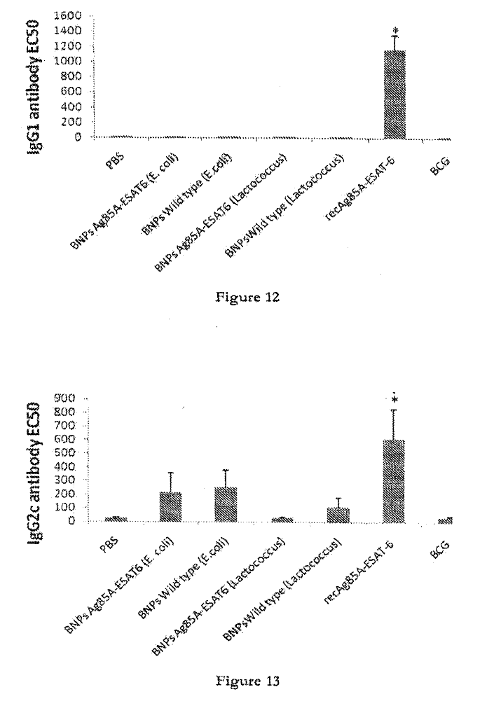

FIGS. 12 and 13 show that, in addition to the specific cell-mediated response, a humoral response was also elicited in mice vaccinated with Ag85A-ESAT-6 polymer particles Compared to BCG vaccine, the IgG2c antibody response was greater with Ag85A-ESAT-6 polymer particles produced in E. coli.

### Discussion

Immunisation with polymer particles displaying an Ag85A-ESAT-6 antigen fusion produced in both E. coli and L. lactis was able to provide immunological protection to animals challenged with M. bovis. This protection conferred a reduced infective load on the animals so vaccinated.

In lungs, the level of protection against Tb infection conferred by immunisation with polymer particles displaying an Ag85A-ESAT-6 antigen fusion was comparable to that of the BCG vaccine. This suggests that the polymer particles of the invention may elicit a protective immunological response to Tb infection, including initial infection and colonisation.

The reduced infection observed in the spleens of mammals immunised with polymer particles displaying an Ag85A-ESAT-6 antigen fusion compared to control mammals also suggests that immunisation with the polymer particles of the invention provides protection against Tb infiltration and disease progression.

Again, the lack of adverse side effects demonstrated that the polymer particles of the invention were well tolerated, safe, and non-toxic.

### INDUSTRIAL APPLICATION

Aspects of the invention described herein, including methods, polymer particles and fusion proteins have utility in therapy and prevention of disease, diagnostics, protein production, biocatalyst immobilisation, and drug delivery.

Those persons skilled in the art will understand that the above description is provided by way of illustration only and that the invention is not limited thereto.

### DOCUMENTS

Anderson, P. “Tuberculosis vaccines—an update” Nature (2007) 5: 484-487
Anthony, C. “The Biochemistry of Methylotrophs” Academic Press, London, United Kingdom (1982)
Ausubel, et al “Current protocols in Molecular Biology” Greene Publishing (1987)
Bäkström, B. T. et al. “Recombinant Escherichia coli produces tailor made biopolyester granules for applications in fluorescence activated cell sorting: functional display of the mouse interleukin-2 and myelin oligodendrocyte glycoprotein” BMC Biotechnology (2007) 7: 3-14
Balance, D. J. et al., “Transformation of Aspergillus nidulans by the orotidine-5′-phosphate decarboxylase gene of Neurospora crassa” Biochem. Biophys. Res. Commun. (1983) 112: 284-289
Barnard et al. “High level recombinant protein expression in Ralstonia eutropha using T7 RNA polymerase based amplification” Protein Expr Puri (2004) 38: 264-71
Barnes and Cave “Current concepts:molecular epidemiology of tuberculosis” New England Journal of Medicine (2003) 349: 1149-1156
Beach, D. and Nurse. P. “High-frequency transformation of the fission yeast Schizosaccharomyces pombe” Nature 290: 140-1421981
Belisle, J. T. et al. “Tuberculosis and the Tubercle Bacillus” ASM Press, Washington D.C. (2005)
Bowie, J. U. et al “Deciphering the message in protein sequences: tolerance to amino acid substitutions Science (1990) 247: 1306-1310
Brockelbank J. A., et al. “Recombinant Escherichia coli Strain Produces a ZZ Domain Displaying Biopolyester Granules Suitable for Immunoglobulin G Purification” Applied and Environmental Microbiology (2006) 72: 7394-7397
Brunschwig, E and Darzins, A. “A two-component T7 system for the overexpression of genes in Pseudomonas aeruginosa” Gene (1992) 111: 35-41
Case et al., Proceedings of the National Academy of Science USA (1979) 76: 5259-5263
Chang et al., Nature, 275: 615 (1978)
DeBoer et al., “The tac promoter: a functional hybrid derived from the trp lac promoters” Proceedings of the National Academy of Science USA (1983) 80: 21-25
Dietrich, J. et al “Prospects of a novel vaccine against tuberculosis” Veterinary Microbiology 112: 163-169
Fleer, R. et al., “Stable multicopy vectors for high-level secretion of recombinant human serum albumin in Kluyveromyces yeasts” Bio/Technology 9: 968-975
Friehs & Reardon “Parameters Influencing the Productivity of Recombinant E. coli Cultivations”. Advances in Biochemical Engineering Technology Vol 48 Springer Verlag (1991)
Goeddel, D. V. et al. “Direct expression in Escherichia coli of a DNA sequence coding for human growth hormone” (1979) Nature 281: 544-548
Goeddel, D. V. “Synthesis of human fibroblast interferon by E. coli” Nucleic Acids Research (1980) 8: 4057-4074
Hess, B. et al. “Cooperation of glycolytic enzymes” Advances in Enzyme Regulation (1968) 7: 149-167.
Hitzeman et al., J. Biol. Chem., 255:2073 (1980)]
Holland, M. J. and Holland, J. P. “Isolation and identification of yeast messenger ribonucleic acids coding for enolase, glyceraldehyde-3-phosphate dehydrogenase, and phosphoglycerate kinase” Biochemistry (1978) 17: 4900-4907
Huang, X. “On global sequence alignment” Computer Application in the Biosciences (1994) 10: 227-235
Kelly, J. M. and Hynes, M. J. “Transformation of Aspergillus niger by the amdS 22 gene of Aspergillus nidulans” EMBO Journal (1985) 4:475-479
Kingsman, A. J. et al., “Replication in Saccharomyces cerevisiae of plasmid pBR313 carrying DNA from the yeast trp1 region” Gene (1979) 7:141-152
Kirby, D. J. et al “PLGA microspheres for the delivery of a novel subunit TB vaccine” Journal of Drug Targeting (2009) 16: 282-293
Louvencourt et al. “Transformation of Kluyveromyces lactis by killer plasmid DNA” Journal of Bacteriology (1983)154: 737-742
Madison, L. L. et al, “Metabolic Engineering of Poly(3-hydroxyalkanoates): From DNA to Plastic”, Microbiology and Molecular Biology Reviews (1999) 63: 21-53
Mather, J. P. “Establishment and characterization of two distinct mouse testicular epithelial cell lines” Biology of Reproduction (1980) 23: 243-251
Mather, J. P. et al. “Culture of Testicular Cells in Hormone-Supplemented Serum-Free Medium” Annals of the N. Y. Academy of Science (1982) 383:44-68
Mitsopoulos G et al. “Tagged library approach to chemical genomics and proteomics” Current Opinion in Chemical Biology (2004) 8:26-32
Mustafa, A. S. “Biotechnology in the development of new vaccines and diagnostic reagents against tuberculosis” Current Pharmaceutical Biotechnology (2001) 2: 157-173
Peters, V. and Rehm, B. H. A. “In vivo enzyme immobilization by use of engineered polyhydroxyalkanoate synthase” Applied and Environmental Microbiology (2006) 72: 1777-83
Rehm, B. H. A. “Polyester synthesis; natural catalysts for plastics” Biochemical Journal (2003) 376: 15-33
Rehm, B. H. A. “Biopolyester particles produced by microbes or using polyester synthases: self assembly and potential applications” Microbial Biotechnology: biological self-assembly systems and biopolymer-based nanostructures Horizon Bioscience (2006)
Sambrook, et al “Molecular Cloning; a Laboratory Manual” (2^{nd} ed) Cold Spring Harbor Press (1987)
Sreekrishna K et al. “High level expression of heterologous proteins in methylotropic yeast Pichia pastoris” Journal of Basic Microbiology (1988) 28: 265-278
Stinchcomb, D. T. et al. “Isolation and characterisation of a yeast chromosomal replicator” Nature (1979) 282: 39-43
Tilburn, J. et al. “Transformation by integration in Aspergillus nidulans” Gene (1983) 26: 205-221
Tschemper, G and Carbon, J. “Sequence of a yeast DNA fragment containing a chromosomal replicator and the TRP1 gene” Gene (1980) 10:157-166
Urlaub, G. and Chasin, L. A. “Isolation of Chinese hamster cell mutants deficient in dihydrofolate reductase activity” Proceedings of the National Academy of Science USA (1980) 77:4216-4220.
Van den Berg, G. et al. “Kluyveromyces as a host for heterologous gene expression: expression and secretion of prochymosin” Bio/Technology (1990) 8: 135-139
Yelton, M. M. et al. “Transformation of Aspergillus nidulans by using a trpC plasmid” Proceedings of the National Academy of Science USA (1984) 81: 1470-1474

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred nonlimiting embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and applicants reserve the right to revise the application or claims to refer specifically to any individual member or any subgroup of members of the Markush group.

1-120. (canceled)

121. A method of eliciting an immune response in a subject, or of immunizing a subject against a pathogen, wherein the method comprises administering to a subject in need thereof a polymer particle comprising one or more fusion polypeptides, wherein at least one of the one or more fusion polypeptides comprises:
i) a particle-forming protein fused to at least one antigen capable of eliciting an immune response; or
ii) a particle-forming protein fused to a binding domain capable of binding at least one antigen capable of eliciting an immune response in a subject.

122. A method according to claim 121 wherein the subject is infected with the pathogen or has been immunized against the pathogen.

123. A method according to claim 121, wherein the binding domain capable of binding an antigen capable of eliciting an immune response binds to an endogenous antigen.

124. A method according to claim 121, wherein the binding domain capable of binding an antigen capable of eliciting an immune response binds to an exogenous antigen.

125. A method according to claim 121 wherein the polymer particle comprises:
i) two or more different antigens; or
ii) two or more different binding domains capable of binding an antigen; or
iii) at least one antigen capable of eliciting an immune response and at least one binding domain capable of binding an antigen capable of eliciting a cell-mediated immune response.

126. A method according to claim 121 wherein the polymer particle comprises one or more fusion polypeptides comprising a particle-forming protein and
i) at least one M. Tuberculosis antigen; or
ii) at least one M. Tuberculosis antigen binding domain; or
iii) at least one hepatitis antigen; or
iv) at least one influenza antigen; or
v) at least one binding domain capable of binding a hepatitis antigen; or
vi) at least one binding domain capable of binding an influenza antigen.

127. A method according to claim 126 wherein the polymer particle comprises an M. tuberculosis ESAT-6 antigen, an M. tuberculosis Ag85A antigen, or both an M. tuberculosis ESAT-6 antigen and an M. tuberculosis Ag85A antigen.

128. A method according to claim 121 wherein the polymer particle further comprises one or more of the following, alone or in any combination:
i. at least one thiolase; and/or
ii. at least one reductase; and/or
iii. at least one polymer synthase; and/or
iv. at least one M. tuberculosis antigen, optionally M. tuberculosis ESAT-6 antigen or M. tuberculosis Ag85A antigen; and/or
v. at least one M. tuberculosis antigen binding domain; or
vi. at least one hepatitis antigen; and/or
vii. at least one influenza antigen; and/or
viii. at least one binding domain capable of binding at least one hepatitis antigen; and/or
ix. at least one binding domain capable of binding at least one influenza antigen; and/or
x. a fusion protein comprising one or more of i) to ix) above.

129. A method of immunizing a subject against tuberculosis, wherein the method comprises administering to a subject in need thereof at least one polymer particle comprising one or more fusion polypeptides, wherein (a) at least one of the fusion polypeptides comprises a particle-forming protein fused to at least one M. tuberculosis antigen, or (b) at least one of the fusion polypeptides comprises a particle-forming protein fused to at least one M. tuberculosis antigen binding domain, or (c) both (a) and (b).

130. A method of immunizing a subject against hepatitis or influenza, wherein the method comprises administering to a subject in need thereof at least one polymer particle comprising one or more fusion polypeptides, wherein (a) at least one of the fusion polypeptides comprises a particle-forming protein fused to at least one hepatitis antigen or at least one influenza antigen, or (b) at least one of the fusion polypeptides comprises a particle-forming protein fused to a binding domain capable of binding to at least one hepatitis antigen or at least one influenza antigen, or (c) both (a) and (b).

131. A method according to claim 129 for immunizing a subject against tuberculosis, wherein the subject is infected with tuberculosis, or has previously been immunized against tuberculosis.

132. A method according to claim 129 for immunizing a subject against tuberculosis, wherein at least one of the polymer particles comprises an M. tuberculosis antigen selected from the group comprising ESAT-6, Ag85A, Ag85B (MPT59), Ag85B, Ag85C, MPT32, MPT51, MPT59, MPT63, MPT64, MPT83, MPB5, MPB59, MPB64, MTC28, Mtb2, Mtb8.4, Mtb9.9, Mtb32A, Mtb39, Mtb41, TB10.4, TB10C, TB11B, TB12.5, TB13A, TB14, TB15, TB15A, TB16, TB16A, TB17, TB18, TB21, TB20.6, TB24, TB27B, TB32, TB32A, TB33, TB38, TB40.8, TB51, TB54, TB64, CFP6, CFP7, CFP7A, CFP7B, CFP8A, CFP8B, CFP9, CFP10, CFP11, CFP16, CFP17, CFP19, CFP19A, CFP19B, CFP20, CFP21, CFP22, CFP22A, CFP23, CFP23A, CFP23B, CFP25, CFP25A, CFP27, CFP28, CFP28B, CFP29, CFP30A, CFP30B, CFP50, CWP32, hspX (alpha-crystalline), APA, Tuberculin purified protein derivative (PPD), ST-CF, PPE68, LppX, PstS-1, PstS-2, PstS-3, HBHA, GroEL, GroEL2, GrpES, LHP, 19 kDa lipoprotein, 71 kDa, RD1-ORF2, RD1-ORF3, RD1-ORF4, RD1-ORF5, RD1-ORF8, RD1-ORF9A, RD1-ORF9B, Rv1984c, Rv0577, Rv1827, BfrB, Tpx. Rv1352, Rv1810, PpiA, Cut2, FbpB, FbpA, FbpC, DnaK, FecB, Ssb, Rp1L, FixA, FixB, AhpC2, Rv2626c, Rv1211, Mdh, Rv1626, Adk, ClpP, SucD, or at least one antigenic portion or T-cell epitope of any of the above mentioned antigens.

133. A method according to claim 130 for immunizing a subject against hepatitis, wherein at least one of the polymer particles comprises a hepatitis C antigen selected from the group comprising C—p22, E1—gp35, E2—gp70, NS1—p7, NS2—p23, NS3—p70, NS4A—p8, NS4B—p27, NS5A—p56/58, and NS5B—p68, or at least one antigenic portion or T-cell epitope of any of the above mentioned antigens.

134. A method according to claim 130 for immunizing a subject against influenza, wherein at least one of the polymer particles comprises an influenza antigen selected from the group comprising PB, PB2, PA, a hemagglutinin (HA) or neuramimidase (NA) protein, NP, M, and NS, or at least one antigenic portion or T-cell epitope of any of the above mentioned antigens.
